(19) United States
(12) Patent Application Publication
(10) Pub. No.: US 2015/0133462 A1
(43) Pub. Date: May 14, 2015

(54) METHODS AND COMPOSITIONS FOR THE TREATMENT OF BCR-ABL POSITIVE LYMPHOBLASTIC LEUKEMIAS

(71) Applicant: ST. JUDE CHILDREN'S RESEARCH HOSPITAL, Memphis, TN (US)

(72) Inventors: Harpreet Singh, Memphis, TN (US); Richard T. Williams, Newbury Park, CA (US); Kiplin R. Guy, Memphis, TN (US)

(21) Appl. No.: 14/403,561

(22) PCT Filed: May 23, 2013
(86) PCT No.: PCT/US13/42459

§ 371 Date: Nov. 24, 2014

Related U.S. Application Data
(60) Provisional application No. 61/650,948, filed on May 23, 2012.

(51) Int. Cl. A61K 31/357; A61K 31/506

(52) U.S. Cl. CPC A61K 31/357; A61K 31/506
USPC 514/252.18; 514/252.19

(57) ABSTRACT

In one aspect, the invention relates to compositions comprising BCR-ABL tyrosine kinase inhibitors and artemisinin analogues, derivatives thereof, or related compounds, which are useful as in treating Philadelphia chromosome positive leukemias; pharmaceutical compositions comprising the compounds; and methods of treating disorders of uncontrolled cellular proliferation associated with BCR-ABL dysfunction using the compounds and compositions. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 61/650,948 filed on May 23, 2012, which is incorporated herein by reference in its entirety.

### STATEMENT REGARDING FEDERALLY SPONSORED RESEARCH

This invention was made in part with government support under grant numbers NIH CA021765 and NIH NS066460 awarded by the National Institute of Health (NIH). The United States government has certain rights in the invention.

### BACKGROUND

Hyperproliferative disorders, such as cancer, are associated with the growth of abnormal, or neoplastic cells. In cancer, this uncontrolled cellular proliferation will often result in unregulated growth of malignant tissue and invasion of adjacent or local cells. Eventually, the malignant cells spread, or metastasize, to lymph nodes or remote areas by traveling through the blood or lymphatic system. Moreover, these abnormal cells are often able to proliferate under conditions where a normal cell would not grow.

Although the etiology of cancer has not been clearly defined, numerous mechanisms and processes have been implicated in disease onset and progression, including both environmental and genetic factors. However, cancer pathogenesis ultimately results from DNA mutations that impact cell growth, differentiation, and/or survival.

Cancer encompasses a broad group of medical conditions and syndromes, and can manifest in almost any organ and/or tissue in the body. As a result, there are many types of cancer, for example, bladder, breast, cervical, colon, hematologic, lung, ovarian, pancreatic, prostate, renal cell, and testicular. Moreover, cancer can present in any age, in many forms, and with varying degrees of invasiveness and aggressiveness.

Traditionally, treatments for cancer have involved surgery, radiation and chemotherapy. Depending on the type and location of the cancer, surgery can be an option for certain patients. Surgery is often a radical step and can invariably carry serious risks for the patient. In addition to surgical complications, there always exists the risk the entire cancer cannot be removed. Radiation therapy involves the use of ionizing radiation to kill cancer cells. Although radiation therapy is effective in killing cancer cells, it carries the risk of concurrently damaging non-cancerous cells. Higher and frequent radiation dosing can have long-term risks, such as secondary malignancies and reproductive effects. Chemotherapy involves administering cytotoxic drugs to the patient, often in a regimen involving a combination of such drugs. While chemotherapy is effective in killing cancer cells, it also harms normal cells that divide rapidly, leading to adverse side effects such as myelosuppression. Chemotherapy is often accompanied by additional side effects, such as severe nausea/vomiting and organ specific toxicities, which can force premature discontinuation of treatment.

Most recently, research has focused on identifying specific cellular targets for therapies. Unlike standard chemotherapy, targeted therapies are designed to only interact with specific molecular targets involved in the pathways and processes. These pathways and processes are used by cancer cells to grow, divide, and spread throughout the body. In the body, tyrosine kinases (TKs) are known to phosphorylate specific tyrosyl residues in specific proteins involved in regulating cell proliferation and differentiation. Unregulated activation of such kinases, resulting from genetic abnormalities (e.g. point mutations; translocation and genomic amplification) in the genes that encode these proteins, can result in unregulated cell growth. For example, BCR-ABL tyrosine kinase (BCR-ABL TK) has been known to activate many pro-growth and cell survival mechanisms, which in turn promote cell proliferation and survival, and contribute to BCR-ABL mediated leukemic transformation of bone marrow progenitor cells. Therefore, BCR-ABL TK-specific inhibition has been the focus of significant drug discovery efforts. BCR-ABL TK-specific inhibitors (BCR-ABL TKIs), such as imatinib, can bind and stabilize the inactive conformation, and have been successful in maintaining certain chronic myelongenous leukemias (CML) in remission to a controlled chronic condition.

However, BCR-ABL TKIs have failed to produce a similar long-term response in related leukemias, such as pediatric and adult Philadelphia chromosome positive (Ph+) acute lymphoblastic leukemia (ALL). While current BCR-ABL-targeted drugs appear to kill Ph+ ALL cells in vitro, these drugs fail to meaningfully maintain low levels of leukemia burden in patients with Ph+ ALL, invariably leading to relapse and poor long-term outcomes. Additionally, such therapy also fails to eradicate persistent disease in patients with either Ph+ ALL or CML. Although additional chemotherapy and/or hematopoietic stem cell transplantation (HSCT) can improve the remission rate for some Ph+ ALL patients, relapses remain common, with an 18- to 24-month survival rate of 64% and an even worse relapse-free survival rate.

Resistance to BCR-ABL TKIs is believed to contribute to the inability to induce adequate clinical responses. A major mechanism of BCR-ABL TKI resistance is thought to relate to mutations that result in the interference of drug binding to the BCR-ABL kinase domain. BCR-ABL mutations confer different levels of resistance, ranging from concentration dependent resistance to complete BCR-ABL TKI resistance. The most clinically significant BCR-ABL kinase domain (KD) mutations are P-loop mutations and the T315I mutation. Significantly, the T315I mutation produces the most common and highest form of resistance of any mutation, resulting in resistance to both imatinib and second generation BCR-ABL TKIs, dasatinib and nilotinib. Nevertheless, differences in clinical responses to BCR-ABL TKIs in patients with Ph+ ALL and CML remain poorly understood, and cannot be exclusively attributed to mutations in the BCR-ABL kinase domain that impair drug binding.

Therefore, defining and countering the factors that underlie the transient response to BCR-ABL TKIs in patients with non-mutated BCR-ABL Ph+ ALL is critical for developing effective therapies that improve long-term survival. Moreover, contemporary medicine advocates co-treatment with agents of complementary mechanisms of action to counteract clinical drug resistance and improve outcomes. Therefore, there remains a need for therapies that are both safe, efficacious and complement targeted therapeutic agents for the treatment of cancer. These needs and other needs are satisfied by the present invention.

### SUMMARY

In accordance with the purpose(s) of the invention, as embodied and broadly described herein, the invention, in one aspect, relates to compounds and compositions useful as therapies in disorders of uncontrolled cellular proliferation, pharmaceutical compositions comprising same, and methods of treating BCR-ABL-mediated disorders using same. For example, compounds capable of counteracting or overcoming cytokine-related drug resistance (e.g., dihydroartemisinin (DHA)), in combination with a BCR-ABL tyrosine kinase inhibitor, can be useful in hematologic cancer therapies (e.g., for treating leukemias or lymphomas).

Disclosed herein are pharmaceutical compositions comprising a therapeutically effective amount of a BCR-ABL tyrosine kinase inhibitor; and a therapeutically effective amount of at least one artemisinin analogue having a structure represented by a formula:

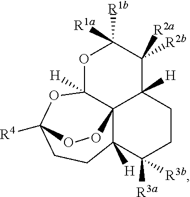

wherein R^{1a} and R^{1b} together are (C═O) or wherein each of R^{1a} and R^{1b} are independently selected from hydrogen, hydroxyl, —NH₂, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), C2-C5 heterocycloalkyl, C3-C6 cycloalkyl, —O(C1-C8 alkyl)C3-C6 cycloalkyl, —O(C1-C8 alkyl)-(C═O)OH, —O(C═O)(C1-C6 alkyl)-(C═O)OH, —O(C1-C8 alkyl)-(C═O)O(C1-C6 alkyl), —O(C═O)(C1-C6 alkyl)-(C═O)O(C1-C6 alkyl), —O(C1-C8 alkyl)-aryl, and —O-aryl; wherein each of aryl, C2-05 heterocycloalkyl, C3-C6 cycloalkyl, and —O(C1-C8 alkyl)C3-C6 cycloalkyl, when present, is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl); wherein R^{2a} and R^{2b} together are (C═CR^{5a}R^{5b}) or wherein each of R^{2a} and R^{2b} are independently selected from hydrogen, C1-C8 alkyl, C1-C8 monohaloalkyl, C1-C8 polyhaloalkyl, —(C1-C8 alkyl)-(C═O)OH, and —O(C═O)(C1-C6 alkyl)-(C═O)O(C1-C6 alkyl); wherein each of R^{5a} and R^{5b} are independently selected from hydrogen, C1-C6 alkyl, C1-C6 monohaloalkyl, and C1-C6 polyhaloalkyl; wherein each of R^{1a} and R^{ab} are independently selected from hydrogen, C1-C8 alkyl, C1-C8 monohaloalkyl, and C1-C8 polyhaloalkyl; and wherein R⁴ is selected from hydrogen, C1-C8 alkyl, C1-C8 monohaloalkyl, C1-C8 polyhaloalkyl, —(C1-C8 alkyl)-(C═O)OH, and —O(C═O)(C1-C6 alkyl)-(C═O)O(C1-C6 alkyl); or a pharmaceutically acceptable salt, hydrate, solvate, or polymorph thereof; and optionally a pharmaceutically acceptable carrier.

Also disclosed herein are methods for the treatment of a Philadelphia chromosome positive leukemia in a patient comprising the step of administering to the patient a therapeutically effective amount of a BCR-ABL kinase inhibitor and a therapeutically effective amount of at least one disclosed artemisinin analogue, or a pharmaceutically acceptable salt, solvate, or polymorph thereof

Also disclosed herein are method for the treatment of a gastrointestinal stromal tumor (GIST) in a patient comprising the step of administering to the patient a therapeutically effective amount of a BCR-ABL kinase inhibitor and a therapeutically effective amount of at least one disclosed artemisinin analogue, or a pharmaceutically acceptable salt, solvate, or polymorph thereof.

Also disclosed herein are methods for the treatment of a Philadelphia chromosome positive leukemia in a patient comprising the step of administering to the patient a therapeutically effective amount of a BCR-ABL kinase inhibitor, and a therapeutically effective amount of at least one disclosed artemisinin analogue, or a pharmaceutically acceptable salt, hydrate, solvate, or polymorph thereof; wherein administering the artemisinin analogue to a patient produces a metabolite in the patient with a structure represented by the formula:

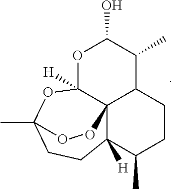

Also disclosed herein are methods for the treatment of a gastrointestinal stromal tumor (GIST) in a patient comprising the step of administering to the patient a therapeutically effective amount of a BCR-ABL kinase inhibitor, and a therapeutically effective amount of at least one disclosed artemisinin analogue, or a pharmaceutically acceptable salt, hydrate, solvate, or polymorph thereof; wherein administering the artemisinin analogue to a patient produces a metabolite in the patient with a structure represented by the formula:

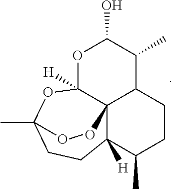

Also disclosed herein are kits comprising a BCR-ABL kinase inhibitor, at least one disclosed artemisinin analogue, or a pharmaceutically acceptable salt, solvate, or polymorph thereof; and instructions for treatment of a disease of uncontrolled cellular proliferation; and wherein the instructions comprise administration of a BCR-ABL kinase inhibitor and the at least one artemisinin analogue to a patient that has become refractory to treatment with a BCR-ABL kinase inhibitor.

Also disclosed are methods for manufacturing a medicament comprising combining at least one disclosed compound with a pharmaceutically acceptable carrier or diluent, wherein the medicament is used to treat a Philadelphia chromosome positive leukemia.

Also disclosed are uses of a disclosed composition or compound for the treatment of a disease of uncontrolled cellular proliferation in a patient.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative in vitro data pertaining to the growth properties of Arf−/−BCR-ABL+preB cells.

FIG. 2 shows representative data pertaining to development of the high-throughput LIC-based phenotypic assay.

FIG. 3 shows a representative schematic of screen used to identify compounds preventing growth of wild type and BCR-ABL^{MUTANT} Ph+ ALL cells in the presence of IL7.

FIG. 4 shows a representative radar plot pertaining to the effect of 15 hematopoietic cytokines on dasatinib potency (EC₅₀) against Arf ^{-/-} BCR-ABL^{WT} LICs.

FIG. 5 shows representative data pertaining to LIC growth inhibition following 72-h treatment with drug or DMSO in the absence (dark gray) or presence (light gray) of 1 ng/mL murine IL7.

FIG. 6 shows representative data pertaining to cell viability of human Ph+ ALL SUPB15 cells following 72-h drug-treatment in absence or presence of 25 ng/mL human IL7.

FIG. 7 shows representative data pertaining to LIC growth inhibition following 72-hr treatment with drug.

FIG. 8 shows representative data pertaining to in vitro leukemia clonal outgrowth following treatment with drug or DMSO.

FIG. 9 shows representative data pertaining to LIC viability following treatment with drug in the presence or absence of IL7.

FIG. 10 shows representative data pertaining to overall survival of host mice receiving Arf^{-/-} BCR-ABL^{WT} LICs injections following treatment with vehicle or drug.

FIG. 11 shows frequency of ATC Level 1 and ATC Level 2 compounds for the 706 compounds submitted to secondary analysis.

FIG. 12 shows representative data pertaining to screening quality metrics for High Throughput (HT) drug screen against BCR-ABL^{WT} LICs.

FIG. 13 shows representative data pertaining to screening quality metrics for High Throughput (HT) drug screen against BCR-ABL^{T315I} LICs.

FIG. 14 shows representative data pertaining to LIC growth inhibition following treatment with drug.

FIG. 15 shows representative data pertaining to the discriminatory power of the phenotypic assay.

FIG. 16 shows representative data pertaining to LIC growth inhibition following 72-hr treatment with artemisinin, artesunate, and dihydroartemisinin.

FIG. 17 shows representative data pertaining to cell viability of human Ph+ ALL SUPB15 cells following 72-h drug-treatment.

FIG. 18 shows representative data pertaining to overall survival of host mice receiving Arf^{-/-} BCR-ABL^{WT} LICs injections following treatment with vehicle or drug.

FIG. 19 shows representative data pertaining to overall survival of host mice receiving Arf^{-/-} BCR-ABL^{WT} LICs injections following treatment with vehicle or drug.

FIG. 20 shows representative data pertaining to overall survival of host mice receiving Arf^{-/-} BCR-ABL^{WT} LICs injections following treatment with vehicle or drug.

FIG. 21 shows representative data pertaining to overall survival of host mice receiving Arf^{-/-} BCR-ABL^{WT} LICs injections following treatment with vehicle or drug.

FIG. 22 shows representative data pertaining to overall survival of nonconditioned immunocompetent C57B1/6J mice receiving LIC injections.

FIG. 23 shows the data relating to the 15 leukemia microenvironment cytokine assay.

FIG. 24 shows structural information for the 706 compounds screened.

FIG. 25 shows the data relating to the clinically used anti-leukemic agents for dose-response screening experiments.

FIG. 26 shows data relating to the dose-response screen for the 706 compounds screened.

FIG. 27 shows the ATC classification for the 706 compounds screened.

FIG. 28 shows drug challenge of IL7 naïve LICs with dasatinib in the presence and absence of IL7 (Panel A), and rechallenge after washout of IL7 and dasatinib (Panel B).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “Philadelphia chromosome” and “Ph” refer to a reciprocal, chromosomal translocation between chromosomes 9 and 22, resulting in the oncogenic BCR-ABL gene fusion, and encodes the BCR-ABL fusion protein. Specifically, this translocation results in a portion of the BCR gene from chromosome 22 (region q11) becoming fused with a portion of the ABL gene from chromosome 9 (region q34), and is designated as t(9;22)(q34;q11) under the International System for Human Cytogenetic Nomenclature (ISCN). Depending on the precise location of the fusion, the molecular weight of the protein can range from 185 to 210 kDa, and the term is inclusive of all variants of this gene. In this context, “Philadelphia chromosome positive” and “Ph+” is understood to represent the presence of this chromosomal defect, resulting in a chimeric BCR-ABL protein and constitutively active BCR-ABL TK activity.

As used herein, the term “BCR-ABL” refers to the fusion oncogene which encodes a chimeric BCR-ABL protein with a constitutively active BCR-ABL tyrosine kinase (TK) activity. In this context, protein tyrosine kinases encoded by the BCR-ABL gene can include, for example BCR-ABL p210 fusion protein (accession number: A1Z199) and BCR-ABL p185 fusion protein (accession number: Q13745). The term BCR-ABL is intended to be inclusive of alternative BCR-ABL gene products and also is inclusive of alternative designations such as BCR-ABL oncogene, BCR-ABL protooncogene, and BCR-ABL oncoprotein used by those skilled in the art.

As used herein, the terms “BCR-ABL tyrosine kinase inhibitor,” “BCR-ABL kinase inhibitor,” “BCR-ABL KI” and “BCR-ABL TKI” refer to any compound or agent that can inhibit BCR-ABL TK activity in an animal, in particular a mammal, for example a human. In this context, an inhibitor is understood to decrease the activity of a BCR-ABL tyrosine kinase compared to the activity in the absence of the exogenously administered compound or agent. The term is intended to include indirectly or directly acting compounds or agents. As used in the art, BCR-ABL TKI generally refers to a class of compounds which are known to inhibit BCR-ABL TK, but may further inhibit alternative signaling pathways, such as for example, Src pathway.

As used herein, the term “BCR-ABL related disorders” refers to disorders or diseases which are associated with or manifest from BCR-ABL-mediated activity, and is intended to be inclusive of mutated forms of BCR-ABL. In this context, disorders associated with BCR-ABL would benefit by direct or indirect BCR-ABL inhibition.

As used herein, the terms “BCR-ABL^{MUTANT}” or “mutant BCR-ABL” refer to a BCR-ABL tyrosine kinase amino acid sequence that differs from wild type BCR-ABL tyrosine kinase or “BCR-ABL^{WT}” by one or more amino acid substitutions, additions or deletions. In this context, an amino acid substitution at position 315 of wild type BCR-ABL with another amino acid would result in a mutant BCR-ABL tyrosine kinase, such as for example T315I, which may be designated as “BCR-ABL^{T315I}”.

As used herein, the term “KIT” refers to the gene which encodes for the c-KIT protein. The term KIT is intended to be inclusive of alternative KIT transcript variants and gene products and also is inclusive of alternative designations such as “proto oncogene c-KIT,” “tyrosine-protein kinase KIT,” and “CD117” used by those skilled in the art.

As used herein, “cytokine” refers to a signaling molecule used in intercellular communication resulting in alteration of cell function, and encompasses lymphokines, interleukins, and chemokines, such as for example, IL7. In this context, “cytokine signaling” is meant to include cell-extrinsic and cell-intrinsic activity mediated by extracellular cytokines or by over-expression of a gene product that mimics constitutive cytokine signaling.

The term “imatinib” as used herein refers to a compound having a structure represented by the formula:

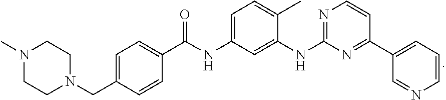 [SMILES: Cc1ccc(NC(=O)c2ccc(CN3CCN(C)CC3)cc2)cc1Nc1nccc(-c2cccnc2)n1]

This compound has been described as inhibiting the activity of BCR-ABL, KIT, and the platelet-derived growth factor receptor (PDGFR).

The term “dasatinib” as used herein refers to a compound having a structure represented by the formula:

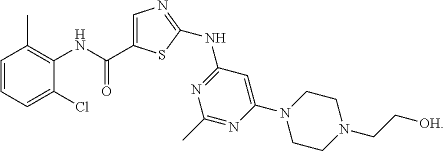 [SMILES: Cc1nc(Nc2ncc(C(=O)Nc3c(C)cccc3Cl)s2)cc(N2CCN(CCO)CC2)n1]

This compound has been described as inhibiting the activity of SRC and SRC-family kinases, BCR-ABL, KIT, PDGFR and ephrin receptor tyrosine kinases.

The term “nilotinib” as used herein refers to a compound having a structure represented by the formula:

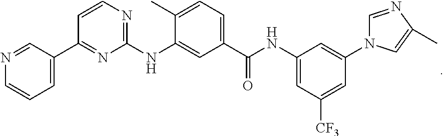 [SMILES: Cc1cn(-c2cc(NC(=O)c3ccc(C)c(Nc4nccc(-c5cccnc5)n4)c3)cc(C(F)(F)F)c2)cn1]

This compound has been described as inhibiting the activity BCR-ABL, KIT, and PDGFR. It is also suspected of inhibiting ABL-related kinase ARG and the ephrin receptor EPHB4.

The term “bosutinib” as used herein refers to a compound having a structure represented by the formula:

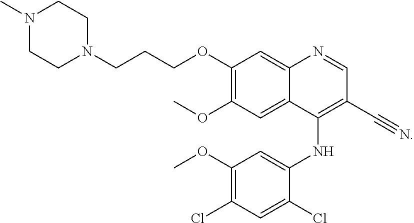 [SMILES: COc1cc(Nc2c(C#N)cnc3cc(OCCCN4CCN(C)CC4)c(OC)cc23)c(Cl)cc1Cl]

This compound has been described as inhibiting the activity of BCR-ABL, SRC-family kinases, and STATS.

The term “ponatinib” as used herein refers to a compound having a structure represented by the formula:

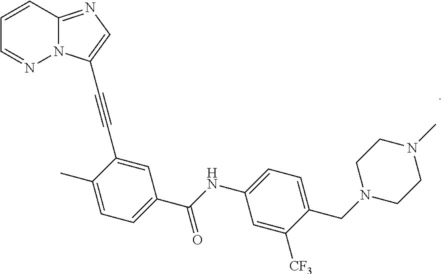 [SMILES: Cc1ccc(C(=O)Nc2ccc(CN3CCN(C)CC3)c(C(F)(F)F)c2)cc1C#Cc1cnc2cccnn12]

This compound has been described as inhibiting the activity of BCR-ABL, FLT3, vascular endothelial growth factors (VEGFRs), fibroblast growth factors (FGFRs), and angiopoietin (Tie2).

The term “bafetinib” as used herein refers to a compound having a structure represented by the formula:

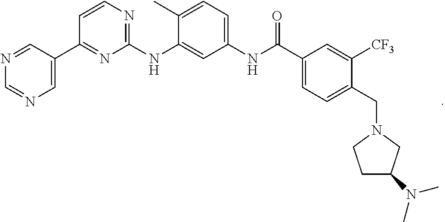 [SMILES: Cc1ccc(NC(=O)c2ccc(CN3CC[C@H](N(C)C)C3)c(C(F)(F)F)c2)cc1Nc1nccc(-c2cncnc2)n1]

This compound has been described as inhibiting the activity of BCR-ABL, LYN, PDGFR, and KIT.

The term “saracatinib” as used herein refers to a compound having a structure represented by the formula:

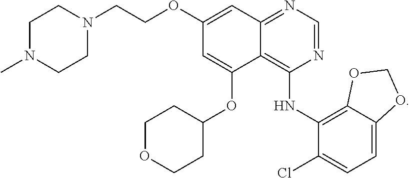 [SMILES: CN1CCN(CCOc2cc(OC3CCOCC3)c3c(Nc4c(Cl)ccc5c4OCO5)ncnc3c2)CC1]

This compound has been described as inhibiting the activity of BCR-ABL and the SRC-family kinases.

The term “tozasertib” as used herein refers to a compound having a structure represented by the formula:

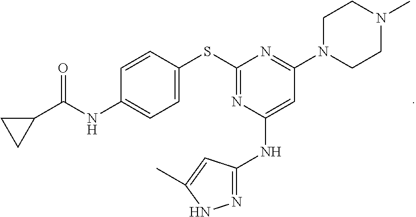 [SMILES: Cc1cc(Nc2cc(N3CCN(C)CC3)nc(Sc3ccc(NC(=O)C4CC4)cc3)n2)n[nH]1]

This compound has been described as inhibiting the activity of BCR-ABL, Aurora kinases, FLT3, and JAK2.

The term “danusertib” as used herein refers to a compound having a structure represented by the formula:

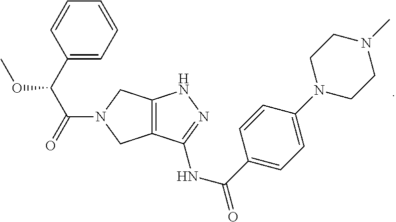

This compound has been described as inhibiting the activity of BCR-ABL and Aurora kinases.

As used herein, the terms “artemisinin analogue,” “artemisinin class,” and “ARTs” can be used interchangeably and refer to a compound structurally related to artemisinin comprising one or more substitutions on artemisinin and generally represented by the formula:

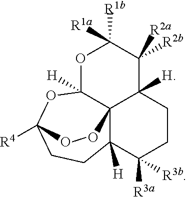

As used herein, the term “artemisinin” refers to a compound having a structure represented by the formula:

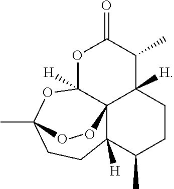

As used herein, the terms “dihydroartemisinin” or “DHA” refer to a compound having a structure represented by the formula:

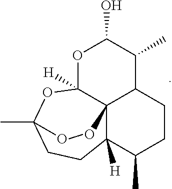

As used herein, the term “artesunate” refers to a compound having a structure represented by the formula:

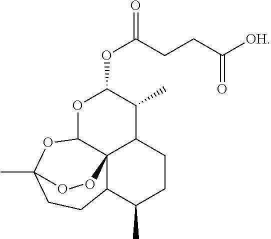 [SMILES: C[C@@H]1CCC2[C@@H](C)[C@@H](OC(=O)CCC(=O)O)OC3OC4(C)CCC1[C@]32OO4]

As used herein, the terms “cancer” or “cancerous” refer to a condition or disorder of uncontrolled cellular proliferation. Examples of cancer include, for example, leukemia, lymphoma, blastoma, carcinoma and sarcoma. More specific examples of such cancers include chronic myeloid leukemia, acute lymphoblastic leukemia, Philadelphia chromosome positive acute lymphoblastic leukemia (Ph+ALL), and gastrointestinal stromal tumors (GIST).

As used herein, the terms “optional” or “optionally” means that the subsequently described event or circumstance may or may not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation associated with a protein kinase dysfunction prior to the administering step. In some aspects of the disclosed method, the subject has been diagnosed with a need for inhibition of a protein kinase prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit a protein kinase. As a further example, “diagnosed with a need for inhibition of a protein kinase” refers to having been subjected to a physical examination by a person of skill, for example, a physician, and found to have a condition characterized by a protein kinase dysfunction. Such a diagnosis can be in reference to a disorder, such as a disorder of uncontrolled cellular proliferation, cancer and the like, as discussed herein. For example, the term “diagnosed with a need for inhibition of protein kinase activity” refers to having been subjected to a physical examination by a person of skill, for example, a physician, and found to have a condition that can be diagnosed or treated by inhibition of protein kinase activity. For example, “diagnosed with a need for treatment of one or more disorders of uncontrolled cellular proliferation associated with a protein kinase dysfunction” means having been subjected to a physical examination by a person of skill, for example, a physician, and found to have one or more disorders of uncontrolled cellular proliferation associated with a protein kinase dysfunction. For example, “diagnosed with a disorder treatable by selective inhibition of BCR-ABL” means having been subjected to a physical examination by a person of skill, for example, a physician, and found to have a condition that can be diagnosed or treated by a compound or composition that can inhibit BCR-ABL. As a further example, “diagnosed with a need for selective inhibition of BCR-ABL” refers to having been subjected to a physical examination by a person of skill, for example, a physician, and found to have a condition characterized by BCR-ABL activity. Such a diagnosis can be in reference to a disorder, such as a disease of uncontrolled cellular proliferation, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to a dysfunction of protein kinase activity or a disorder related to BCR-ABL activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target histamine receptor, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition) , and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. In one aspect, an ECₛₒ can refer to the concentration of a substance that is required for 50% agonism or activation in an in vitro assay. In a further aspect, EC₅₀ refers to the concentration of agonist or activator that provokes a response halfway between the baseline and maximum response. For example, an ECₛₒ for compound or agent can be determined in an in vitro or cell-based assay system. Such in vitro assay systems frequently utilize a cell line that either expresses endogenously a target of interest, or has been transfected with a suitable expression vector that directs expression of a target of interest.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. For example, an IC₅₀ can refer to the concentration of a substance that is required for 50% inhibition in vivo or the inhibition is measured in vitro, as further defined elsewhere herein. Alternatively, IC₅₀ refers to the half maximal (50%) inhibitory concentration (IC) of a substance. For example, the IC₅₀ for a compound can be determined using murine leukemia initiating cells (LICs) transfected either the wild-type (WT) allele of human p185^{BCR-ABL} or mutant BCR-ABL allele p185^{T315I}.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” refers to a non-aromatic carbon-based moiety. Aliphatic can include both acyclic or cyclic moieties (e.g., alkyl and cycloalkyl) and can include both saturated and unsaturated moieties (e.g., alkyl, alkenyl, and alkynyl).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of from 1 to 24 carbon atoms, for example from 1 to 12 carbons, from 1 to 8 carbons, from 1 to 6 carbons, or from 1 to 4 carbons, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The cycloalkyl group can be substituted or unsubstituted. The cycloalkyl group can be substituted with one or more groups including, but not limited to, alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or —OA¹-(OA²)ₐ-OA³, where “a” is an integer of from 1 to 200 and A¹, A², and A³ are alkyl and/or cycloalkyl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ- or -(A¹O(O)C-A²-OC(O))ₐ-, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A2O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, pyrazolopyrimidinyl, 1,2-oxazol-4-yl, 1,2-oxazol-5-yl, 1,3-oxazolyl, 1,2,4-oxadiazol-5-yl, 1,2,3-triazolyl, 1,3-thiazol-4-yl, pyridinyl, and pyrimidin-5-yl.

The term “heterocycle,” as used herein refers to single and multi-cyclic aromatic or non-aromatic ring systems in which at least one of the ring members is other than carbon. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “heterocycloalkyl” as used herein is a non-aromatic carbon-based ring composed of at least two carbon atoms and at least one non-carbon heteroatom. For example, the non-carbon heteroatom can include, but is not limited to, oxygen, nitrogen, sulphur, phosphorus and the like. Examples of heterocycloalkyl groups include, aziridine, oxirane, thiirane, azetidine, oxetane, thietane, pyrrolidine, tetrahydrofuran, tetrahydrothiophene, piperidine, tetrahydro-2H-pyran, tetrahydro-2H-thiopyran, azepane, oxepane, thiepane, azocane, oxocane, thiocane, pyrazolidine, imidazolidine, diazetidine, hexahydropyridazine, piperazine, diazepane, oxazinane, oxazepane, oxazolidine, oxazetinde, and the like. The heterocycloalkyl group can be substituted or unsubstituted. The heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “organic residue” defines a carbon containing residue, i.e., a residue comprising at least one carbon atom, and includes but is not limited to the carbon-containing groups, residues, or radicals defined herein above. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

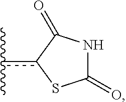

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Likewise, amides with an N-hydrogen can exist in an equilibrium of the amide form and the imidic acid form. Unless stated to the contrary, the invention includes all such possible tautomers.

In some aspects, a structure of a compound can be represented by a formula:

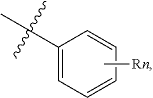

which is understood to be equivalent to a formula:

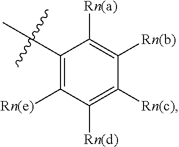

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain instances of the above defined terms may occur more than once in the structural formulae, and upon such occurrence each term shall be defined independently of the other.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (ee). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. Artemisinin Analogues

In various aspects, the invention relates to pharmaceutical compositions comprising a therapeutically effective amount of a BCR-ABL tyrosine kinase inhibitor; and a therapeutically effective amount of at least one artemisinin analogue having a structure represented by a formula:

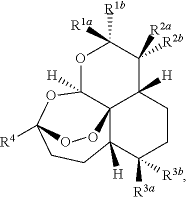

wherein R^{1a} and R^{1b} together are (C═O) or wherein each of R^{1a} and R^{1b} are independently selected from hydrogen, hydroxyl, —NH₂, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), C2-C5 heterocycloalkyl, C3-C6 cycloalkyl, —O(C1-C8 alkyl)C3-C6 cycloalkyl, —O(C1-C8 alkyl)-(C═O)OH, —O(C═O)(C1-C6 alkyl)-(C═O)OH, —O(C1-C8 alkyl)-(C═O)O(C1-C6 alkyl), —O(C═O)(C1-C6 alkyl)-(C═O)O(C1-C6 alkyl), —O(C1-C8 alkyl)-aryl, and —O-aryl; wherein each of aryl, C2-C5 heterocycloalkyl, C3-C6 cycloalkyl, and —O(C1-C8 alkyl)C3-C6 cycloalkyl, when present, is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl); wherein R^{2a} and R^{2b} together are (C═CR^{5a}R^{5b}) or wherein each of R^{2a} and R^{2b} are independently selected from hydrogen, C1-C8 alkyl, C1-C8 monohaloalkyl, C1-C8 polyhaloalkyl, —(C1-C8 alkyl)-(C═O)OH, and —O(C═O)(C1-C6 alkyl)-(C═O)O(C1-C6 alkyl); wherein each of R^{5a} and R^{5b} are independently selected from hydrogen, C1-C6 alkyl, C1-C6 monohaloalkyl, and C1-C6 polyhaloalkyl; wherein each of R^{3a} and R^{3b} are independently selected from hydrogen, C1-C8 alkyl, C1-C8 monohaloalkyl, and C1-C8 polyhaloalkyl; and wherein R⁴ is selected from hydrogen, C1-C8 alkyl, C1-C8 monohaloalkyl, C1-C8 polyhaloalkyl, —(C1-C8 alkyl)-(C═O)OH, and —O(C═O)(C1-C6 alkyl)-(C═O)O(C1-C6 alkyl); or a pharmaceutically acceptable salt, hydrate, solvate, or polymorph thereof; and optionally a pharmaceutically acceptable carrier.

It is understood that the disclosed compounds can be used in connection with the disclosed methods, compositions, kits, and uses.

1. R^{1A} and R^{1B} Groups

In one aspect, R^{1a} and R^{1b} together are (C═O). In a further aspect, R^{1a} and R^{1b} are hydrogen. In a yet further aspect, each of R^{1a} and R^{1b} are independently selected from hydrogen, hydroxyl, —NH₂, C1-C8 alkyl, C1-C8 alkoxy, C1-C8 monohaloalkyl, and C1-C8 polyhaloalkyl. In a still further aspect, each of R^{1a} and R^{1b} are independently selected from hydrogen, hydroxyl, —NH₂, C1-C6 alkyl, C1-C6 alkoxy, C1-C6 monohaloalkyl, and C1-C6 polyhaloalkyl. In an even further aspect, each of R^{1a} and R^{1b} are independently selected from hydrogen, hydroxyl, —NH₂, C1-C3 alkyl, C1-C3 alkoxy, C1-C3 monohaloalkyl, and C1-C3 polyhaloalkyl.

In a further aspect, each of R^{1a} and R^{1b} are independently selected from hydrogen, hydroxyl, and C1-C8 alkoxy. In a yet further aspect, each of R^{1a} and R^{1b} are independently selected from hydrogen, hydroxyl, and C1-C6 alkoxy. In a still further aspect, each of R^{1a} and R^{1b} are independently selected from hydrogen, hydroxyl, and C1-C3 alkoxy.

In a further aspect, each of R^{1a} and R^{1b} are independently selected from hydrogen, hydroxyl, methoxy, ethoxy, propoxy, isopropoxy, butoxy, isobutoxy, sec-butoxy, and tert-butoxy. In a yet further aspect, each of R^{1a} and R^{1b} are independently selected from hydrogen, hydroxyl, methoxy, and ethoxy. In a still further aspect, each of R^{1a} and R^{1b} are independently selected from hydrogen, hydroxyl, and methoxy. In an even further aspect, each of R^{1a} and R^{1b} are independently selected from hydrogen, hydroxyl, and ethoxy.

In a further aspect, R^{1a} is hydrogen and R^{1b} is selected from hydrogen, hydroxyl, and C1-C8 alkoxy. In a yet further aspect, R^{1a} is hydrogen and R^{1b} is selected from hydrogen, hydroxyl, and C1-C6 alkoxy. In a still further aspect, R^{1a} is hydrogen and R^{1b} is selected from hydrogen, hydroxyl, and C1-C3 alkoxy. In an even further aspect, R^{1a} is hydrogen and R^{1b} is selected from hydrogen, hydroxyl, methoxy, ethoxy, propoxy, isopropoxy, butoxy, isobutoxy, sec-butoxy, and tert-butoxy. In a still a further aspect, R^{1a} is hydrogen and R^{1b} is selected from hydrogen, hydroxyl, methoxy, and ethoxy. In a yet further aspect, R^{1a} is hydrogen and R^{1b} is selected from hydrogen, hydroxyl, and methoxy. In an even further aspect, R^{1a} is hydrogen and R^{1b} is selected from hydrogen, hydroxyl, and ethoxy.

In a further aspect, R^{1b} is hydrogen and R^{1a} is selected from hydrogen, hydroxyl, and C1-C8 alkoxy. In a still further aspect, R^{1b} is hydrogen and R^{1a} is selected from hydrogen, hydroxyl, and C1-C6 alkoxy. In a yet further aspect, R^{1b} is hydrogen and R^{1a} is selected from hydrogen, hydroxyl, and C1-C3 alkoxy. In an even further aspect, R^{1b} is hydrogen and R^{1a} is selected from hydrogen, hydroxyl, methoxy, ethoxy, propoxy, isopropoxy, butoxy, isobutoxy, sec-butoxy, and tert-butoxy. In a still further aspect, R^{1b} is hydrogen and R^{1a} is selected from hydrogen, hydroxyl, methoxy, and ethoxy. In a yet further aspect, R^{1b} is hydrogen and R^{1a} is selected from hydrogen, hydroxyl, and methoxy. In an even further aspect, R^{1b} is hydrogen and R^{1a} is selected from hydrogen, hydroxyl, and ethoxy.

In a further aspect, each of R^{1a} and R^{1b} are independently selected from NH(C1-C6 alkyl), and —N(C1-C6 alkyl)(C1-C6 alkyl). In a still further aspect, each of R^{1a} and R^{1b} are independently selected from —NH(C1-C3 alkyl), and —N(C1-C6 alkyl)(C1-C3 alkyl). In a yet further aspect, each of R^{1a} and R^{1b} are independently selected from —NHCH₃, —NHCH₂CH₃, —N(CH₃)₂, —N(CH₃)(CH₂CH₃), and —N(CH₂CH₃)₂.

In a further aspect, R^{1a} is hydrogen and R^{1b} is selected from —NH(C1-C6 alkyl), and —N(C1-C6 alkyl)(C1-C6 alkyl). In a still further aspect, R^{1a} is hydrogen and R^{1b} is selected from —NH(C1-C3 alkyl), and —N(C1-C6 alkyl)(C1-C3 alkyl). In a yet further aspect, R^{1a} is hydrogen and R^{1b} is selected from —NHCH₃, —NHCH₂CH₃, —N(CH₃)₂, —N(CH₃)(CH₂CH₃), and —N(CH₂CH₃)₂.

In a further aspect, R^{1b} is hydrogen and R^{1a} is selected from —NH(C1-C6 alkyl), and —N(C1-C6 alkyl)(C1-C6 alkyl). In a still further aspect, R^{1b} is hydrogen and R^{1a} is selected from —NH(C1-C3 alkyl), and —N(C1-C6 alkyl)(C1-C3 alkyl). In a yet further aspect, R^{1b} is hydrogen and R^{1a} is selected from —NHCH₃, —NHCH₂CH₃, —N(CH₃)₂, —N(CH₃)(CH₂CH₃), and —N(CH₂CH₃)₂.

In a further aspect, each of R^{1a} and R^{1b} are independently selected from C2-C5 heterocycloalkyl, C3-C6 cycloalkyl, and —O(C1-C8 alkyl)C3-C6 cycloalkyl; and wherein C2-C5 heterocycloalkyl, C3-C6 cycloalkyl, and —O(C1-C8 alkyl)C3-C6 cycloalkyl; and wherein R^{1b} is substituted with 0-3 groups selected from hydrogen, halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, each of R^{1a} and R^{1b} are independently selected from C2-C5 heterocycloalkyl, C3-C6 cycloalkyl, and —O(C1-C8 alkyl)C3-C6 cycloalkyl; and wherein C2-C5 heterocycloalkyl, C3-C6 cycloalkyl, and —O(C1-C8 alkyl)C3-C6 cycloalkyl, when present, is unsubstituted.

In a further aspect, R^{1a} is hydrogen and R^{1b} is selected from azetidinyl, pyrrolidinyl, piperidinyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, and cyclohexylethyl; and wherein R^{1b} is substituted with 0-3 groups selected from hydrogen, halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, R^{1a} is hydrogen and R^{1b} is selected from azetidinyl, pyrrolidinyl, piperidinyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, and cyclohexylethyl; and wherein R^{1b}, when not hydrogen, is unsubstituted. In a yet further aspect, R^{1a} is hydrogen and R^{1b} is a group selected from the following:

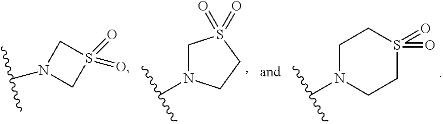 [SMILES: *N1CS(=O)(=O)C1 ; *N1CCS(=O)(=O)C1 ; "," ; "and" ; *N1CCS(=O)(=O)CC1 ; "."]

In a further aspect, each of R^{1b} and R^{1a} are independently selected from C2-C5 heterocycloalkyl, C3-C6 cycloalkyl, and —O(C1-C8 alkyl)C3-C6 cycloalkyl; and wherein C2-C5 heterocycloalkyl, C3-C6 cycloalkyl, and —O(C1-C8 alkyl)C3-C6 cycloalkyl; and wherein R^{1b} is substituted with 0-3 groups selected from hydrogen, halogen, hydroxyl, —NH₂, —(C═O)OH, C1-C8 alkyl, C1-C8 alkoxy, C1-C8 monohaloalkyl, C1-C8 polyhaloalkyl, NH(C1-C6 alkyl), N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, each of R^{1b} and R^{1a} are independently selected from C2-C5 heterocycloalkyl, C3-C6 cycloalkyl, and —O(C1-C8 alkyl)C3-C6 cycloalkyl; and wherein C2-C5 heterocycloalkyl, C3-C6 cycloalkyl, and —O(C1-C8 alkyl)C3-C6 cycloalkyl, when present, is unsubstituted.

In a further aspect, R^{1b} is hydrogen and R^{1a} is selected from azetidinyl, pyrrolidinyl, piperidinyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, and cyclohexylethyl; and wherein R^{1b} is substituted with 0-3 groups selected from hydrogen, halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, R^{1b} is hydrogen and R^{1a} is selected from azetidinyl, pyrrolidinyl, piperidinyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, and cyclohexylethyl; and wherein R^{1b}, when not hydrogen, is unsubstituted. In yet a further aspect, R^{1b} is hydrogen and R^{1a} is a group selected from the following:

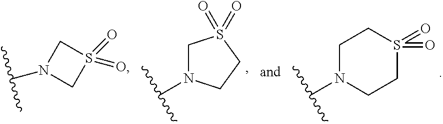 [SMILES: *N1CS(=O)(=O)C1 ; *N1CCS(=O)(=O)C1 ; "," ; "and" ; *N1CCS(=O)(=O)CC1 ; "."]

In a further aspect, R¹a is hydrogen and R^{1b} is selected from —O(C1-C8 alkyl)-(C═O)OH, —O(C═O)(C1-C6 alkyl)-(C═O)OH, —O(C1-C8 alkyl)-(C═O)O(C1-C6 alkyl), and —O(C═O)(C1-C6 alkyl)-(C═O)O(C1-C6 alkyl). In a still further aspect, R^{1a} is hydrogen and R^{1b} is selected from —O(C1-C6 alkyl)-(C═O)OH, —O(C═O)(C1-C6 alkyl)-(C═O)OH, —O(C1-C6 alkyl)-(C═O)O(C1-C6 alkyl), and —O(C═O)(C1-C6 alkyl)-(C═O)O(C1-C6 alkyl). In a yet further aspect, R^{1a} is hydrogen and R^{1b} is selected from —O(C1-C3 alkyl)-(C═O)OH, —O(C═O)(C1-C3 alkyl)-(C═O)OH, —O(C1-C3 alkyl)-(C═O)O(C1-C3 alkyl), and —O(C═O)(C1-C3 alkyl)-(C═O)O(C1-C3 alkyl). In an even further aspect, R^{1a} is hydrogen and R^{1b} is selected from —O(C1-C8 alkyl)-(C═O)OH, and, —O(C1-C8 alkyl)-(C═O)O(C1-C6 alkyl). In a still further aspect, R^{1a} is hydrogen and R^{1b} is selected from —O(C1-C6 alkyl)-(C═O)OH, and , —O(C1-C6 alkyl)-(C═O)O(C1-C6 alkyl). In a yet further aspect, R¹a is hydrogen and R^{1b} is selected from —O(C1-C3 alkyl)-(C═O)OH, and , —O(C1-C3 alkyl)-(C═O)O(C1-C3 alkyl).

In a further aspect, R^{1a} is hydrogen and R^{1b} is selected from:

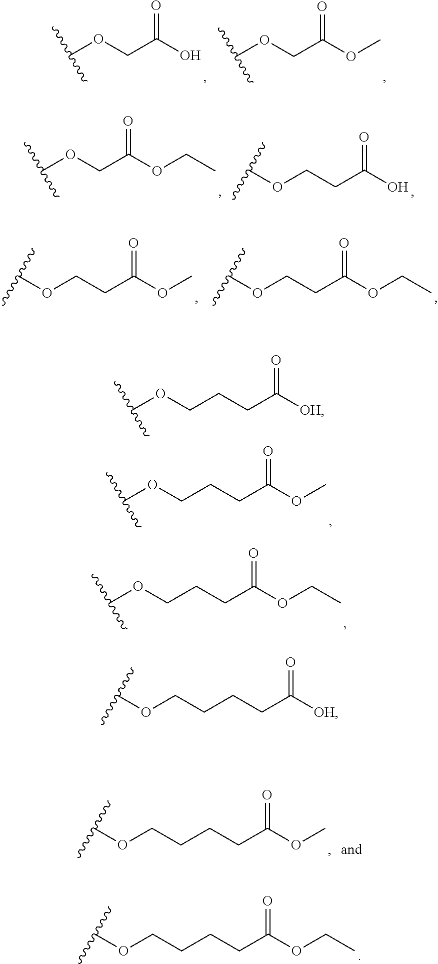

In a further aspect, R^{1a} is hydrogen and R^{1b} is selected from:

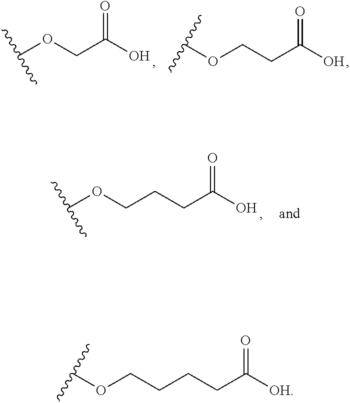 [SMILES: *OCC(=O)O ; *OCCC(=O)O ; *OCCCC(=O)O ; *OCCCCC(=O)O]

In a further aspect, R^{1a} is hydrogen and R^{1b} is:

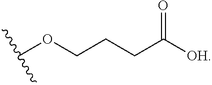 [SMILES: *OCCCC(=O)O]

In a further aspect, R^{1b} is hydrogen and R^{1a} is selected from —O(C1-C8 alkyl)-(C═O)OH, —O(C═O)(C1-C6 alkyl)-(C═O)OH, —O(C1-C8 alkyl)-(C═O)O(C1-C6 alkyl), and —O(C═O)(C1-C6 alkyl)-(C═O)O(C1-C6 alkyl). In a still further aspect, R^{1b} is hydrogen and R^{1a} is selected from —O(C1-C6 alkyl)-(C═O)OH, —O(C═O)(C1-C6 alkyl)-(C═O)OH, —O(C1-C6 alkyl)-(C═O)O(C1-C6 alkyl), and —O(C═O)(C1-C6 alkyl)-(C═O)O(C1-C6 alkyl). In a yet further aspect, R^{1b} is hydrogen and R^{1a} is selected from —O(C1-C3 alkyl)-(C═O)OH, —O(C═O)(C1-C3 alkyl)-(C═O)OH, —O(C1-C3 alkyl)-(C═O)O(C1-C3 alkyl), and —O(C═O)(C1-C3 alkyl)-(C═O)O(C1-C3 alkyl). In an even further aspect, R^{1b} is hydrogen and R^{1a} is selected from —O(C1-C8 alkyl)-(C═O)OH, and , —O(C1-C8 alkyl)-(C═O)O(C1-C6 alkyl). In a still further aspect, R^{1b} is hydrogen and R^{1a} is selected from —O(C1-C6 alkyl)-(C═O)OH, and, —O(C1-C6 alkyl)-(C═O)O(C1-C6 alkyl). In a yet further aspect, R^{1b} is hydrogen and R^{1a} is selected from —O(C1-C3 alkyl)-(C═O)OH, and , —O(C1-C3 alkyl)-(C═O)O(C1-C3 alkyl).

In a further aspect, R^{1b} is hydrogen and R^{1a} is selected from:

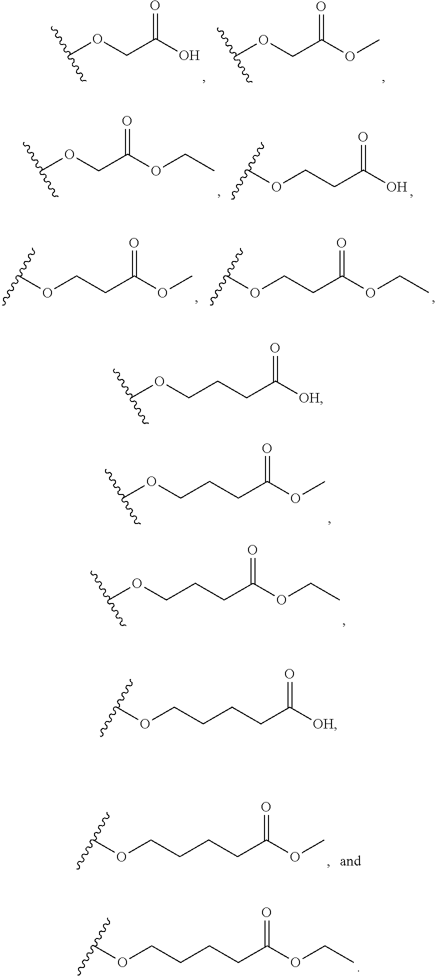

In a further aspect, R^{1b} is hydrogen and R^{1a} is selected from:

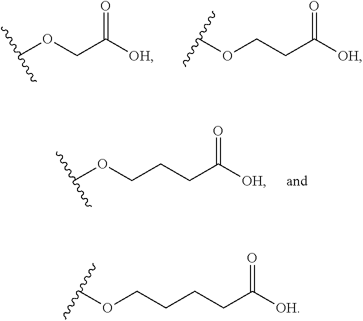 [SMILES: *OCC(=O)O ; *OCCC(=O)O ; *OCCCC(=O)O ; "and" ; *OCCCCC(=O)O]

In a further aspect, R^{1b} is hydrogen and R^{1a} is:

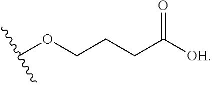 [SMILES: *OCCCC(=O)O]

In a further aspect, R^{1a} is hydrogen and R^{1b} is selected from —O(C1-C8 alkyl)-aryl, —O-aryl; and aryl; and wherein R^{1b} is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, R^{1a} is hydrogen and R^{1b} is selected from —O(C1-C8 alkyl)-aryl, —O-aryl; and aryl; and wherein R^{1b} is unsubstituted.

In a further aspect, R^{1b} is hydrogen and R^{1a} is selected from —O(C1-C8 alkyl)-aryl, —O-aryl; and aryl; and wherein R^{1b} is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, R^{1b} is hydrogen and R^{1a} is selected from —O(C1-C8 alkyl)-aryl, —O-aryl; and aryl; and wherein R^{1b} is unsubstituted.

In a further aspect, R^{1a} is hydrogen and R^{1b} is selected from —O(C1-C8 alkyl)-aryl and —O-aryl; and wherein R^{1b} is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a still further aspect, R^{1a} is hydrogen and R^{1b} is selected from —OCH₂-phenyl, —O(CH₂)₂-phenyl, —O(CH₂)₃-phenyl, and —O-aryl; and wherein R^{1b} is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl).

In a further aspect, R^{1a} is hydrogen and R^{1b} is selected from:

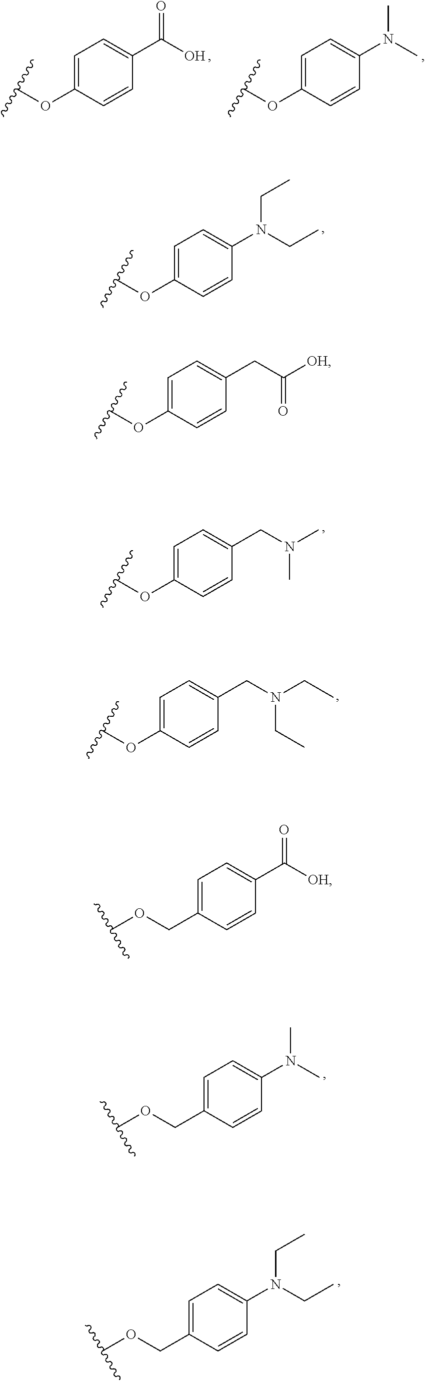

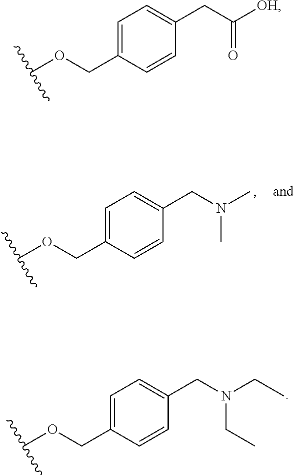 [SMILES: *OCc1ccc(CC(=O)O)cc1 ; *OCc1ccc(CN(C)C)cc1 ; *OCc1ccc(CN(CC)CC)cc1]

In a further aspect, R^{1a} is hydrogen and R^{1b} is selected from:

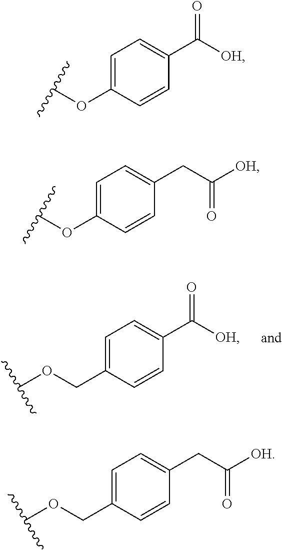 [SMILES: *Oc1ccc(C(=O)O)cc1 ; *Oc1ccc(CC(=O)O)cc1 ; *OCc1ccc(C(=O)O)cc1 ; "and" ; *OCc1ccc(CC(=O)O)cc1]

In a further aspect, R^{1a} is hydrogen and R^{1b} is selected from:

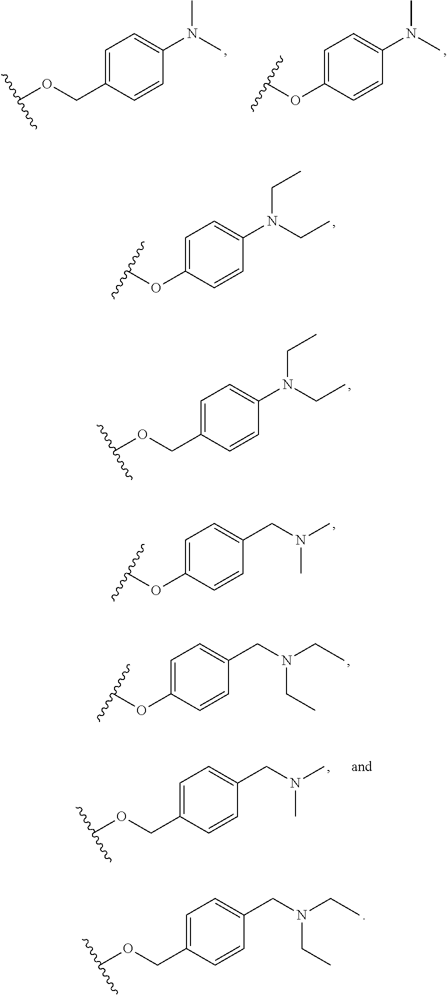

In a further aspect, R^{1a} is hydrogen and R^{1b} is:

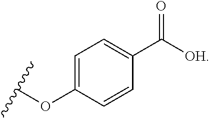 [SMILES: *Oc1ccc(C(=O)O)cc1]

In a further aspect, R^{1a} is hydrogen and R^{1b} is:

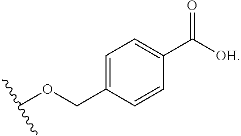 [SMILES: *OCc1ccc(C(=O)O)cc1]

In a further aspect, R^{1a} is hydrogen and R^{1b} is selected from —O(C1-C8 alkyl)-aryl and —O-aryl; and wherein R^{1b} is unsubstituted.

In a further aspect, R^{1b} is hydrogen and R^{1a} is selected from —O(C1-C8 alkyl)-aryl and —O-aryl; and wherein R^{1b} is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl). In a yet further aspect, R^{1b} is hydrogen and R^{1a} is selected from —OCH₂-phenyl, —O(CH₂)₂-phenyl, —O(CH₂)₃-phenyl, and —O-aryl; and wherein R^{1b} is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl).

In a further aspect, R^{1b} is hydrogen and R^{1a} is selected from:

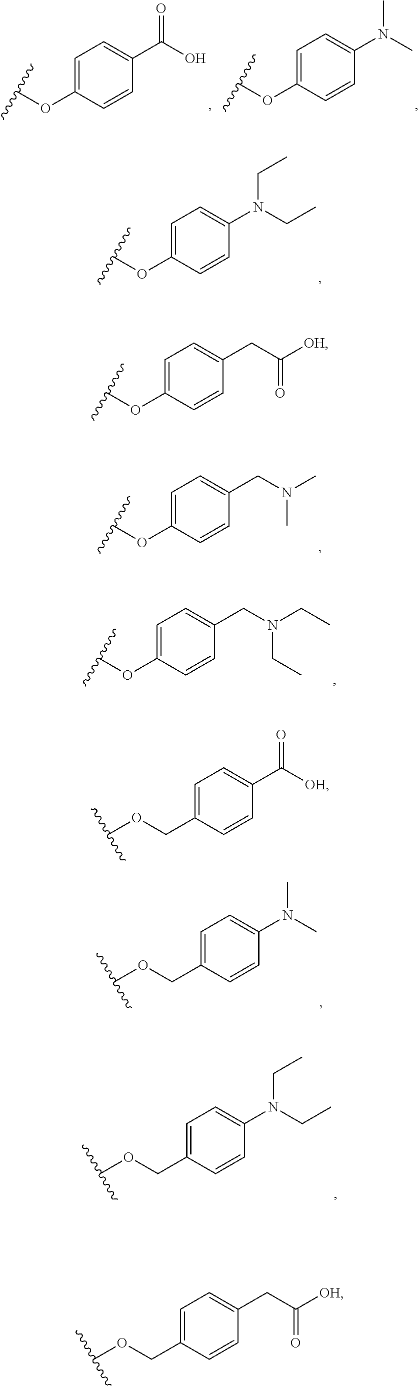

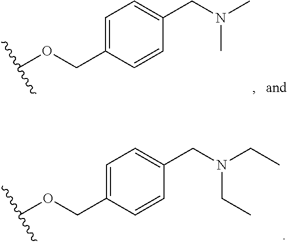 [SMILES: *OCc1ccc(CN(C)C)cc1 ; *OCc1ccc(CN(CC)CC)cc1]

In a further aspect, R^{1b} is hydrogen and R^{1a} is selected from:

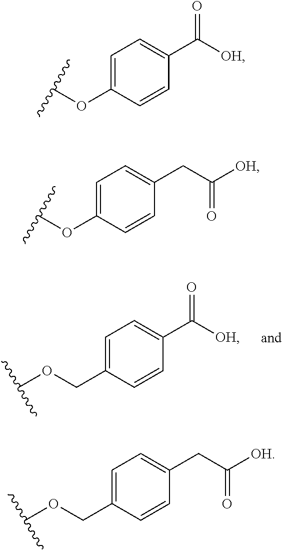 [SMILES: *Oc1ccc(C(=O)O)cc1 ; *Oc1ccc(CC(=O)O)cc1 ; *OCc1ccc(C(=O)O)cc1 ; "and" ; *OCc1ccc(CC(=O)O)cc1]

In a further aspect, R^{1b} is hydrogen and R^{1a} is selected from: [symbol]
,and

In a further aspect, R^{1b} is hydrogen and R^{1a} is:

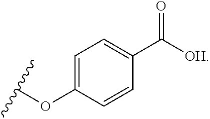 [SMILES: *Oc1ccc(C(=O)O)cc1]

In a further aspect, R^{1b} is hydrogen and R^{1a} is:

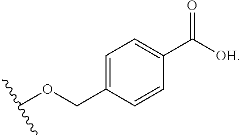 [SMILES: *OCc1ccc(C(=O)O)cc1]

In a further aspect, R^{1b} is hydrogen and R^{1a} is selected from —O(C1-C8 alkyl)-aryl and —O-aryl; and wherein R^{1b} is unsubstituted.

2. R^{2A} and R^{2B} Groups

In one aspect, R^{2a} and R^{2b} together are (C═CH₂). In a further aspect, each of R^{2a} and R^{2b} is independently selected from hydrogen, —(C1-C8 alkyl)-(C═O)OH, and —O(C═O)(C1-C6 alkyl)-(C═O)O(C1-C6 alkyl).

In a further aspect, each of R^{2a} and R^{2b} is independently selected from hydrogen and a moiety having a structure represented by the formula:

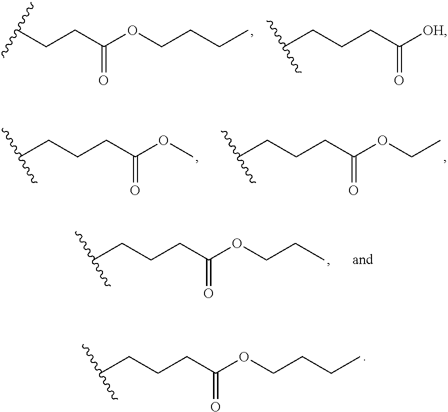

In a further aspect, each of R^{2a} and R^{2b} is independently selected from hydrogen and a moiety having a structure represented by the formula:

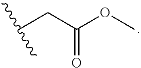 [SMILES: *CC(=O)OC]

In a further aspect, R^{2a} is hydrogen and R^{2b} is selected from hydrogen, —(C1-C8 alkyl)-(C═O)OH, and —O(C═O)(C1-C6 alkyl)-(C═O)O(C1-C6 alkyl). In a still further aspect, R^{2a} is hydrogen and R^{2b} is selected from hydrogen and a moiety having a structure represented by the formula:

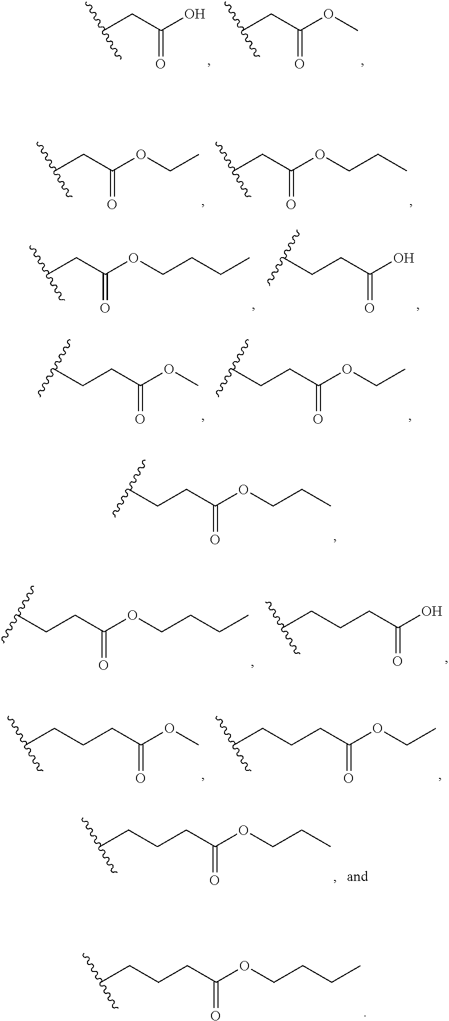

In a further aspect, R^{2a} is hydrogen and R^{2b} is selected from hydrogen and a moiety having a structure represented by the formula:

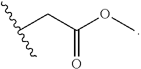 [SMILES: *CC(=O)OC]

In a further aspect, R^{2b} is hydrogen and R^{2a} is selected from hydrogen, —(C1-C8 alkyl)-(C═O)OH, and —O(C═O)(C1-C6 alkyl)-(C═O)O(C1-C6 alkyl). In a still further aspect, R^{2b} is hydrogen and R^{2a} is selected from hydrogen and a moiety having a structure represented by the formula:

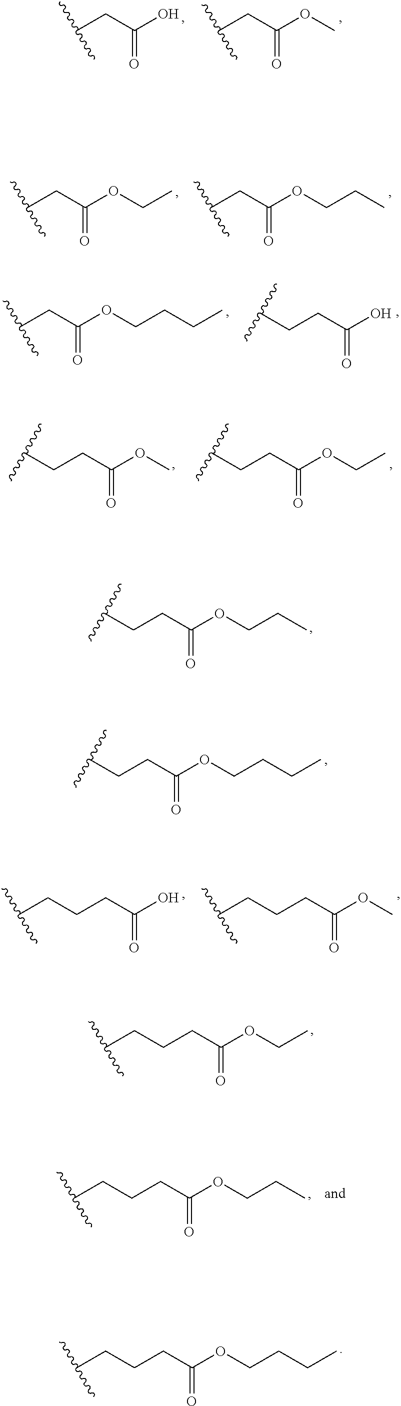

In a further aspect, R^{2b} is hydrogen and R^{2a} is selected from hydrogen and a moiety having a structure represented by the formula:

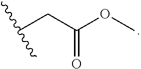 [SMILES: *CC(=O)OC]

In a further aspect, each of R^{2a} and R^{2b} is hydrogen. In a still further aspect, each of R^{2a} and R^{2b} is methyl. In a yet further aspect, each of R^{2a} and R^{2b} is independently selected from hydrogen, C1-C6 alkyl, C1-C6 monohaloalkyl, and C1-C6 polyhaloalkyl. In an even further aspect, each of R^{2a} and R^{2b} is independently selected from hydrogen, C1-C3 alkyl, C1-C3 monohaloalkyl, and C1-C3 polyhaloalkyl.

In a further aspect, each of R^{2a} and R^{2b} is independently selected from hydrogen and C1-C8 alkyl. In a still further aspect, each of R^{2a} and R^{2b} is independently selected from hydrogen and C1-C6 alkyl. In a yet further aspect, each of R^{2a} and R^{2b} is independently selected from hydrogen and C1-C3 alkyl.

In a further aspect, each of R^{2a} and R^{2b} is independently selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, each of R^{2a} and R^{2b} is independently selected from hydrogen, methyl, ethyl, propyl, isopropyl, and tert-butyl. In a yet further aspect, each of R^{2a} and R^{2b} is independently selected from hydrogen, methyl, ethyl, propyl, and isopropyl. In an even further aspect, each of R^{2a} and R^{2b} is independently selected from hydrogen and methyl.

In a further aspect, R^{2a} is hydrogen and R^{2b} is selected from hydrogen, C1-C6 alkyl, C1-C6 monohaloalkyl, and C1-C6 polyhaloalkyl. In a still further aspect, R^{2a} is hydrogen and R^{2b} is selected from hydrogen, C1-C3 alkyl, C1-C3 monohaloalkyl, and C1-C3 polyhaloalkyl. In a yet further aspect, R^{2a} is hydrogen and R^{2b} is selected from hydrogen and C1-C8 alkyl. In an even further aspect, R^{2a} is hydrogen and R^{2b} is selected from hydrogen and C1-C6 alkyl. In a still further aspect, R^{2a} is hydrogen and R^{2b} is selected from hydrogen and C1-C3 alkyl.

In a further aspect, R^{2a} is hydrogen and R^{2b} is selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, R^{2a} is hydrogen and R^{2b} is selected from hydrogen, methyl, ethyl, propyl, isopropyl, and tert-butyl. In a yet further aspect, R^{2a} is hydrogen and R^{2b} is selected from hydrogen, methyl, ethyl, propyl, and isopropyl. In a yet further aspect, R^{2a} is hydrogen and R^{2b} is selected from hydrogen and methyl. In an even further aspect, R^{2a} is hydrogen and R^{2b} is methyl.

In a further aspect, R^{2b} is hydrogen and R^{2a} is selected from hydrogen, C1-C6 alkyl, C1-C6 monohaloalkyl, and C1-C6 polyhaloalkyl. In a still further aspect, R^{2b} is hydrogen and R^{2a} is selected from hydrogen, C1-C3 alkyl, C1-C3 monohaloalkyl, and C1-C3 polyhaloalkyl. In a yet further aspect, R^{2b} is hydrogen and R^{2a} is selected from hydrogen and C1-C8 alkyl. In an even further aspect, R^{2b} is hydrogen and R^{2a} is selected from hydrogen and C1-C6 alkyl. In a still further aspect, R^{2b} is hydrogen and R^{2a} is selected from hydrogen and C1-C3 alkyl.

In a further aspect, R^{2b} is hydrogen and R^{2a} is selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, R^{2b} is hydrogen and R^{2a} is selected from hydrogen, methyl, ethyl, propyl, isopropyl, and tert-butyl. In a yet further aspect, R^{2b} is hydrogen and R^{2a} is selected from hydrogen, methyl, ethyl, propyl, and isopropyl. In an even further aspect, R^{2b} is hydrogen and R^{2a} is selected from hydrogen and methyl. In a still further method, R^{2b} is hydrogen and R^{2a} is methyl.

3. R^{3A} and R^{3B} Groups

In one aspect, each of R^{3a} and R^{3b} is hydrogen. In a further aspect, each of R^{1a} and R^{3b} is independently selected from hydrogen, C1-C6 alkyl, C1-C6 monohaloalkyl, and C1-C6 polyhaloalkyl. In a still further aspect, each of R^{3a} and R^{3b} is independently selected from hydrogen, C1-C3 alkyl, C1-C3 monohaloalkyl, and C1-C3 polyhaloalkyl. In a yet further aspect, each of R^{3a} and R^{3b} is independently selected from hydrogen and C1-C8 alkyl. In an even further aspect, each of R^{3a} and R^{3b} is independently selected from hydrogen and C1-C6 alkyl. In a still further aspect, each of R^{3a} and R^{3b} is independently selected from hydrogen and C1-C3 alkyl.

In a further aspect, each of R^{3a} and R^{3b} is independently selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, each of R^{3a} and R^{3b} is independently selected from hydrogen, methyl, ethyl, propyl, isopropyl, and tert-butyl. In a yet further aspect, each of R^{3a} and R^{3b} is independently selected from hydrogen, methyl, ethyl, propyl, and isopropyl. In an even further aspect, each of R^{3a} and R^{3b} is independently selected from hydrogen and methyl.

In a further aspect, R^{3a} is hydrogen and R^{3b} is selected from hydrogen, C1-C6 alkyl, C1-C6 monohaloalkyl, and C1-C6 polyhaloalkyl. In a still further aspect, R^{3a} is hydrogen and R^{3b} is selected from hydrogen, C1-C3 alkyl, C1-C3 monohaloalkyl, and C1-C3 polyhaloalkyl. In a yet further aspect, R^{3a} is hydrogen and R^{3b} is selected from hydrogen and C1-C8 alkyl. In an even further aspect, R^{3a} is hydrogen and R^{3b} is selected from hydrogen and C1-C6 alkyl. In a still further aspect, R^{3a} is hydrogen and R^{3b} is selected from hydrogen and C1-C3 alkyl.

In a further aspect, R^{3a} is hydrogen and R^{3b} is selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, R^{3a} is hydrogen and R^{3b} is selected from hydrogen, methyl, ethyl, propyl, isopropyl, and tert-butyl. In a yet further aspect, R^{3a} is hydrogen and R^{3b} is selected from hydrogen, methyl, ethyl, propyl, and isopropyl. In an even further aspect, R^{3a} is hydrogen and R^{3b} is selected from hydrogen and methyl. In a still further aspect, R^{3a} is hydrogen and R^{3b} is methyl.

In a further aspect, R^{3b} is hydrogen and R^{3a} is selected from hydrogen, C1-C6 alkyl, C1-C6 monohaloalkyl, and C1-C6 polyhaloalkyl. In a still further aspect, R^{3b} is hydrogen and R^{3a} is selected from hydrogen, C1-C3 alkyl, C1-C3 monohaloalkyl, and C1-C3 polyhaloalkyl. In a yet further aspect, R^{3b} is hydrogen and R¹a is selected from hydrogen and C1-C8 alkyl. In an even further aspect, R^{3b} is hydrogen and R^{3a} is selected from hydrogen and C1-C6 alkyl. In a still further aspect, R^{3b} is hydrogen and R^{3a} is selected from hydrogen and C1-C3 alkyl.

In a further aspect, R^{3b} is hydrogen and R^{3a} is selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, R^{3b} is hydrogen and R^{3a} is selected from hydrogen, methyl, ethyl, propyl, isopropyl, and tert-butyl. In a yet further aspect, R^{3b} is hydrogen and R^{3a} is selected from hydrogen,

methyl, ethyl, propyl, and isopropyl. In an even further aspect, R^{3b} is hydrogen and R^{3a} is selected from hydrogen and methyl. In a still further aspect, R^{3b} is hydrogen and R^{3a} is methyl.

4. R⁴ Groups

In one aspect, R⁴ is selected from —(C1-C8 alkyl)-(C═O)OH and —O(C═O)(C1-C6 alkyl)-(C═O)O(C1-C6 alkyl). In a further aspect, R⁴ is selected from a group having a structure represented by a formula: [symbol]
and

In a further aspect, R⁴ is:

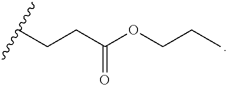 [SMILES: *CCC(=O)OCCC]

In a further aspect, R⁴ is selected from hydrogen, C1-C6 alkyl, C1-C6 monohaloalkyl, and C1-C6 polyhaloalkyl. In a still further aspect, R⁴ is selected from hydrogen, C1-C3 alkyl, C1-C3 monohaloalkyl, and C1-C3 polyhaloalkyl. In a yet further aspect, R⁴ is selected from hydrogen and C1-C8 alkyl. In an even further aspect, R⁴ is selected from hydrogen and C1-C6 alkyl. In a still further aspect, R⁴ is selected from hydrogen and C1-C3 alkyl.

In a further aspect, R⁴ is selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, R⁴ is selected from hydrogen, methyl, ethyl, propyl, isopropyl, and tert-butyl. In a yet further aspect, R⁴ is selected from hydrogen, methyl, ethyl, propyl, and isopropyl. In an even further aspect, R⁴ is selected from hydrogen and methyl. In a still further aspect, R⁴ is hydrogen. In a yet further

[symbol]

aspect, R⁴ is methyl.

5. R^{5A} and R^{5B} Groups

In one aspect, each of R^{5a} and R^{5b} are hydrogen. In a further aspect, each of R^{5a} and R^{5b} is independently selected from hydrogen, C1-C6 alkyl, C1-C6 monohaloalkyl, and C1-C6 polyhaloalkyl. In a still further aspect, each of R^{5a} and R^{5b} is independently selected from hydrogen, C1-C3 alkyl, C1-C3 monohaloalkyl, and C1-C3 polyhaloalkyl. In a yet further aspect, each of R^{5a} and R^{5b} is independently selected from hydrogen and C1-C8 alkyl. In an even further aspect, each of R^{5a} and R^{5b} is independently selected from hydrogen and C1-C6 alkyl. In a still further aspect, each of R^{5a} and R^{5b} is independently selected from hydrogen and C1-C3 alkyl.

In a further aspect, each of R^{5a} and R^{5b} is independently selected from hydrogen, methyl, ethyl, propyl, isopropyl, tert-butyl, sec-butyl, isobutyl, neopentyl, isopentyl, sec-pentyl, tert-pentyl, methylbutyl, dimethylbutyl, methylpentyl, and dimethylpentyl. In a still further aspect, each of R^{5a} and R^{5b} is independently selected from hydrogen, methyl, ethyl, propyl, isopropyl, and tert-butyl. In a yet further aspect, each of R^{5a} and R^{5b} is independently selected from hydrogen, methyl, ethyl, propyl, and isopropyl. In an even further aspect, each of R^{5a} and R^{5b} is independently selected from hydrogen and methyl.

6. Exemplary Artemisinin Analogue Compounds

In one aspect, the invention relates to pharmaceutical compositions comprising a at least one artemisinin analogue selected from:

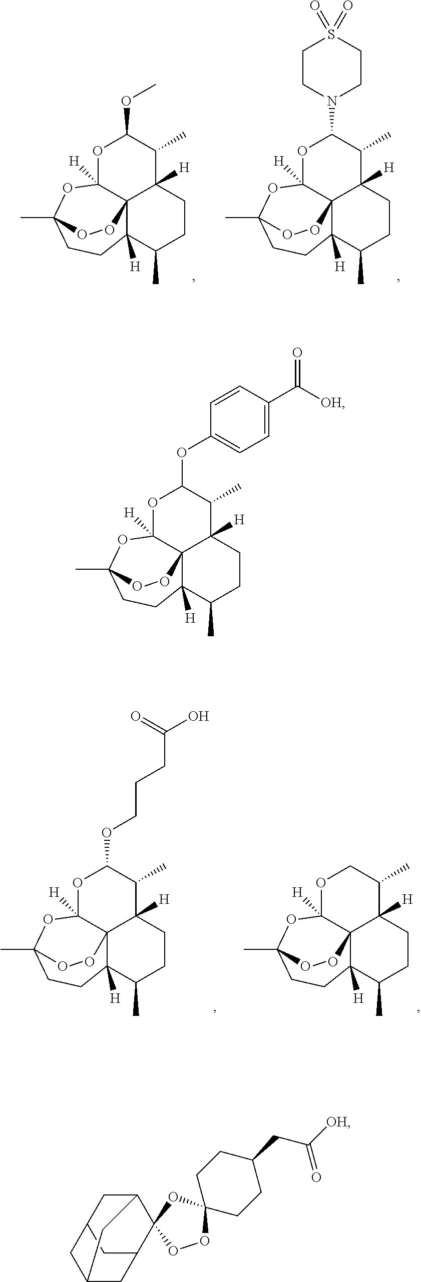

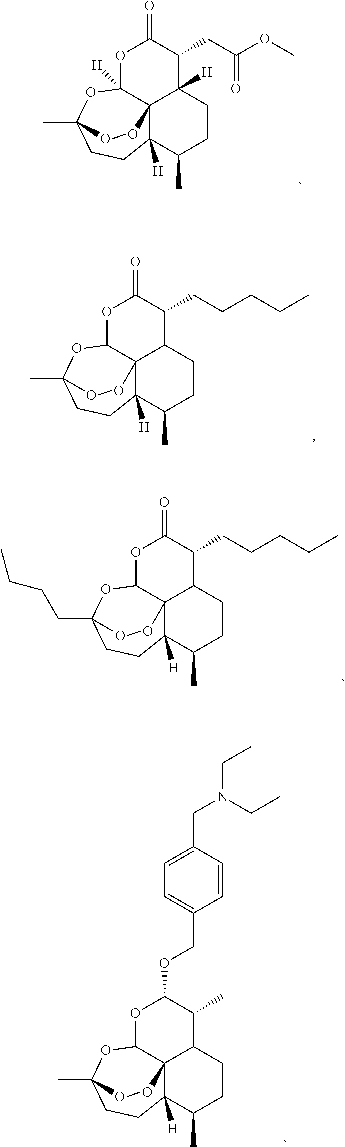

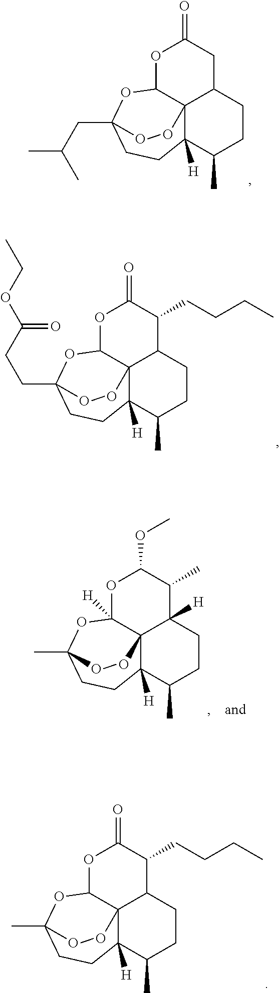

In one aspect, the artemisinin analogue is selected from:

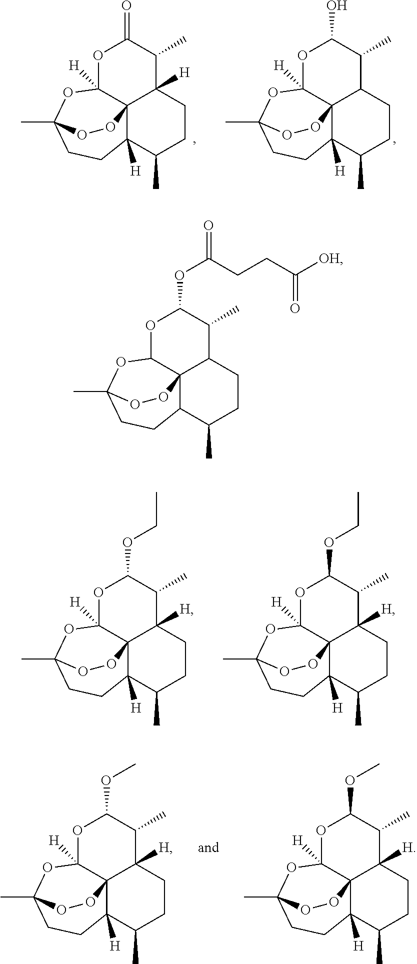

In one aspect, the artemisinin analogue is:

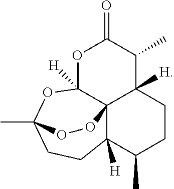

In one aspect, the artemisinin analogue is:

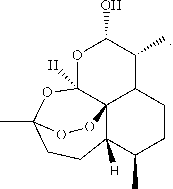

In one aspect, the artemisinin analogue is:

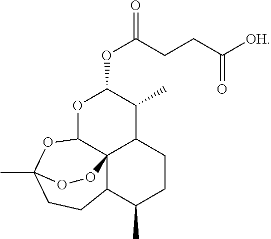 [SMILES: C[C@@H]1CCC2[C@@H](C)[C@@H](OC(=O)CCC(=O)O)OC3OC4(C)CCC1[C@]32OO4]

In one aspect, the artemisinin analogue is:

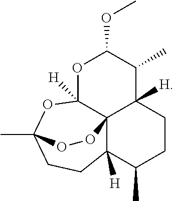

### C. BCR-ABL Tyrosine Kinase Inhibitors

In various aspects, the invention relates to compositions comprising a therapeutically effective amount of a BCR-ABL tyrosine kinase inhibitor. In a further aspect, the BCR-ABL tyrosine kinase inhibitor is selected from imatinib, dasatinib, nilotinib, bosutinib, ponatinib, bafetinib, saracatinib, tozasertib and rebastinib. In a still further aspect, the BCR-ABL tyrosine kinase inhibitor is selected from imatinib, dasatinib, nilotinib, bosutinib, ponatinib, bafetinib, saracatinib, and tozasertib.

In a further aspect, the BCR-ABL tyrosine kinase inhibitor is imatinib. In a still further aspect, the BCR-ABL tyrosine kinase inhibitor is dasatinib. In a yet further aspect, the BCR-ABL tyrosine kinase inhibitor is nilotinib. In an even further aspect, the BCR-ABL tyrosine kinase inhibitor is bosutinib. In a still further aspect, the BCR-ABL tyrosine kinase inhibitor is ponatinib. In a yet further aspect, the BCR-ABL tyrosine kinase inhibitor is bafetinib. In an even further aspect, the BCR-ABL tyrosine kinase inhibitor is saracatinib. In a still further aspect, the BCR-ABL tyrosine kinase inhibitor is tozasertib.

In a further aspect, the BCR-ABL tyrosine kinase inhibitor is selected from a BCR-ABL selective inhibitor, a dual BCR-ABL and Src-family kinases inhibitor, Aurora kinase inhibitor, Kit inhibitor, ATP competitive inhibitor and non-ATP competitive BCR-ABL inhibitor. In a still further aspect, the BCR-ABL tyrosine kinase inhibitor is a selective BCR-ABL inhibitor. In a yet further aspect, the BCR-ABL tyrosine kinase inhibitor is a dual BCR-ABL and Src-family kinases inhibitor. In an even further aspect, the BCR-ABL tyrosine kinase inhibitor is a Aurora kinase inhibitor. In a yet further aspect, the BCR-ABL tyrosine kinase inhibitor is a KIT inhibitor. In a still further aspect, the BCR-ABL tyrosine kinase inhibitor is a ATP competitive BCR-ABL inhibitor. In an even further aspect, the BCR-ABL tyrosine kinase inhibitor is a non-ATP competitive BCR-ABL inhibitor.

### D. Pharmaceutical Compositions

In one aspect, the invention relates to pharmaceutical compositions comprising the disclosed compounds. That is, a pharmaceutical composition can be provided comprising a therapeutically effective amount of at least one disclosed compound and a pharmaceutically acceptable carrier.

In certain aspects, the disclosed pharmaceutical compositions comprise the disclosed compounds (including pharmaceutically acceptable derivatives (e.g., salt(s)) thereof) as an active ingredient, a pharmaceutically acceptable carrier, and, optionally, other therapeutic ingredients or adjuvants. The compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The disclosed compounds can be administered by oral, parenteral (e.g., intramuscular, intraperitoneal, intravenous, intracerebroventricular, intracisternal injection or infusion, subcutaneous injection, or implant), by inhalation spray, nasal, vaginal, rectal, sublingual, or topical routes of administration and can be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans. The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions encompass any composition made by admixing a compound of the present invention and a pharmaceutically acceptable carrier.

As used herein, the term “pharmaceutically acceptable salts” refers to salts prepared from pharmaceutically acceptable non-toxic bases or acids. When a disclosed compound is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids,” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable derivatives thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof

Pharmaceutical compositions can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment of the disclosed conditions, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The disclosed pharmaceutical compositions can further comprise other therapeutically active compounds, as discussed further herein, which are usually applied in the treatment of the above mentioned pathological conditions.

In a further aspect, a pharmaceutical composition can comprise a therapeutically effective amount of any one or more disclosed compound and a pharmaceutically acceptable carrier. In a further aspect, a pharmaceutical composition can comprise a therapeutically effective amount of one or more product of any disclosed method and a pharmaceutically acceptable carrier. In one aspect, the invention relates to a method for manufacturing a medicament comprising combining at least one disclosed compound or at least one product of a disclosed method with a pharmaceutically acceptable carrier or diluent.

It is understood that the disclosed compositions can be prepared from the disclosed compounds. It is also understood that the disclosed compositions can be employed in the disclosed methods of using.

### E. Methods of Using the Compounds and Compositions

BCR-ABL tyrosine kinase (BCR-ABL TK) has been known to activate many pro-growth and cell survival mechanisms, which in turn promote cell proliferation and survival, and contribute to BCR-ABL mediated leukemic transformation of bone marrow progenitor cells. BCR-ABL TK-specific inhibition has previously been established as an effective therapeutic target. BCR-ABL tyrosine kinase shifts between a catalytically active open conformation and an inactive closed one. The structure of BCR-ABL contains two flexible loops, the ATP-binding P-loop (P-loop) and the activation loop (A-loop). These loops have specific arrangements in the inactive conformation of BCR-ABL that stabilize the basal conformation. In the inactive form the A-loop blocks the catalytic site and prevents BCR-ABL activity. BCR-ABL TK-specific inhibitors (BCR-ABL TKIs), such as imatinib, have been observed to bind and stabilize the inactive conformation, and have reduced previously poor chronic myelongenous leukemia (CML) outcomes to a controlled chronic condition. However, BCR-ABL TKIs have failed to produce a similar long-term response in related leukemias, such as pediatric and adult Ph+acute lymphoblastic leukemia (ALL). While current BCR-ABL-inhibition agents appear to kill Ph+ ALL cells in vitro, these drugs fails to meaningfully reduce leukemia burden in patients with Ph+ ALL, invariably leading to relapse and poor long-term outcomes.

Resistance to BCR-ABL TKIs is believed to contribute to the inability to induce adequate clinical responses. A major mechanism of BCR-ABL TKI resistance is thought to relate to mutations that result in the interference of binding to the BCR-ABL kinase domain. BCR-ABL mutations can confer different levels of resistance, ranging from concentration dependent resistance to complete BCR-ABL TKIs resistance. The most clinically significant mutations are P-loop mutations and the T315I mutation. Significantly, the T315I mutation produces the most common and highest form of resistance of any mutation, resulting in resistance to both imatinib and second generation BCR-ABL TKIs, dasatinib and nilotinib. Moreover, differences in clinical responses to BCR-ABL TKIs in patients with Ph+ ALL and CML remain poorly understood. Without wishing to be bound by a particular theory, these differences cannot be exclusively attributed to mutations in the BCR-ABL kinase domain that weaken drug binding.

Previously, a role has been reported for host cytokines of the common gamma chain receptor family in modulating primary imatinib resistance, but many alternative host-derived growth factors have similarly been proposed. Currently, no consensus exists on the relative importance of cytokines that control the cellular responses to BCR-ABL-targeted agents. Interleukin 7 (IL7) is a multifunctional cytokine essential for human T cell and post-fetal B cell development and is a major contributor to the regulation and maintenance of mature T cells. This non-redundant cytokine is constitutively produced by stromal cells in bone marrow, spleen, lymph nodes and thymus, as well as other tissues. IL7-rich niches have been observed to harbor early persistent drug-refractory leukemias in humans and mice. Without wishing to be bound by a particular theory, in these niches, BCR-ABL mutations are believed to be eventually acquired under continuous drug exposures, causing secondary drug resistance. Moreover, the responsiveness to extrinsic cytokines increases in persistent (drug-refractory) human Ph+ leukemia samples collected from BCR-ABL TKI-treated patients. In keeping with the critical homeostatic role of IL7 in human immune-reconstitution, IL7 levels dramatically vary in inverse proportion to an individual patient's absolute lymphocyte counts (ALC). Without wishing to be bound by a particular theory, contemporary BCR-ABL TKI-combined protocols deploy high-dose corticosteroids, conventional chemotherapy agents, allo-HSCT, and immune-suppressants, all of which induce profound iatrogenic-lymphopenias, and activate the immune IL7-axis thus likely blunting dasatinib efficacy. Moreover, the relatively robust dasatinib survival benefit in human patients with prolonged lymphocytosis may arise from their inherently low levels of IL7, and vice versa. Without wishing to be bound by a particular theory, it is believed that reducing the IL7-related activity through pharmacological intervention in diseases harboring BCR-ABL defects or mutations can have an important therapeutic benefit. Thus, IL7 provides an attractive, BCR-ABL-independent target for therapeutic intervention.

Additionally, human Ph+ ALL patients with increased levels of pSTAT5, a latent transcription factor whose phosphorylation is triggered by IL7, have been reported to respond poorly to imatinib therapy. Without wishing to be bound by a particular theory, c-MYC, a strong proto-oncogene, may play a role in IL7-induced BCR-ABL TKI resistance because expression of c-MYC can be induced by activated STAT5, in turn induced by IL7-JAK signaling. This phenomenon has been implicated in other stroma-induced drug resistance as well as used as an explanation for resistance to imatinib. Moreover, STAT5 activation and c-MYC expression is reportedly necessary to sustain BCR-ABL transformed cells. This extensive family of signaling proteins regulates important cell processes that, when dysregulated, lead to typical tumorigenic phenotype features. Therefore, inhibition at any point along this signaling cascade may provide clinical benefit to patients with pathologies that are driven by overactive signaling in this pathway. Without wishing to be bound by a particular theory, it is believed that inhibition of c-MYC activity, thereby countering the protective effects of IL7, can resolve and rescue the IL7-induced BCR-ABL TKI resistance that is a consequence of these mutations.

1. Treatment Methods

The compounds disclosed herein are useful for treating, preventing, ameliorating, controlling or reducing the risk of a variety of disorders of uncontrolled cellular proliferation. In one aspect, the disorder of uncontrolled cellular proliferation is associated with the BCR-ABL pathway. In a further aspect, the disorder of uncontrolled cellular proliferation is associated with a Philadelphia chromosome positive leukemia. In a further aspect, the BCR-ABL dysfunction is a mutation of the BCR-ABL gene. In one aspect, the disorder of uncontrolled cellular proliferation is associated with a gastrointestinal stromal tumor (GIST).

Examples of disorders of uncontrolled cellular proliferation include cancers such as leukemias, lymphomas, and solid tumors. In one aspect, the cancer can be a cancer selected from cancers of the gastrointestinal tract, hematologic, colon, rectum, liver, omentum, breast, kidney, lymphatic system, stomach, lung, pancreas, liver and skin. In a further aspect, the cancer is selected from leukemia and gastrointestinal stroma tumor.

Examples of disorders associated with BCR-ABL include leukemias, chronic myeloid leukemia (CML), acute lymphoblastic leukemia (ALL), and Philadelphia chromosome positive acute lymphoblastic leukemia (Ph+ALL). It can be appreciated that additional cancers, such as those associated with tyrosine kinase dysfunction, may benefit from using the present invention, including, for example, carcinomas, colon, kidney, liver, lung, pancreas, stomach, thyroid, testis, testicular seminomas, squamous cell carcinoma, and other hematologic tumors.

a. Treatment of a Philadilphia Chromosome Positive Leukemia

In one aspect, the invention relates to a method for the treatment of a Philadelphia chromosome positive leukemia in a patient comprising the step of administering to the patient a therapeutically effective amount of a BCR-ABL kinase inhibitor and a therapeutically effective amount of at least one disclosed artemisinin analogue, or a pharmaceutically acceptable salt, solvate, or polymorph thereof.

In a further aspect, the BCR-ABL tyrosine kinase inhibitor and the artemisinin analogue are co-administered. In a still further aspect, the BCR-ABL tyrosine kinase inhibitor is administered prior to administration of the artemisinin analogue. In a yet further aspect, the artemisinin analogue is administered prior to administration of the BCR-ABL tyrosine kinase inhibitor.

In a further aspect, the BCR-ABL tyrosine kinase inhibitor is administered in a first period; and the BCR-ABL tyrosine kinase inhibitor and the artemisinin analogue are co-administered in a second period. In a still further aspect, the BCR-ABL tyrosine kinase inhibitor administered in a first period and the BCR-ABL tyrosine kinase inhibitor administered in a second period are not the same. In a yet further aspect, the BCR-ABL tyrosine kinase inhibitor administered in a first period and the BCR-ABL tyrosine kinase inhibitor administered in a second period are the same.

In a further aspect, the Philadelphia chromosome positive leukemia after the first treatment period is refractory to treatment by the BCR-ABL tyrosine kinase inhibitor. In a still further aspect, the Philadelphia chromosome positive leukemia during the first treatment period becomes refractory to treatment by the BCR-ABL tyrosine kinase inhibitor.

In a further aspect, the Philadelphia chromosome positive leukemia comprises a mutation. In a yet further aspect, the Philadelphia chromosome positive leukemia after the first treatment period comprises a mutation selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, I418S, I418V, S438C, P441L, E450A, E450G, E450K, E450V, E453K, E453V, E459G, E459K, M472I, P480L, F486S, D504D, G514S, T240T, K247R, F311V, and E499E. In a still further aspect, the mutation is T315I.

In a further aspect, the BCR-ABL tyrosine kinase inhibitor is selected from imatinib, dasatinib, nilotinib, bosutinib, ponatinib, bafetinib, saracatinib, tozasertib and rebastinib. In a yet further aspect, the BCR-ABL tyrosine kinase inhibitor is selected from imatinib, dasatinib, nilotinib, bosutinib, ponatinib, bafetinib, saracatinib, and tozasertib. In a still further aspect, the BCR-ABL tyrosine kinase inhibitor is imatinib. In yet a further aspect, the BCR-ABL tyrosine kinase inhibitor is dasatinib. In an even further aspect, the BCR-ABL tyrosine kinase inhibitor is nilotinib. In a still further aspect, the BCR-ABL tyrosine kinase inhibitor is bosutinib. In a yet further aspect, the BCR-ABL tyrosine kinase inhibitor is ponatinib. In an even further aspect, the BCR-ABL tyrosine kinase inhibitor is bafetinib. In a still further aspect, the BCR-ABL tyrosine kinase inhibitor is saracatinib. In a yet further aspect, the BCR-ABL tyrosine kinase inhibitor is tozasertib.

In a further aspect, the BCR-ABL tyrosine kinase inhibitor is selected from a BCR-ABL selective inhibitor, a dual BCR-ABL and Src-family kinases inhibitor, Aurora kinase inhibitor, Kit inhibitor, ATP competitive inhibitor and non-ATP competitive BCR-ABL inhibitor. In a still further aspect, the BCR-ABL tyrosine kinase inhibitor is a selective BCR-ABL inhibitor. In a yet further aspect, the BCR-ABL tyrosine kinase inhibitor is a dual BCR-ABL and Src-family kinases inhibitor. In an even further aspect, the BCR-ABL tyrosine kinase inhibitor is a Aurora kinase inhibitor. In a yet further aspect, the BCR-ABL tyrosine kinase inhibitor is a KIT inhibitor. In a still further aspect, the BCR-ABL tyrosine kinase inhibitor is a ATP competitive BCR-ABL inhibitor. In an even further aspect, the BCR-ABL tyrosine kinase inhibitor is a non-ATP competitive BCR-ABL inhibitor.

In further aspect, the patient has been diagnosed with a need for treatment of a Philadelphia chromosome positive leukemia prior to the administering step. In a still further aspect, the method further comprises the step of identifying a patient in need of treatment of a Philadelphia chromosome positive leukemia.

In a further aspect, the patient has been diagnosed with a Philadelphia chromosome positive leukemia refractory to treatment with a BCR-ABL kinase inhibitor. In a still further aspect, the refractory Philadelphia chromosome positive leukemia comprises a BCR-ABL kinase domain mutation. In a yet further aspect, the refractory Philadelphia chromosome positive leukemia comprises a BCR-ABL mutation. In an even further aspect, the mutation is selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, 1418S, I418V, S438C, P441L, E450A, E450G, E450K, E450V, E453K, E453V, E459G, E459K, M472I, P480L, F486S, D504D, G514S, T240T, K247R, F311V, and E499E. In a still further aspect, the mutation is T315I.

In a further aspect, the Philadelphia chromosome positive leukemia is selected from acute lymphoblastic leukemia, acute myelogenous leukemia, and chronic myelogenous leukemia.

In a further aspect, the Philadelphia chromosome positive leukemia is chronic myelogenous leukemia (“CML”). In a yet further aspect, the chronic myelogenous leukemia is chronic phase CML. In an even further aspect, the chronic myelogenous leukemia is lymphoid blast phase CML. In a still further aspect, the chronic myelogenous leukemia is myeloid blast phase CML. In a yet further aspect, the chronic myelogenous leukemia is accelerated phase CML.

In a further aspect, the Philadelphia chromosome positive leukemia is acute lymphoblastic leukemia. In a still further aspect, the a Philadelphia chromosome positive leukemia is acute myelogenous leukemia.

In one aspect, the invention relates to a method for the treatment of a Philadelphia chromosome positive leukemia in a patient comprising the step of administering to the patient a therapeutically effective amount of a BCR-ABL tyrosine kinase inhibitor, and a therapeutically effective amount of at least one disclosed artemisinin analogue, or a pharmaceutically acceptable salt, hydrate, solvate, or polymorph thereof; wherein administering the artemisinin analogue to a patient produces a metabolite in the patient with a structure represented by the formula:

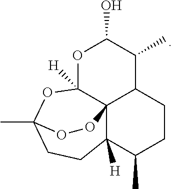

In a further aspect, the BCR-ABL tyrosine kinase inhibitor and the artemisinin analogue are co-administered. In a still further aspect, the BCR-ABL tyrosine kinase inhibitor is administered prior to administration of the artemisinin analogue. In a yet further aspect, the artemisinin analogue is administered prior to administration of the BCR-ABL tyrosine kinase inhibitor.

In a further aspect, the BCR-ABL tyrosine kinase inhibitor is administered in a first period; and the BCR-ABL tyrosine kinase inhibitor and the artemisinin analogue are co-administered in a second period. In a still further aspect, the BCR-ABL tyrosine kinase inhibitor administered in a first period and the BCR-ABL tyrosine kinase inhibitor administered in a second period are not the same. In a yet further aspect, the BCR-ABL tyrosine kinase inhibitor administered in a first period and the BCR-ABL tyrosine kinase inhibitor administered in a second period are the same.

In a further aspect, the Philadelphia chromosome positive leukemia after the first treatment period is refractory to treatment by the BCR-ABL tyrosine inhibitor. In a still further aspect, the Philadelphia chromosome positive leukemia during the first treatment period becomes refractory to treatment by the BCR-ABL tyrosine inhibitor.

In a further aspect, the Philadelphia chromosome positive leukemia after the first treatment period comprises a mutation selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, I418S, I418V, S438C, P441L, E450A, E450G, E450K, E450V, E453K, E453V, E459G, E459K, M472I, P480L, F486S, D504D, G514S, T240T, K247R, F311V, and E499E. In a still further aspect, the mutation is T315I.

In a further aspect, the BCR-ABL tyrosine kinase inhibitor is selected from imatinib, dasatinib, nilotinib, bosutinib, ponatinib, bafetinib, saracatinib, tozasertib and rebastinib. In a yet further aspect, the BCR-ABL tyrosine kinase inhibitor is selected from imatinib, dasatinib, nilotinib, bosutinib, ponatinib, bafetinib, saracatinib, and tozasertib. In a still further aspect, the BCR-ABL tyrosine kinase inhibitor is imatinib. In a yet further aspect, the BCR-ABL tyrosine kinase inhibitor is dasatinib. In an even further aspect, the BCR-ABL tyrosine kinase inhibitor is nilotinib. In a still further aspect, the BCR-ABL tyrosine kinase inhibitor is bosutinib. In a yet further aspect, the BCR-ABL tyrosine kinase inhibitor is ponatinib. In an even further aspect, the BCR-ABL tyrosine kinase inhibitor is bafetinib. In a still further aspect, the BCR-ABL tyrosine kinase inhibitor is saracatinib. In a yet further aspect, the BCR-ABL tyrosine kinase inhibitor is tozasertib.

In a further aspect, the BCR-ABL tyrosine kinase inhibitor is selected from a BCR-ABL selective inhibitor, a dual BCR-ABL and Src-family kinases inhibitor, Aurora kinase inhibitor, Kit inhibitor, ATP competitive inhibitor and non-ATP competitive BCR-ABL inhibitor. In a still further aspect, the BCR-ABL tyrosine kinase inhibitor is a selective BCR-ABL inhibitor. In a yet further aspect, the BCR-ABL tyrosine kinase inhibitor is a dual BCR-ABL and Src-family kinases inhibitor. In an even further aspect, the BCR-ABL tyrosine kinase inhibitor is a Aurora kinase inhibitor. In a yet further aspect, the BCR-ABL tyrosine kinase inhibitor is a KIT inhibitor. In a still further aspect, the BCR-ABL tyrosine kinase inhibitor is a ATP competitive BCR-ABL inhibitor. In an even further aspect, the BCR-ABL tyrosine kinase inhibitor is a non-ATP competitive BCR-ABL inhibitor.

In a further aspect, the patient has been diagnosed with a need for treatment of a Philadelphia chromosome positive leukemia prior to the administering step. In a still further aspect, the method further comprises the step of identifying a patient in need of treatment of a Philadelphia chromosome positive leukemia.

In a further aspect, the patient has been diagnosed with a Philadelphia chromosome positive leukemia refractory to treatment with a BCR-ABL kinase inhibitor. In a still further aspect, the refractory Philadelphia chromosome positive leukemia comprises a BCR-ABL kinase domain mutation. In a yet further aspect, the refractory Philadelphia chromosome positive leukemia comprises a BCR-ABL mutation. In an even further aspect, the mutation is selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, I418S, I418V, S438C, P441L, E450A, E450G, E450K, E450V, E453K, E453V, E459G, E459K, M472I, P480L, F486S, D504D, G514S, T240T, K247R, F311V, and E499E. In a still further aspect, the mutation is T315I.

In a further aspect, the Philadelphia chromosome positive leukemia is selected from acute lymphoblastic leukemia, acute myelogenous leukemia, and chronic myelogenous leukemia. In a still further aspect, the a Philadelphia chromosome positive leukemia is chronic myelogenous leukemia (“CML”). In a yet further aspect, the chronic myelogenous leukemia is chronic phase CML. In an even further aspect, the chronic myelogenous leukemia is lymphoid blast phase CML. In a still further aspect, the chronic myelogenous leukemia is myeloid blast phase CML. In a yet further aspect, the chronic myelogenous leukemia is accelerated phase CML.

In a further aspect, the Philadelphia chromosome positive leukemia is acute lymphoblastic leukemia. In a still further aspect, the Philadelphia chromosome positive leukemia is acute myelogenous leukemia.

b. Treatment of a Gastrointestinal Stromal Tumor

In one aspect, the present invention relates to methods for the treatment of a gastrointestinal stromal tumor (GIST) in a patient comprising the step of administering to the patient a therapeutically effective amount of a BCR-ABL kinase inhibitor and a therapeutically effective amount of at least one disclosed artemisinin analogue, or a pharmaceutically acceptable salt, solvate, or polymorph thereof

In a further aspect, the BCR-ABL tyrosine kinase inhibitor and the artemisinin analogue are co-administered. In a still further aspect, the BCR-ABL tyrosine kinase inhibitor is administered prior to administration of the artemisinin analogue. In a yet further aspect, the artemisinin analogue is administered prior to administration of the BCR-ABL tyrosine kinase inhibitor.

In a further aspect, the BCR-ABL tyrosine kinase inhibitor is administered in a first period; and the BCR-ABL tyrosine kinase inhibitor and the artemisinin analogue are co-administered in a second period. In a still further aspect, the BCR-ABL tyrosine kinase inhibitor administered in a first period and the BCR-ABL tyrosine kinase inhibitor administered in a second period are not the same. In a yet further aspect, the BCR-ABL tyrosine kinase inhibitor administered in a first period and the BCR-ABL tyrosine kinase inhibitor administered in a second period are the same.

In a further aspect, the after the first treatment period is refractory to treatment by the BCR-ABL tyrosine kinase inhibitor. In a still further aspect, the gastrointestinal stromal tumor during the first treatment period becomes refractory to treatment by the BCR-ABL tyrosine kinase inhibitor.

In one aspect, the invention relates to a method for the treatment of a gastrointestinal stromal tumor (GIST) in a patient comprising the step of administering to the patient a therapeutically effective amount of a BCR-ABL tyrosine kinase inhibitor, and a therapeutically effective amount of at least one disclosed artemisinin analogue, or a pharmaceutically acceptable salt, hydrate, solvate, or polymorph thereof; wherein administering the artemisinin analogue to a patient produces a metabolite in the patient with a structure represented by the formula:

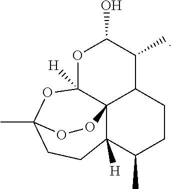

In a further aspect, the BCR-ABL tyrosine kinase inhibitor and the artemisinin analogue are co-administered. In a still further aspect, the BCR-ABL tyrosine kinase inhibitor is administered prior to administration of the artemisinin analogue. In a yet further aspect, the artemisinin analogue is administered prior to administration of the BCR-ABL tyrosine kinase inhibitor.

In a further aspect, the BCR-ABL tyrosine kinase inhibitor is administered in a first period; and the BCR-ABL tyrosine kinase inhibitor and the artemisinin analogue are co-administered in a second period. In a still further aspect, the BCR-ABL tyrosine kinase inhibitor administered in a first period and the BCR-ABL tyrosine kinase inhibitor administered in a second period are not the same. In a yet further aspect, the BCR-ABL tyrosine kinase inhibitor administered in a first period and the BCR-ABL tyrosine kinase inhibitor administered in a second period are the same.

In a further aspect, the gastrointestinal stromal tumor after the first treatment period is refractory to treatment by the BCR-ABL tyrosine inhibitor. In a still further aspect, the stromal tumor during the first treatment period becomes refractory to treatment by the BCR-ABL tyrosine inhibitor.

In a further aspect, the gastrointestinal stromal tumor after the first treatment period comprises a mutation. In a still further aspect, the mutation is a KIT mutation.

In a further aspect, the BCR-ABL tyrosine kinase inhibitor is selected from imatinib, dasatinib, nilotinib, bosutinib, ponatinib, bafetinib, saracatinib, tozasertib and rebastinib. In a yet further aspect, the BCR-ABL tyrosine kinase inhibitor is selected from imatinib, dasatinib, nilotinib, bosutinib, ponatinib, bafetinib, saracatinib, and tozasertib. In a still further aspect, the BCR-ABL tyrosine kinase inhibitor is imatinib. In a yet further aspect, the BCR-ABL tyrosine kinase inhibitor is dasatinib. In an even further aspect, the BCR-ABL tyrosine kinase inhibitor is nilotinib. In a still further aspect, the BCR-ABL tyrosine kinase inhibitor is bosutinib. In a yet further aspect, the BCR-ABL tyrosine kinase inhibitor is ponatinib. In an even further aspect, the BCR-ABL tyrosine kinase inhibitor is bafetinib. In a still further aspect, the BCR-ABL tyrosine kinase inhibitor is saracatinib. In a yet further aspect, the BCR-ABL tyrosine kinase inhibitor is tozasertib.

In a further aspect, the BCR-ABL tyrosine kinase inhibitor is selected from a BCR-ABL selective inhibitor, a dual BCR-ABL and Src-family kinases inhibitor, Aurora kinase inhibitor, Kit inhibitor, ATP competitive inhibitor and non-ATP competitive BCR-ABL inhibitor. In a still further aspect, the BCR-ABL tyrosine kinase inhibitor is a selective BCR-ABL inhibitor. In a yet further aspect, the BCR-ABL tyrosine kinase inhibitor is a dual BCR-ABL and Src-family kinases inhibitor. In an even further aspect, the BCR-ABL tyrosine kinase inhibitor is a Aurora kinase inhibitor. In a yet further aspect, the BCR-ABL tyrosine kinase inhibitor is a KIT inhibitor. In a still further aspect, the BCR-ABL tyrosine kinase inhibitor is a ATP competitive BCR-ABL inhibitor. In an even further aspect, the BCR-ABL tyrosine kinase inhibitor is a non-ATP competitive BCR-ABL inhibitor.

In a further aspect, the patient has been diagnosed with a need for treatment of a gastrointestinal stromal tumor prior to the administering step. In a still further aspect, the method further comprises the step of identifying a patient in need of treatment of a gastrointestinal stromal tumor.

In a further aspect, the patient has been diagnosed with a gastrointestinal stromal tumor refractory to treatment with a BCR-ABL kinase inhibitor. In a still further aspect, the refractory gastrointestinal stromal tumor comprises a mutation. In a yet further aspect, the mutation is a KIT mutation.

2. Manufacture of a Medicament

In one aspect, the invention relates to use of at least one disclosed compound in the manufacture of a medicament for the treatment of an uncontrolled cellular proliferative disorder in a patient. In a further aspect, the use is in the manufacture of a medicament for the treatment of a Philadelphia chromosome positive leukemia. In a further aspect, the use is in the manufacture of a medicament for the treatment of a gastrointestinal stromal cancer.

In a further aspect, the invention relates to a method for the manufacture of a medicament to overcome BCR-ABL tyrosine kinase inhibitor drug resistance in a patient comprising combining a therapeutically effective amount of a disclosed compound or product of a disclosed method with a pharmaceutically acceptable carrier or diluent.

In various aspect, the invention relates methods for the manufacture of a medicament for modulating the activity IL7 (e.g., treatment of one or more Philadelphia chromosome positive leukemias associated with cytokine-related BCR-ABL tyrosine kinase inhibitor drug resistance) in patients comprising combining one or more disclosed compounds, products, or compositions or a pharmaceutically acceptable salt, solvate, hydrate, or polymorph thereof, with a pharmaceutically acceptable carrier. It is understood that the disclosed methods can be performed with the disclosed compounds, products, and pharmaceutical compositions. It is also understood that the disclosed methods can be employed in connection with the disclosed methods of using.

It is understood that the disclosed methods can be performed with the disclosed compounds, products, and pharmaceutical compositions. It is also understood that the disclosed methods can be employed in connection with the disclosed methods of using.

3. Use of Compounds

It is understood that the disclosed uses can be employed in connection with the disclosed compounds, methods, compositions, and kits.

4. Kits

In one aspect, the invention relates to a kit comprising a BCR-ABL kinase inhibitor, at least one disclosed artemisinin analogue, or a pharmaceutically acceptable salt, solvate, or polymorph thereof; and instructions for treatment of a disease of uncontrolled cellular proliferation; and wherein the instructions comprise administration of a BCR-ABL kinase inhibitor and the at least one artemisinin analogue to a patient that has become refractory to treatment with a BCR-ABL kinase inhibitor.

In a further aspect, the BCR-ABL kinase inhibitor and the at least one disclosed artemisinin analogue are co-formulated. In a still further aspect, the BCR-ABL kinase inhibitor and the at least one disclosed artemisinin analogue are co-packaged

In a further aspect, the instructions further comprise co-administration of the BCR-ABL tyrosine kinase inhibitor and the artemisinin analogue. In a still further aspect, the instructions further comprise administration of the BCR-ABL tyrosine kinase inhibitor is prior to administration of the artemisinin analogue. In a yet further aspect, the instructions further comprise administration of the artemisinin analogue prior to administration of the BCR-ABL tyrosine kinase inhibitor.

In a further aspect, the instructions further comprise administration of the BCR-ABL kinase inhibitor and the at least one disclosed artemisinin analogue to a patient with a Philadelphia chromosome positive leukemia.

In a further aspect, the instructions further comprise administration of the BCR-ABL tyrosine kinase inhibitor is administered in a first period; and co-administration of the BCR-ABL tyrosine kinase inhibitor and the artemisinin analogue in a second period. In a still further aspect, the instructions further comprise that the BCR-ABL tyrosine kinase inhibitor administered in a first period and the BCR-ABL tyrosine kinase inhibitor administered in a second period are not the same. In a yet further aspect, the instructions further comprise that the BCR-ABL tyrosine kinase inhibitor administered in a first period and the BCR-ABL tyrosine kinase inhibitor administered in a second period are the same.

In a further aspect, the instructions further comprise administration of the BCR-ABL kinase inhibitor and the at least one disclosed artemisinin analogue to a patient with a Philadelphia chromosome positive leukemia in the first treatment period.

In a further aspect, the instructions further comprise administration to a patient that is refractory to treatment by the BCR-ABL tyrosine inhibitor after the first treatment period. In a still further aspect, the instructions further comprise that the Philadelphia chromosome positive leukemia after the first treatment period comprises a mutation selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, I418S, I418V, S438C, P441L, E450A, E450G, E450K, E450V, E453K, E453V, E459G, E459K, M472I, P480L, F486S, D504D, G514S, T240T, K247R, F311V, and E499E. In a yet further aspect, the mutation is T315I.

In a further aspect, the BCR-ABL tyrosine kinase inhibitor is selected from imatinib, dasatinib, nilotinib, bosutinib, ponatinib, bafetinib, saracatinib, tozasertib and rebastinib. In a yet further aspect, the BCR-ABL tyrosine kinase inhibitor is selected from imatinib, dasatinib, nilotinib, bosutinib, ponatinib, bafetinib, saracatinib, and tozasertib. In a still further aspect, the BCR-ABL tyrosine kinase inhibitor is imatinib. In a yet further aspect, the BCR-ABL tyrosine kinase inhibitor is dasatinib. In an even further aspect, the BCR-ABL tyrosine kinase inhibitor is nilotinib. In a still further aspect, the BCR-ABL tyrosine kinase inhibitor is bosutinib. In a yet further aspect, the BCR-ABL tyrosine kinase inhibitor is ponatinib. In an even further aspect, the BCR-ABL tyrosine kinase inhibitor is bafetinib. In a still further aspect, the BCR-ABL tyrosine kinase inhibitor is saracatinib. In a yet further aspect, the BCR-ABL tyrosine kinase inhibitor is tozasertib.

In a further aspect, the instructions further comprise the patient has been diagnosed with a need for treatment of a Philadelphia chromosome positive leukemia prior to the administering step. In a still further aspect, the instructions further comprise identifying a patient in need of treatment of a Philadelphia chromosome positive leukemia.

In a further aspect, the instructions further comprise the patient has been diagnosed with a Philadelphia chromosome positive leukemia refractory to treatment with a BCR-ABL kinase inhibitor. In a still further aspect, the refractory Philadelphia chromosome positive leukemia comprises a BCR-ABL kinase domain mutation. In a yet further aspect, the refractory Philadelphia chromosome positive leukemia comprises a BCR-ABL mutation. In an even further aspect, the mutation is selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, I418S, I418V, S438C, P441L, E450A, E450G, E450K, E450V, E453K, E453V, E459G, E459K, M472I, P480L, F486S, D504D, G514S, T240T, K247R, F311V, and E499E. In a still further aspect, the mutation is T315I.

In a further aspect, the Philadelphia chromosome positive leukemia is selected from acute lymphoblastic leukemia, acute myelogenous leukemia, and chronic myelogenous leukemia. In a still further aspect, the a Philadelphia chromosome positive leukemia is chronic myelogenous leukemia (“CML”). In a yet further aspect, the chronic myelogenous leukemia is chronic phase CML. In an even further aspect, the chronic myelogenous leukemia is lymphoid blast phase CML. In a still further aspect, the chronic myelogenous leukemia is myeloid blast phase CML. In a yet further aspect, the chronic myelogenous leukemia is accelerated phase CML.

In a further aspect, the Philadelphia chromosome positive leukemia is acute lymphoblastic leukemia. In a still further aspect, the Philadelphia chromosome positive leukemia is acute myelogenous leukemia.

In a further aspect, the instructions further comprise identifying a patient in need of treatment of a gastrointestinal stromal tumor. In a still further aspect, instructions further comprise the patient has been diagnosed with a gastrointestinal stromal tumor refractory to treatment with a BCR-ABL kinase inhibitor. In a yet further aspect, the refractory gastrointestinal stromal tumor comprises a mutation. In an even further aspect, the refractory gastrointestinal stromal tumor comprises a KIT mutation.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

### F. Experimental

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. General Methods

a. Production and Culture of Murine Leukemia Initiating Cells (LICs)

Replication-defective mouse stem cell virus (MSCV) retroviral vectors co-expressing either the wild-type (WT) allele of human p185^{BCR-ABL} or mutant BCR-ABL alleles p185^{T315I}or p185^{F317L}, and either green fluorescent protein (GFP) or luciferase, were packaged into replication-incompetent ecotropic virions. Whole bone marrow cell suspensions from Arf^{-/-} young adult mice were infected to produce Arf ^{-/- p}185+ pre-B cells. After initial establishment on autologous stromal cell layers over 7 days, transformed pre-B cells were briefly expanded in the absence of a stromal layer for 2 days in liquid culture in basal culture media (BCM10) (RPMI1640 supplemented with 10% Hyclone fetal calf serum, 4 mM glutamine, 100 Units/mL penicillin, 100 μg/mL streptomycin, and 55 μM beta-mercaptoethanol), and cryopreserved. Prior to use in any assays, Arf^{-/-} p185^{WT} and Arf^{-/-} p185^{T315I} pre-B cells (BCR-ABL^{WT} leukemia initiating cells (LICs) and BCR-ABL^{T315I} LICs, respectively) were thawed and allowed to recover in BCM10 for 3 days.

b. In Vivo Adoptive Leukemia Transfer PH+ Model

Mice were housed in an American Association of Laboratory Animal Care (AALAC)-accredited facility and treated on Institutional Animal Care and Use Committee (IACUC)-approved protocols in accordance with NIH guidelines. Adoptive cell transfers were performed by injecting LICs into tail veins of healthy, non-conditioned, immune-competent 10 to 12-week-old IL7^{-/-} or IL7^{-/-}C57B1/6J mice (Jackson Labs, Bar Harbor, Me.). Animals were observed daily and sacrificed when moribund (dehydration, ruffled fur, poor mobility, respiratory distress). Survival curves were generated using GraphPad Prism Version 5.0 (La Jolla, Calif.). The Mantel-Cox test was applied to pairwise comparisons of survival data.

C. Development of LIC-Based Screening Assay

Before use in any in vitro assay, LICs had to pass the following quality control parameters: test thawing (benchmark: 95% viability at 24 h); PCR genotyping to confirm Arf^{-/-} status; mycoplasma testing (negative by Takara Bio Mycoplasma PCR assay, Clontech, Madison Wis.); sequence verification of BCR-ABL allele; and in vitro dasatinib potency confirmation.

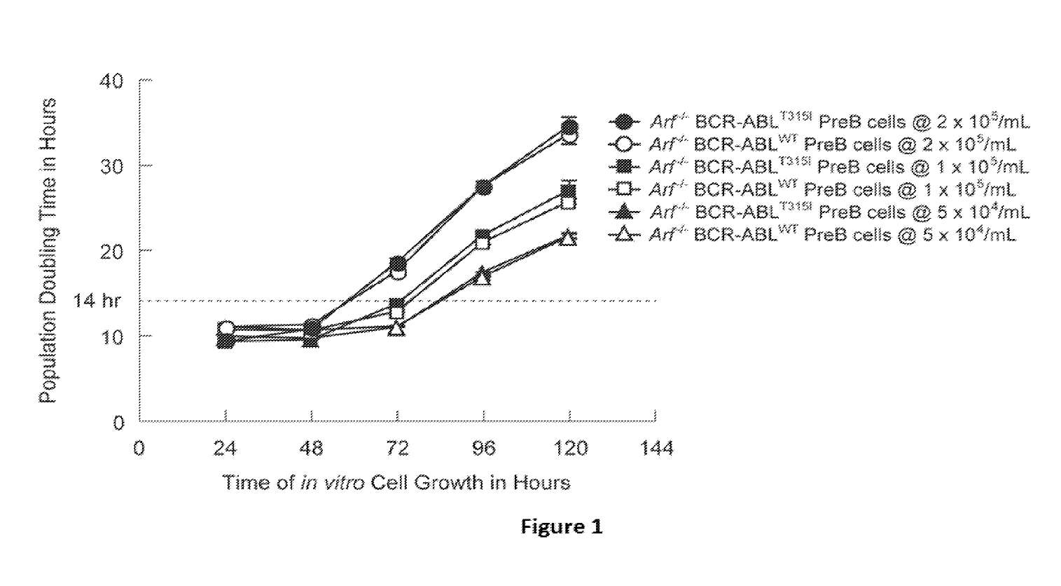

The maximum dimethyl sulfoxide (DMSO) concentration tolerated by LICs in culture was determined to be 0.2% (by volume) and was maintained at ≦0.1% (by volume) in all subsequent work. LICs were plated in 384-well micro-titer plates at a cellular density of 5×10⁴ per mL (1250 LICs per well in 25 μL BCM10) and confirmed to give exponential growth during first 72 h. FIG. 1 shows the growth properties of Arf^{-/-} p185^{WT} (filled symbols) and Arf^{-/-} P185^{T315I} preB (empty symbols) populations during in vitro cell culture. Both cell types were plated at multiple cell dilutions (n=3 for each condition, time=0 h) in 6-well plates and serially followed daily for 5 days to evaluate absolute viable cell count, total viability, doubling times, and total population doublings, using the Beckman Coulter cell counter (trypan blue staining) and fluorescence-activated cell sorting (FACS) analysis in parallel. Both cell types had identical growth properties. Only the comparison for population doubling time in hours (+/−sd) for 3 relevant plating densities is presented. FACS cell-cycle analysis confirmed that a population doubling time less than 14 h was sufficient to maintain cells in exponential growth (>50% cells in S-phase).

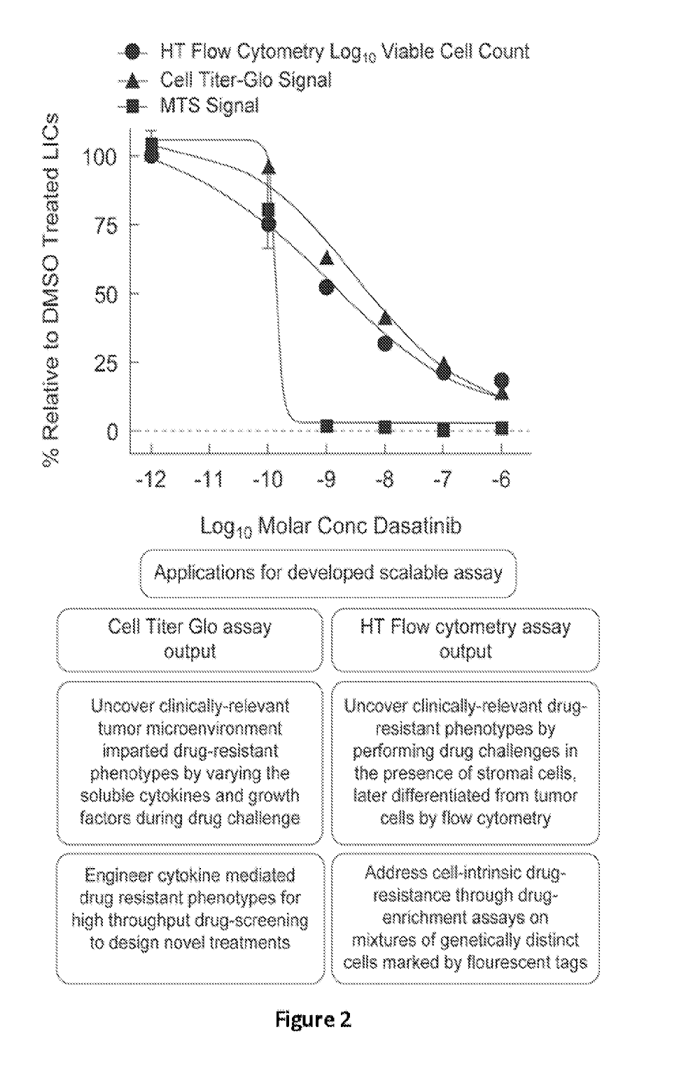

To select assay reagents, three high throughput screening (HTS) relevant cellular assays were evaluated according to manufacturer's instructions. As represented in FIG. 2, BCR-ABL^{WT} LICs plated at 5×10⁴ per mL were challenged with 6 log-fold concentration range of dasatinib or DMSO in 384-well microplates. After a 72 h drug challenge, LIC growth was measured by the 3 cellular readouts: CellTiter-Glo® (CTG) assay (ATP content), MTS (3-(4,5-dimethylthiazol-2-yl)-5-(3-carboxymethoxyphenyl)-2-(4-sulfophenyl)-2H-tetrazolium) assay (viability), and high-throughput flow cytometry (total viability and viable cell counts by DAPI exclusion). Measured assay readouts at various drug concentrations (x-axis) were normalized to DMSO-treated LICs and graphed (+/−sd) on y-axis. Total viable LIC counts were determined by FACS, reflecting dasatinib-induced phenotypic changes across a 6 log-fold drug-concentration, were closely paralleled by the CTG cellular assay output but not by the commonly used MTS assay.

The luminescent CTG (CellTiter-Glo; Promega Madison, Wis.) assay, which monitors cellular ATP content, was inherently more sensitive than the colorimetric MTS assay, which monitors cellular reductive capacity. The CTG assay accurately detected low number of LICs producing results comparable to those obtained by flow cytometry, and provided ease of use in HTS (no prolonged incubation step required between reagent addition and assay readout). When carried out after 72 h, the CTG assay had a 10,000 fold dynamic range, low signal variance (Z′=0.9 in semi-automated mode, and Z′=0.8 in fully automated mode), displayed minimal drift or edge effects, and had robust inter-plate and inter-day reproducibility.

d. Forward Cytokine Phenotypic Screen

Lyophilized murine and human recombinant cytokines (R&D Systems, Minneapolis, Minn.) were diluted with 1 mg/mL bovine serum albumin (BSA) in sterile phosphate buffered saline (PBS) to a final concentration 20 μg/mL and stored at 4° C. Anti-leukemia drugs, BCR-ABL-TKIs, and non-BCR-ABL-specific-TKIs (LC labs, Sigma), were solubilized in DMSO to obtain 10 mM stocks concentrations. Subsequent serial titrations were prepared using DMSO in 0.5 mL vials or 384-well drug master plates, and stored at −20° C.

To study the impact of murine cytokines on dasatinib action, LIC stocks containing different concentrations of candidate cytokines were prepared in multi-channel sterile reservoirs and plated into 384-well plates. Drug delivery to cell suspensions was accomplished with a V & P Scientific (San Diego, Calif.) pin. For murine-human comparisons, human SUP-B 15 Ph+ ALL cells (DSMZ cell culture collection, Germany) in McCoy's media containing 20% fetal calf serum and penicillin/streptomycin (Invitrogen, Carlsbad, Calif.), were plated at 12,500 cells in 25 μL per well in black clear-bottom 384-well microtiter plates. Drugs were transferred by pin-transfer. After 72 h incubation at 37° C., 20% (by volume) MTS assay reagent (CellTiter 96 Aqueous One Solution, Promega, Madison, Wis.) was added, microtiter plates were incubated for 75 min at 37° C., and read colorimetrically at 490 nm.

e. High-Throughput (HT) Phenotypic Drug Screening

An IL7 concentration of 0.85 ng/mL was confirmed using cytokine titration assays to confer near-maximal and reproducible resistance against nilotinib and imatinib in BCR-ABL^{WT} LICs. A primary screening of 5600 compounds including FDA approved drugs was then carried out at a fixed dose (10 μM). Active compounds were retested to establish dose-response relationships using a multi-step, 2-fold dilution scheme (Table 1). Multiple control assay plates were set-up during all screening runs to allow for inter-plate and inter-day comparisons at 0, 24, 48, and 72 hrs. Table 1 below shows steps used in the optimized high-throughput screening assay.

TABLE 1

| Step | Parameter | Value | Description |
|---|---|---|---|
| 1 | Plate cells | 25 μL | Plate LICs 5 × 10⁴ per mL (25 μl/well) in BCM10 + 0.85 |
| | | | ng/mL IL7 into 384-well opaque bottom white |
| | | | microplates using Wellmate (Matrix) |
| 2 | Primary drug screening | 25 nL | 1A-1I: Nine dasatinib concentrations (100 nM, 30 nM, |
| | controls in columns 1-2 | | 10 nM, 3 nM, 1 nM, 0.3 nM, 0.1 nM, 0.03 nM and 0.01 |
| | and 13-14 | | nM)-control for IL7-and BCR-ABL mutation- |
| | | | imparted resistance against BCR-ABL-TKIs |
| | | | 2A-2I: Nine staurosporine concentrations (300 nM, 100 |
| | | | nM, 60 nM, 30 nM, 10 nM, 6 nM, 3 nM, 1 nM, 0.6 |
| | | | nM)-non-BCR-ABL-specific reference control in the |
| | | | presence of IL7 |
| | Secondary drug screening | 25 nL | 13A-13P, 1J, 2J: DMSO 0.1% (Max Signal OR |
| | controls in columns | | Negative Control); 14A-14P: staurosporine at 300 nM |
| | 21-24 | | (Min Signal OR Positive Control); 1K-1P, 2K-2P: 40 |
| | | | μM dasatinib |
| | | | 21A-21P: DMSO 0.1% (Max Signal); 22A-22P, 23K- |
| | | | 24P, 24K-24P: staurosporine at 300 nM (Min Signal) |
| | | | 23A-23J: Staurosporine-10 log-fold dilutions (3 μM to |
| | | | 0.3 pM)-non-BCR-ABL-specific reference control in |
| | | | the presence of IL7 |
| | | | 24A-24J: Dasatinib: 10 log-fold dilutions (1 μM to 0.1 |
| | | | pM)-control for IL7-and BCR-ABL mutation- |
| | | | imparted BCR-ABL-KI drug-resistance |
| 3 | Add test compounds in | 25 nL | Primary screening: Single final drug concentration of 10 |
| | columns 3-12 and | | μM |
| | 15-24 in primary; | | Secondary screening: Triplicate ten half-log serial |
| | columns 1-20 | | dilutions (5 μM to 2 nM final concentration) of primary |
| | in secondary screen | | screening hits to determine their half-maximal |
| | | | inhibitory concentrations (IC₅₀) against LICs |
| | | | 384-well master drug-stock plates prepared at 1000× in |
| | | | DMSO. Drug delivered to assay plates by pin transfer |
| | | | (using V&P Scientific pin tool, 10 H pins), giving a final |
| | | | drug concentration of 1× and 0.1% DMSO in all control |
| | | | and test wells of assay plates |
| 4 | Incubation 1 | 72 h | Liconics incubator, 8% CO₂, 37° C. (std. LIC culture |
| | | | conditions) |
| 5 | Incubation 2 | 20 min | Room temp, low light (instructions as per Promega) |
| 6 | Add reagent | 25 μL | CellTiter-Glo ® (Promega) pre-equilibrated to RT |
| | | | before use |
| 7 | Incubation 3 | 25 min | Room temp, low light (per Promega) |
| 8 | Assay readout | ATP | Envision, Luminescent mode-ATP levels as a measure |
| | | levels | of LIC number and viable growth in 72 h-Signal |
| | | | measured in RLUs |
| 9 | Data processing | Pipeline | % inhibition by test compound = 100* (Log₁₀Test |
| | | Pilot | Signal − Log₁₀Min Signal mean)/( Log₁₀Min Signal |
| | | | mean − Log₁₀Max Signal mean) |
| | | | Z-prime (Z′) = 1-[(3*stdev of Log₁₀ Max Signal + |
| | | | 3*stdev of Log₁₀ Min Signal)/(Log₁₀Max Signal mean − |
| | | | Log₁₀Min Signal mean)] Z′ is a dimensionless |
| | | | calculation to assess assay quality-for a good assay |
| | | | Z′ ≧ 0.5, and for a perfect assay Z′ = 1 |

f. In Vivo Preclinical Therapeutic Studies

For in vivo use, dasatinib (LC Labs, Woburn, Mass.) in citric acid (pH 3.1), and DHA (Avachem, San Antonio, Tex.) in 0.5% carboxy-methylcellulose/0.5% Tween 80/0.5% benzyl alcohol, were administered by oral gavage. In toxicity studies, DHA was ranged up to 300 mg/kg as a single or split dose for 5 days/week over a six week time period. Repeated single doses of 300 mg/kg (5 days/week) were determined to induce no significant weight loss, lethargy, seizures or deaths. During therapeutic studies, animal weights were monitored daily to ensure no significant body weight reductions.

g. Bioluminescent Imaging and Evaluation of Cures

Bioluminescent imaging and analysis were performed using a Xenogen IVIS-200 system and Living Image software 3.01 (Caliper Life Sciences, Hopkinton, Mass.). Total bioluminescent flux measurements (photons/second) were quantified over the whole animal body. Recipient mice that remained clinically healthy 12 months after terminating therapy and had no detectable bioluminescent signal in vivo were designated “long-term survivors.”

h. Assessment of LIC Viability

Viability of LICs was estimated after counterstaining approximately 5×10⁵ LICs (in 0.5 mL volume) with 35 μL of a propidium iodide (PI) solution (0.25 mg/mL in PBS). The percentage of viable cells was calculated by electronic gating on PI-negative cells on PI versus forward scatter dot plots, with forward scatter signals consistent with LICs, and comparison of this number with total cell number. Data were collected and analyzed on a BD Biosciences LSR II flow cytometer (San Jose, Calif.), using BD Biosciences FACS DiVa software.

i. Assessment of Cell Cycle Percentages

For determination of percentages in cell cycle phases, 5×10⁵ LICs were centrifuged, washed once with PBS, recentrifuged, and cell pellets were resuspended in 0.5 mL PI staining solution (0.05 mg/mL PI, 0.1% sodium citrate, 0.1% Triton X100). This process lysed the cells and stained the nuclear DNA with PI. Samples were then treated with DNAse-free RNAse (0.2 mg/mL in 10 M Tris-HCl/15 mM NaCl, pH 7.5) for 30 min at room temperature, filtered, and analyzed for red fluorescence from a PI-labeled DNA on BD Biosciences FACS Calibur flow cytometer (San Jose, Calif.) by using BD Biosciences CellQuest Pro software. The percentages of cells within each cell cycle phase were computed using the computer program ModFit (Verity Software House, Topsham, Me.).

j. Drug/Cytokine Washout and Cytokine Recovery Studies

For washout studies, BCR-ABL^{WT} LICs were diluted to a density of 1×10⁵ LICs/mL in BCM-10 containing no or 5 mg/mL IL7, and treatment with either dasatinib or 0.1% DMSO by volume (non-drug treated controls) was initiated at time 0 h in triplicate 100 mm petri dishes. Daily monitoring for expected drug-induced changes was performed by naked-eye microscopy from 0 to 72 h. At the 72 h time point, after harvesting under identical conditions, total LIC density and viability percentage for all samples were determined using Beckman Coulter Vi-cell (trypan blue) in triplicates. Each sample was spun down in 50 mL BD Falcon™ tubes; the supernatant was discarded and LIC pellet resuspended in freshly prepared BCM10 (not containing any cytokine or drug). This washing step was repeated four times to ensure complete removal of the drug and/or cytokine. After the last wash, each pellet was resuspended to 1×10⁶ total LICs per mL master stocks. Aliquots from master stocks were taken for (i) flow assessment of viability and cell cycle percentages (as described above) and (ii) manual CTG assay using 25 μL sample per well in 384-well microplates (in triplicates), as an independent determination for LIC growth. For assessment of extracellular cytokine on recovery of LICs that were previously challenged with dasatinib (0 to 72 h), washed master stocks were diluted to 5×10⁵ total LICs per mL in BCM10 with or without 5 ng/mL IL7, plated in 6 well plates (approximately 10 cm²), and incubated at 37° C. from 72 to 144 h for recovery, during which time daily naked-eye microscopic examinations were performed. At 144 h, for each sample total LIC density and viability percentage assessment by Beckman, viability and cell cycle percentages by flow assessment and independent CTG evaluations were performed as described for the 72 h time point. Total viable LICs in a sample were calculated as a product of absolute LIC count determined by Beckman and absolute sample viability determined by FACS analysis.

k. Dasatinib Re-Challenge Studies

Further studies were performed to evaluate the possibility of a genetically drug-resistant subpopulation that could undergo selection with dasatinib/IL7 co-treatment during 0-72 h of dasatinib/cytokine washout studies above. To this aim, BCR-ABL^{WT} LICs were treated with 100 nM dasatinib or 0.1% DMSO in the absence or presence of 5 ng/mL IL7 in 100 mm dishes from 0-72 h, as described above. In addition, a dasatinib challenge (first-time challenge) across 6 log-fold concentration in the presence or absence of 5 ng/mL IL7 was also performed using CTG assay during this time period (0-72 h). At 72 h, dasatinib/IL7 co-treated LICs from 100 mm dishes were washed (as described above) and immediately challenged (second challenge) from 72 to 144 h with six log-fold concentration range of dasatinib in 384-well microplates either in the presence or absence of 5 ng/mL IL7 using CTG assay.

l. Description of Chemical Screening Library

The screening library consisted of 5600 (approximately 3200 unique) approved drugs and chemicals with known biological activity (bioactives). The library was assembled from 3 commercial suppliers: Microsource, Prestwick, and Sigma. The Microsource compounds included the following: (a) the Spectrum collection, which contains 2000 biologically active and structurally diverse compounds, including known drugs, experimental bioactives, and pure natural products; (b) the U.S. Drug Collection, which contains 1040 drugs that have reached clinical trials in the US and have been assigned USAN or US Pharmacopeia status; and (c) the Killer collection, which contains a reference set of 160 synthetic and natural toxic substances (http://www.msdiscovery.com/index.html). The Prestwick compounds include 1120 small molecules selected for high chemical and pharmacologic diversity. Ninety percent of the collection is composed of known marketed drugs, and the remainder includes bioactive alkaloids or related substances. Sigma LOPAC1280 (Library of Pharmacologically Active Compounds) collection reflects the most commonly screened targets in the drug discovery community, including marketed drugs, failed development candidates, and “gold standards” that have well-characterized activities (http://www.sigmaaldrich.com/chemistry/drug-discovery/validation-libraries.html).

m. Data Processing, Quality Control and Hit Scoring Criteria

All data processing and visualization was performed using custom programs written in the Pipeline Pilot platform (Accelrys, v.7.5) and the R program 6.6. The R drc package was used to fit sigmoidal curves. Receiver operating characteristic (ROC) statistics were computed using the ROCR package.

The quality of the primary drug-screening studies, performed in parallel against BCR-ABL^{WT} and BCR-ABL^{T315I} LICs, was evaluated by multiple methods including, but not limited to, identification of known antileukemic agents, identification of multiple compound replicates intentionally included in the 5600 collection, and Z′ and other screening quality metrics. An empirically determined, statistically significant but liberal cut-off of >10% activity was chosen to include agents of relatively lower activity considering the high-risk nature of Ph+ ALL and the drug-rich nature of the library as well as to allow for a more complete structure-activity relationship and therapeutic class analysis after subsequent potency determination through secondary screening.

The discriminatory power of the phenotypic assay was assessed using receiver operating characteristic (ROC) statistics. A total of 165 compounds were selected to sample the primary assay according to the distribution of observed activities.

n. Cluster Analysis of Hits Using Therapeutic Classes

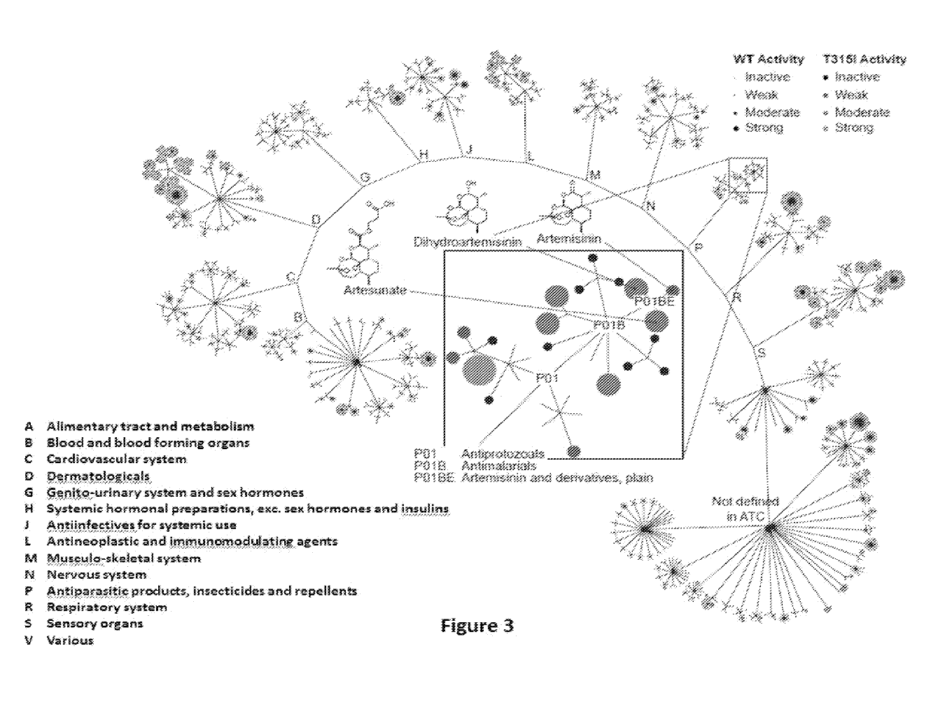

The therapeutic cluster graph was generated by first assigning each compound an Anatomical Therapeutic Chemical Classification System (ATC). This classification system groups drugs into a five level hierarchy according to therapeutic indication and chemical properties. The first level indicates anatomical main group and consists of one letter; the second level indicates therapeutic main group and consists of two digits; the third level indicates therapeutic or pharmacological subgroup and consists of one letter; the fourth level indicates chemical, therapeutic or pharmacological subgroup and consists of one letter; and the fifth level indicates the chemical substance and consists of two digits. The resulting seven characters constitute an ATC code, and a compound can be assigned multiple ATC codes. ATC codes can be depicted as a network graph by defining a node to represent each level in the ATC code and then connecting nodes according to parent-child relationships (e.g., first-level node A is the parent of second level nodes A01, A02, etc.). Compounds without an ATC code were assigned based on similarity to known agents, or were assigned to custom codes located under the parent V99 (labeled as “Not defined in ATC” in FIG. 3). The resulting network, as shown in FIG. 3, was visualized in Cytoscape® (v 2.8.1) using the yFiles circular layout algorithm.

Descriptions and legends for data presented in the following sections and figures are shown in Table 2 below.

TABLE 2

| Figure Data | Description | Legend |
|---|---|---|
| Host Factors | The effect of 15 | Host Factor: cytokine name |
| | leukemia-microenvironment | Conc: cytokine concentration |
| | relevant cytokines | Hill: hill slope |
| | on dasatinib | EC₅₀: dasatinib EC₅₀ (nM) |
| | potency (EC₅₀) | EC₅₀CI: = EC₅₀ 95% confidence interval |
| | against Arf-/-BCR- | r²: r-squared of the non-linear regression |
| | ABLWT LICs | |
| Structure | Structural | Reg Number: St. Jude registration root number |
| | information for 706 | Molsmiles: molecular SMILES |
| | compounds | Synonym: alternate names for compound |
| Screen | List of 15 | Agent: Name of clinical anti-leukemic agent |
| Summary (1) | clinically-used | WT EC50: mouse WT BCR-ABL B-cell EC₅₀(mM) |
| | conventional anti-leukemic | WT EC50 CI: WT EC₅₀ 95% confidence interval |
| | agents validated during | WT Act Class: WT activity classification |
| | dose-response screening | WT Curve Score: grade for quality of WT dose- |
| | experiments | response curve |
| | | T3151 EC50: mouse T315I BCR-ABL B-cell |
| | | EC₅₀(mM) |
| | | T315I EC50 CI: T315I EC₅₀ 95% confidence interval |
| | | T315I Act Class: T315I activity classification |
| | | T315I Curve Score: grade for quality of the T3151 |
| | | dose-response curve |
| Screen | Complete dose-response | WT EC50: mouse WT BCR-ABL B-cell EC₅₀(mM) |
| Summary (2) | screening data for 706 | WT EC50 CI: WT EC₅₀ 95% confidence interval |
| | compounds | WT Act Class: WT activity classification |
| | | WT Curve Score: grade for quality of WT dose-response curve |
| | | T315I EC50: mouse T315I BCR-ABL B-cell |
| | | EC₅₀(mM) |
| | | T315I EC50 CI: T315I EC₅₀ 95% confidence interval |
| | | T315I Act Class: T315I activity classification |
| | | T315I Curve Score: grade for quality of the T315I |
| | | dose-response curve |
| ATC | Anatomical | ATC L1: ATC 1st level (anatomical main group) |
| Classification | Therapeutic | ATC L2: ATC 2nd level (therapeutic main group) |
| | Chemical (ATC) | ATC L3: ATC 3rd level (therapeutic/pharmacological subgroup) |
| | Classification for | ATC L4: ATC 4th level |
| | 706 compounds | (chemical/therapeutic/pharmacological subgroup) |

2. Identification of Which Leukemia-Microenvironment Cytokines Induce Drug-Resistance Against BCR-ABL-TKIs

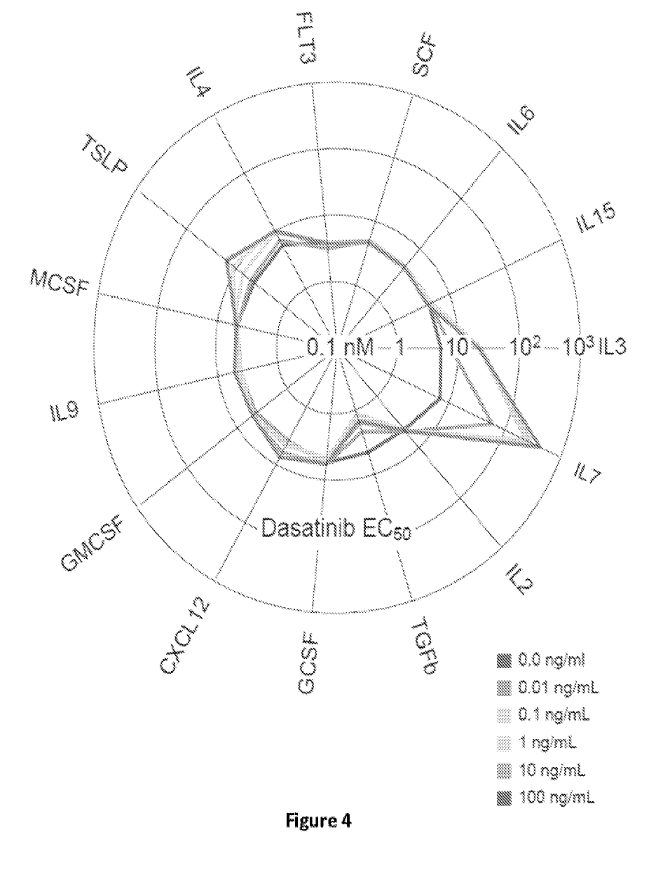

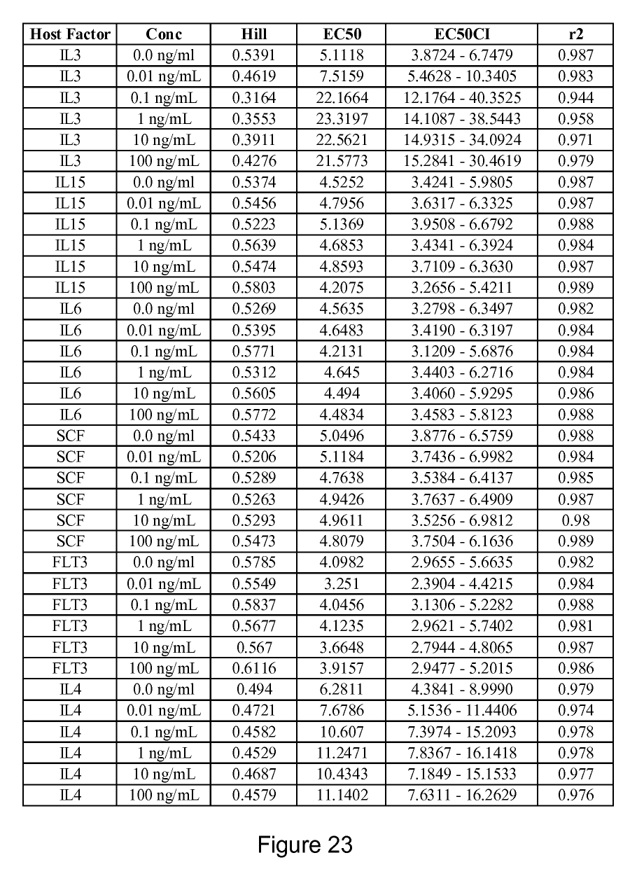

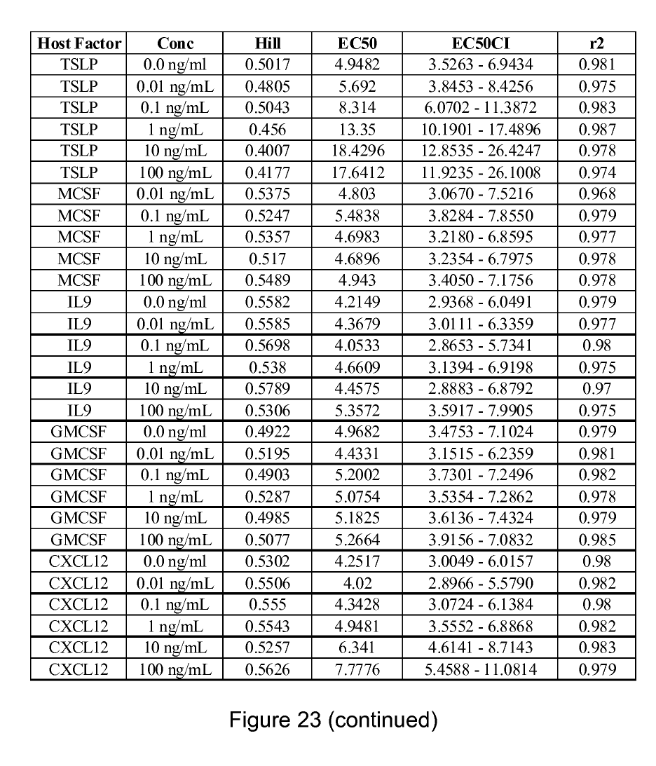

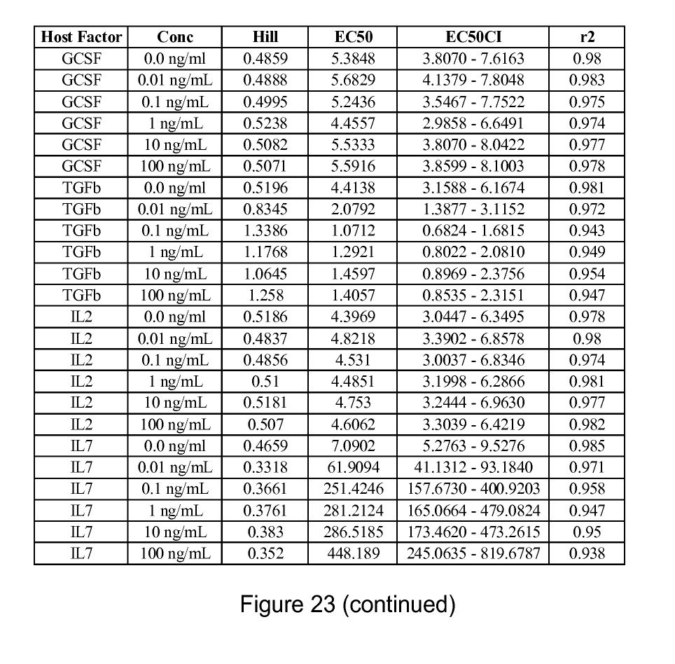

BCR-ABL^{WT} LICs are transformed primary Arf^{-/-} pre-B cells that retain phenotypic and signaling properties essential for suspended leukemic blasts to interact with the surrounding host-environment not typically present in established leukemic cell lines that have been extensively subjected to long term culture. As shown in FIG. 23, fifteen leukemia-microenvironment cytokines were evaluated for their ability to protect murine BCR-ABL^{WT} LICs from dasatinib, the most potent clinical BCR-ABL inhibitor. CTG assay measured LIC populations after 72-h dasatinib exposures in presence of increasing concentrations of indicated cytokines. FIG. 4 shows a radar plot representing the effect of the fifteen hematopoietic cytokines on dasatinib potency against Arf^{-/-} BCR-ABL^{WT} LICs. Each spoke in FIG. 4 represents an individual cytokine and each line graph represents dasatinib proliferation inhibition EC₅₀(nM) at the indicated titration of the parent cytokine. IL7 was observed to provide the strongest protection against dasatinib. In addition, IL3, thymic stromal lymphopoietin (TSLP, IL7-like cytokine), and IL4 appeared to confer some resistance, but with greatly reduced potency. In contrast, TGF-β appeared to enhance dasatinib activity.

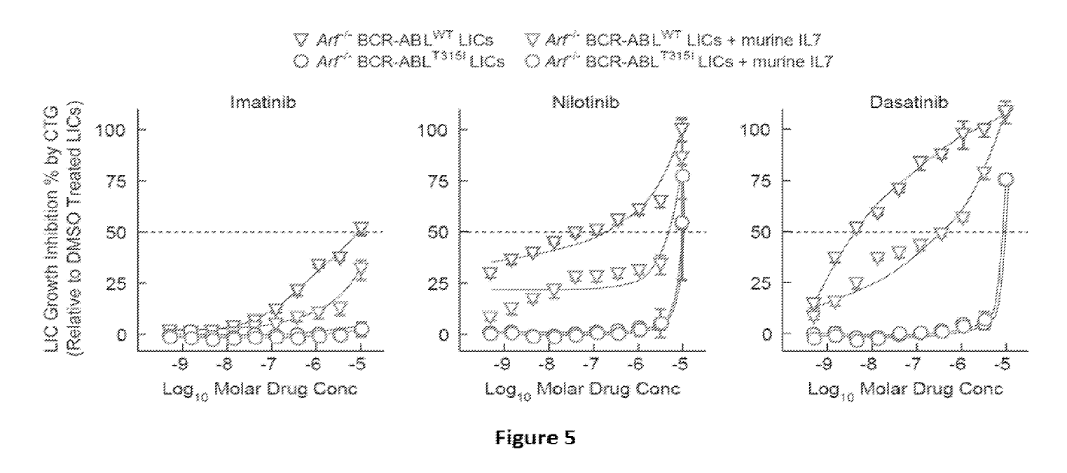

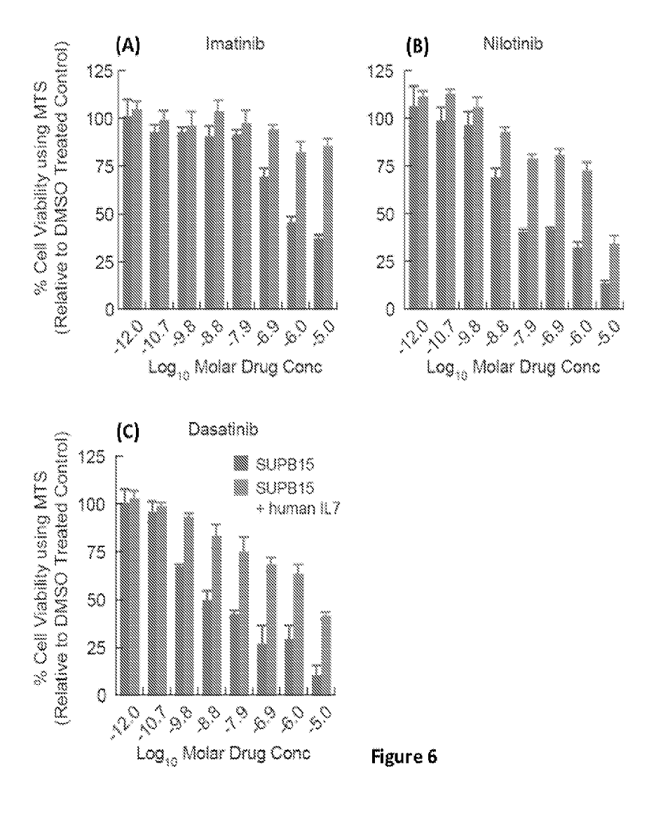

As represented in FIG. 5, Arf^{-/-} BCR-ABL^{WT} (triangles) and Art BCR-ABL^{T315I} (circles) LICs were treated with drug or DMSO for 72 hours in the absence (dark gray) or presence (light gray) of 1 ng/mL murine IL7. LIC growth inhibition was measured by CTG assay, normalized to DMSO-treated LICs and fit using nonlinear regression (+/−sd). As represented in FIG. 6, Human Ph+ ALL SUPB15 cells were also treated with drug or DMSO for 72 hours in absence or presence of 25 ng/mL human IL7. IL7 was observed to attenuate the potency of all FDA-approved BCR-ABL-TKIs against murine BCR-ABL^{WT} LICs and the human Ph+ ALL cell line SUP-B 15.

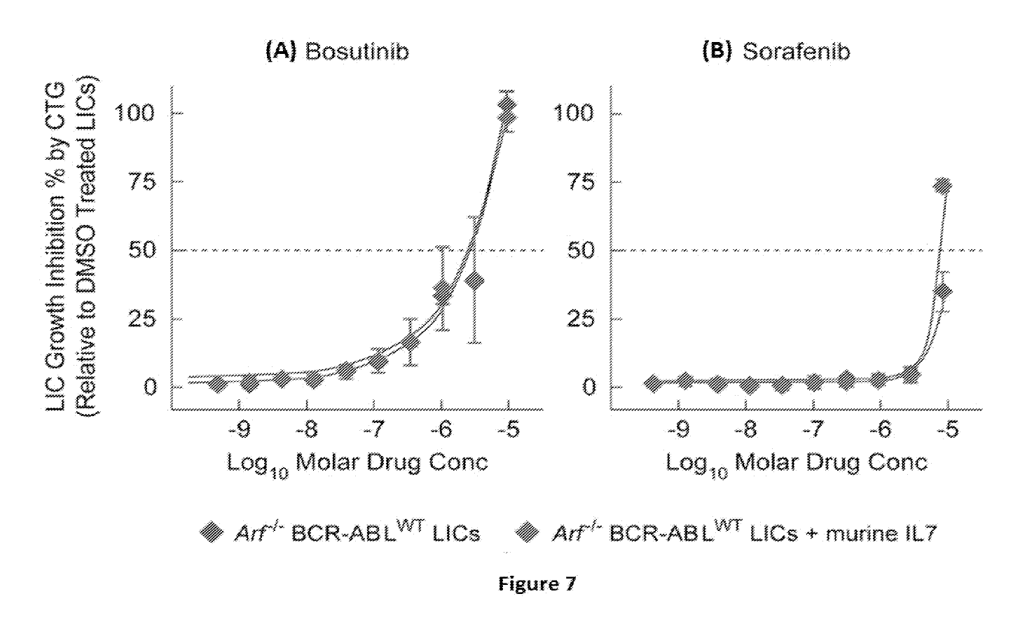

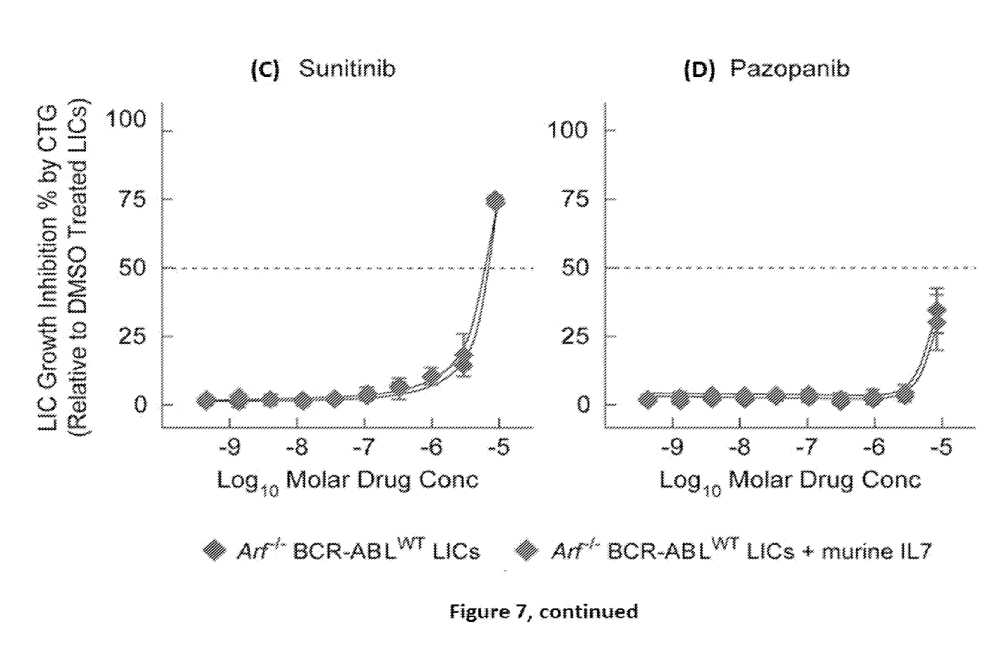

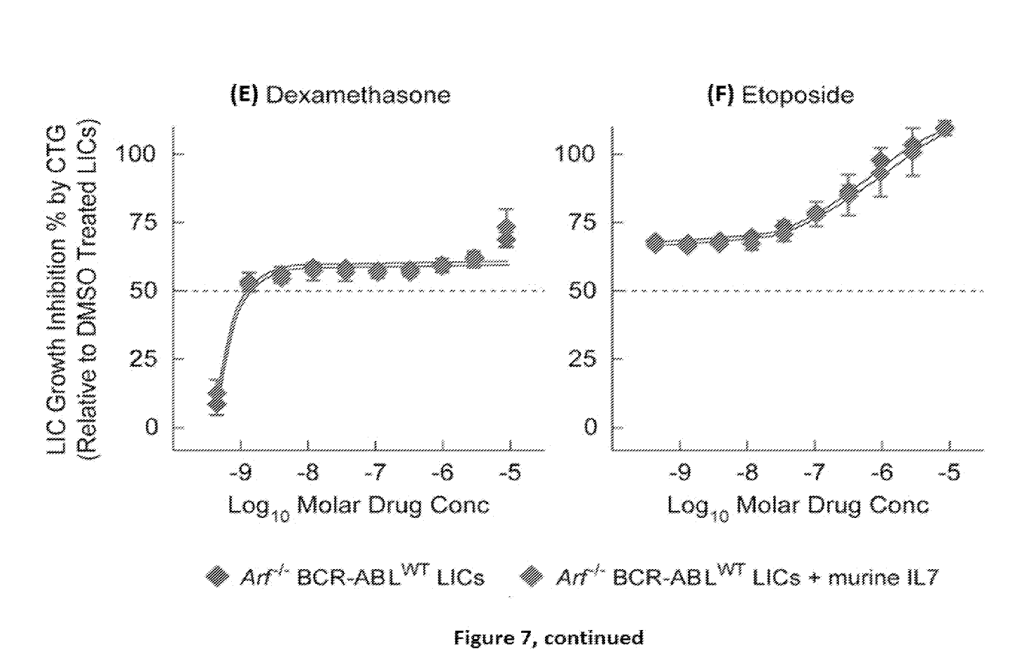

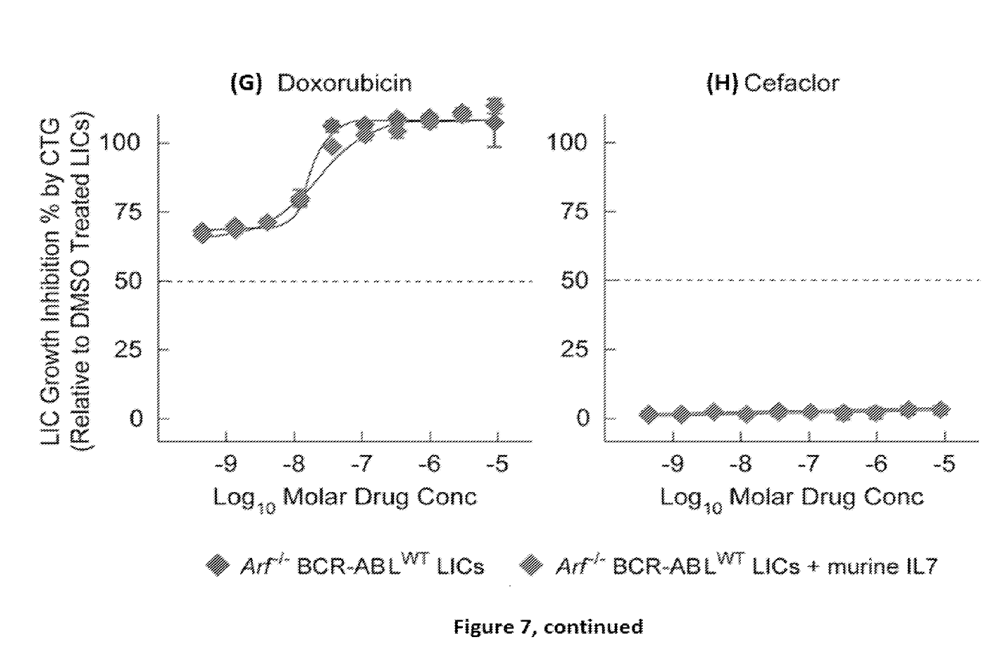

As represented in FIG. 7, Arf^{-/-} BCR-ABL^{WT} LICs were treated with drug or DMSO (no drug) for 72 hour in the absence (dark gray) or presence (light gray) of high (10 ng/mL) murine IL7 to evaluate IL7-mediated protection conferred against non-BCR-ABL-specific, multi-targeted drugs (FIG. 7, panels A-D) and conventional cytotoxic anti-leukemic clinical drugs (FIG. 7, panels E-G). The drug cefaclor (FIG. 7, panel H), an antibiotic often used in leukemia clinics, was used as a non anti-leukemic drug control. For each drug concentration, LIC growth was measured by CTG assay and average values were normalized to DMSO-treated LICs and graphed (+/−sd). IL7 was not observed to exhibit the same effect on potency of these tested drugs in BCR-ABL^{WT} LICs. Without wishing to be bound by a particular theory, bosutinib, which has shown activity against BCR-ABL, is believed to counter any IL7-mediated protection through its ability to inhibit STATS, which is thought to be necessary to sustain BCR-ABL transformed cells. These results indicate that IL7 appears to play a role in driving a specific cellular response that can provide resistance to BCR-ABL-TKIs.

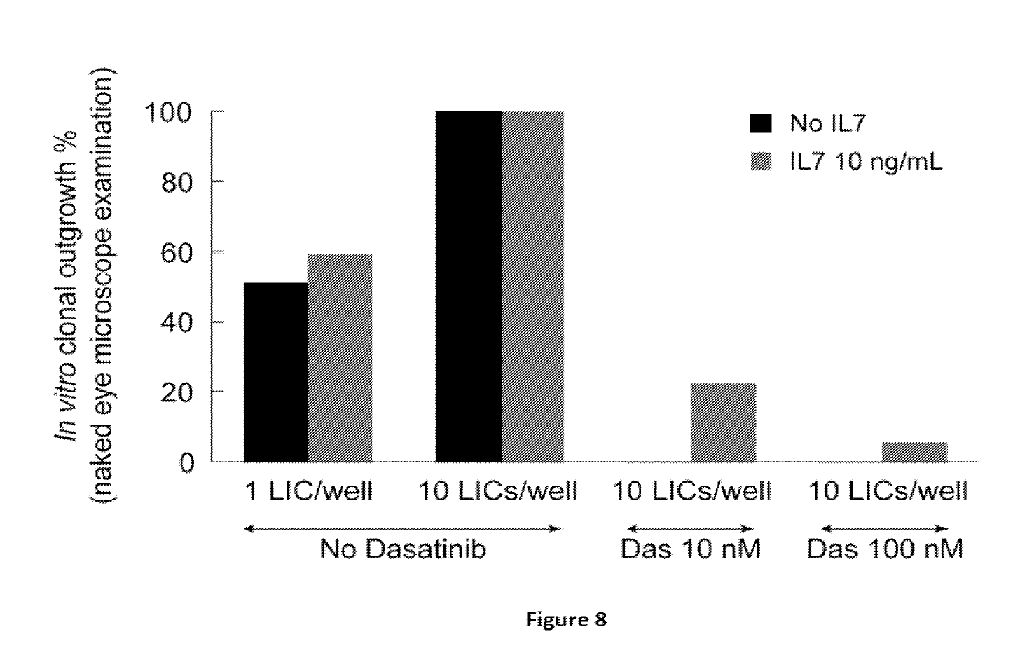

The specificity was further confirmed using three additional in vitro studies. As represented in FIG. 8, 1 or 10 viable BCR-ABL^{WT} LICs per well, sorted by single-cell flow cytometry into 3 clear-bottom 384-well plates for each condition, were treated (t=0) with DMSO (0.1% by volume) or dasatinib in the absence (dark gray) or presence (light gray) of IL7 (5 ng/mL) to evaluate in vitro leukemia clonal outgrowth. After 8 days of incubation, naked-eye microscopic examination was performed. Wells showing significant positive growth (approximately ≧50 LICs per well) were scored positive and percentage of positive wells for each condition indicated on the x-axis was calculated. These results suggests the presence of IL7 during prolonged dasatinib treatment can rescue the clonal outgrowth capacity of Arf^{-/-} BCR-ABL^{WT} LICs.

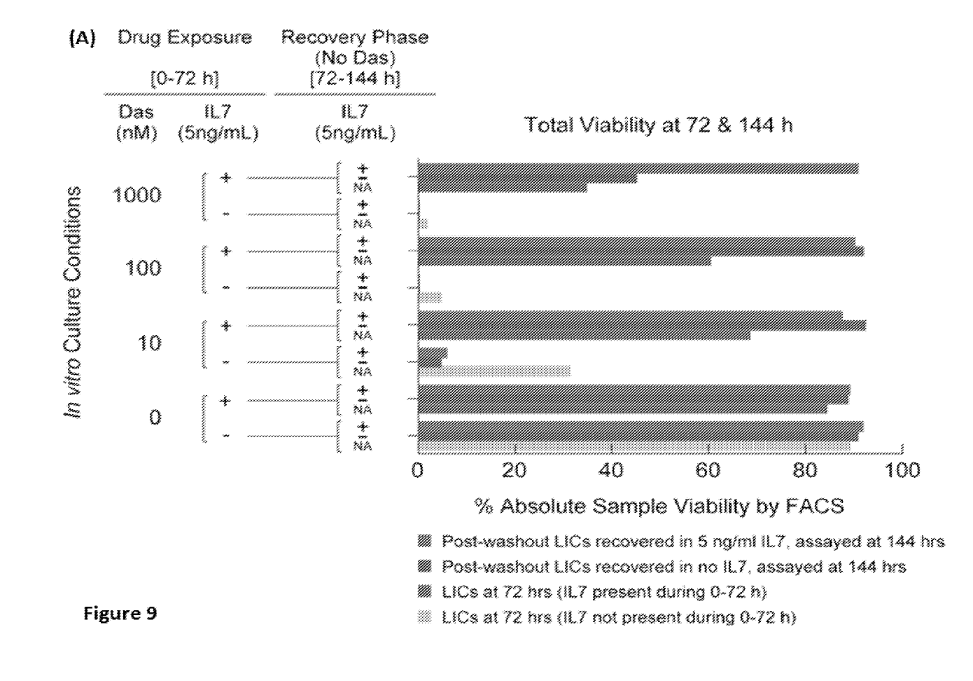

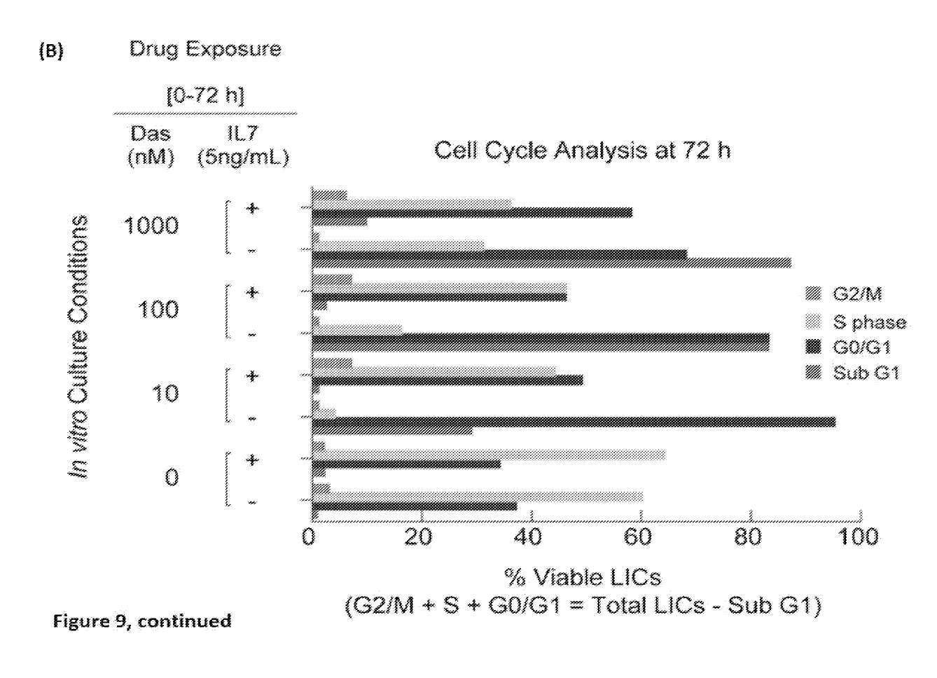

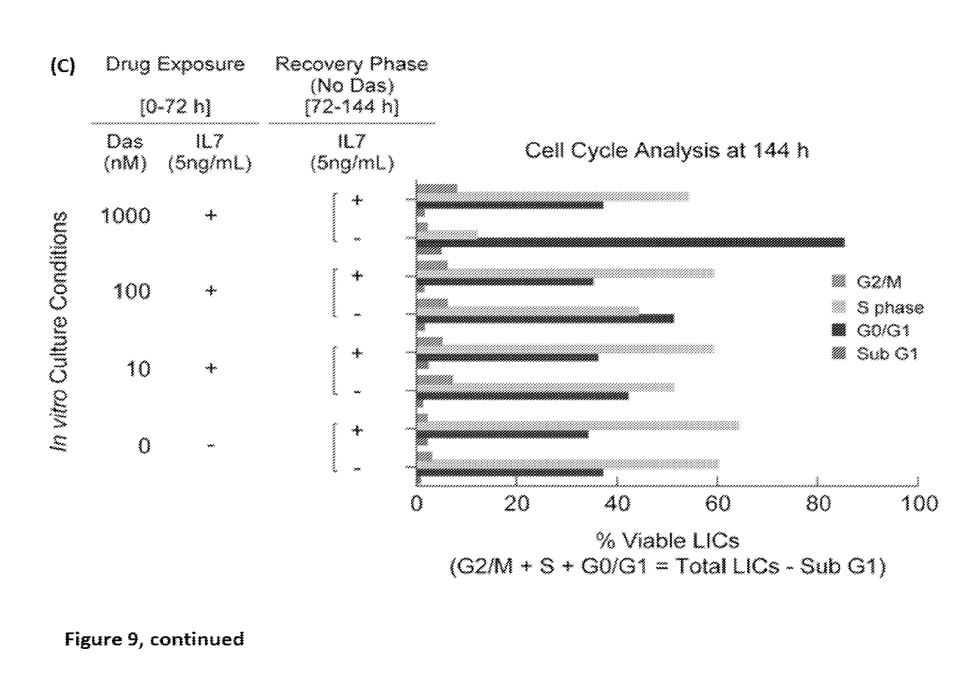

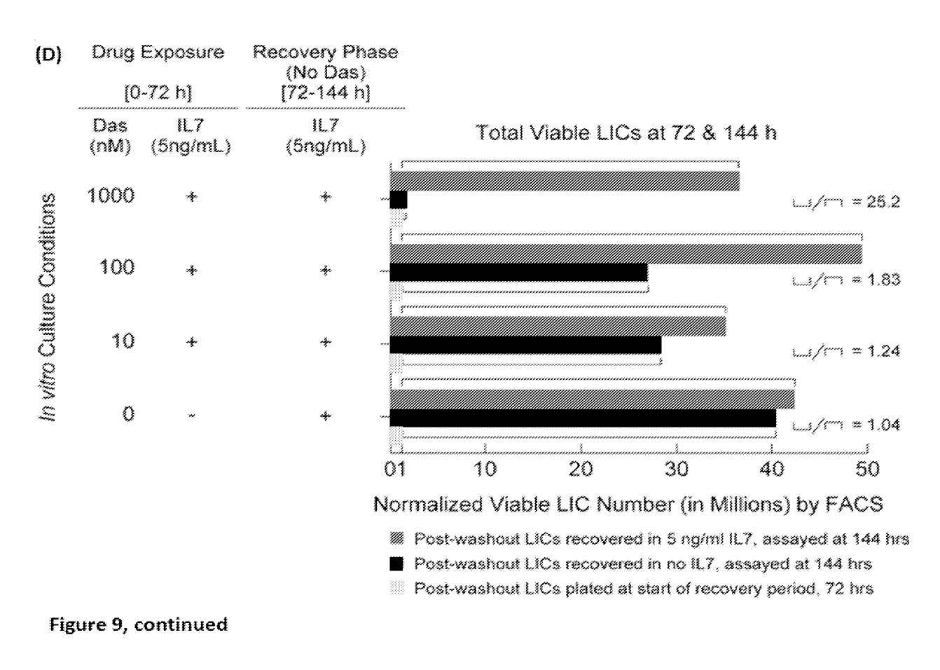

To assess protection of BCR-ABL^{WT} LICs by IL7 during dasatinib exposures (0 to 72 h) and in the post-exposure recovery period (72 to 144 h, no dasatinib), drug/cytokine washout and cytokine recovery studies were performed by the methods described above. Cell growth trends by CTG readout corroborated the findings from trypan blue (Beckman) and DAPI staining (FACS). FIG. 9a shows the total sample viabilities (DAPI staining, FACS) at the end of exposure period (light gray=IL7; dark gray=No IL7) and recovery period (black=IL7; gray=No IL7), and depicts IL7 protection during dasatinib exposures. The few LICs surviving dasatinib exposures in the absence of IL7, later deteriorated and died during recovery period, irrespective of IL7, suggesting that BCR-ABL inhibitor exposures can commit BCR-ABL+ cells to death. IL7 was observed to improve recovery of dasatinib/IL7 co-treated LICs at 1000 nM, but not at 100 nM and 10 nM dasatinib concentration, most likely due to over-confluent growth in presence of IL7. FIG. 9b shows cell cycle changes (FACS) at the end of exposure period. Incremental dasatinib doses, presumably resulting in incremental BCR-ABL inhibition, appears to induce cell cycle arrest and apoptosis. Without wishing to be bound by a particular theory, IL7 appears to protect LICs from these changes. FIG. 9c shows the cell cycle analysis at the end of recovery period. FIG. 9d shows conditions showing positive LIC growth during recovery period, normalized for equal number of LICs plated at start of recovery. The ratio of fold LIC-change in the presence (FIG. 9d, face-down bracket) to that in the absence (FIG. 9d, face-up bracket) of IL7 was observed to increase in LICs with higher previous BCR-ABL inhibition from higher dasatinib concentrations during exposure period. Without wishing to be bound by a particular theory, these results suggest transient pharmacologic BCR-ABL inhibition increases LIC responsiveness to cell-extrinsic IL7.

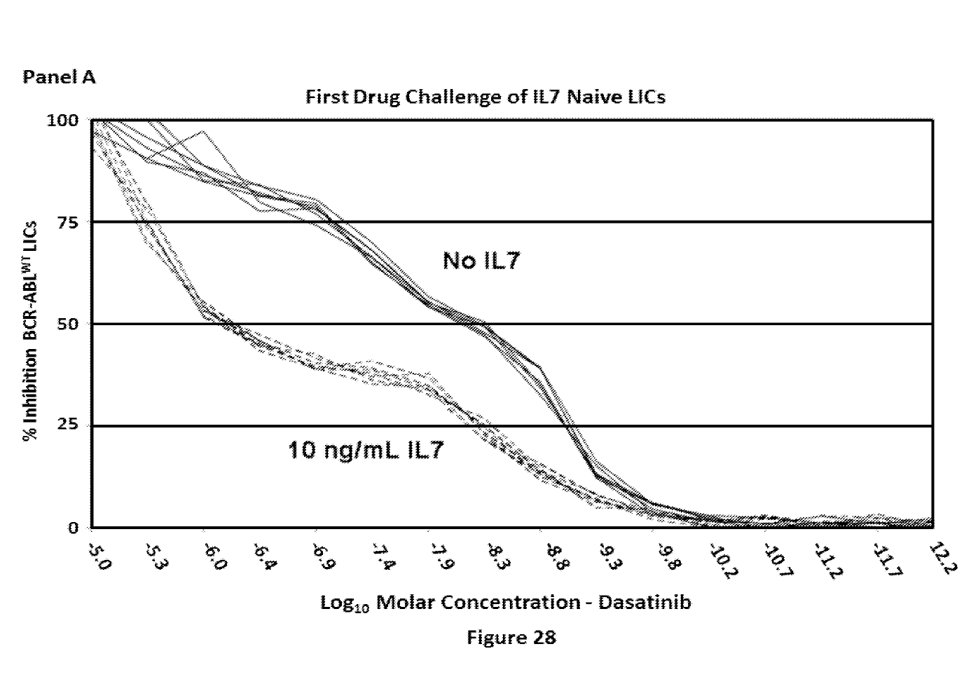

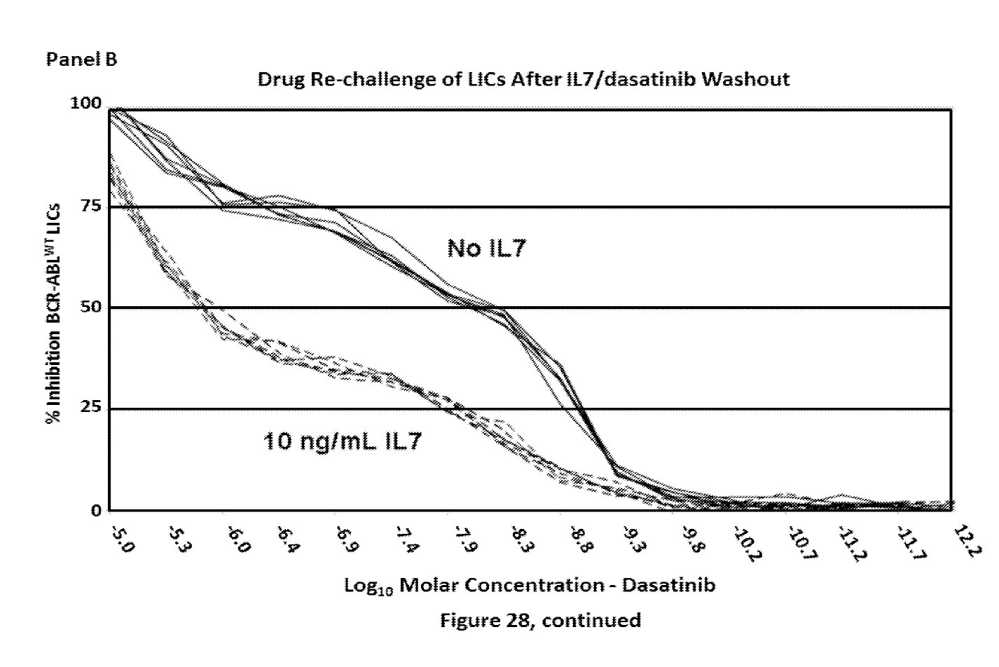

Additional re-challenge studies were performed to evaluate the possibility of a genetically drug-resistant subpopulation that could undergo selection with dasatinib/IL7 co-treatment during 0-72 hr of the dasatinib/cytokine washout studies described above. At 144 hrs, dasatinib was observed to exhibit a similar potency against LICs in the absence of IL7, irrespective of previous dasatinib exposure. Furthermore, potency of dasatinib appeared to be blunted, to a similar extent, during both the first and second drug challenges only when extracellular IL7 was present (FIG. 28, Panels A and B). These results suggest that removing IL7 can completely reverse its protective effects and restores dasatinib potency. Without wishing to be bound by a particular theory, these findings also suggest that IL7 exposure drives both the location of sequestration and the acquisition of resistance.

3. Physiological Levels of IL7 Confer an Aggressive, Dasatinib-Resistant Phenotype to PL+ All In Vivo

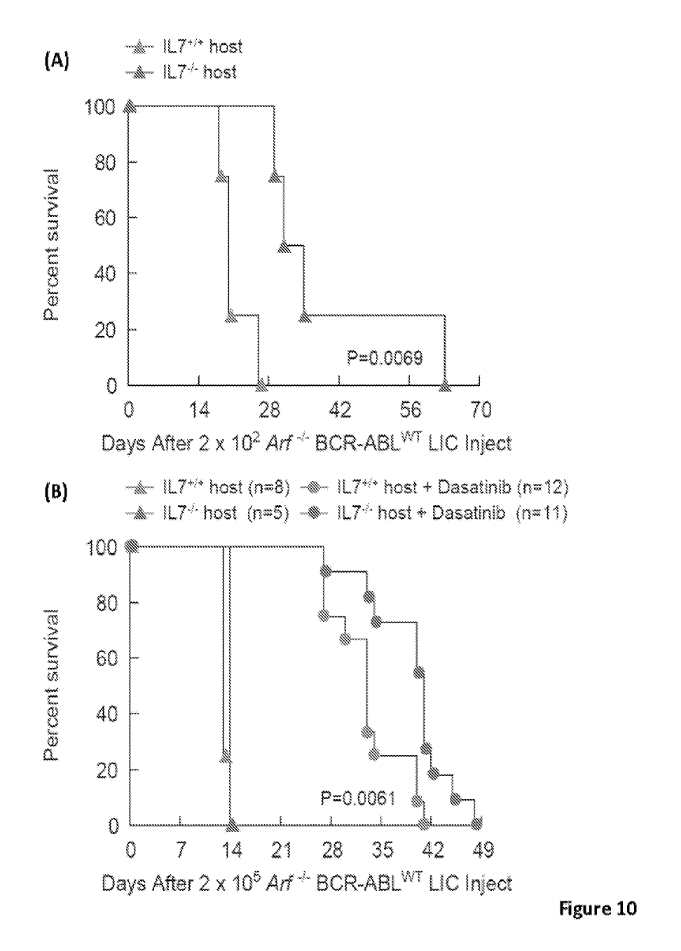

The effect of IL7 on leukemia progression and dasatinib responsiveness in vivo was studied in IL7^{+/+} and IL7^{-/-} B16 host-mice. FIG. 10 shows the Kaplan-Meier survival curves showing overall survival in IL7^{+/+} and IL7^{-/-} B16 host-mice receiving day 0 tail vein injection of 2×10² Arf ^{-/-} BCR-ABL^{WT} LICs (FIG. 10, panel A, vehicle treated) or 2×10⁵ Arf^{-/-} BCR-ABL^{WT} LICs(FIG. 10, panel B, vehicle or drug treated). Median survival in 2×10² Arf^{-/-} BCR-ABL^{WT} LICs injections (“low burden model”) was 20 days in IL7^{+/+} (n=4) versus 33 days in IL7^{-/-} mice (n=4)(P=0.0069). In 2×10⁵ Arf^{-/-} BCR-ABL^{WT} LICs injections (“high burden model”), survivals of vehicle-treated IL7^{+/+} and IL7^{-/-} mice were comparable. Dasatinib treatment caused a median survival of 33 days in IL7^{+/+} (n=12) versus 41 days in IL7^{-/-} (n=11) mice, P=0.0061. The data is an average of 2 independent experiments in which vehicle or dasatinib therapy was initiated on day 7 or 9. IL7^{+/+}, but not IL7^{-/-}, host mice injected with relatively small numbers (200) of BCR-ABL^{WT} LICs (“low burden model”) showed decreased survival (FIG. 10, panel A), suggesting that murine Ph+ ALL responds to host-IL7 with accelerated progression. Injecting higher numbers (200,000) of LICs (“high burden model” (FIG. 10, panel B), appears to overcome this trend, suggesting the phenomena is most important during initial establishment and/or expansion of the leukemic cell population. Treatment of animals in the high burden model revealed a shortened survival benefit from dasatinib monotherapy of BCR-ABL^{WT}-driven leukemias. Without wishing to be bound by a particular theory, a single host-cytokine can induce meaningful BCR-ABL-TKI drug resistance in vivo. From a therapeutic perspective, these findings suggest that in vivo responses to dasatinib therapy in Ph+ ALL are modulated by cell-extrinsic IL7, independent of BCR-ABL KD mutations. The IL7-imparted primary protection, coupled with higher disease burdens in Ph+ ALL compared to CML in the clinical setting, can contribute to greater BCR-ABL-TKI refractoriness in Ph+ ALL relative to CML.

4. High-Throughout Screens Agaisnt Primary (IL7-Induced) and Acquired (BCR-ABL-Mutation) BCR-ABL-TKI Drug-Resistant Phenotypes

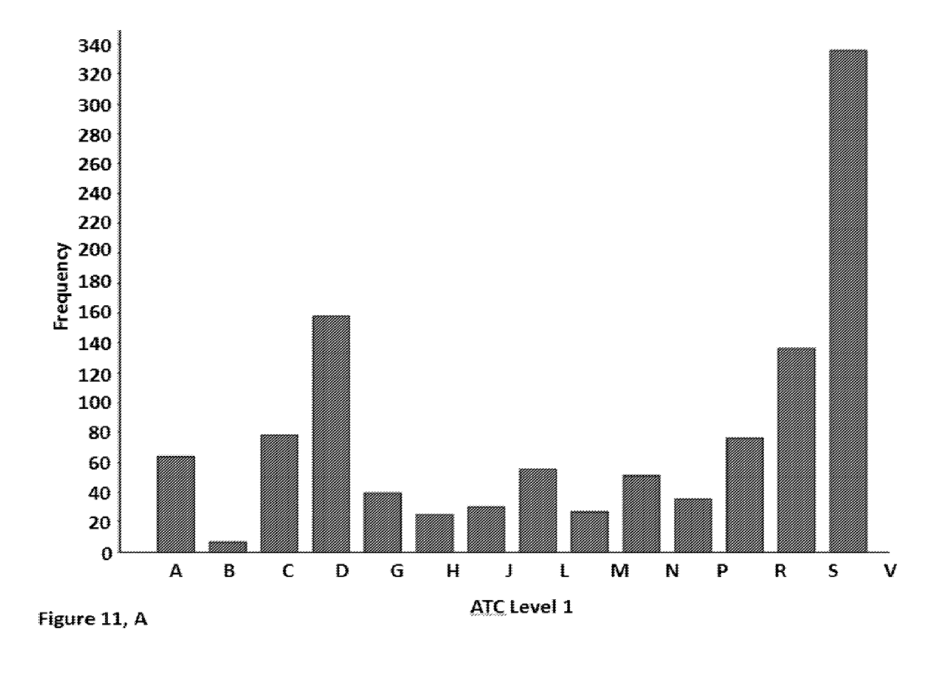

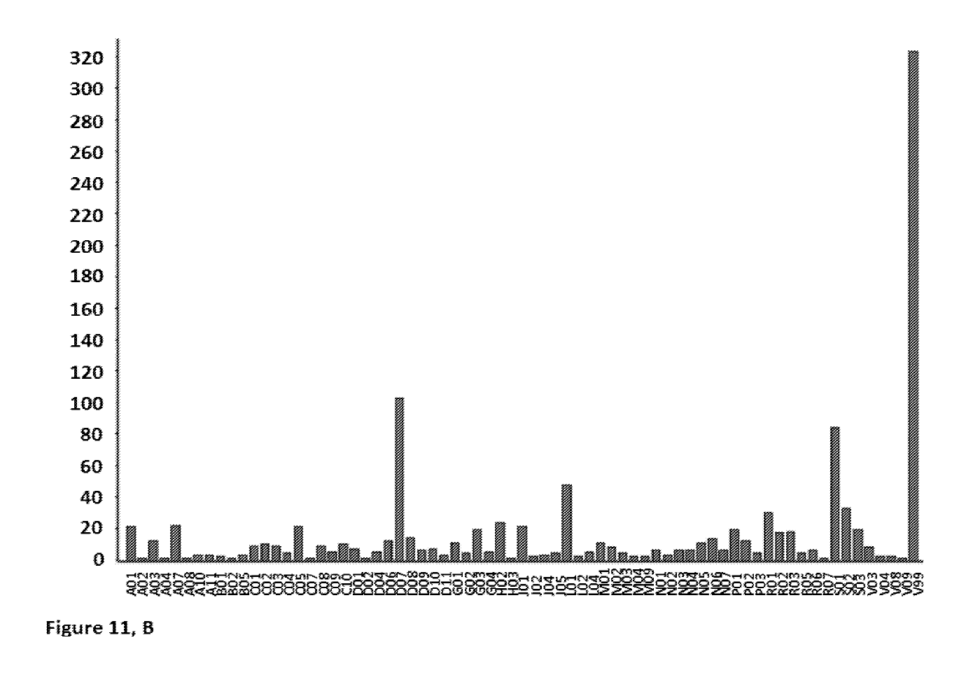

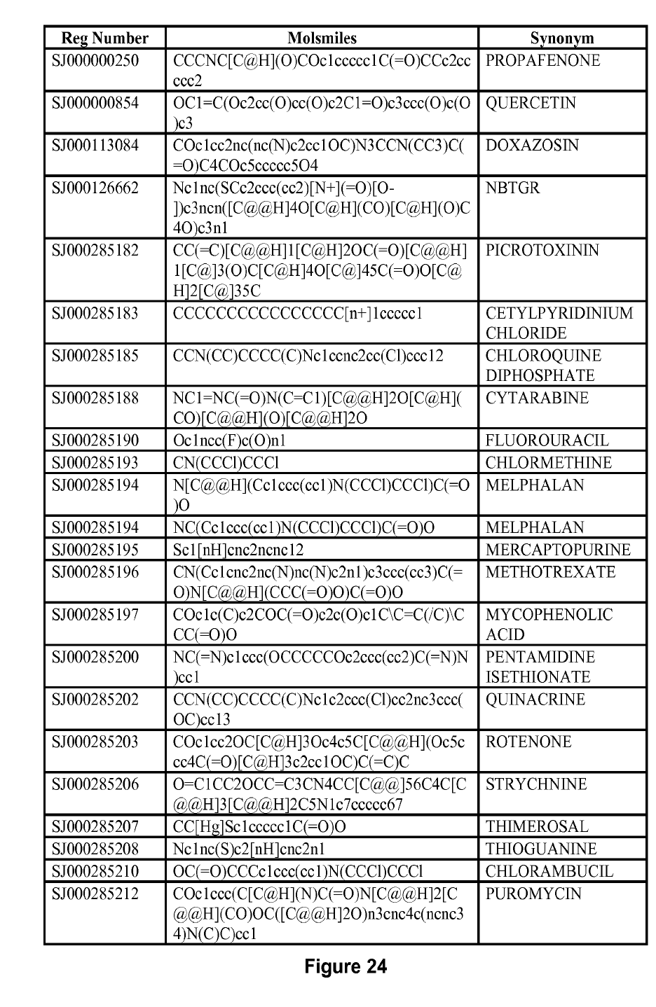

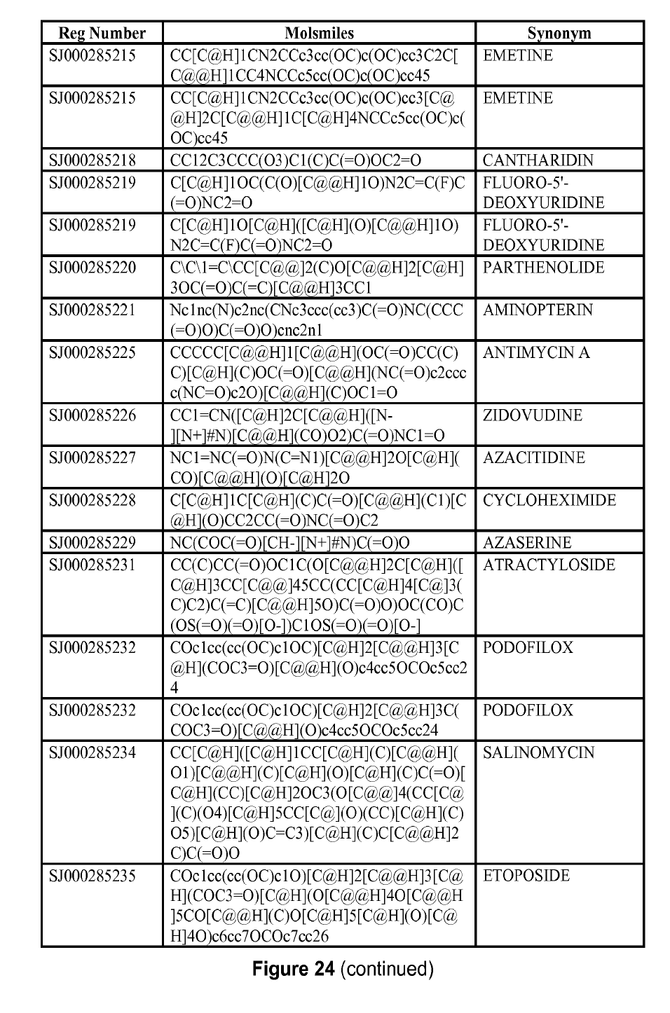

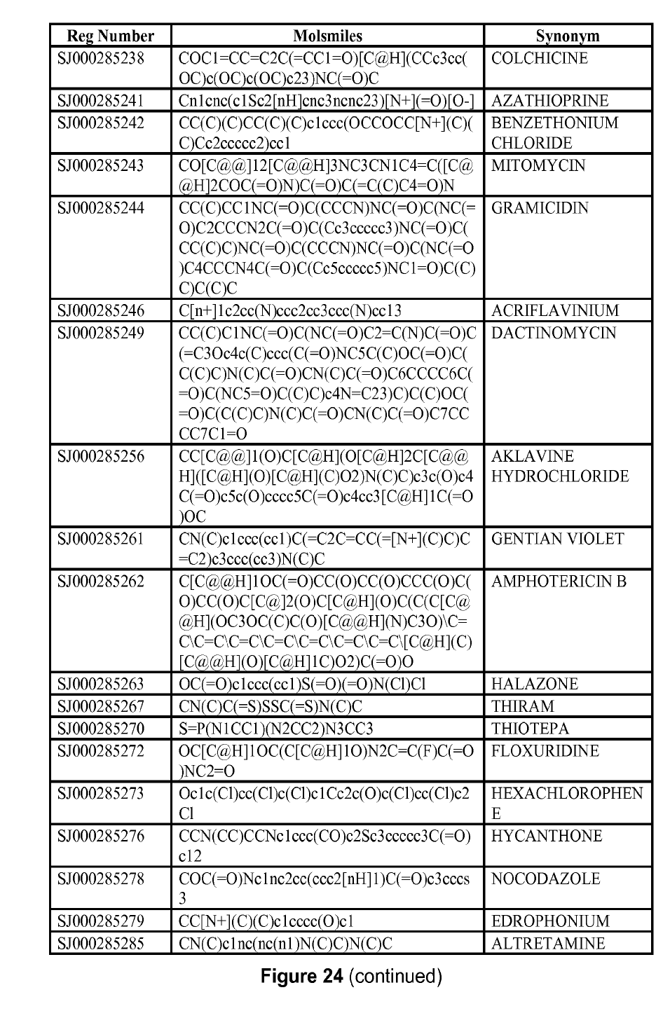

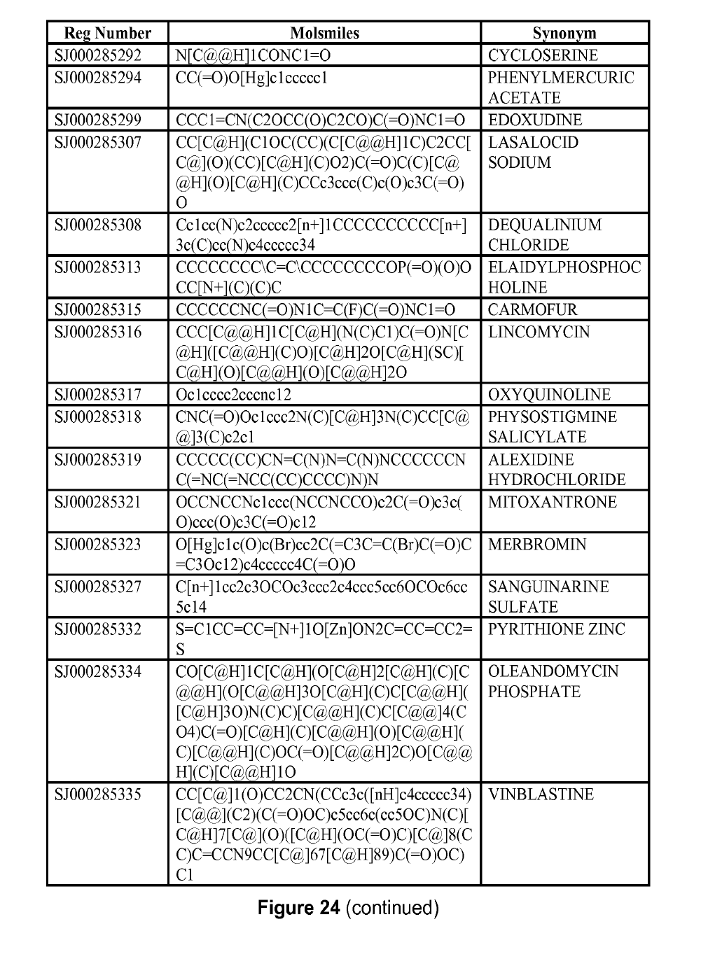

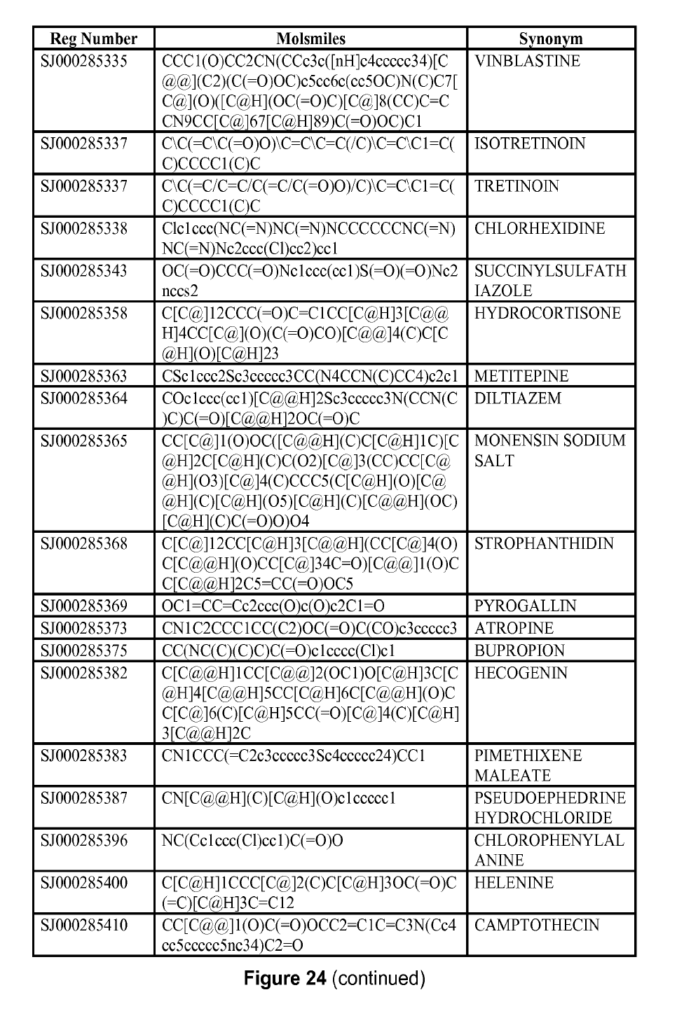

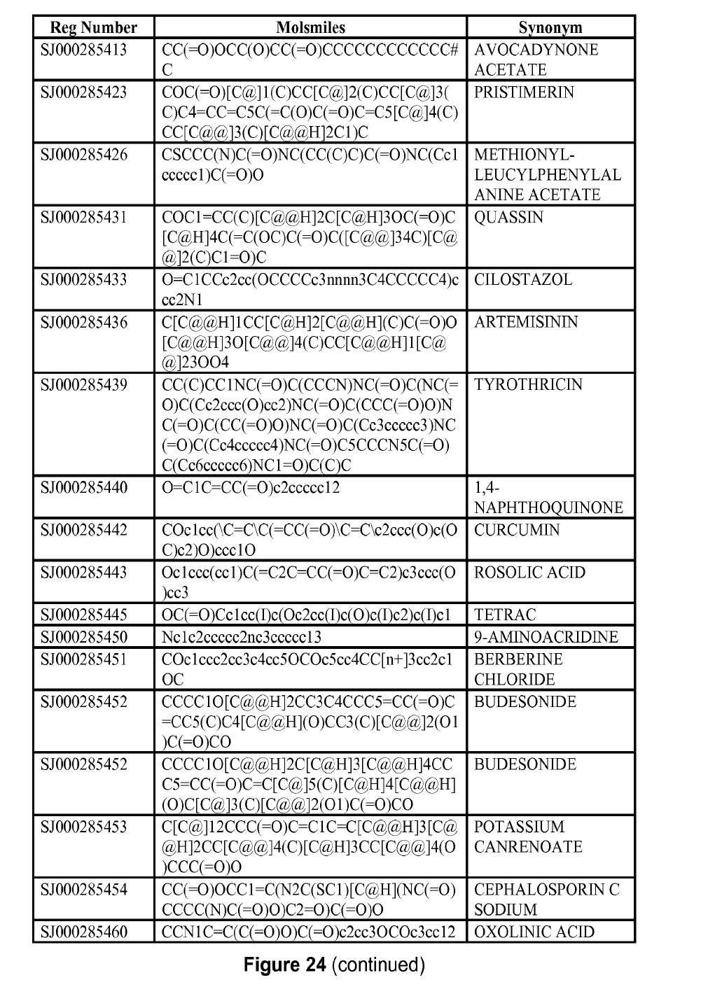

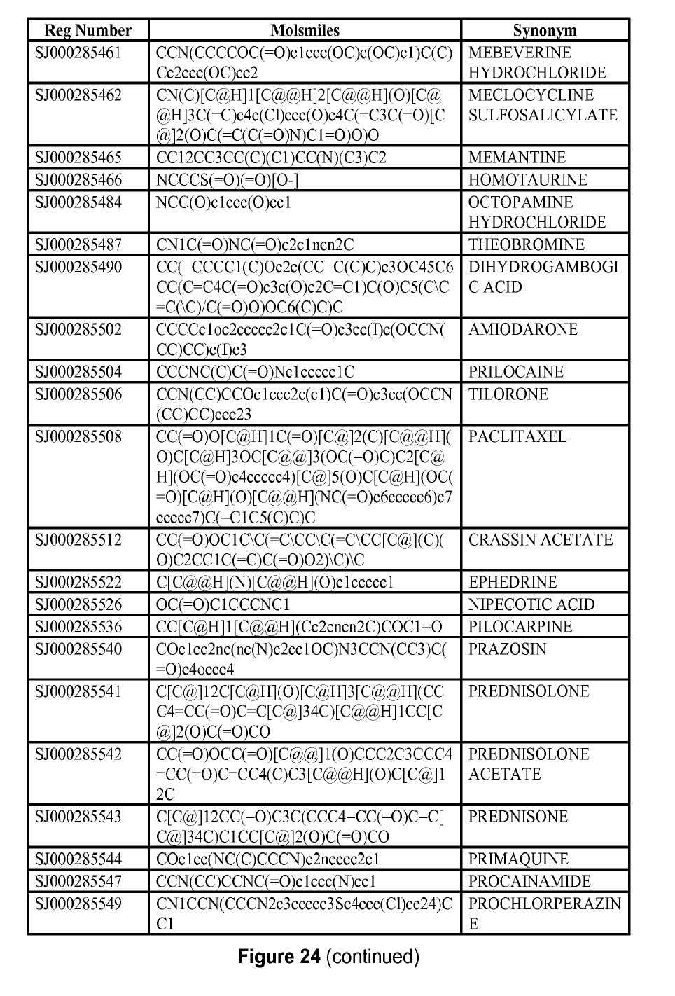

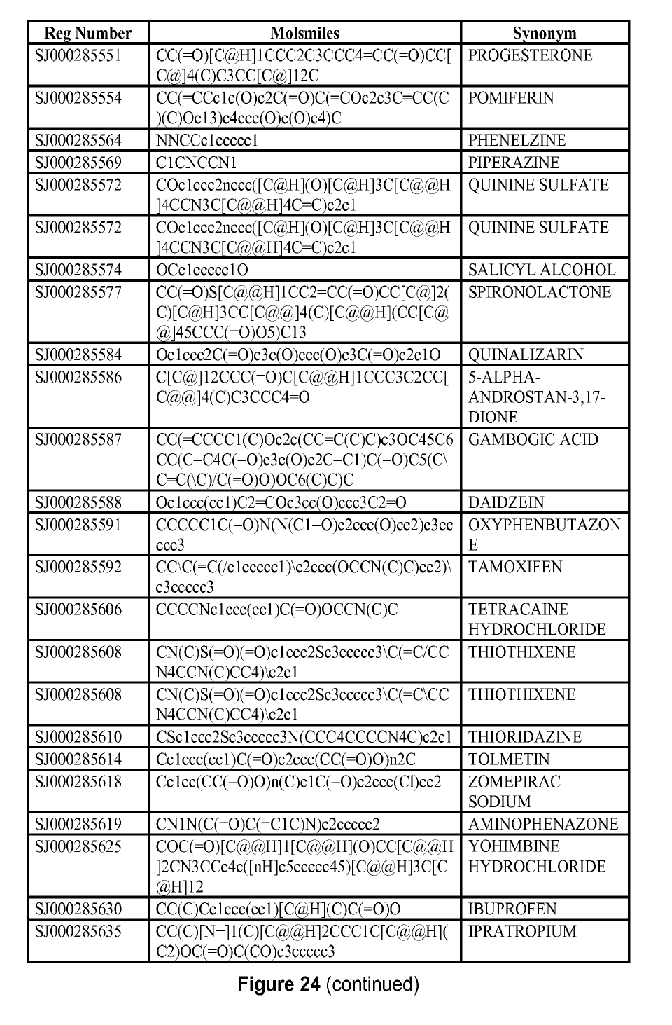

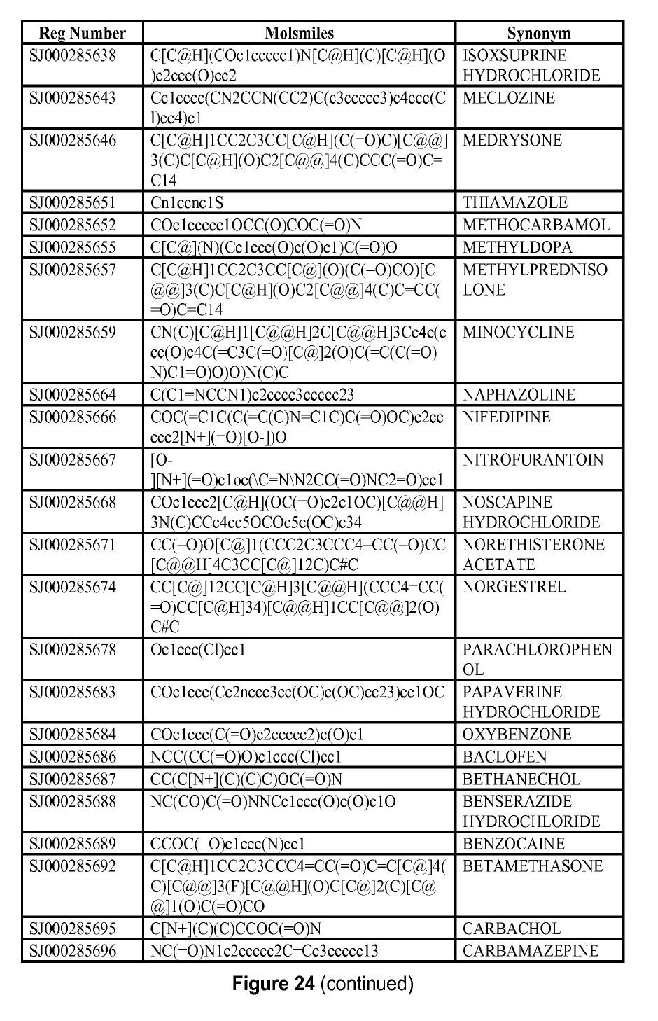

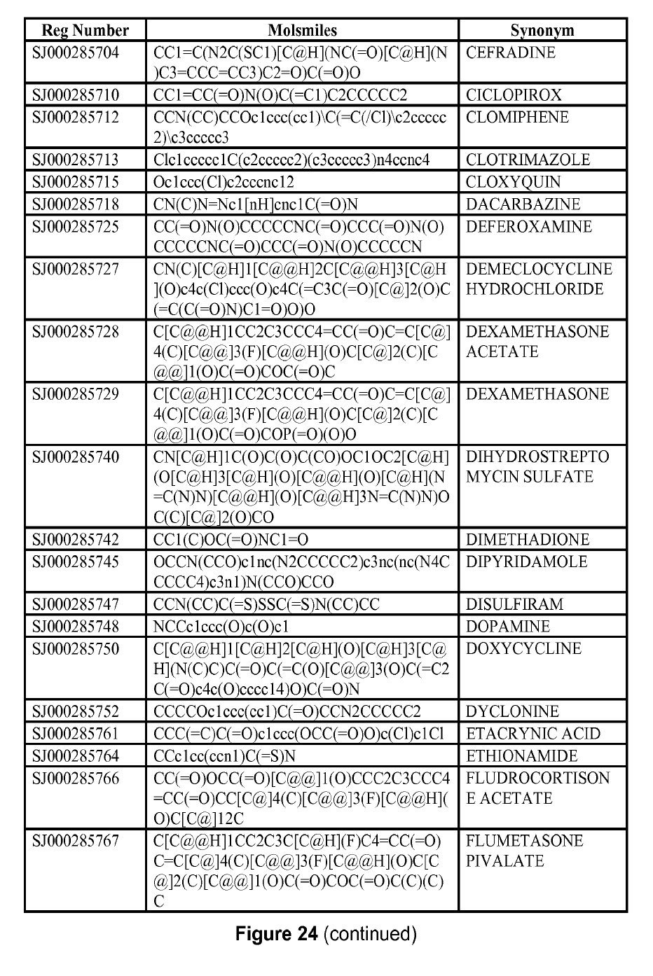

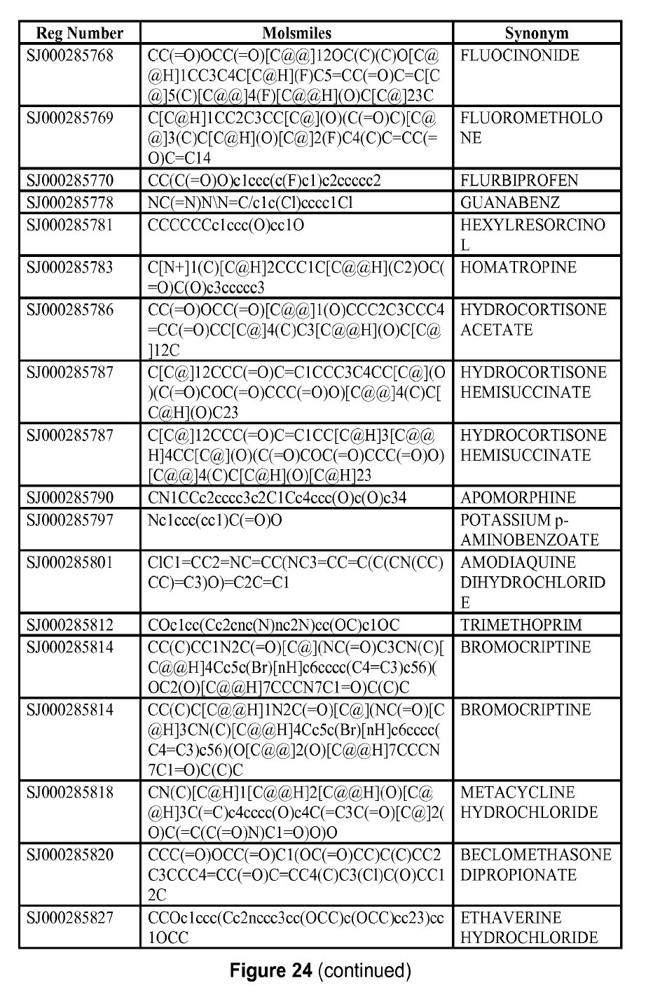

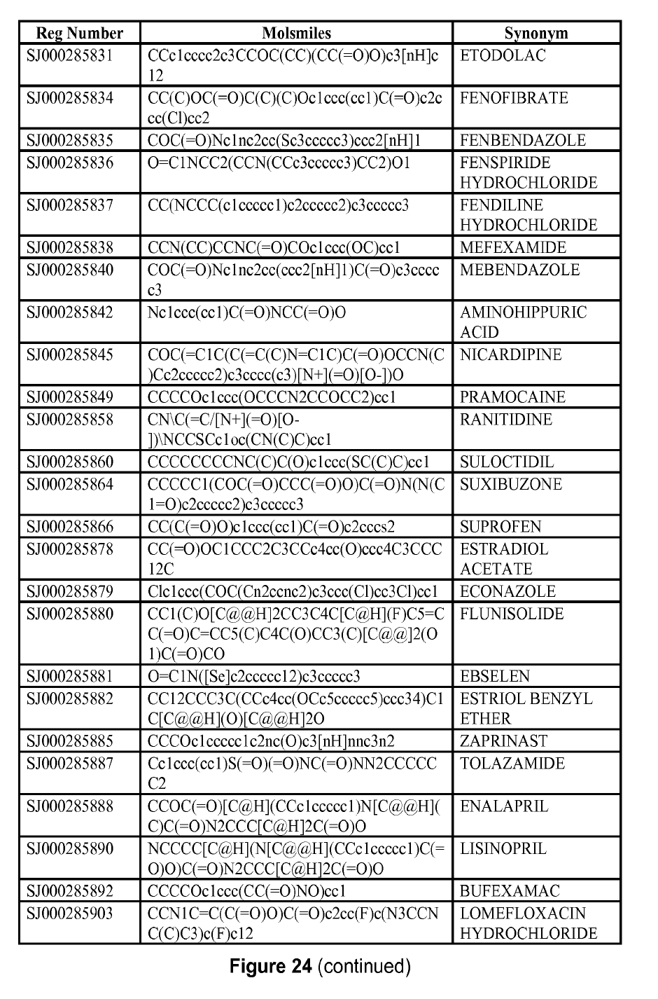

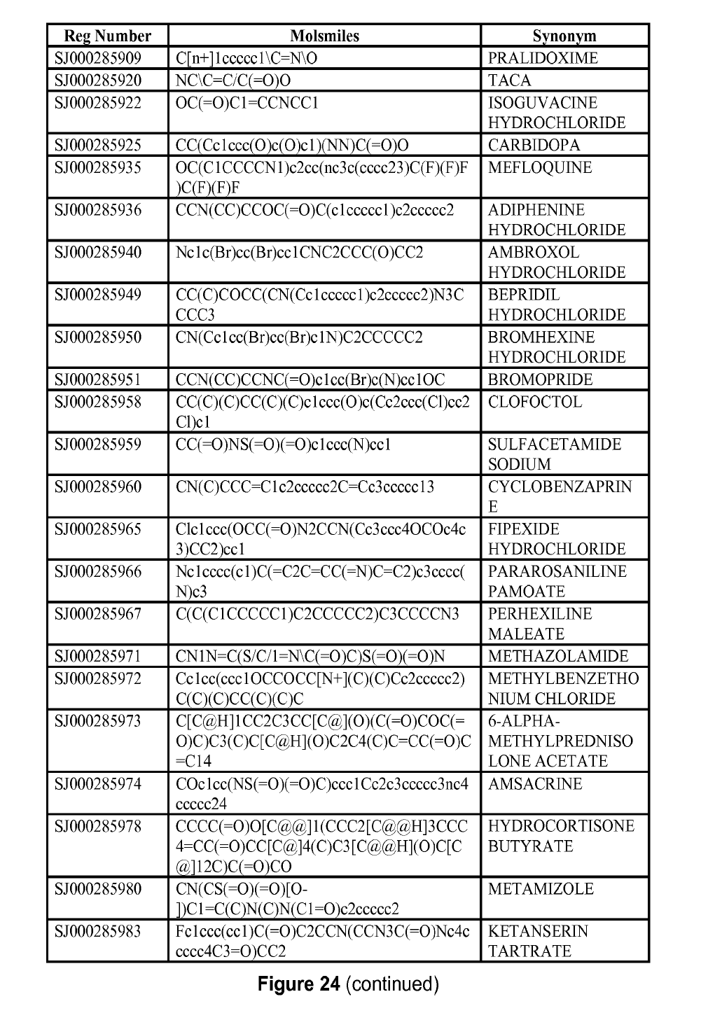

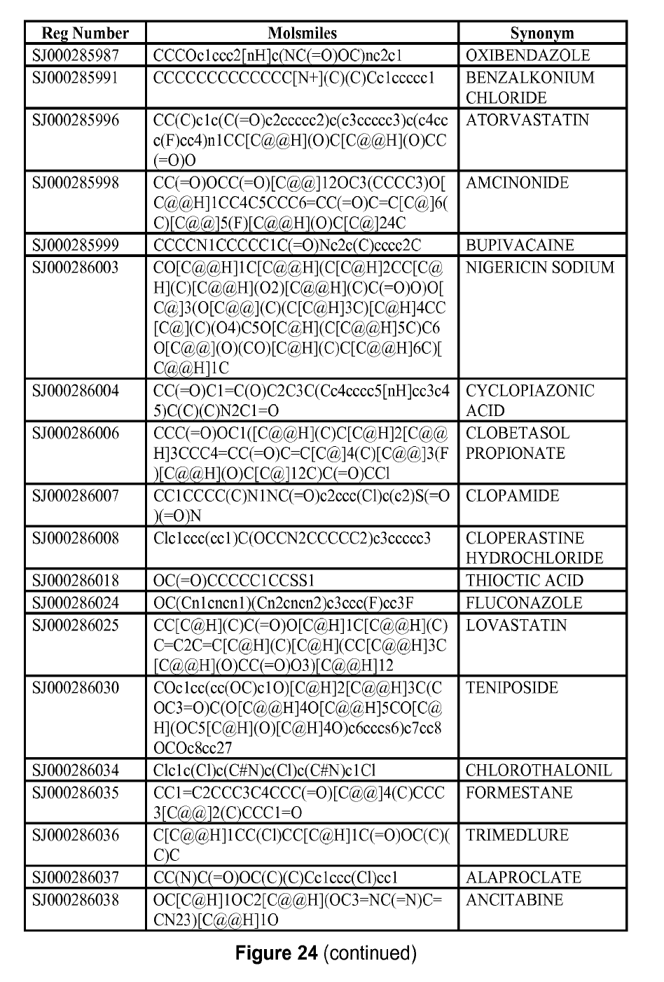

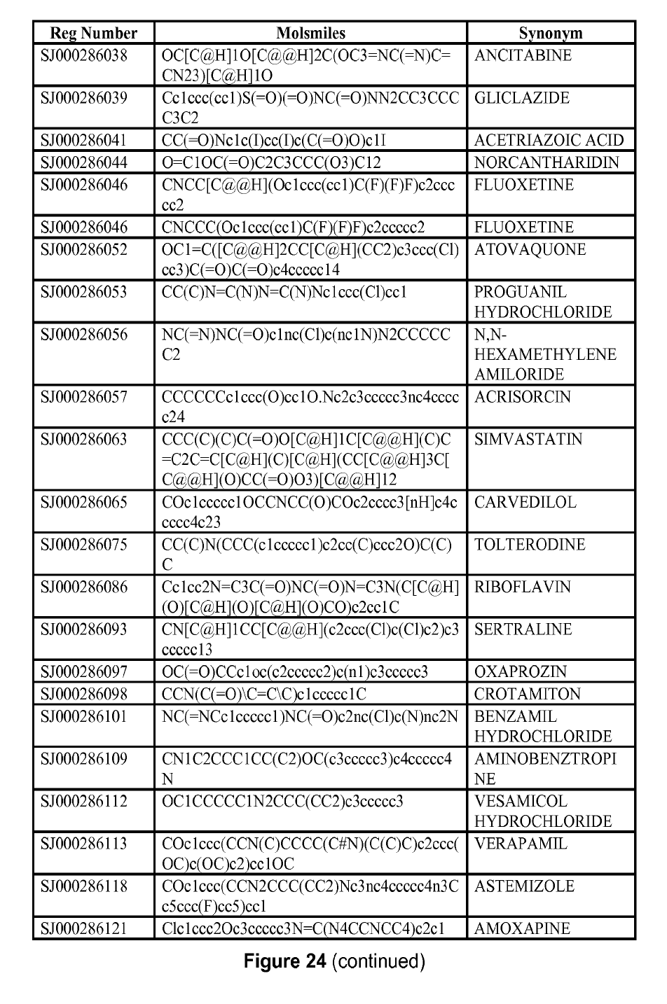

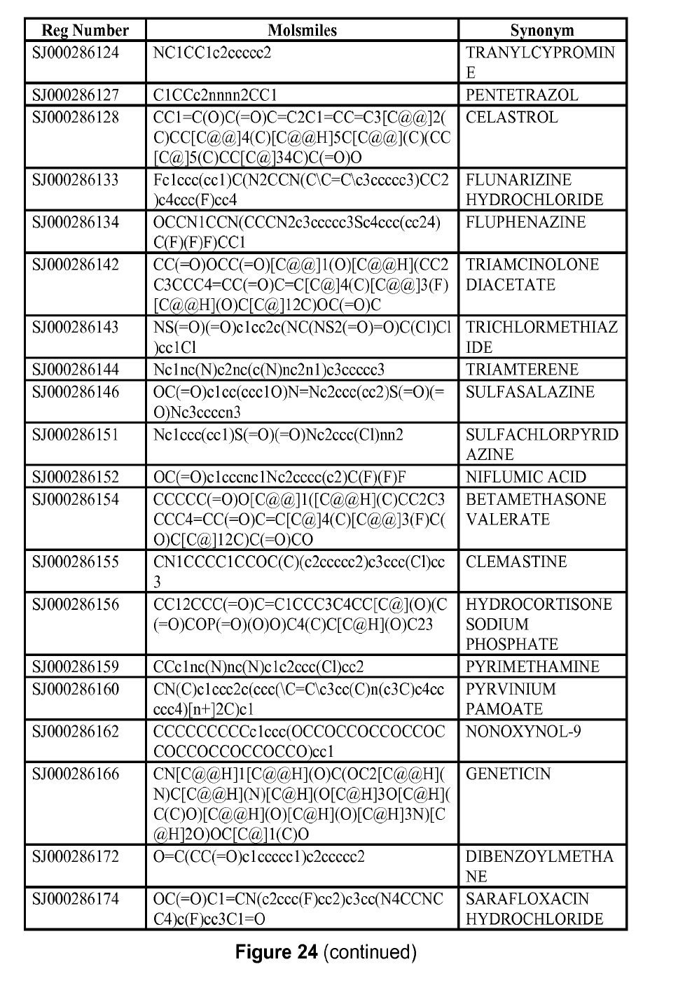

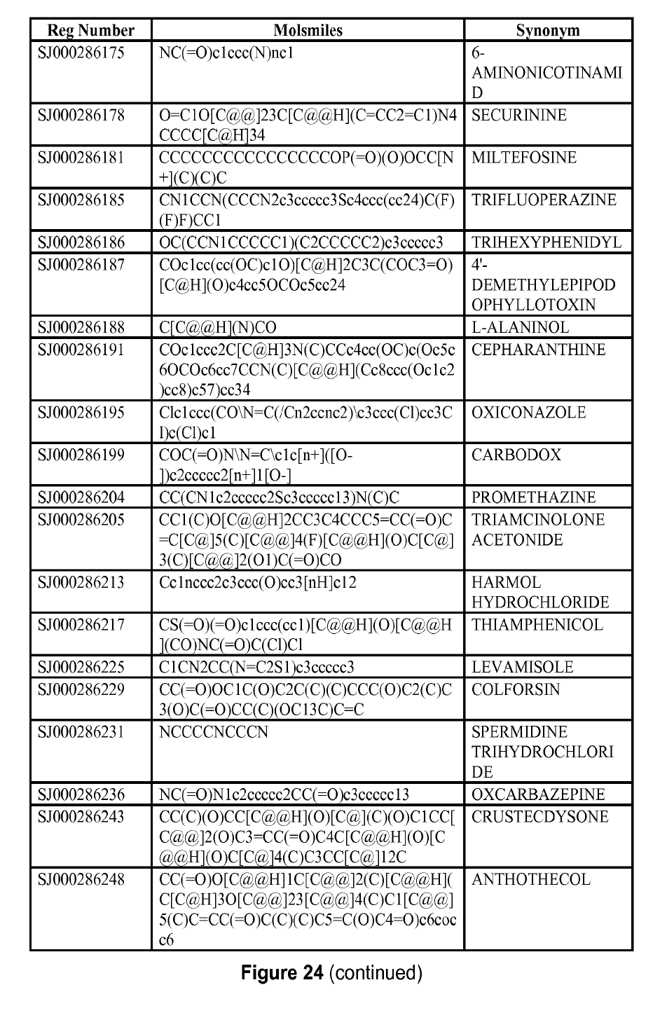

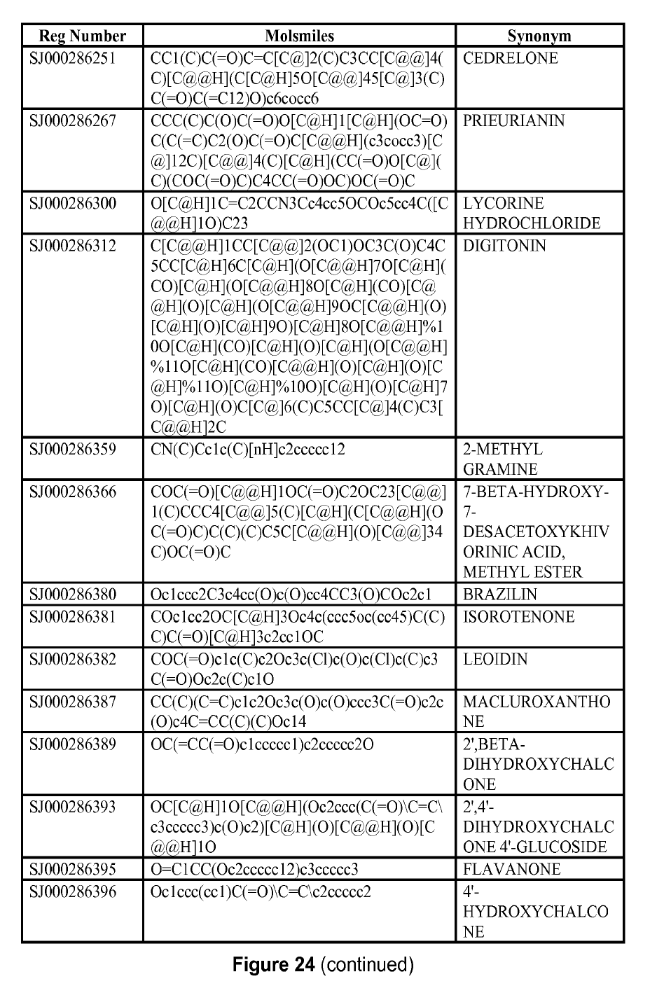

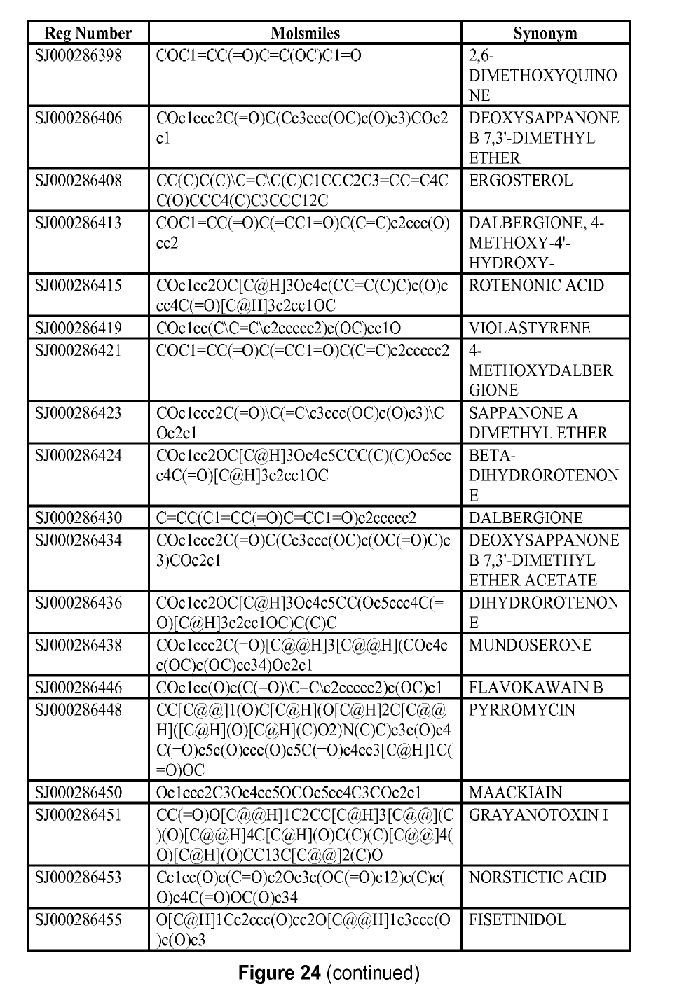

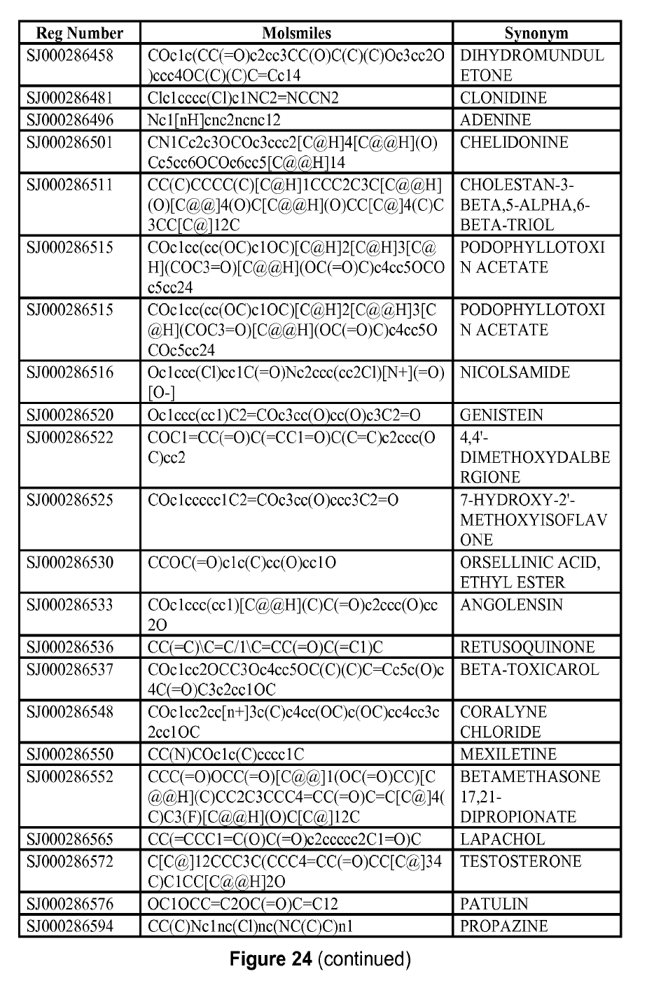

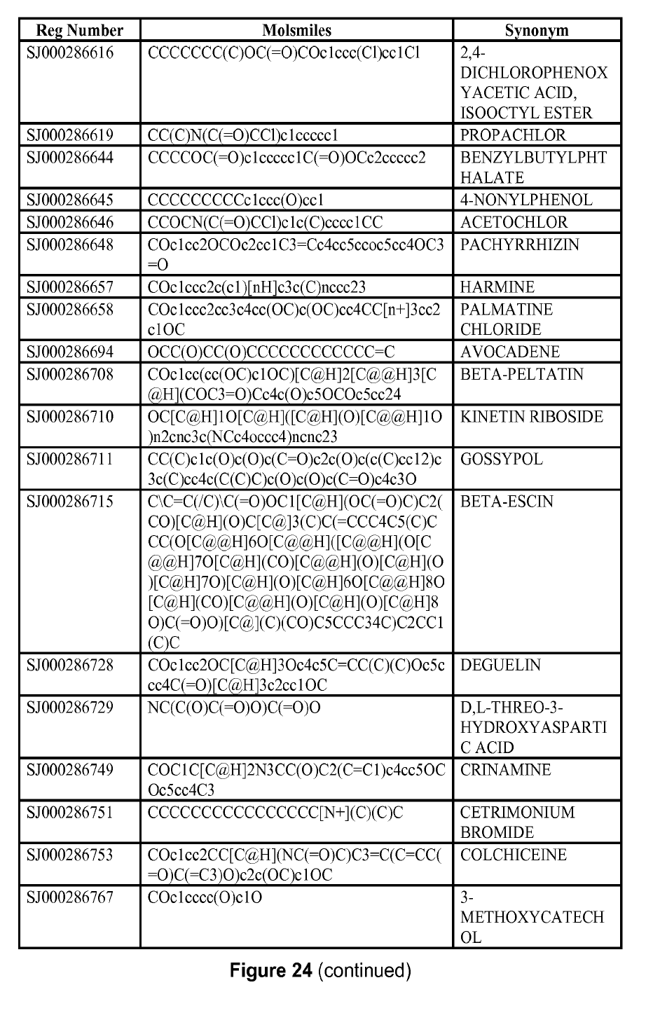

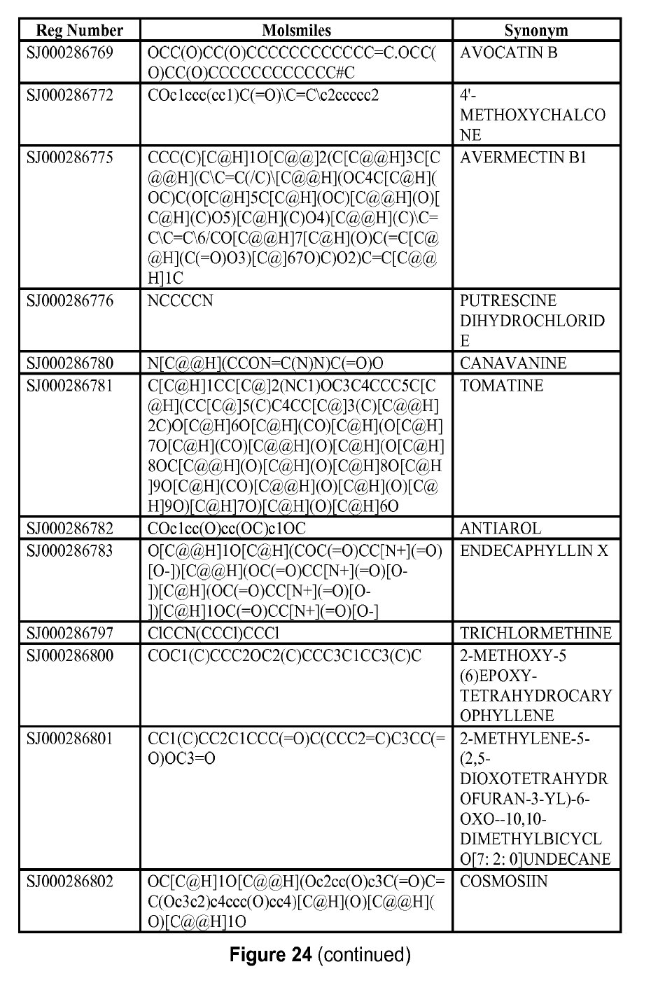

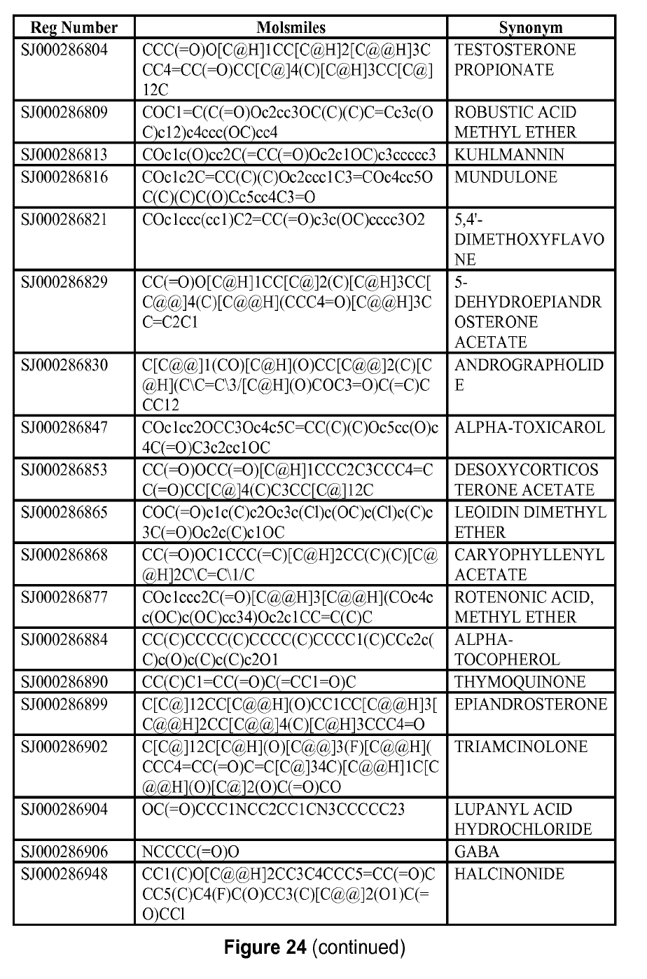

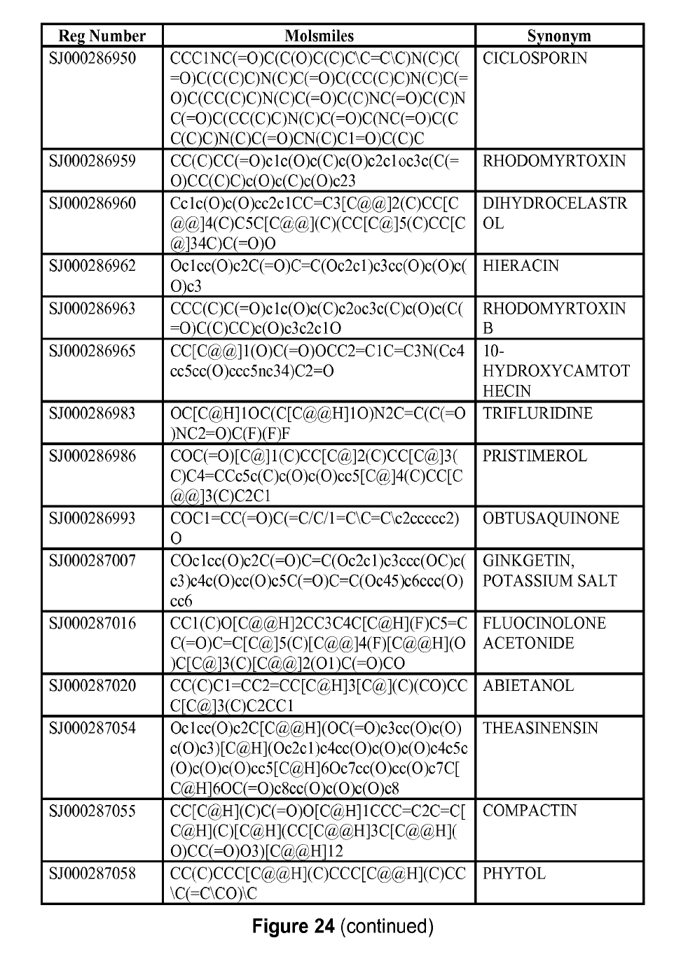

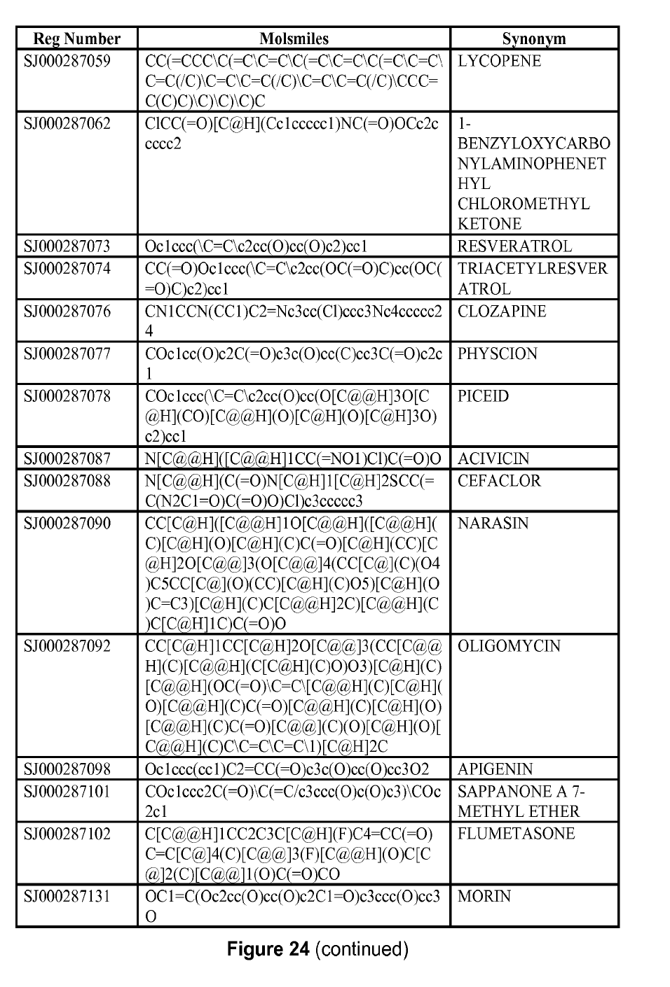

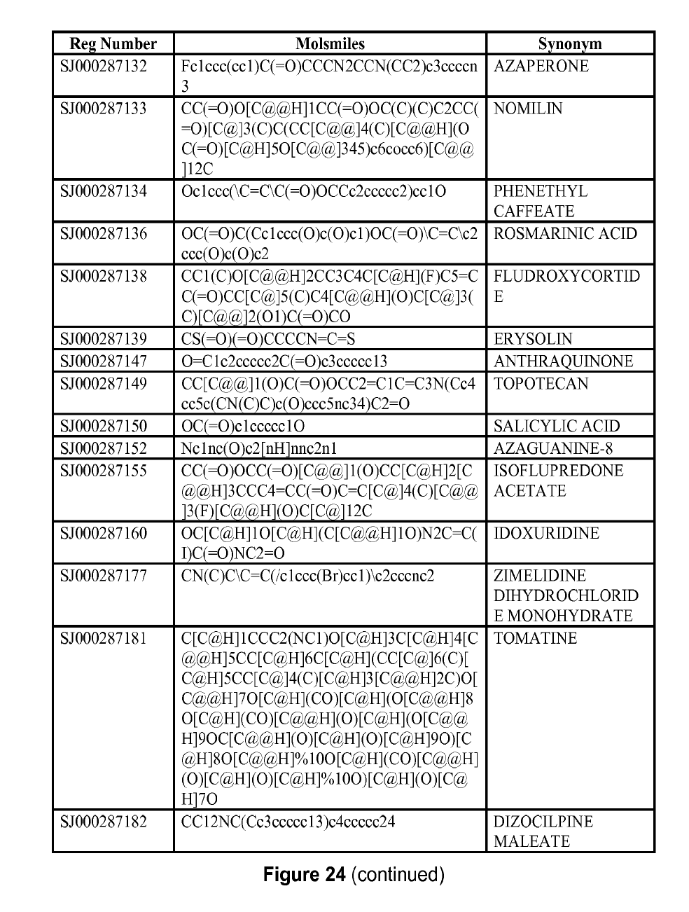

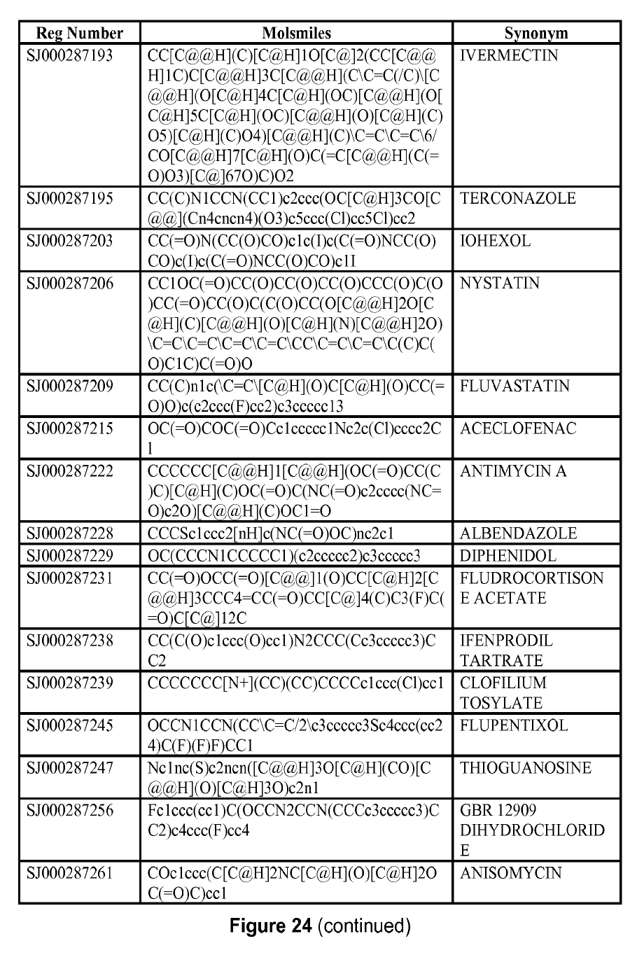

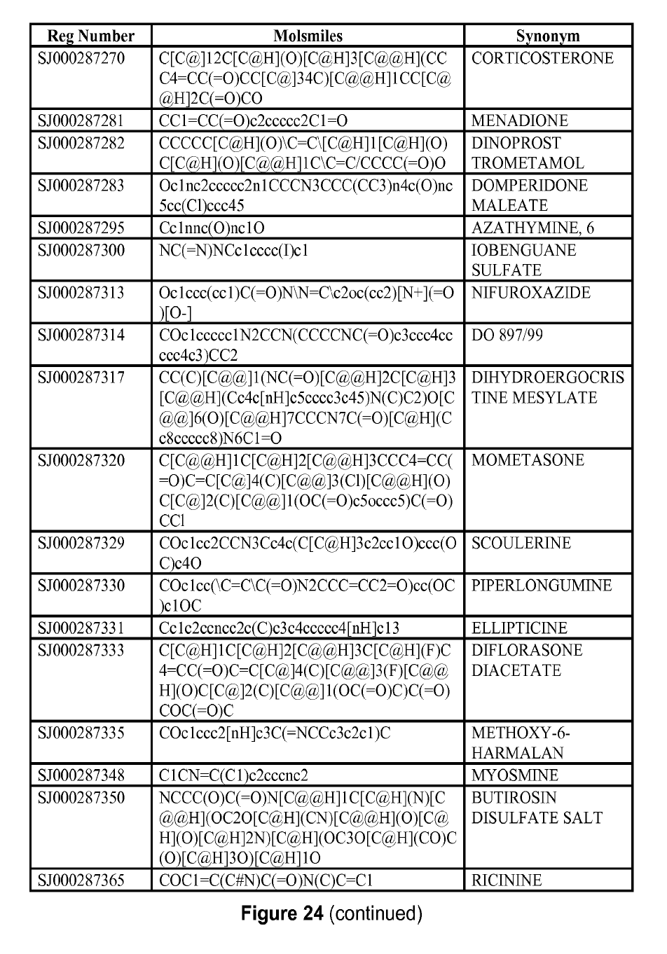

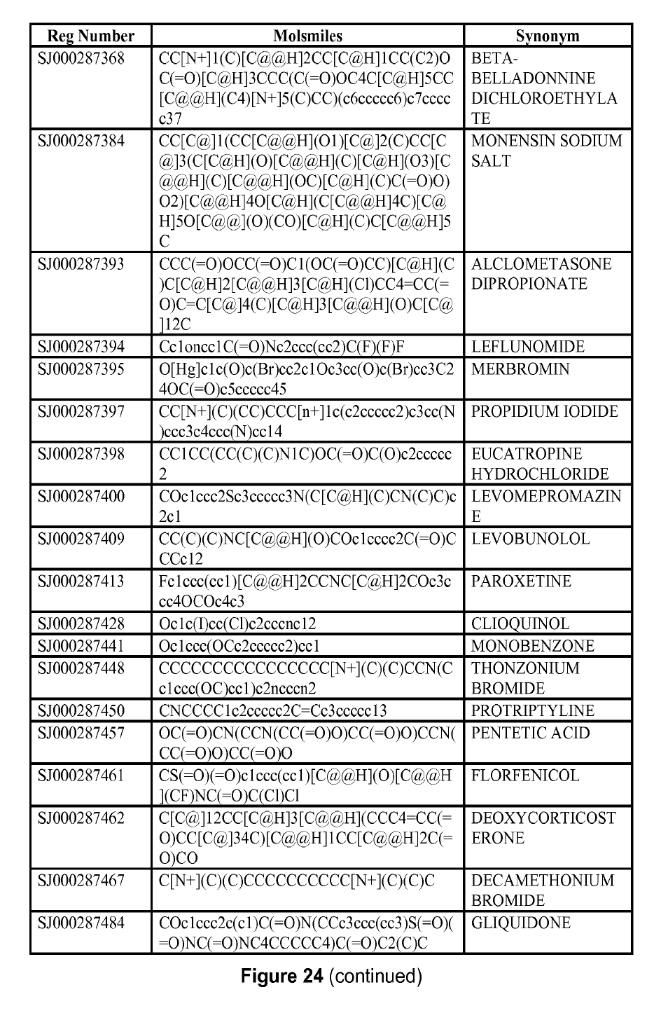

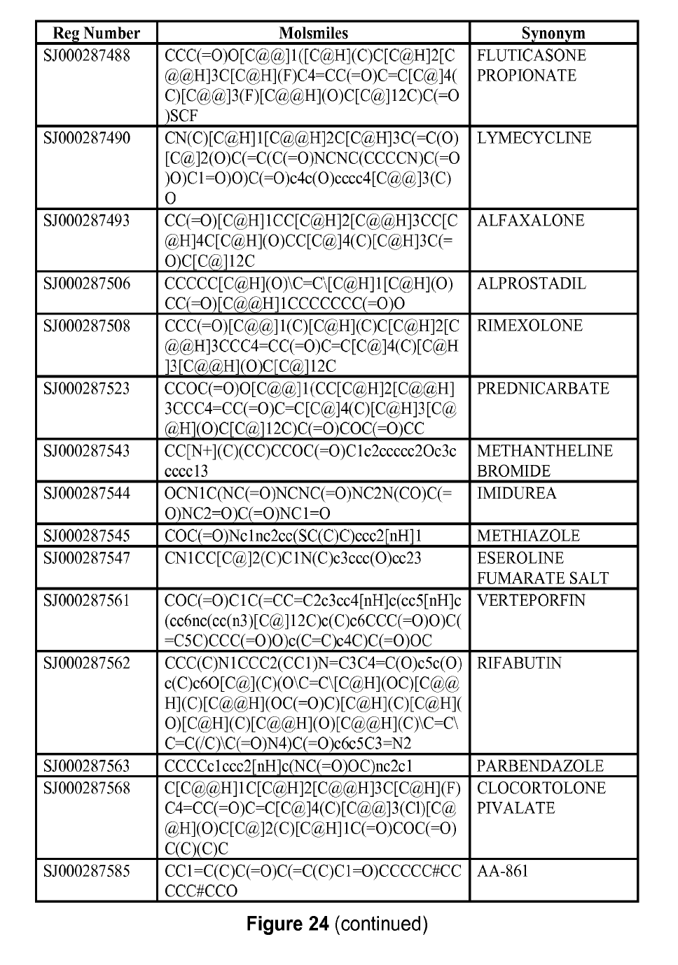

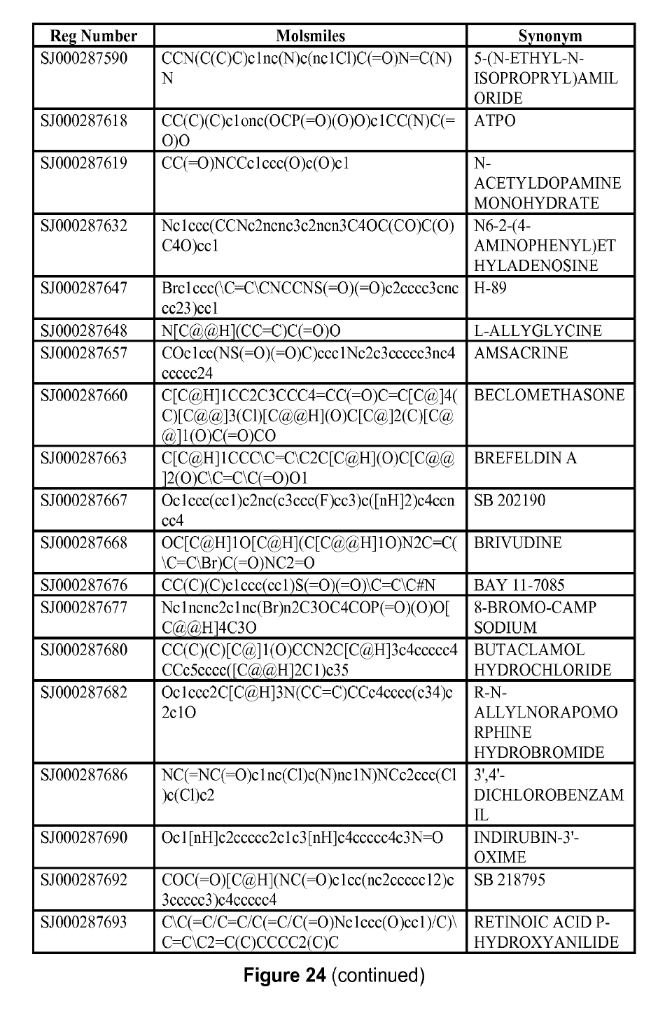

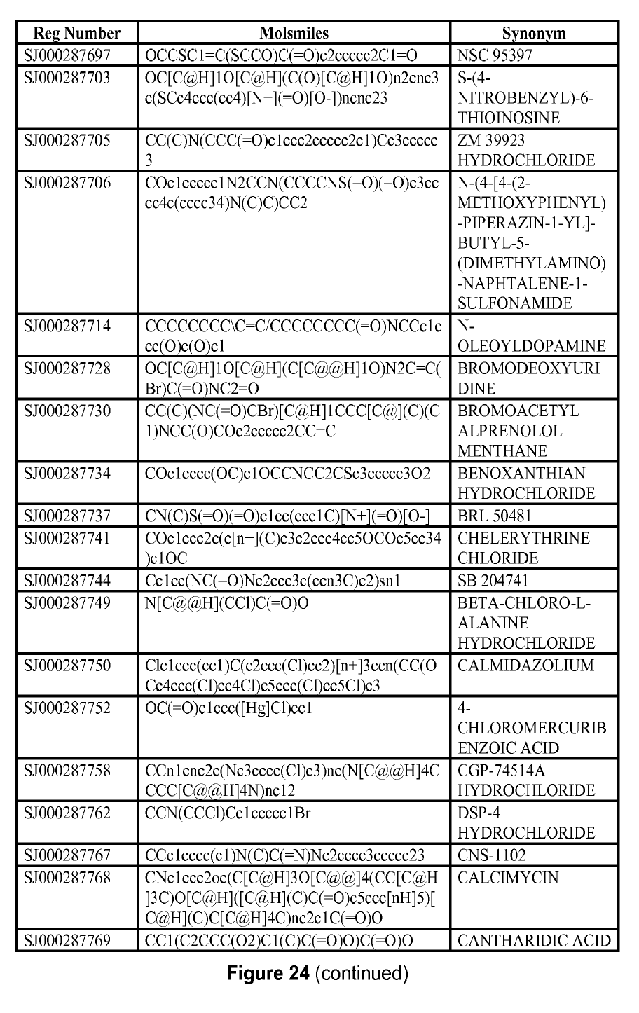

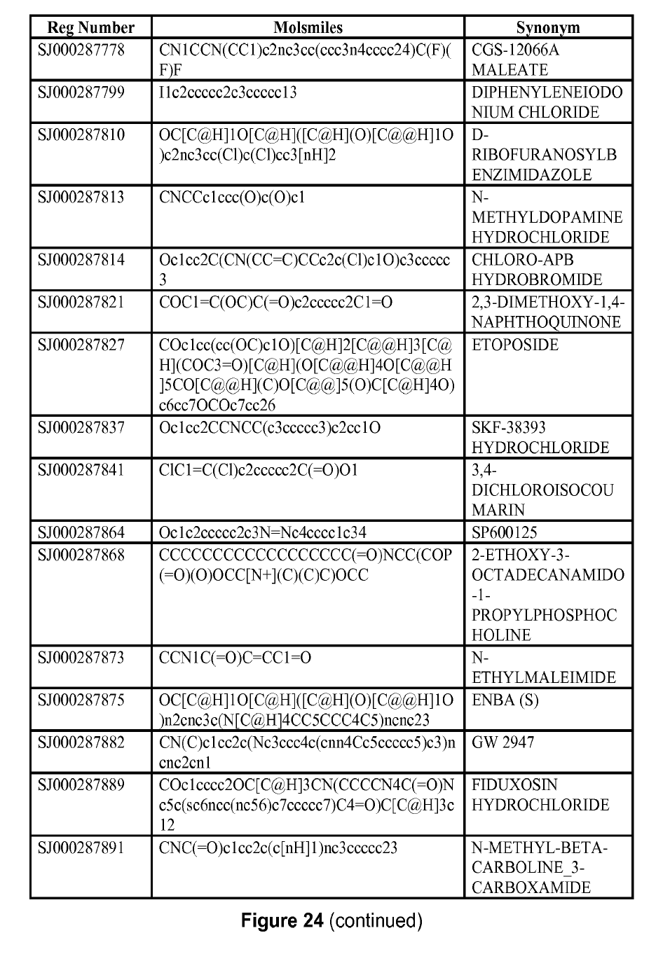

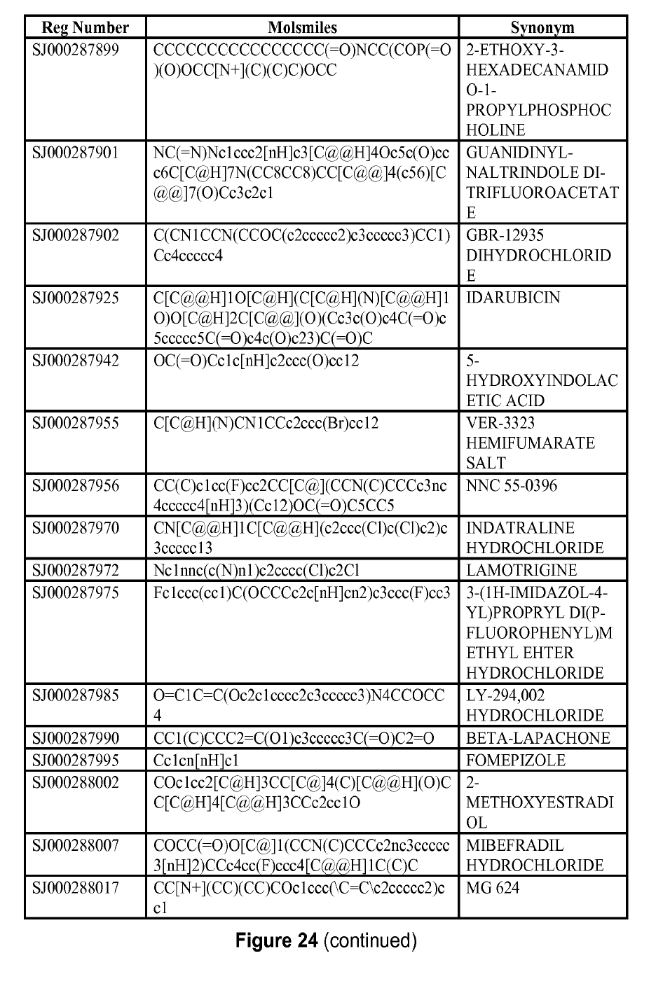

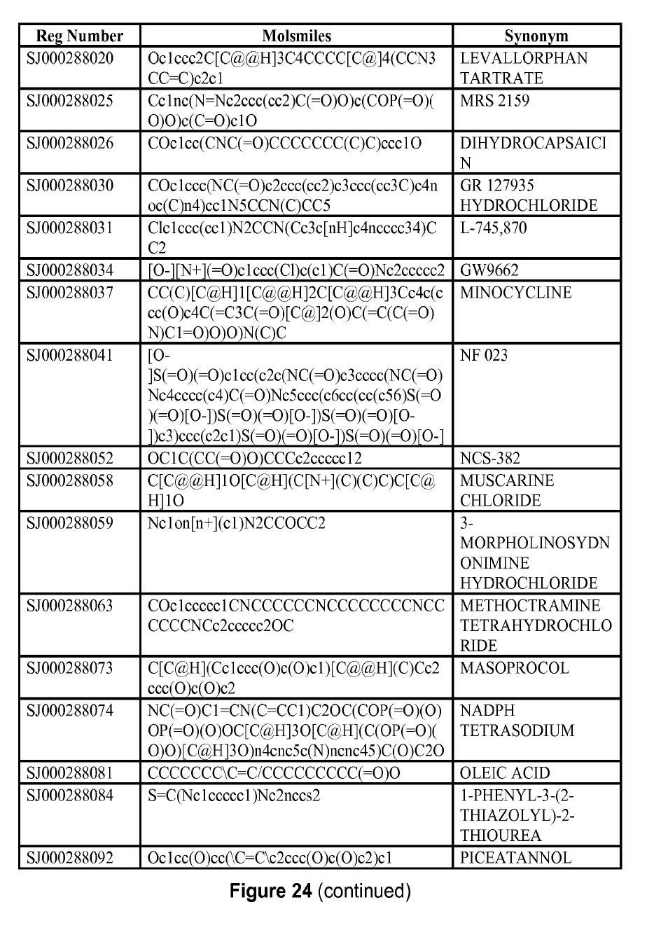

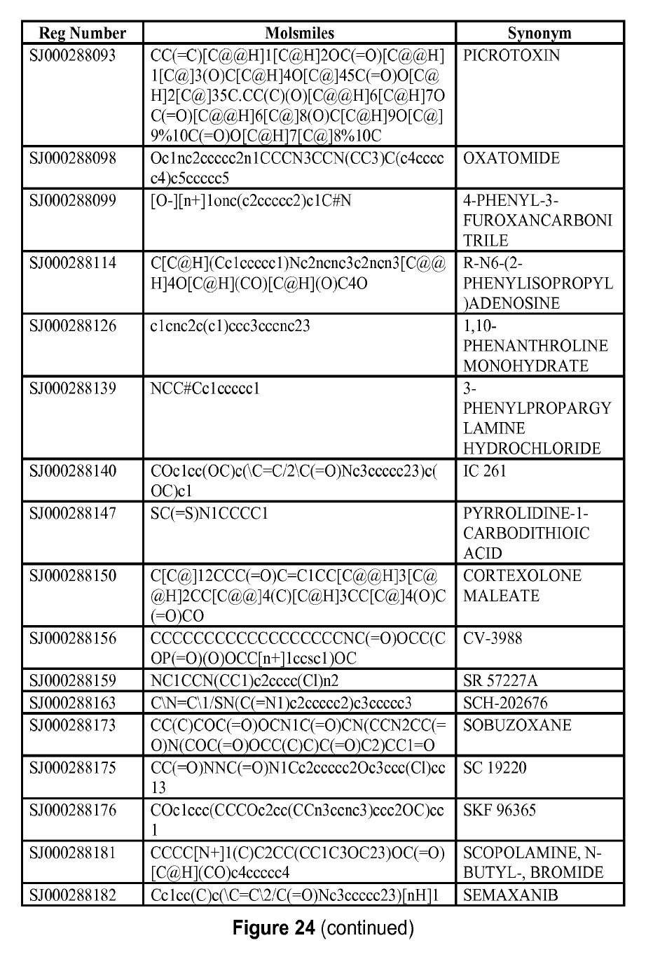

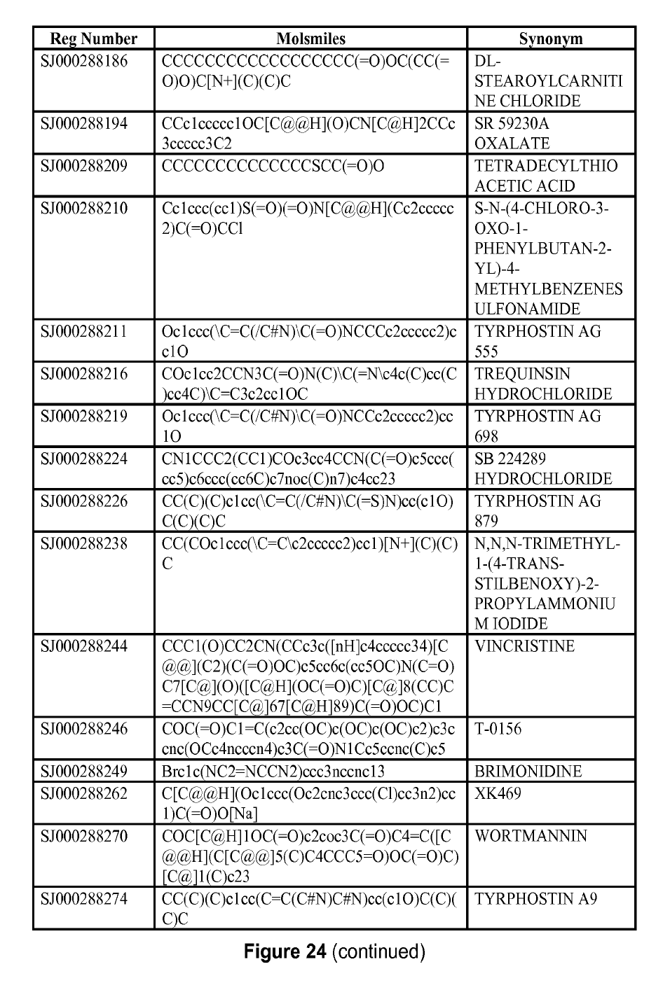

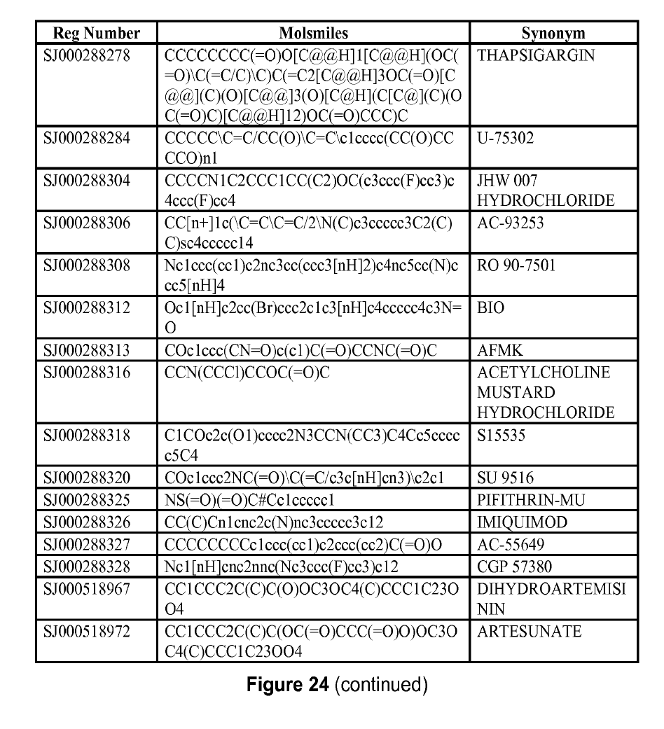

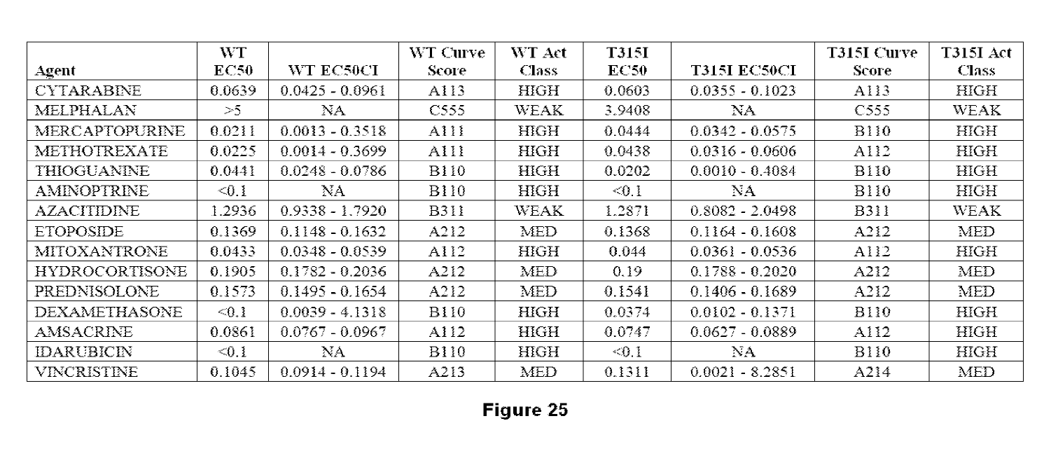

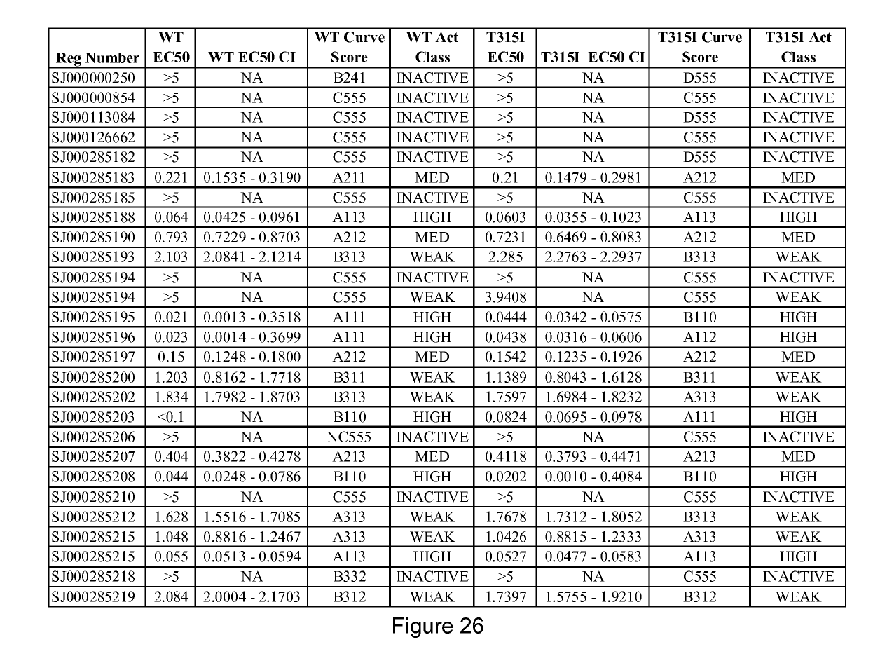

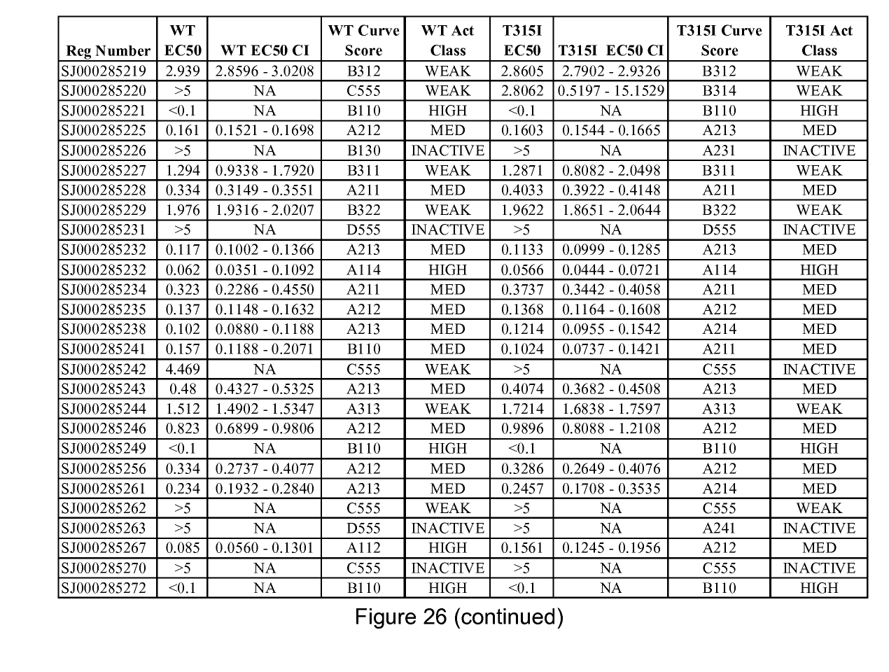

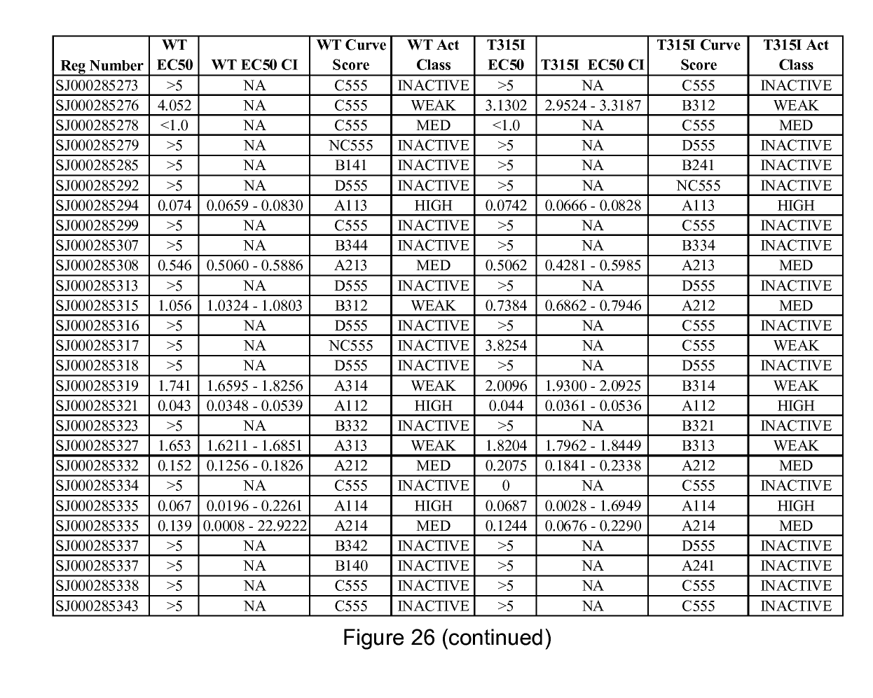

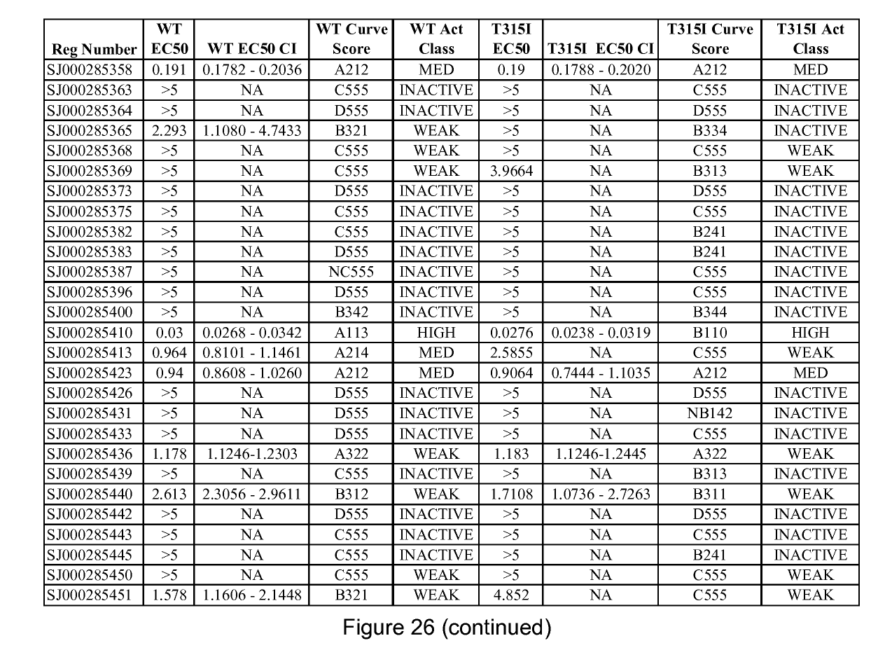

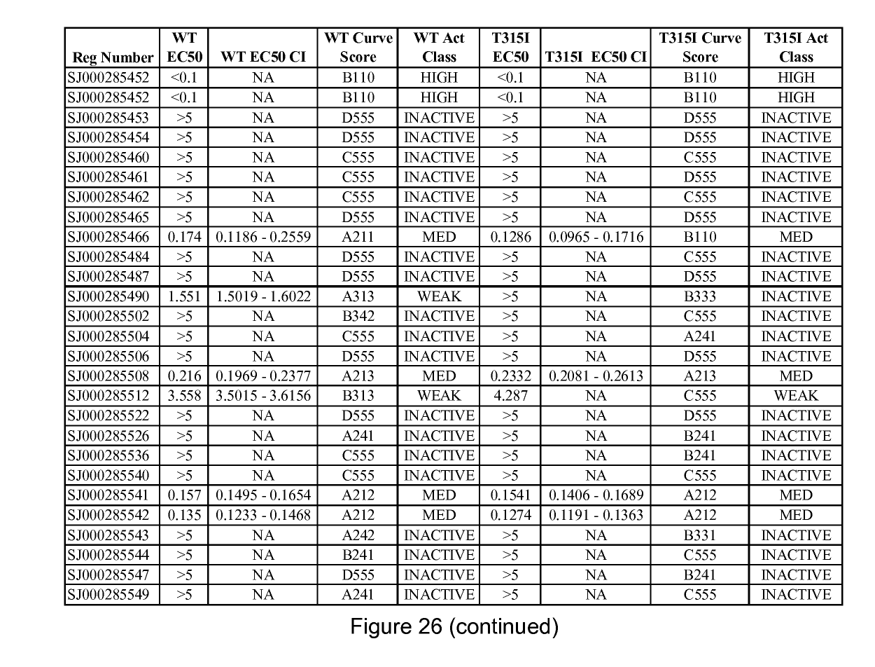

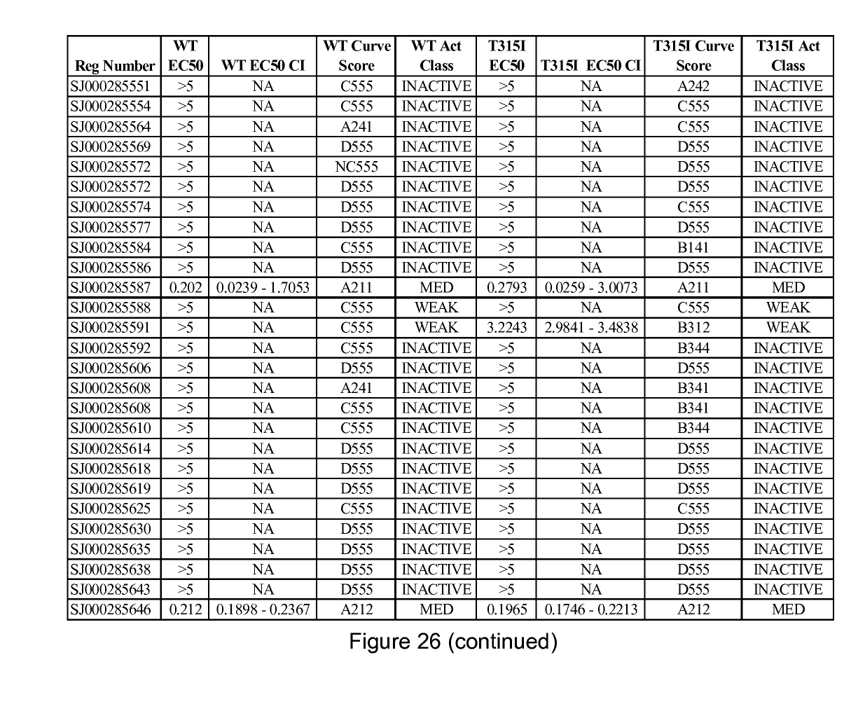

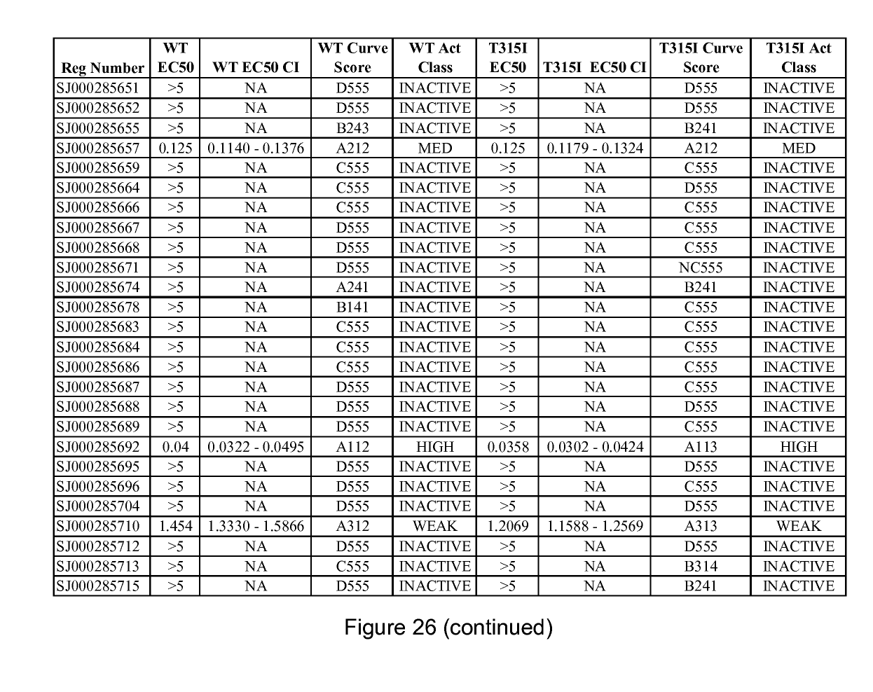

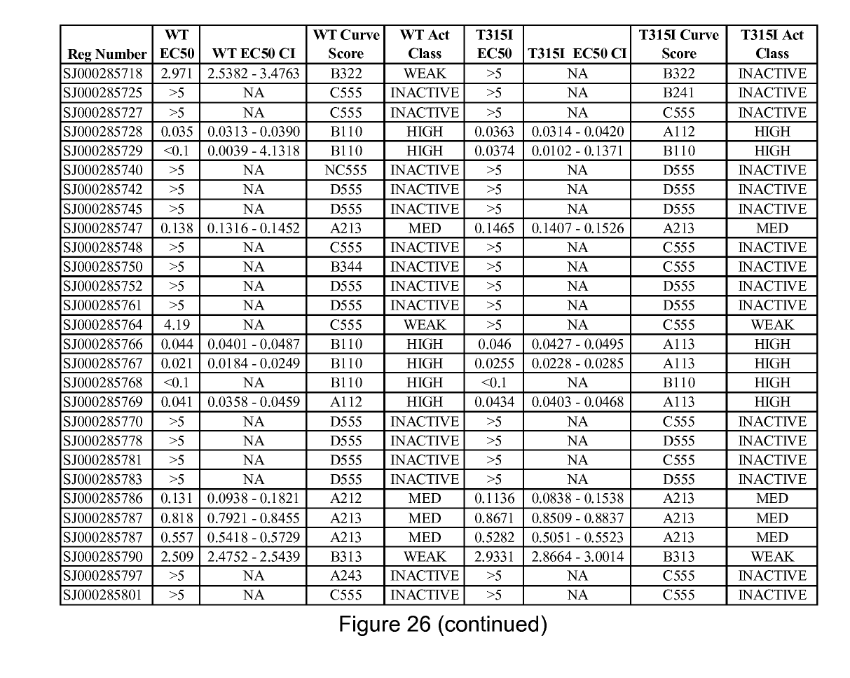

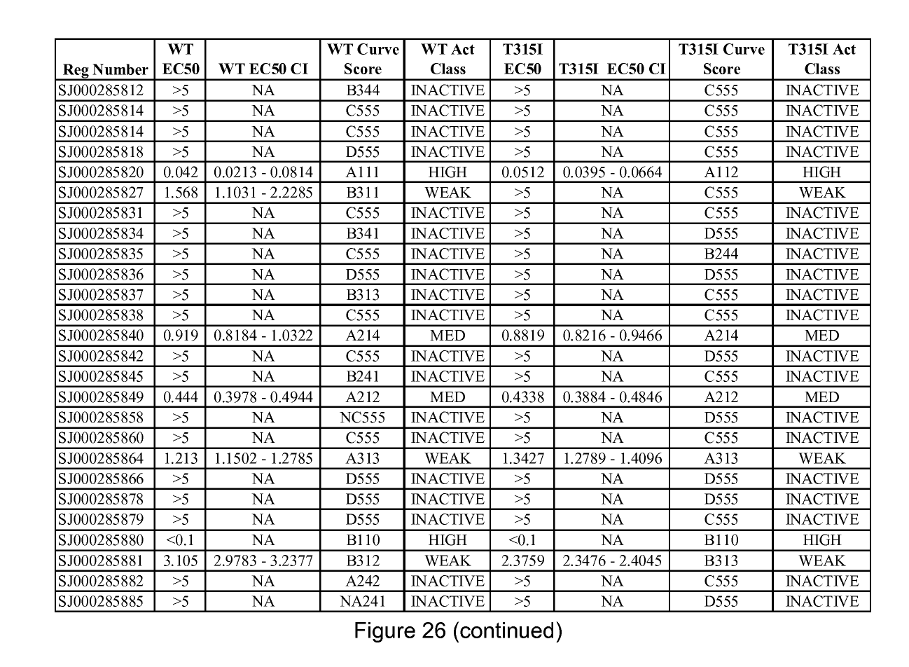

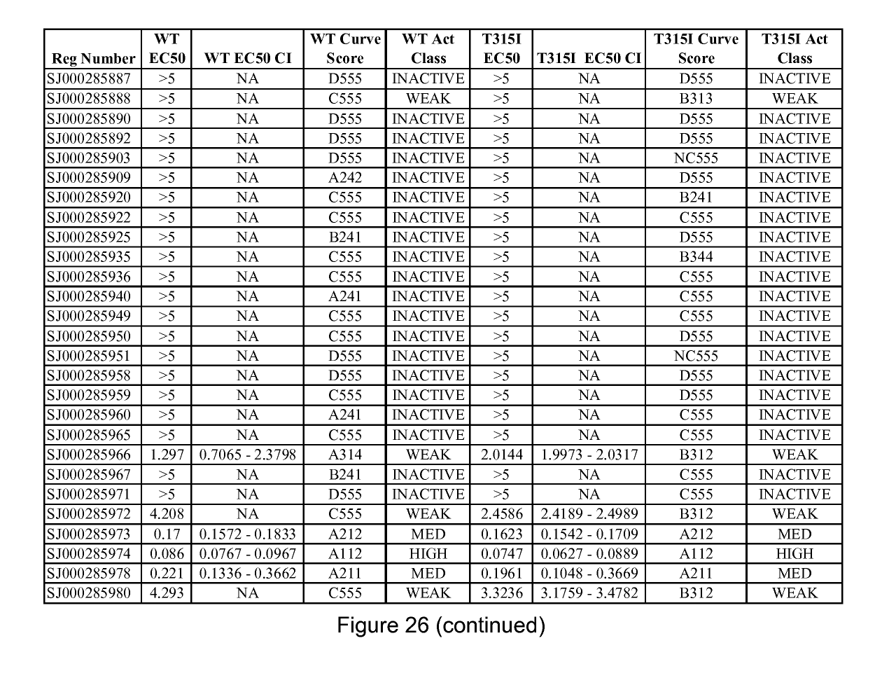

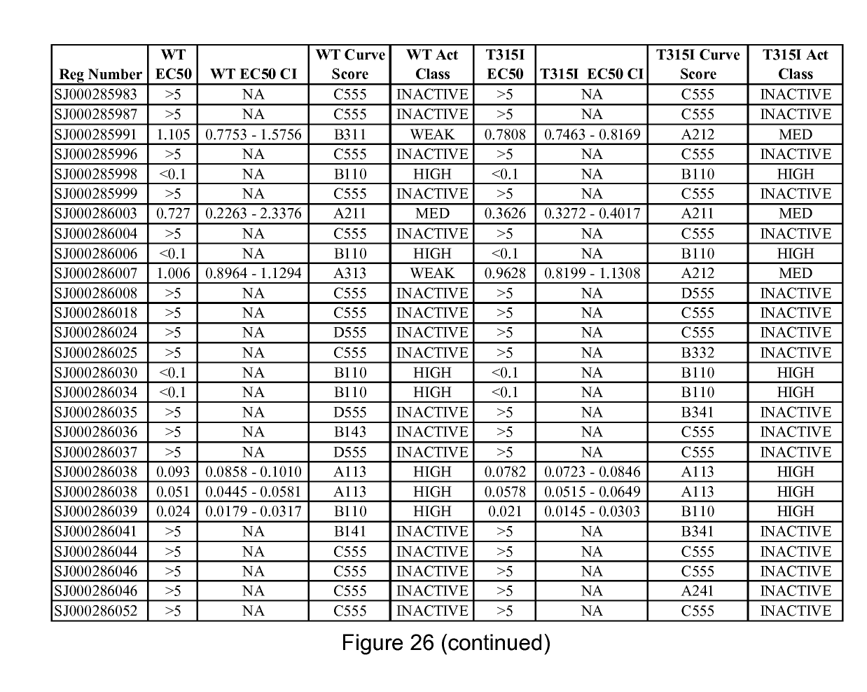

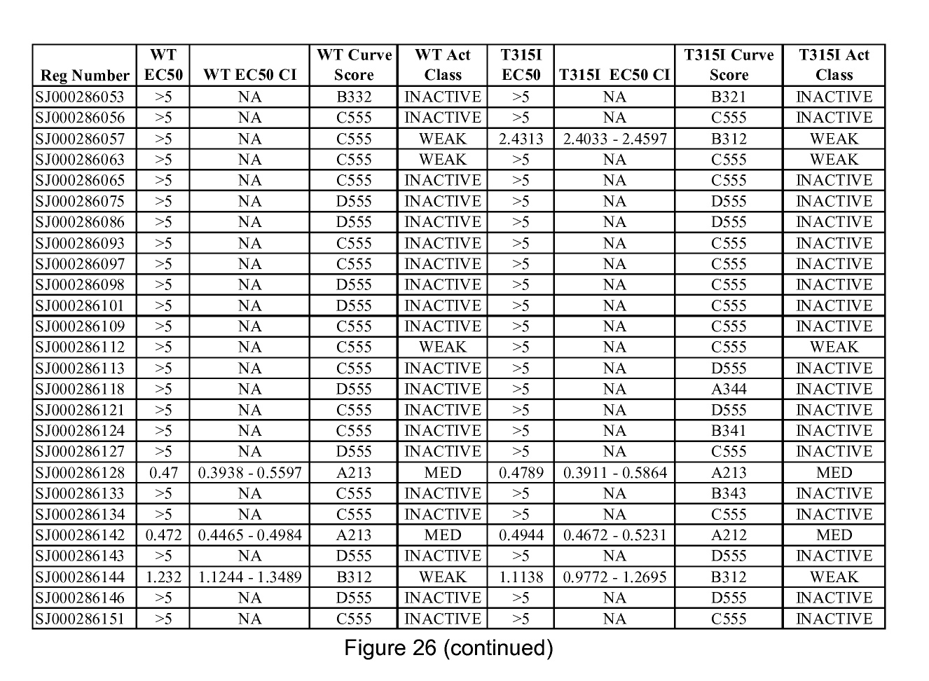

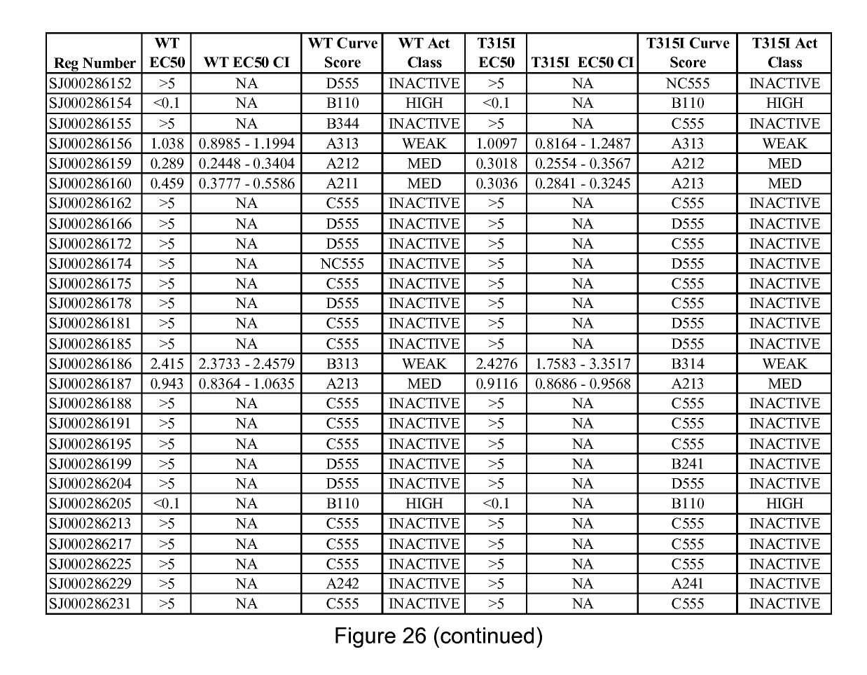

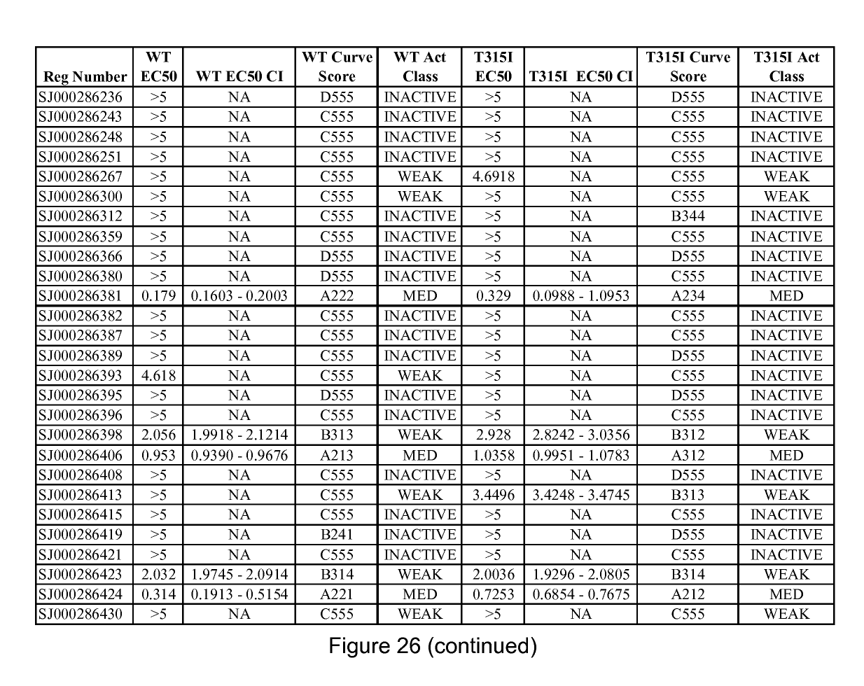

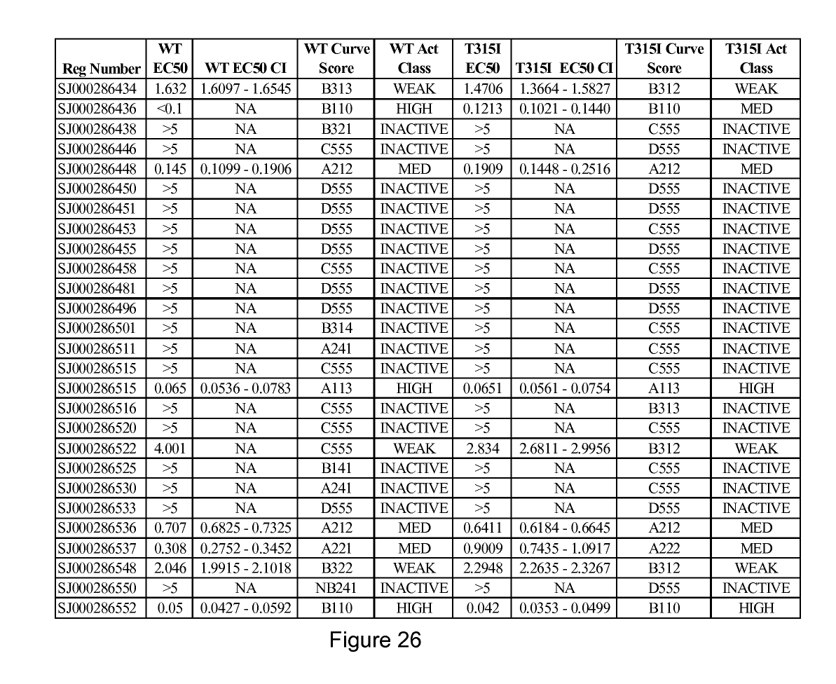

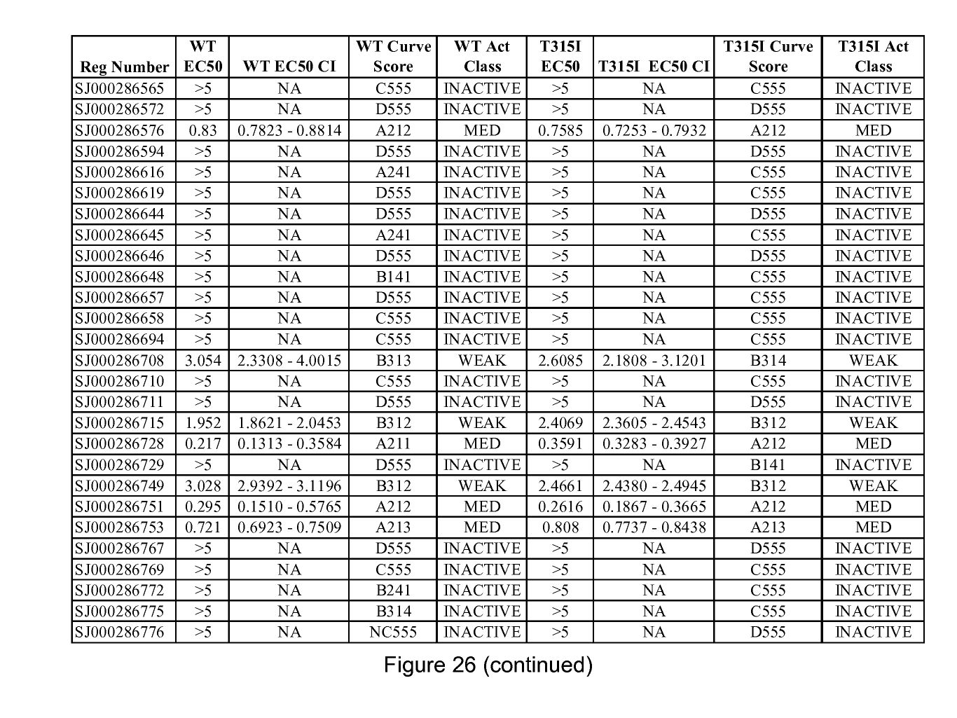

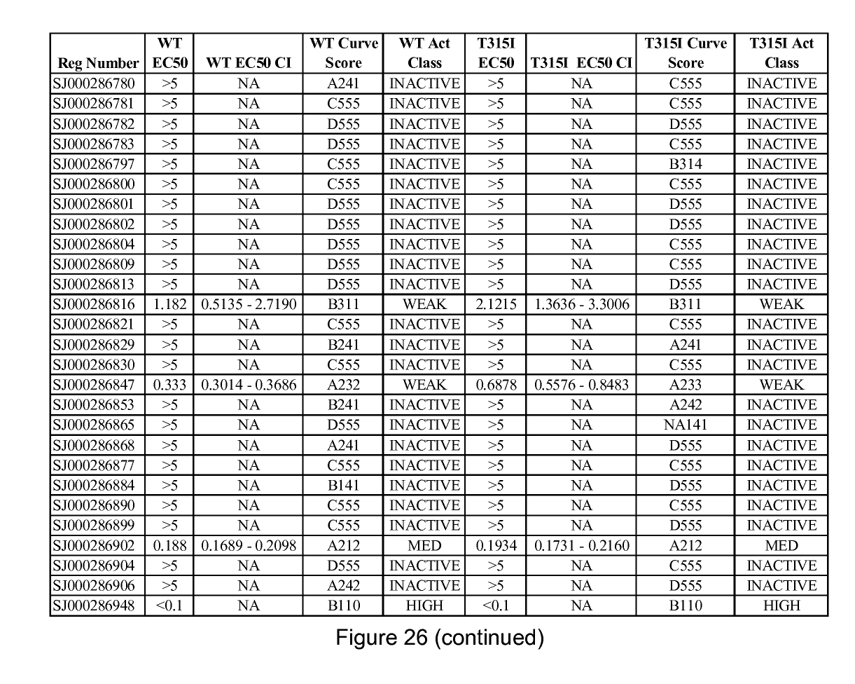

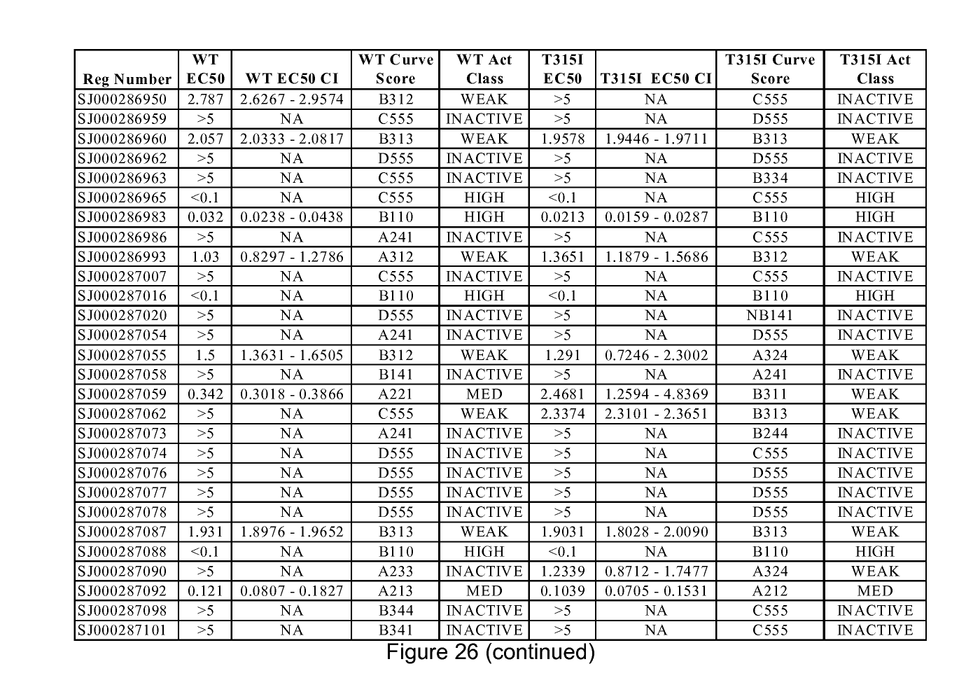

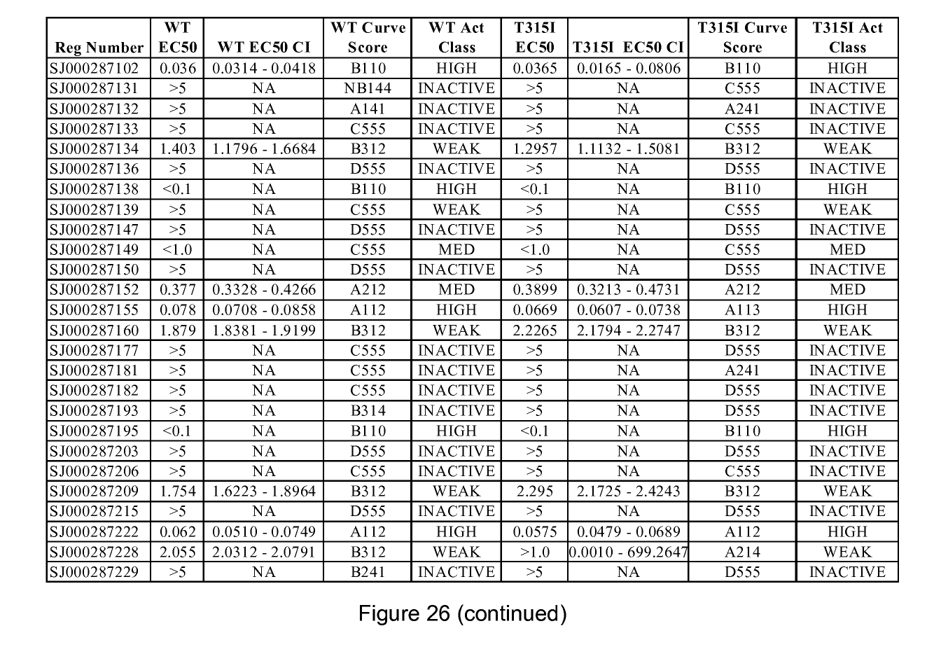

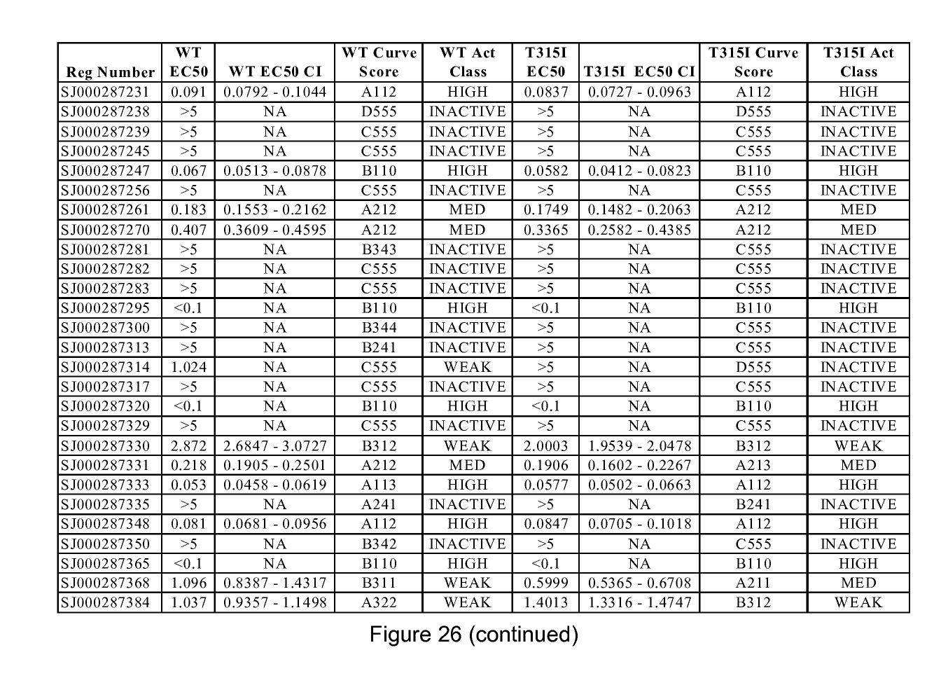

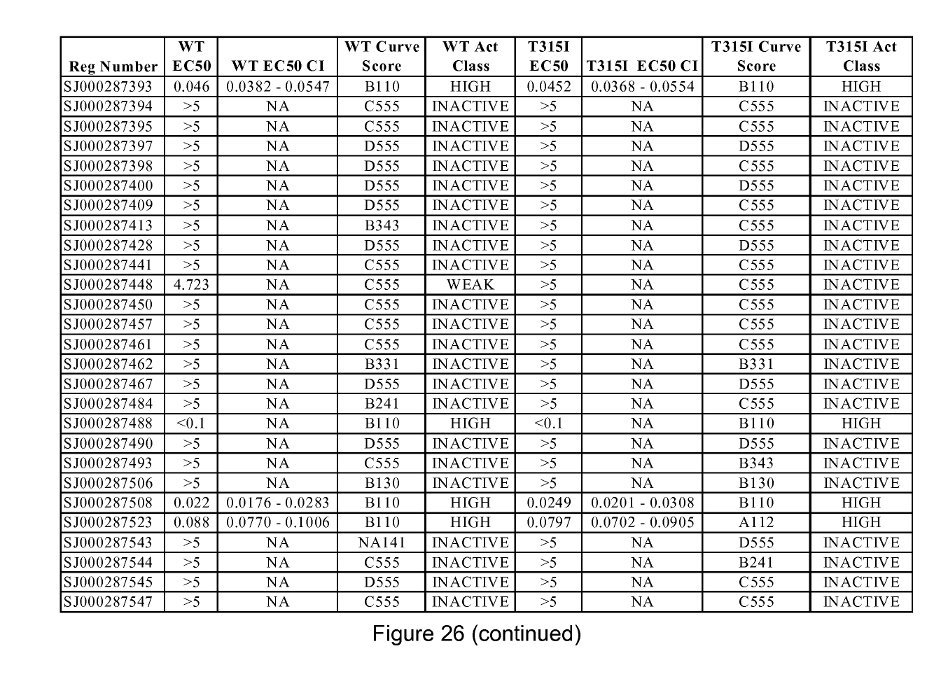

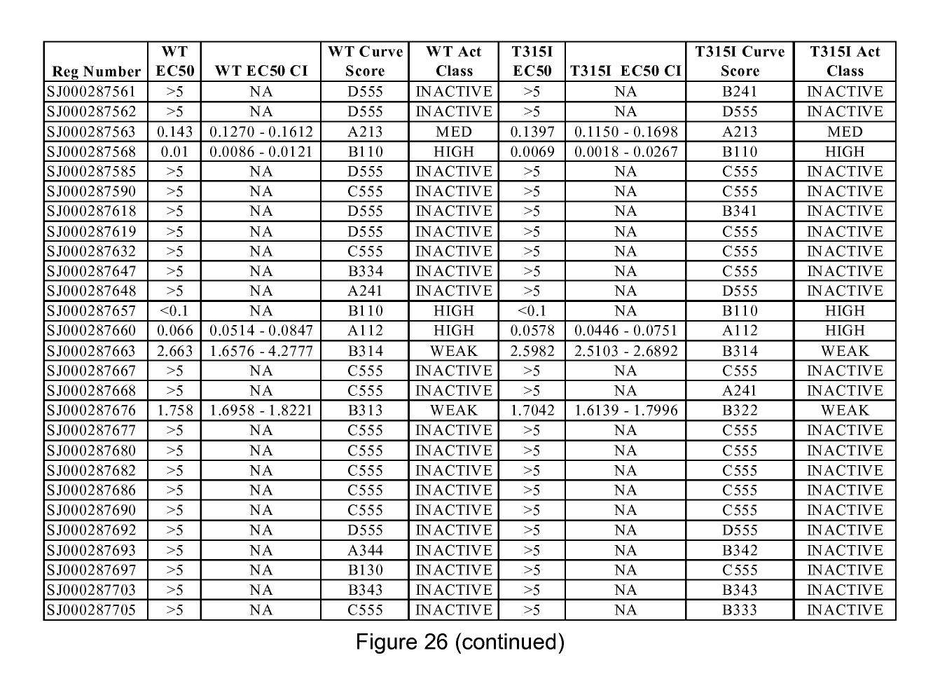

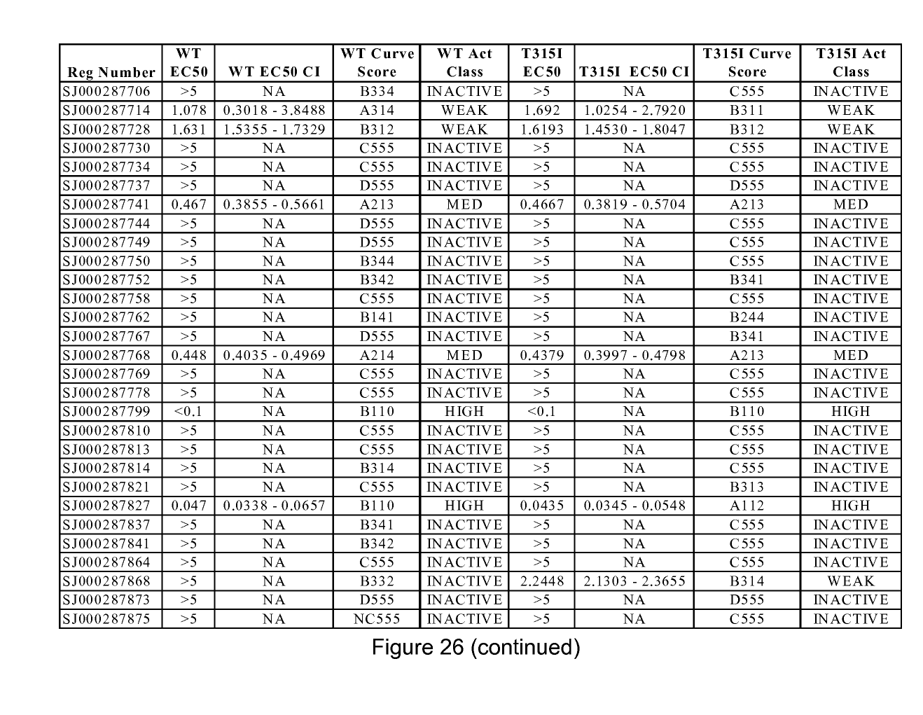

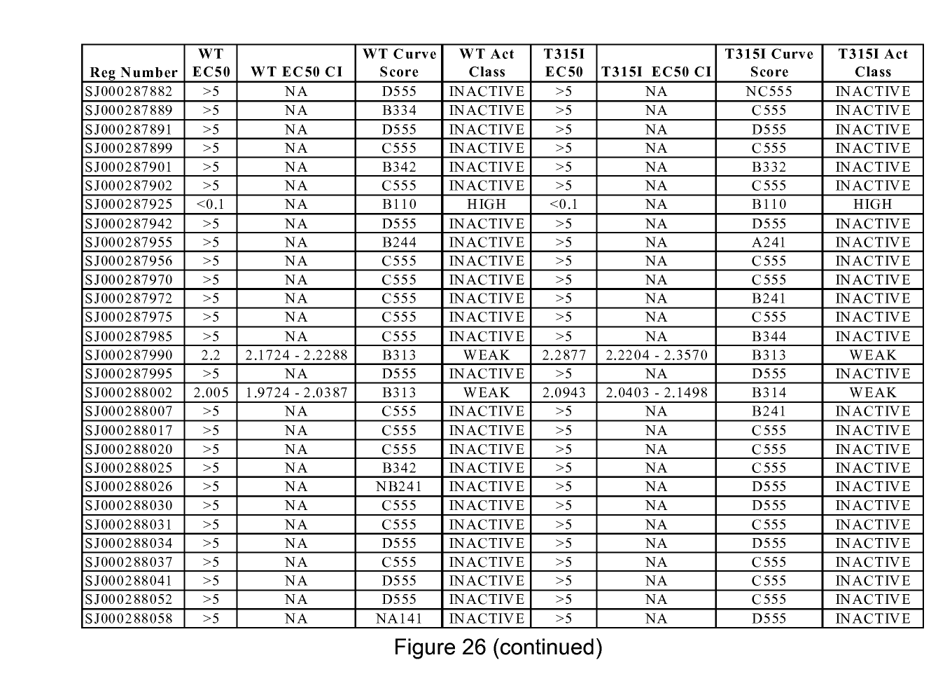

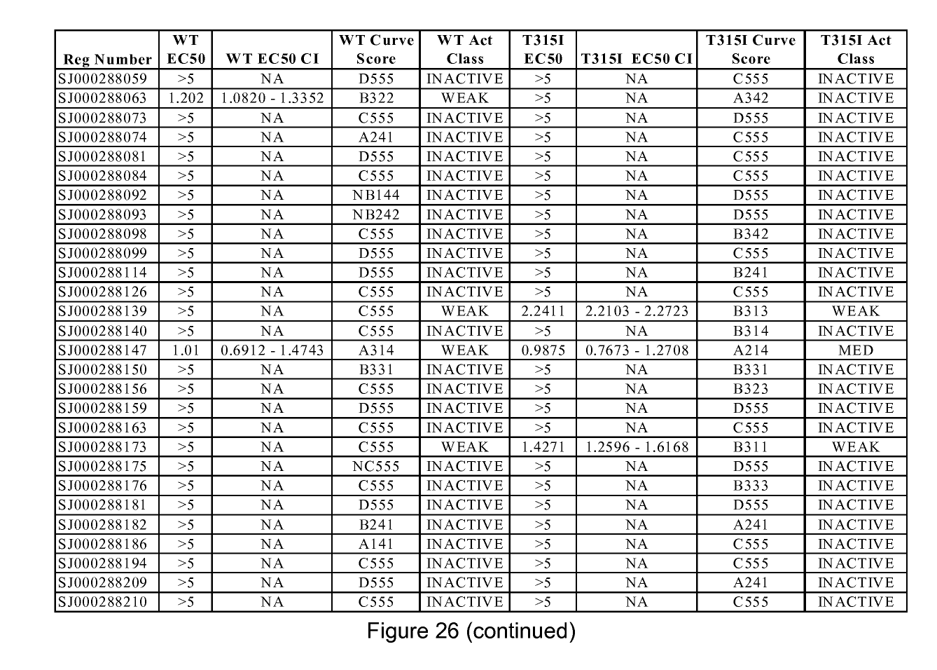

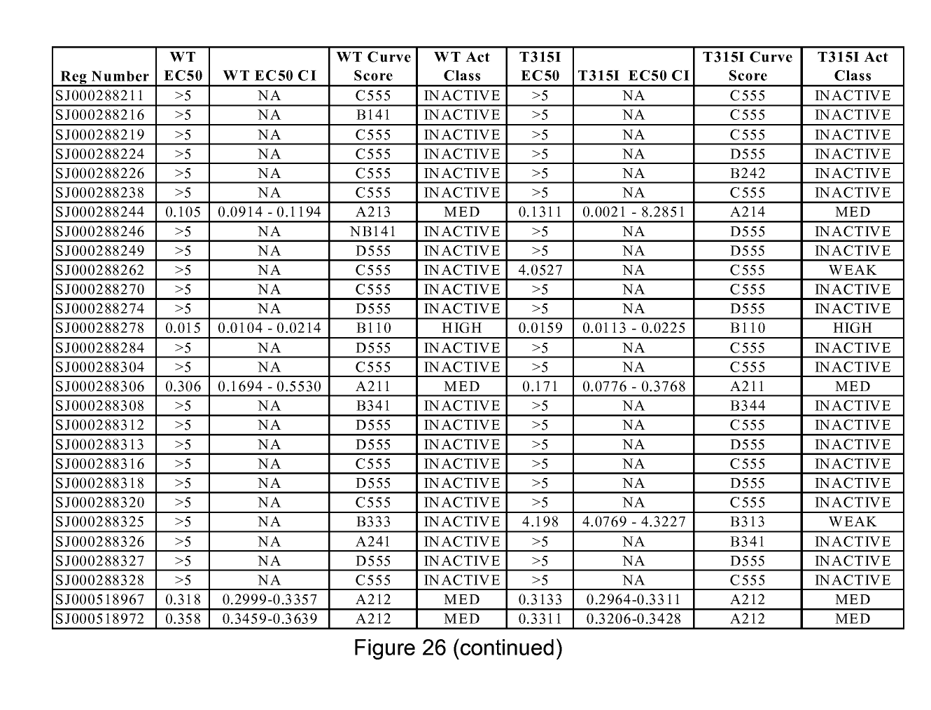

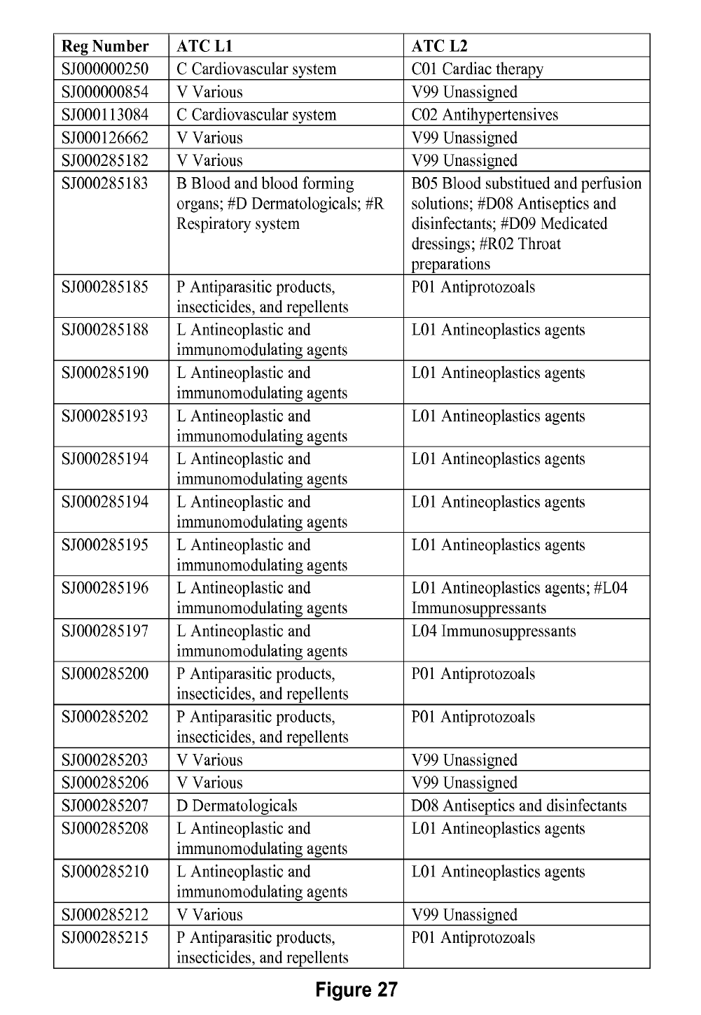

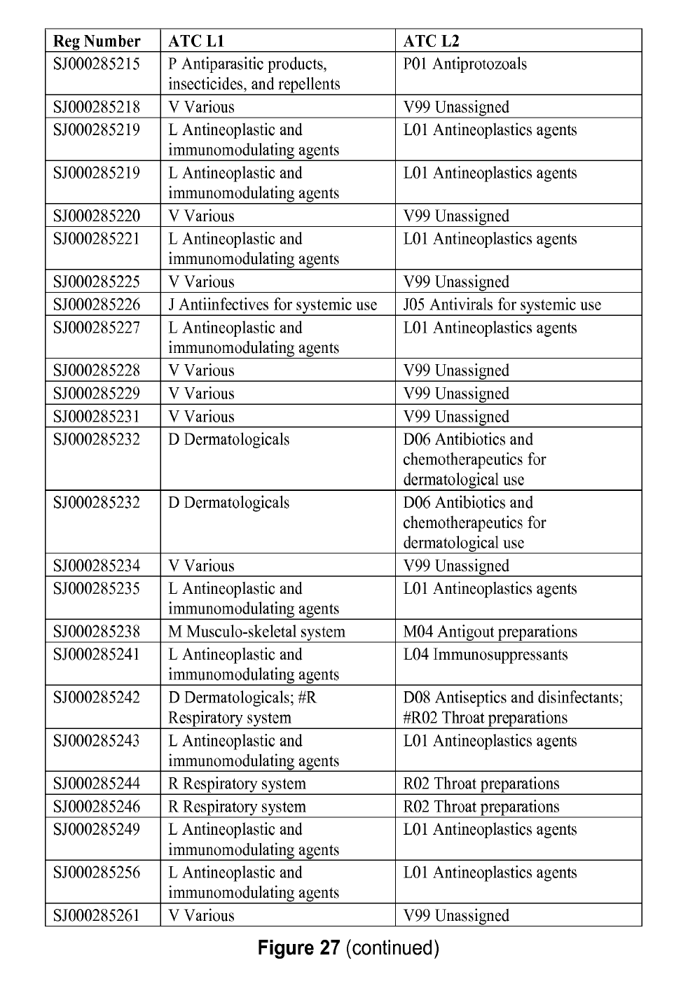

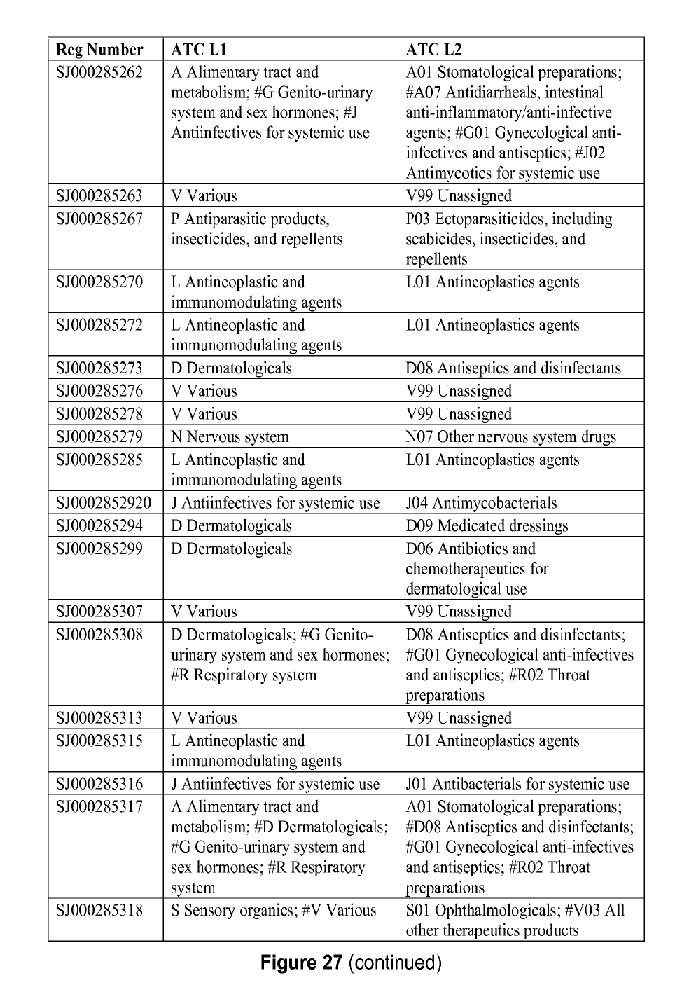

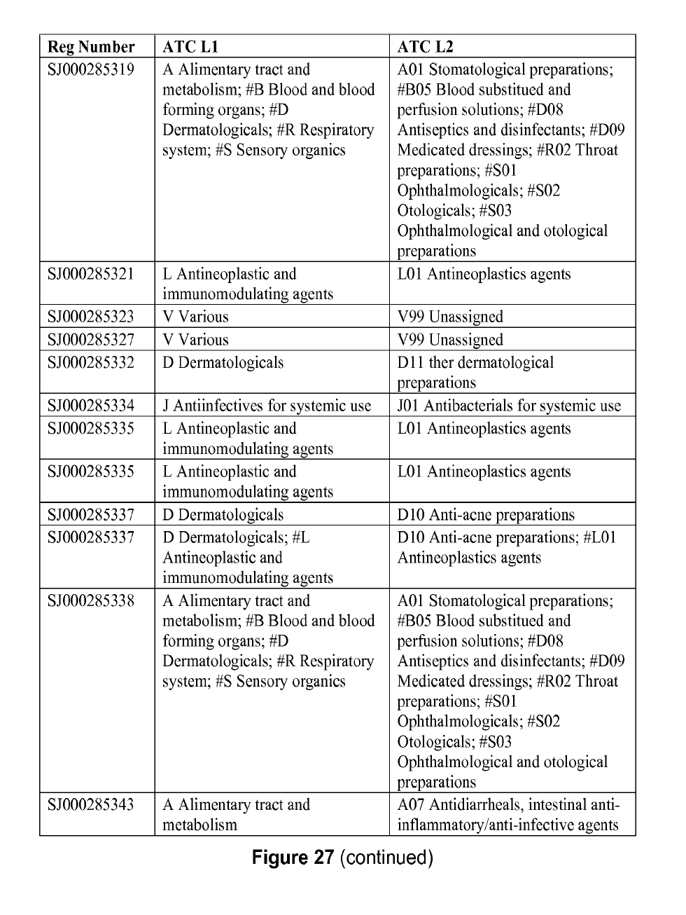

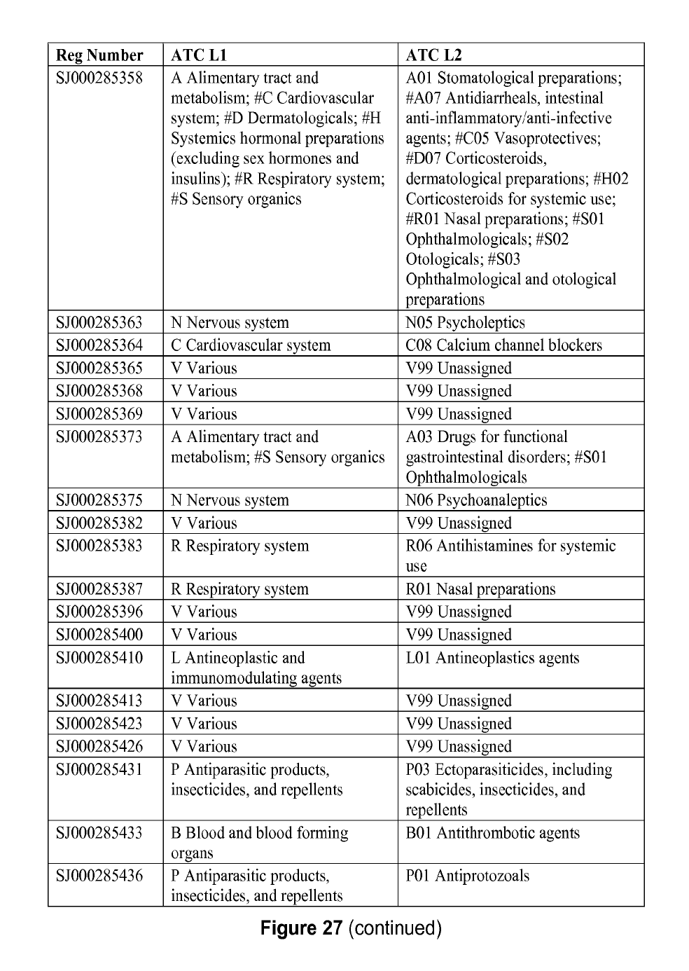

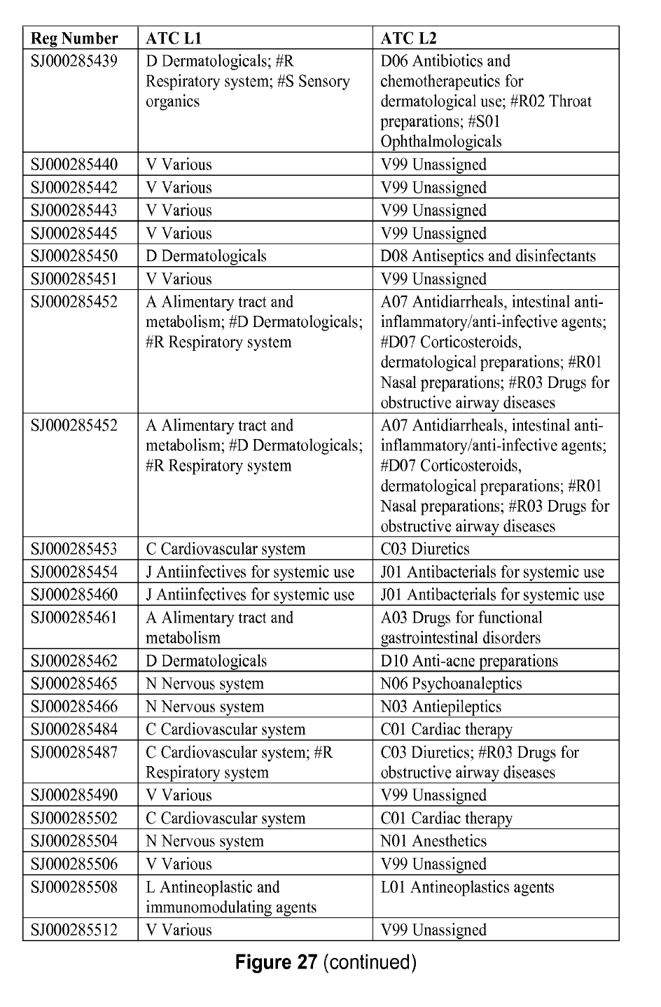

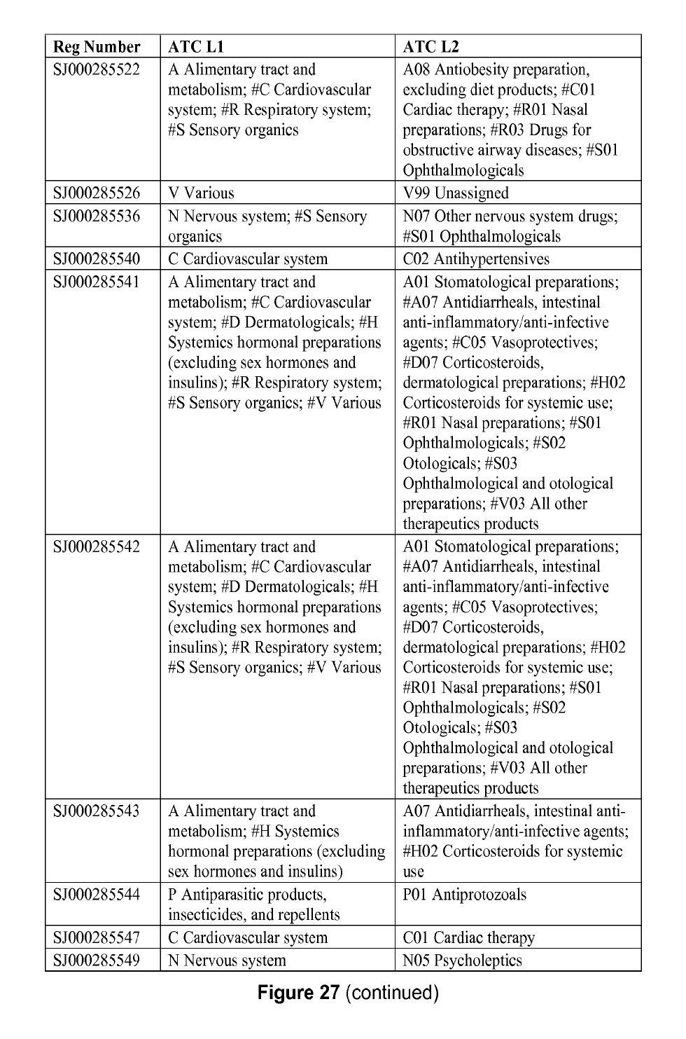

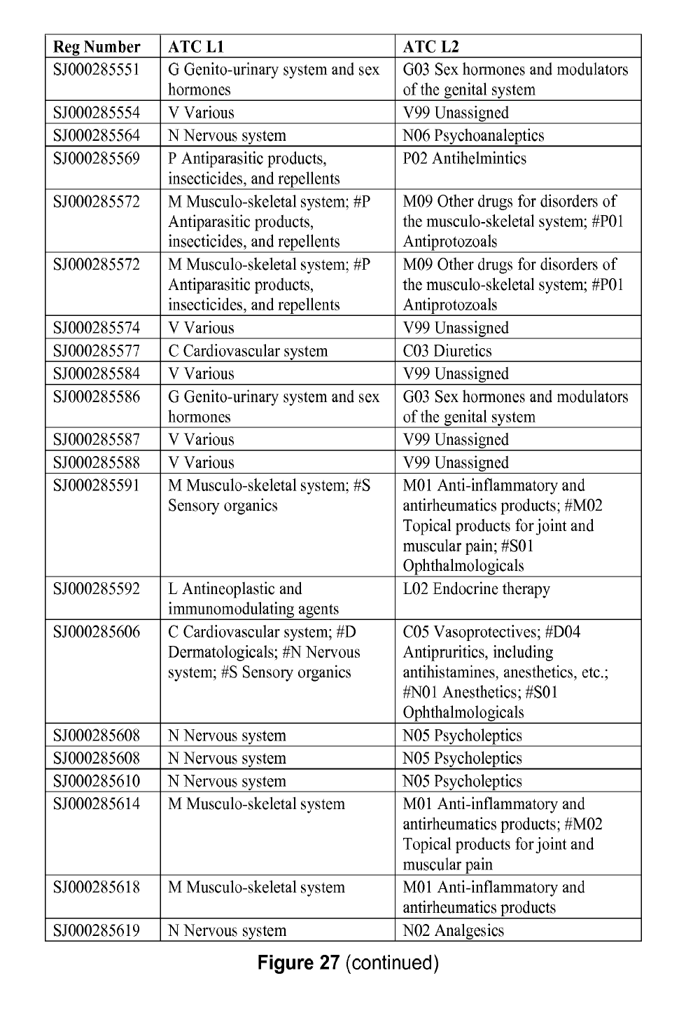

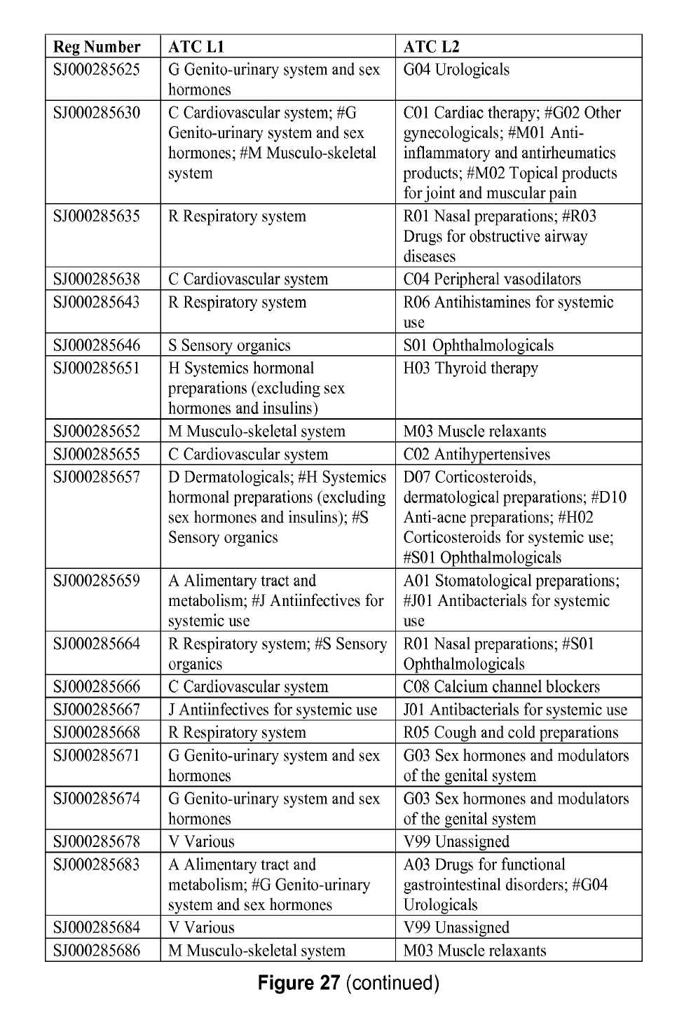

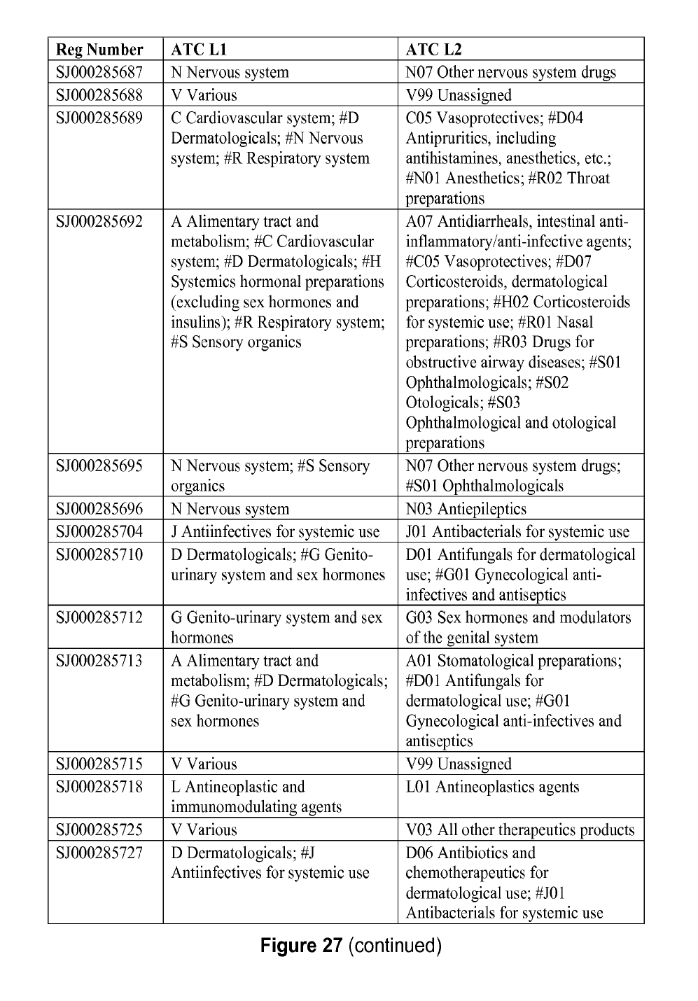

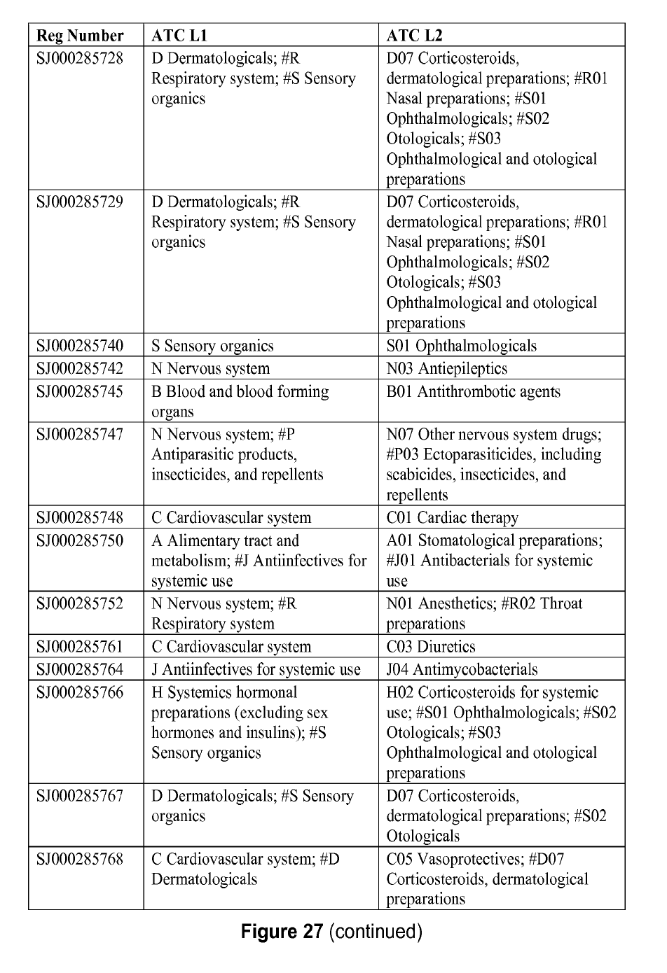

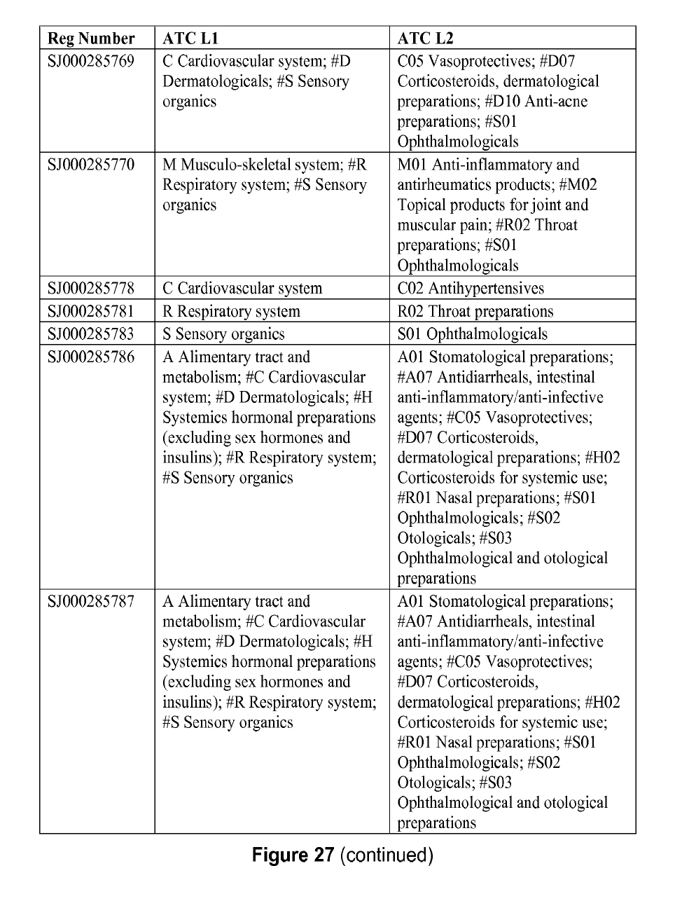

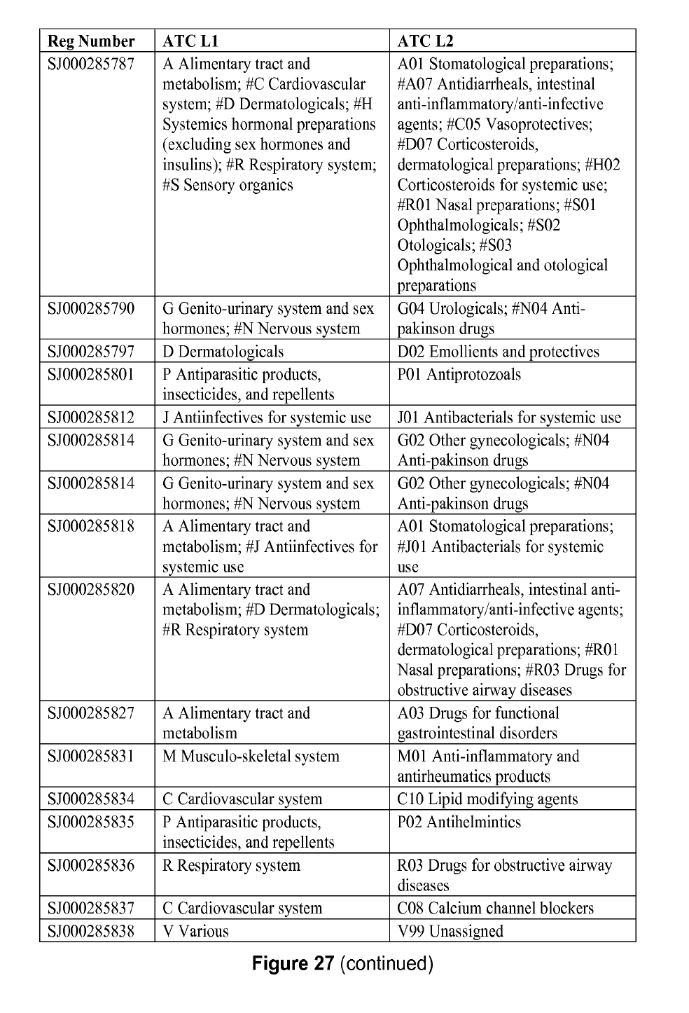

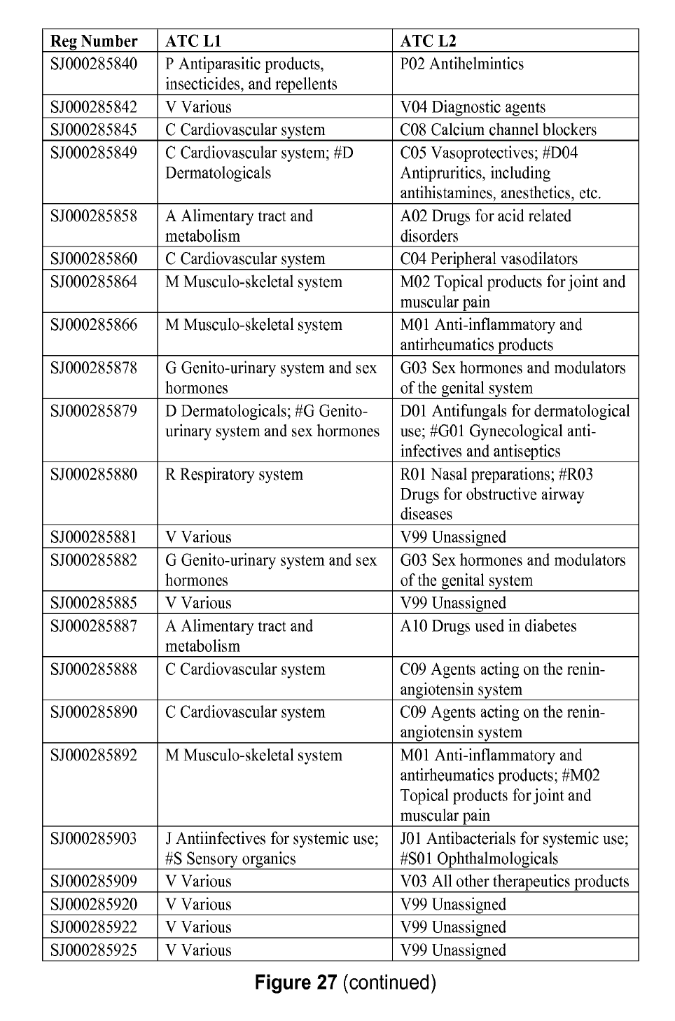

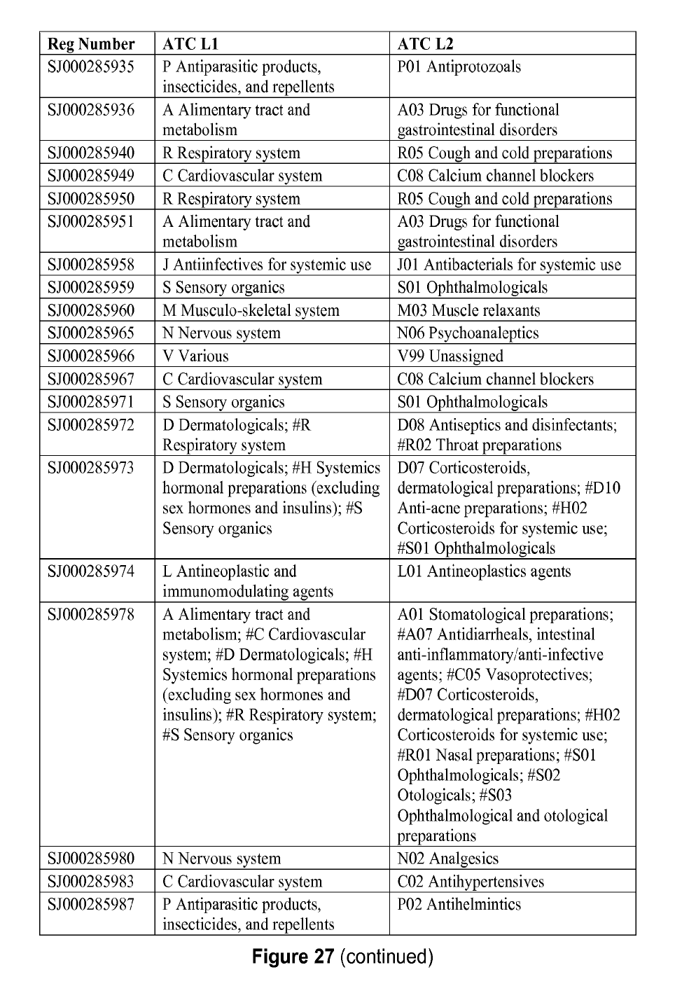

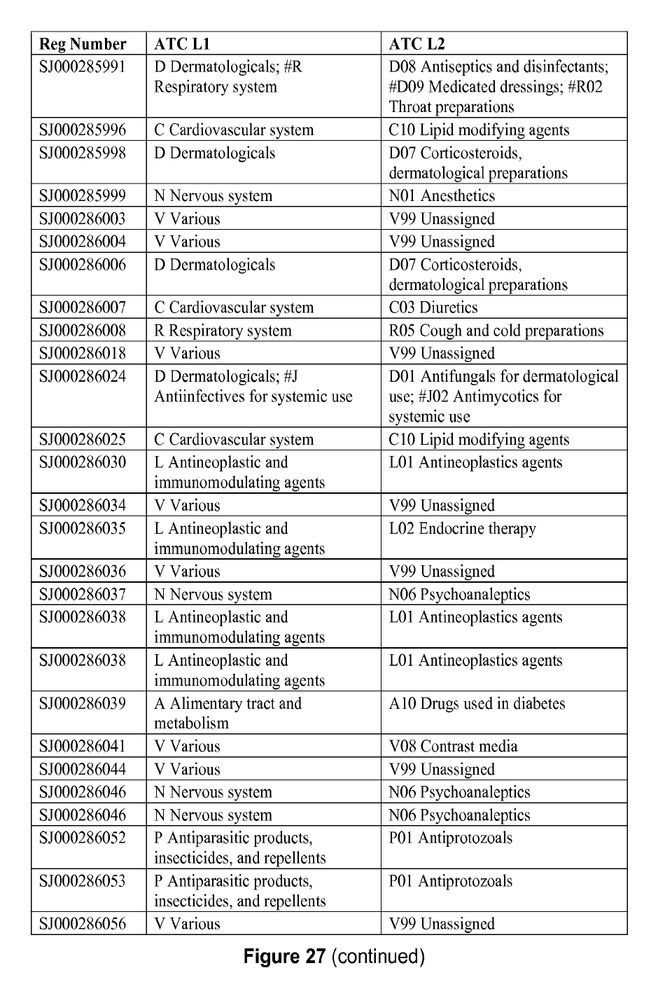

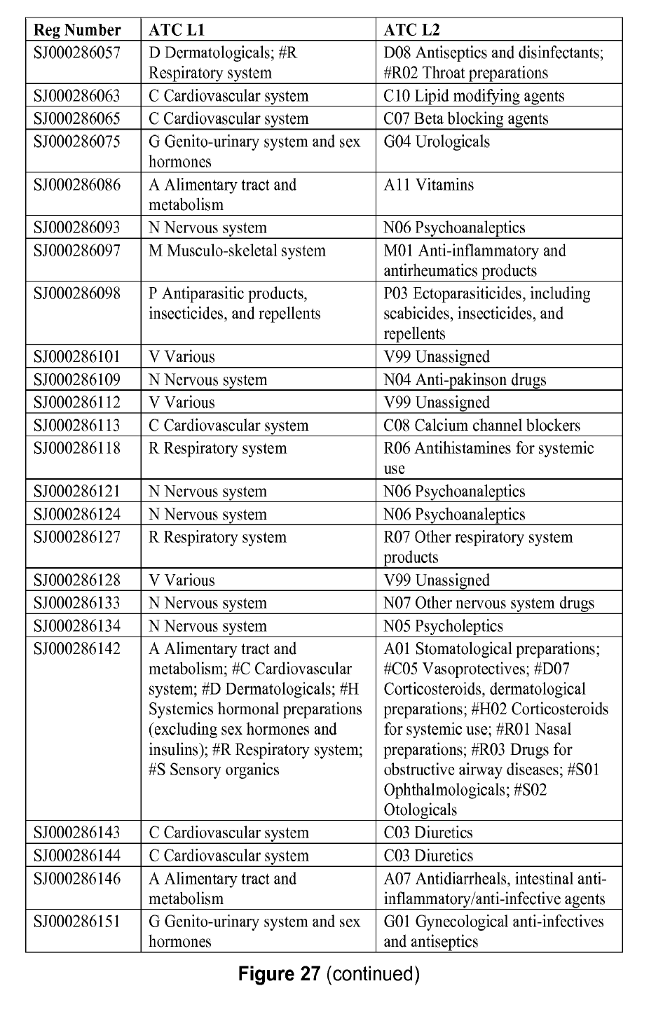

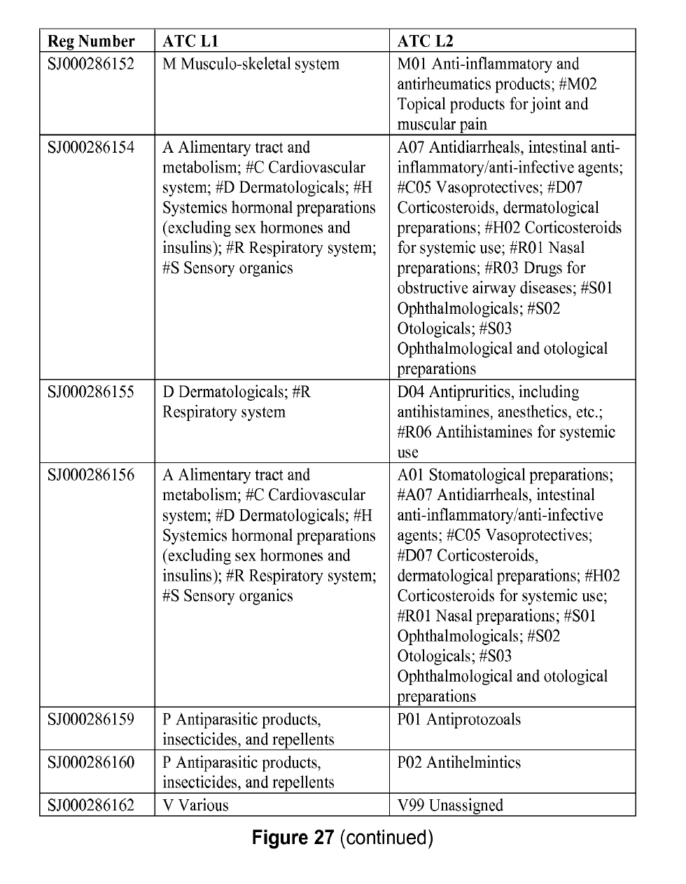

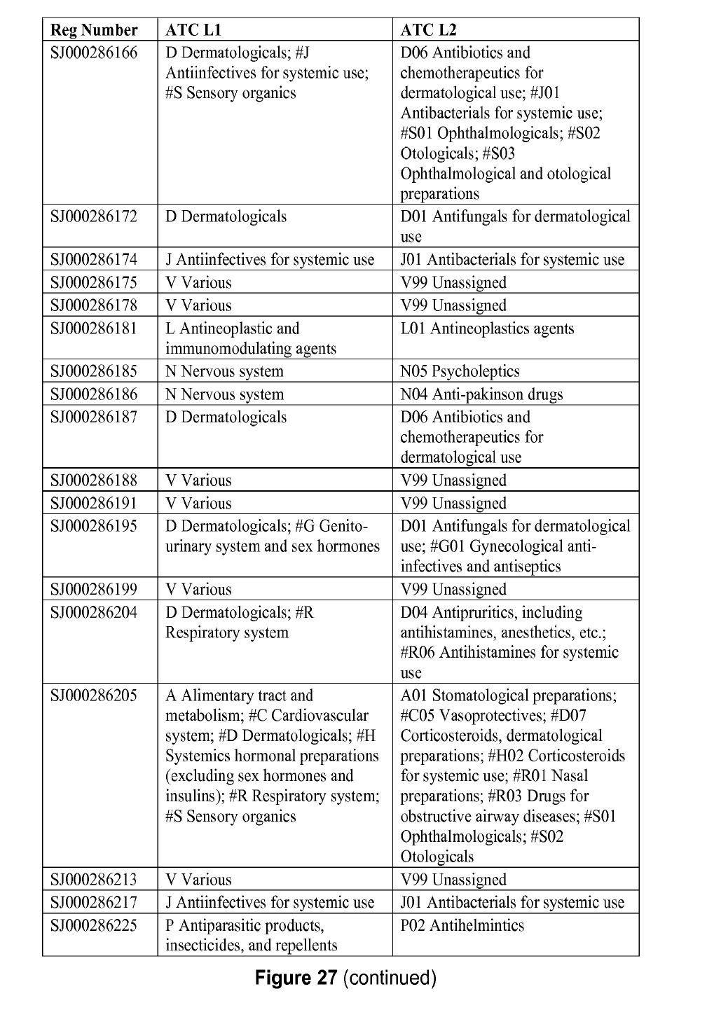

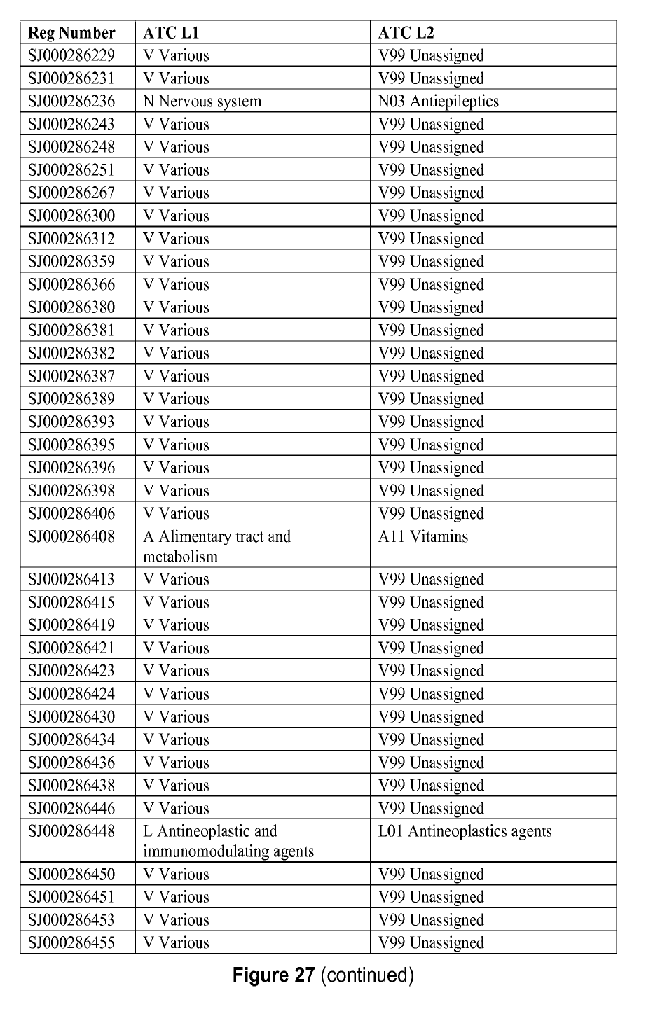

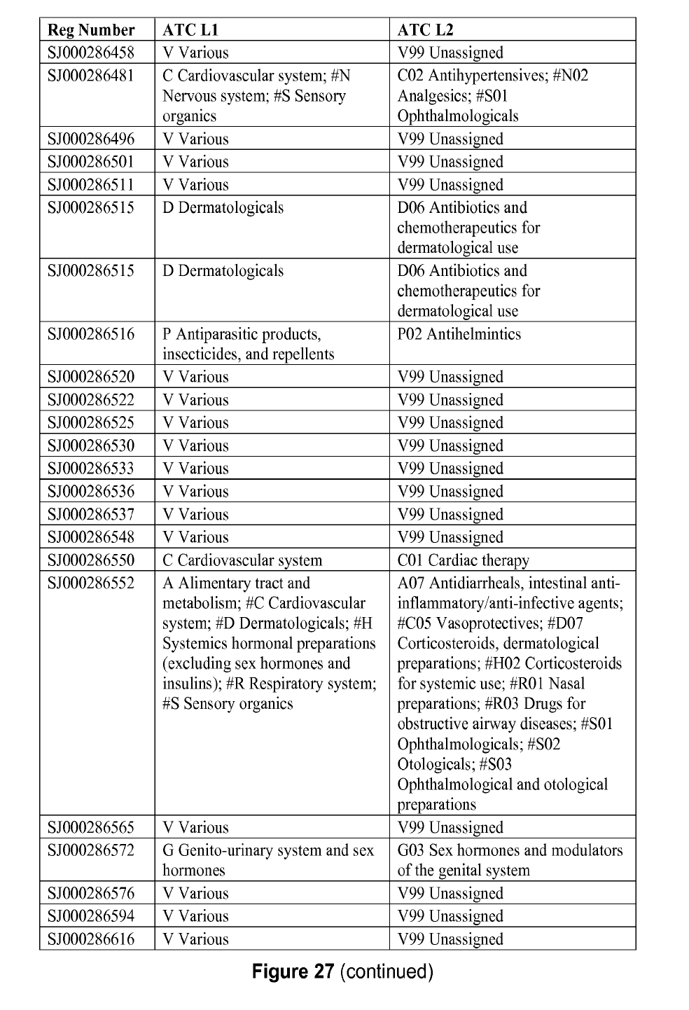

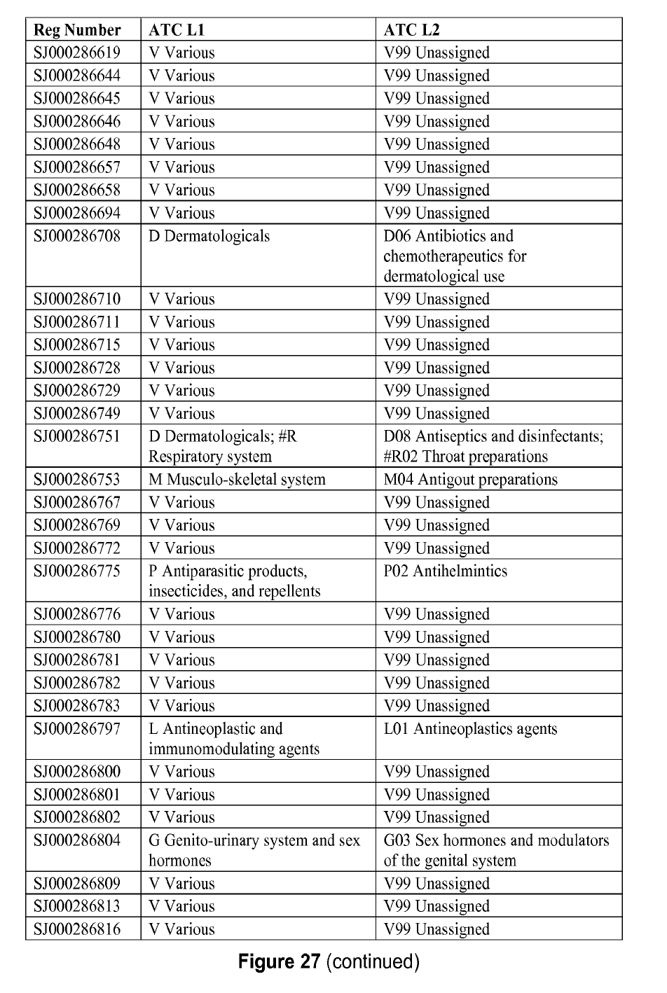

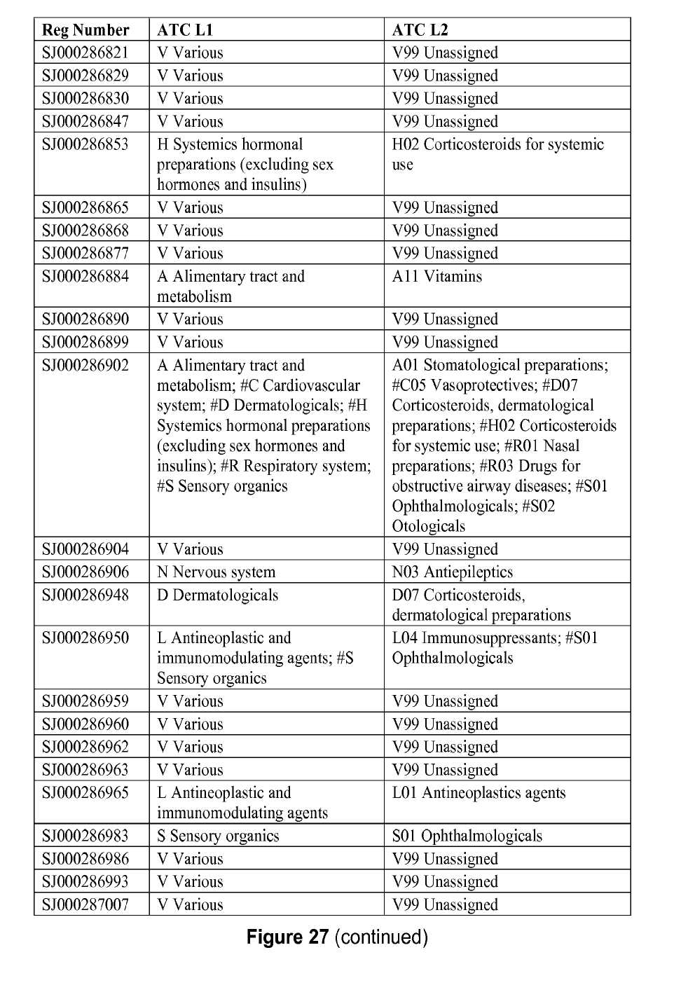

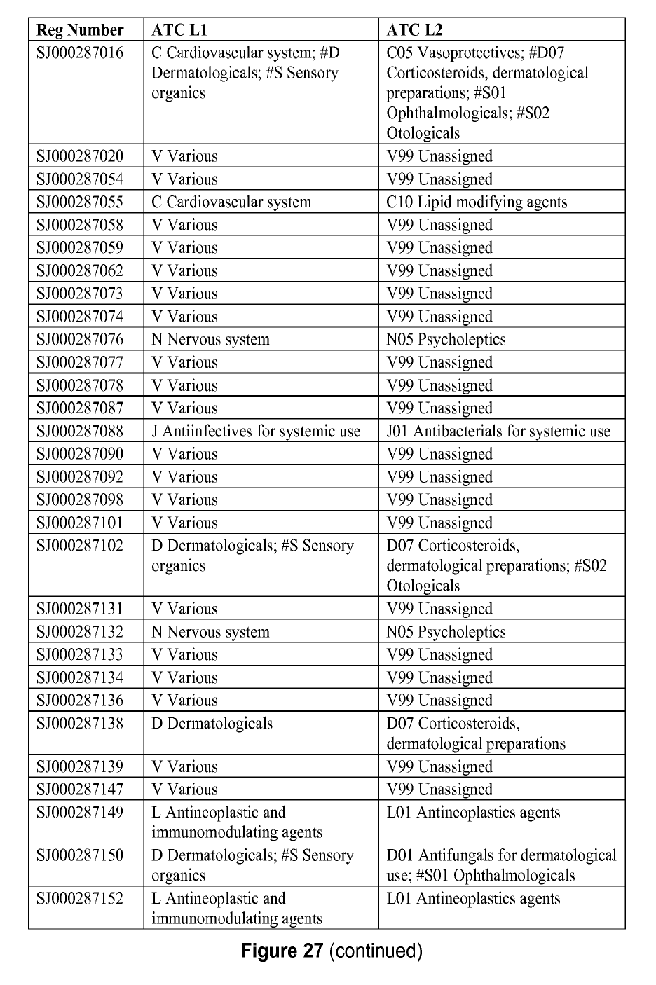

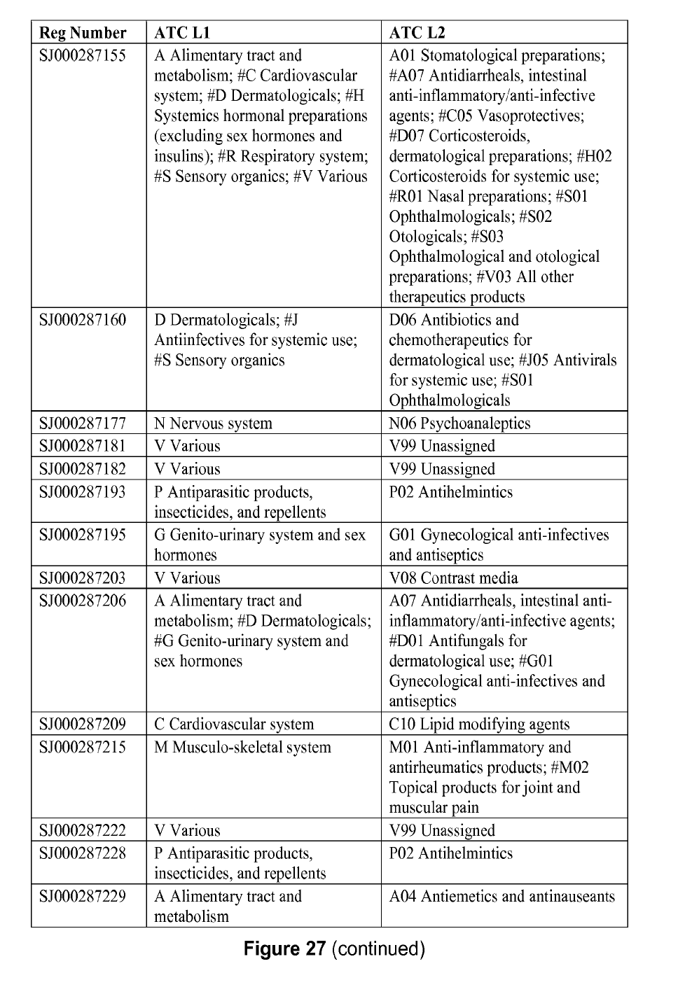

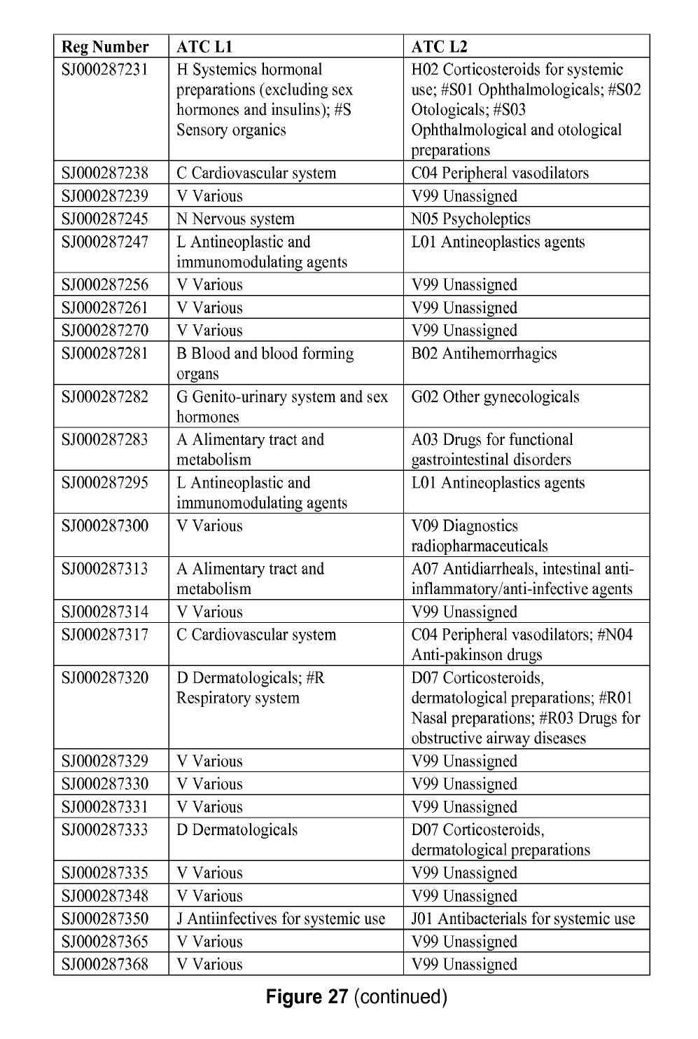

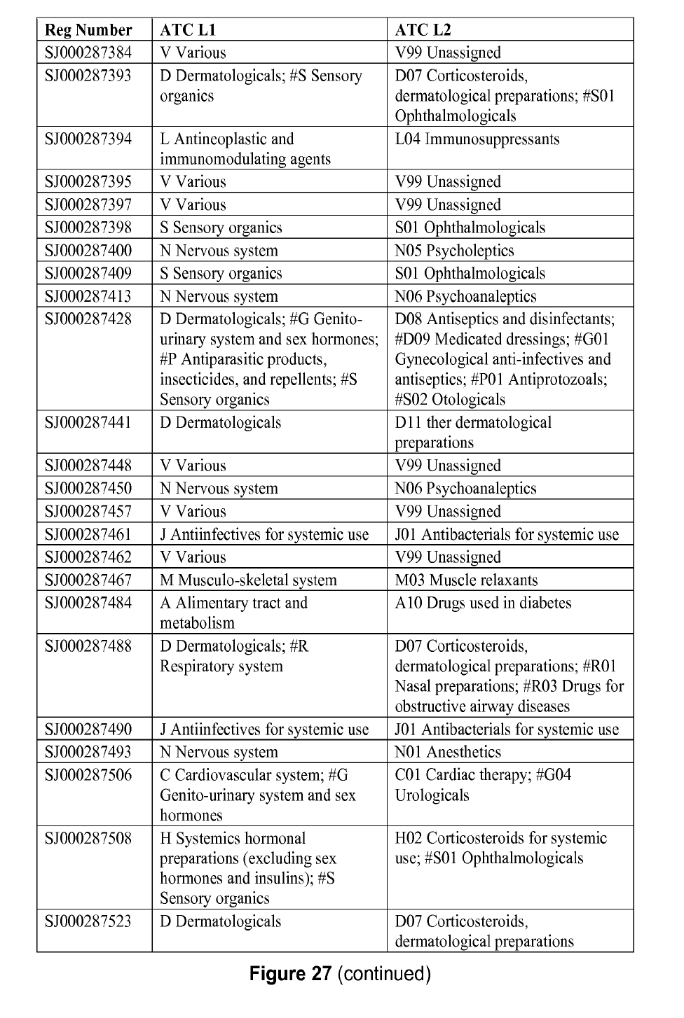

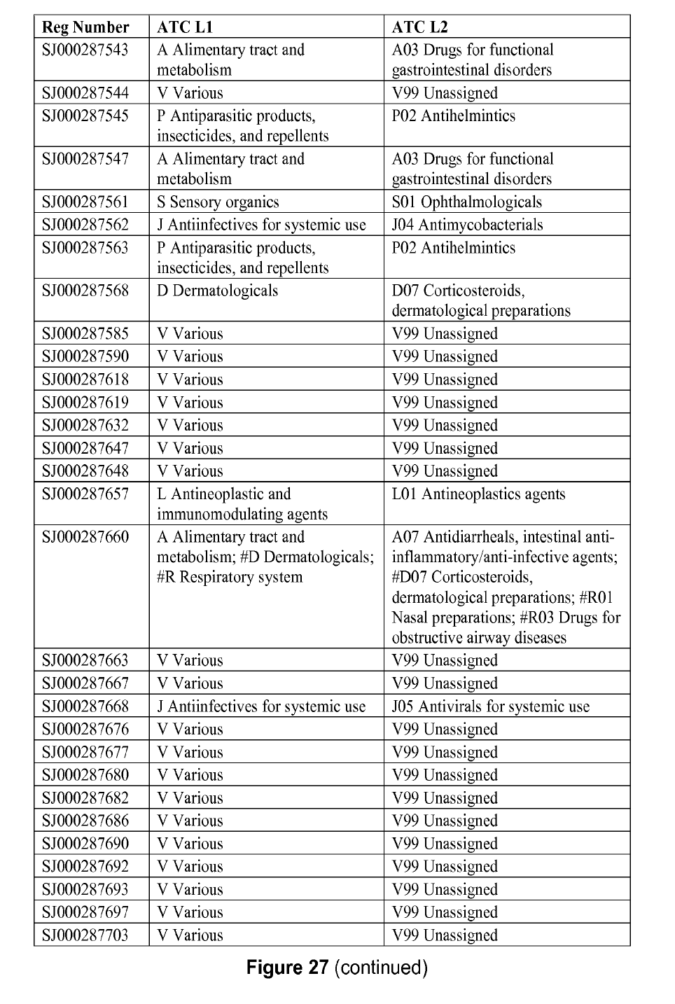

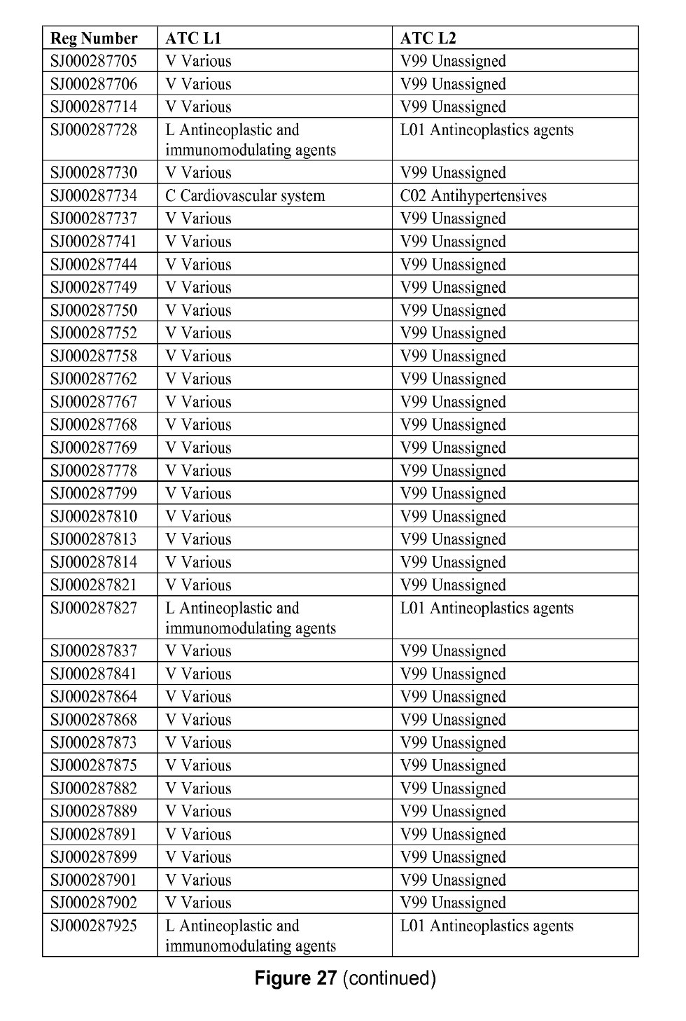

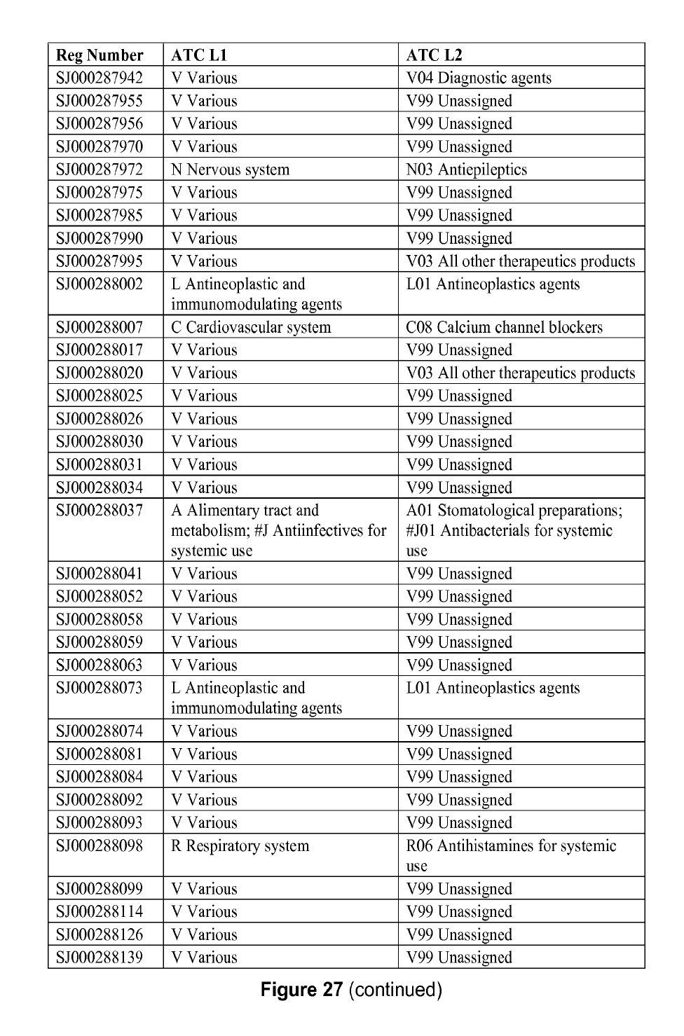

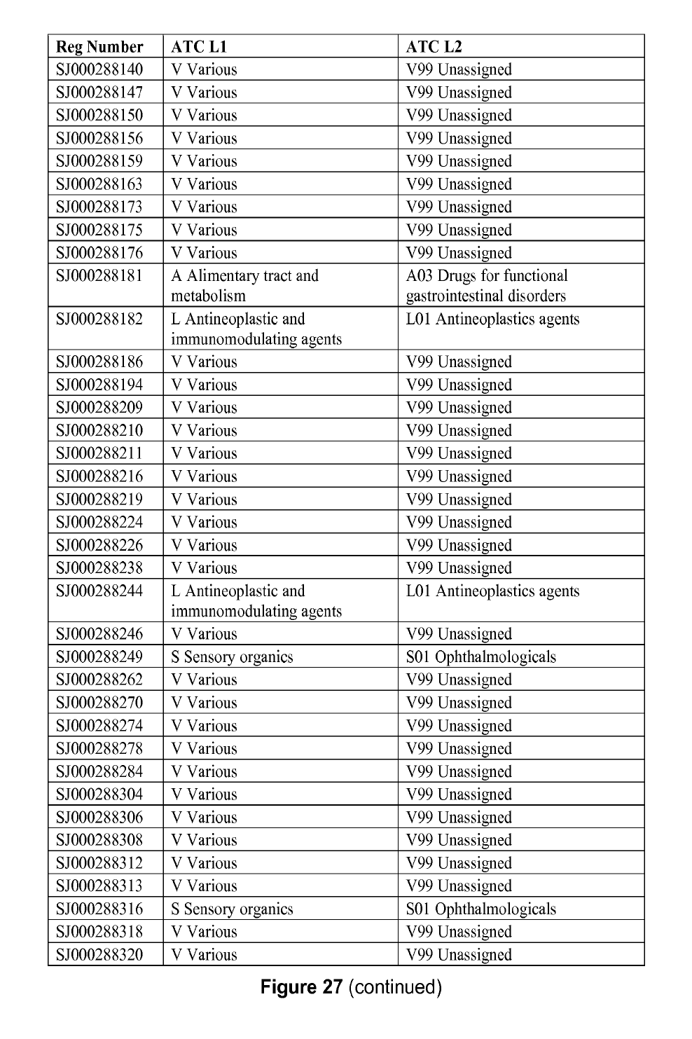

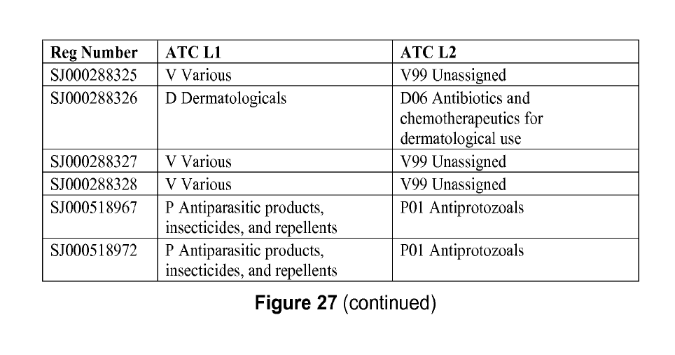

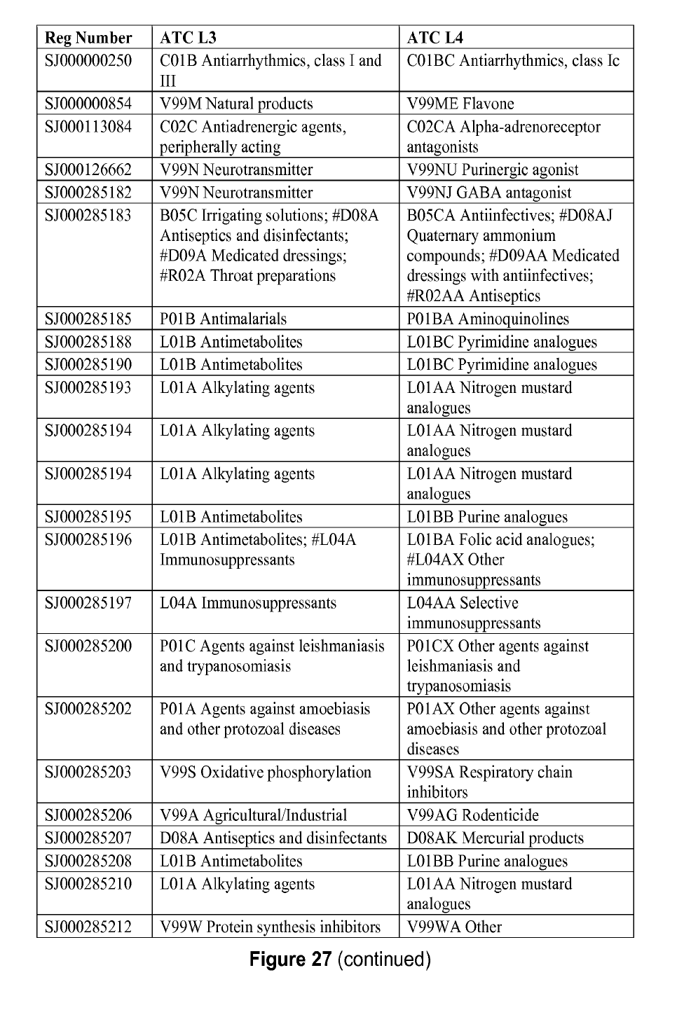

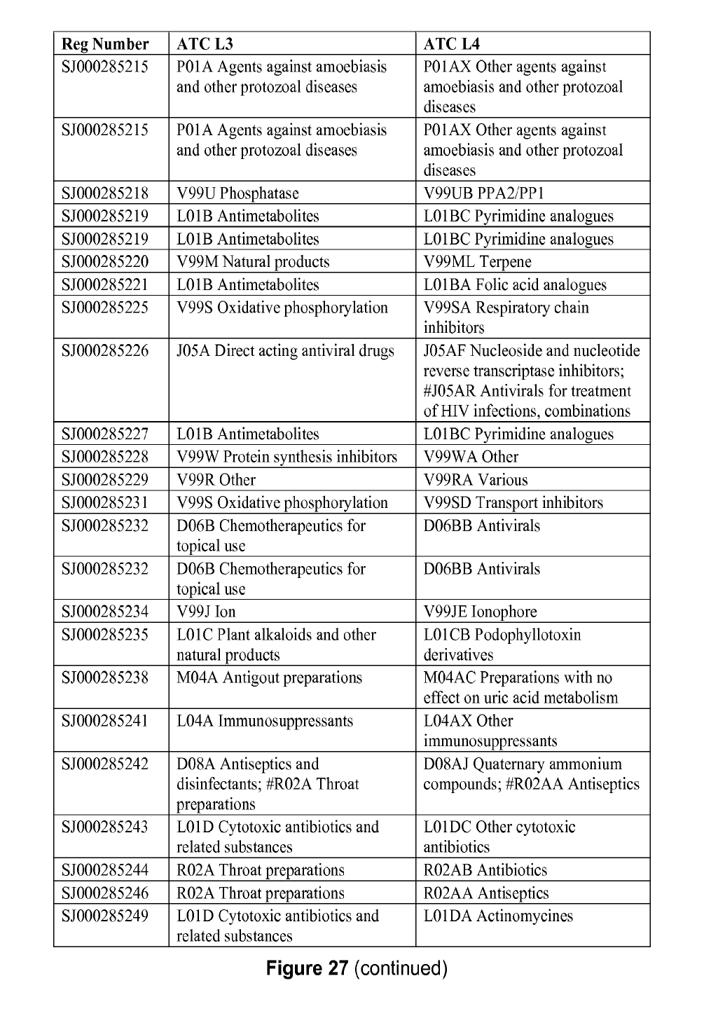

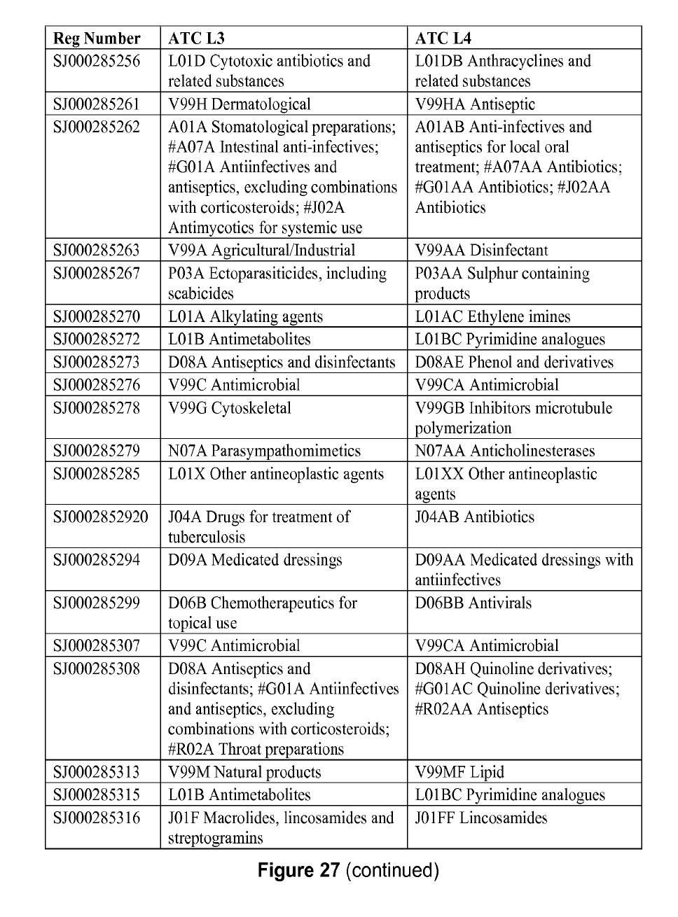

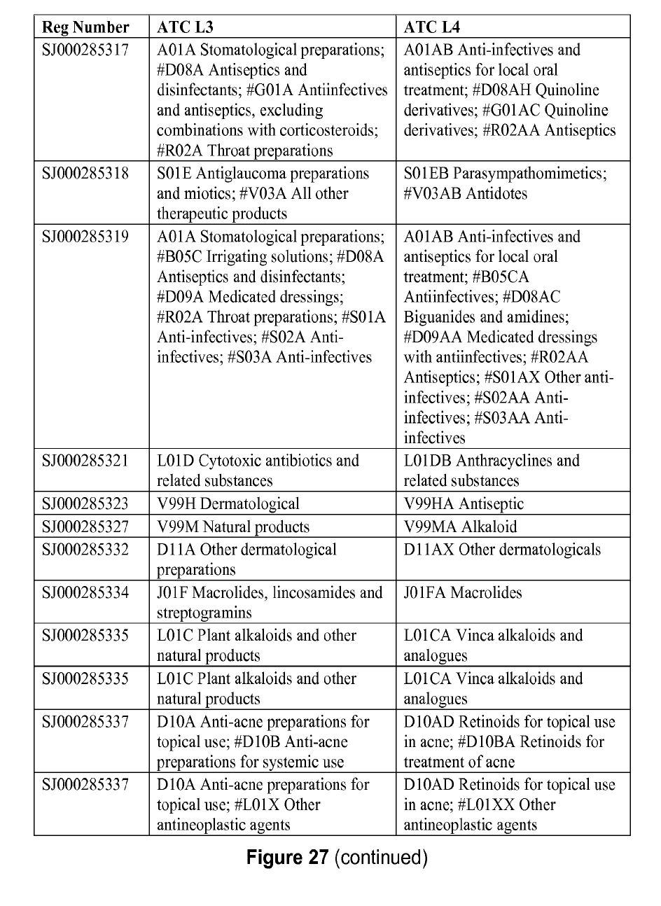

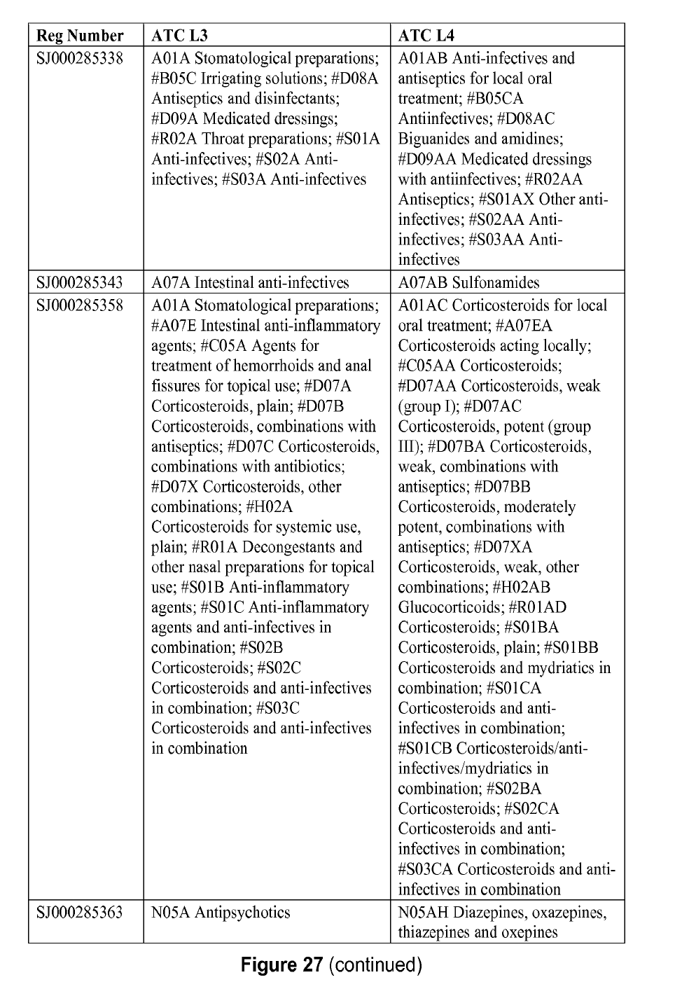

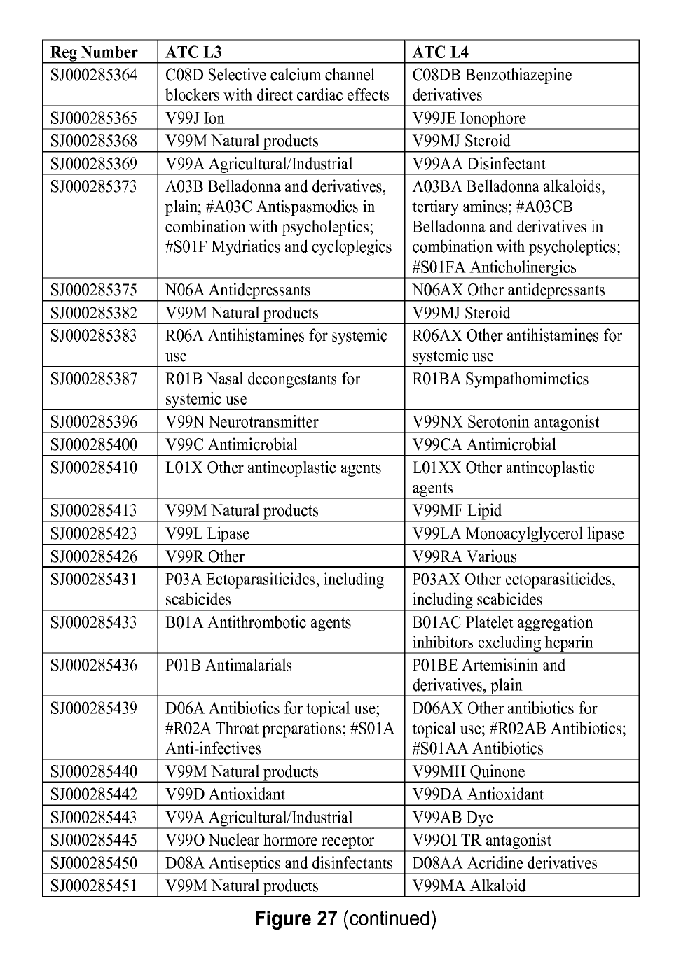

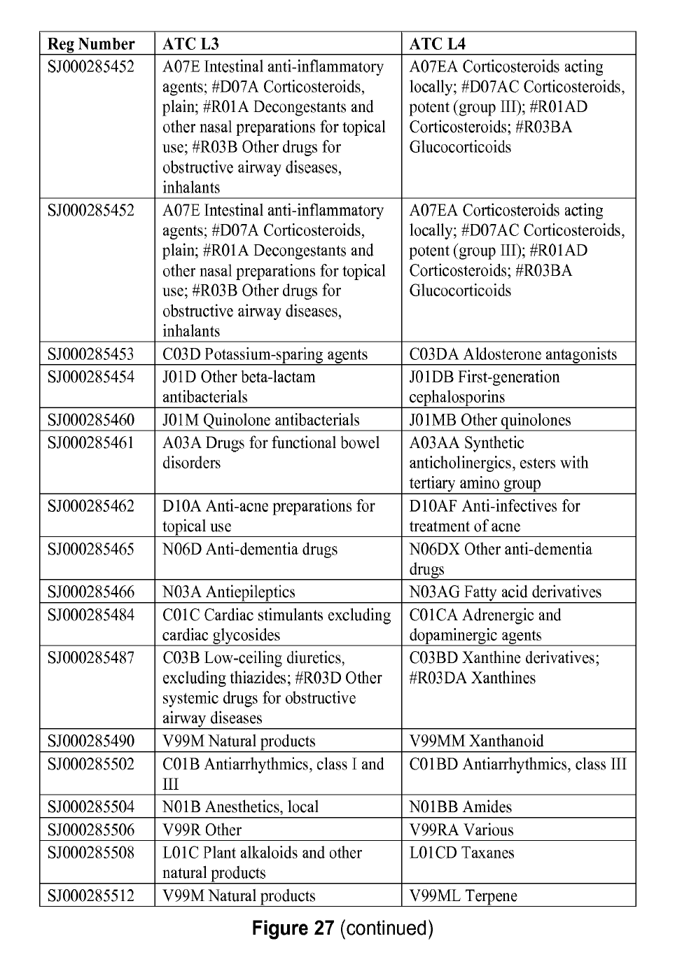

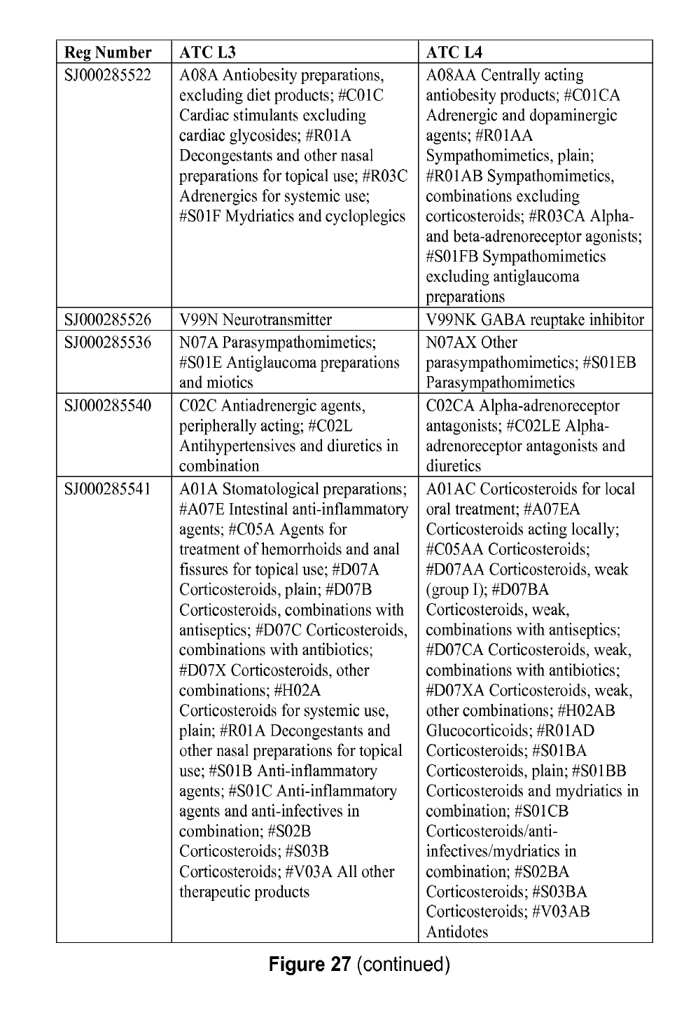

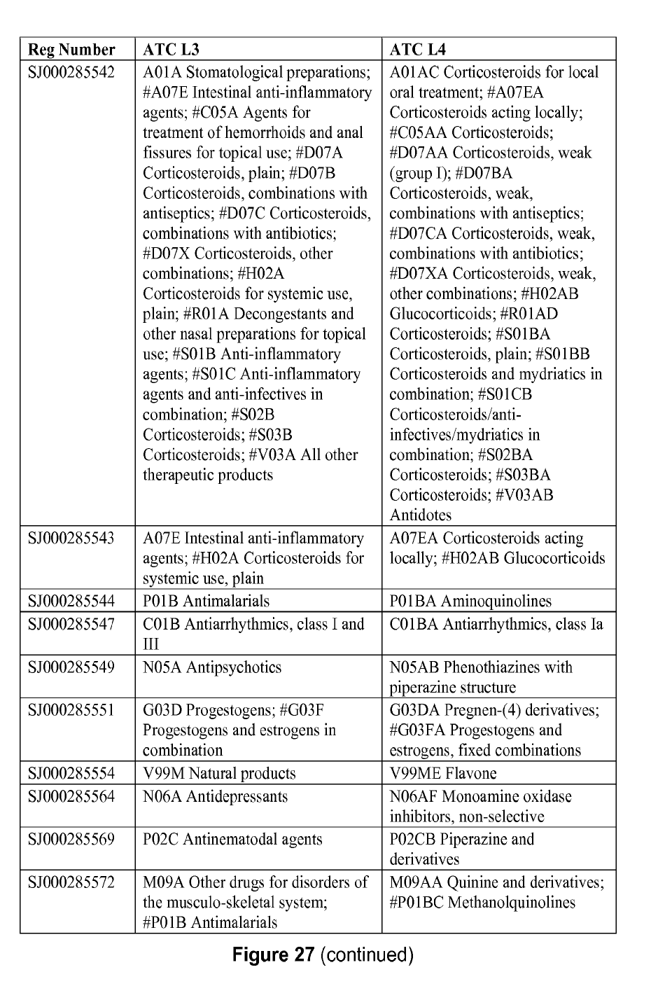

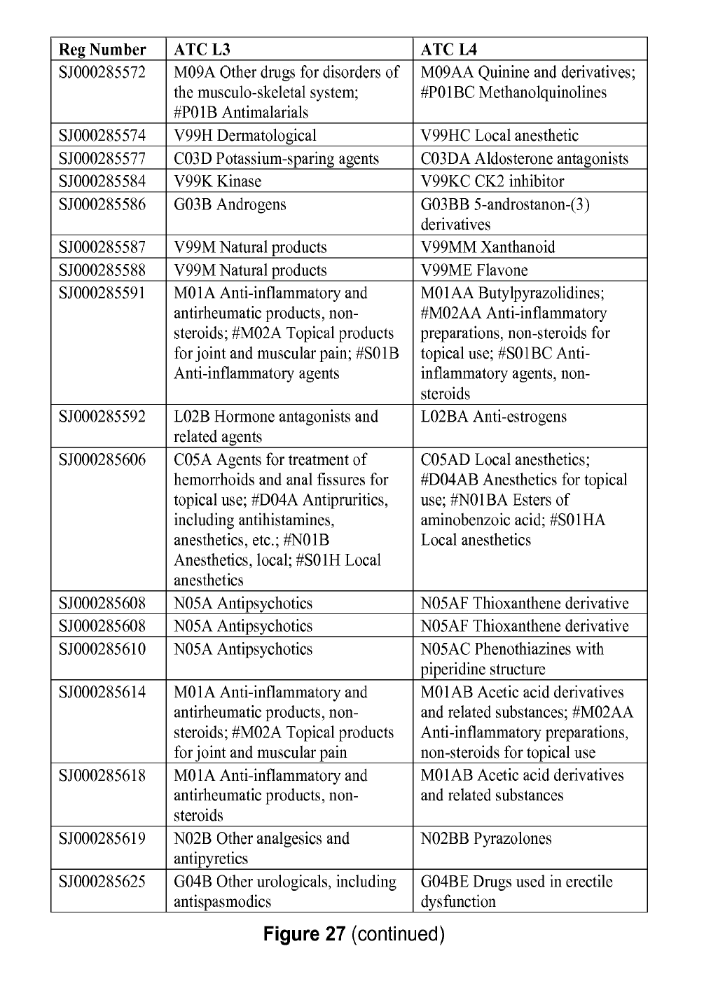

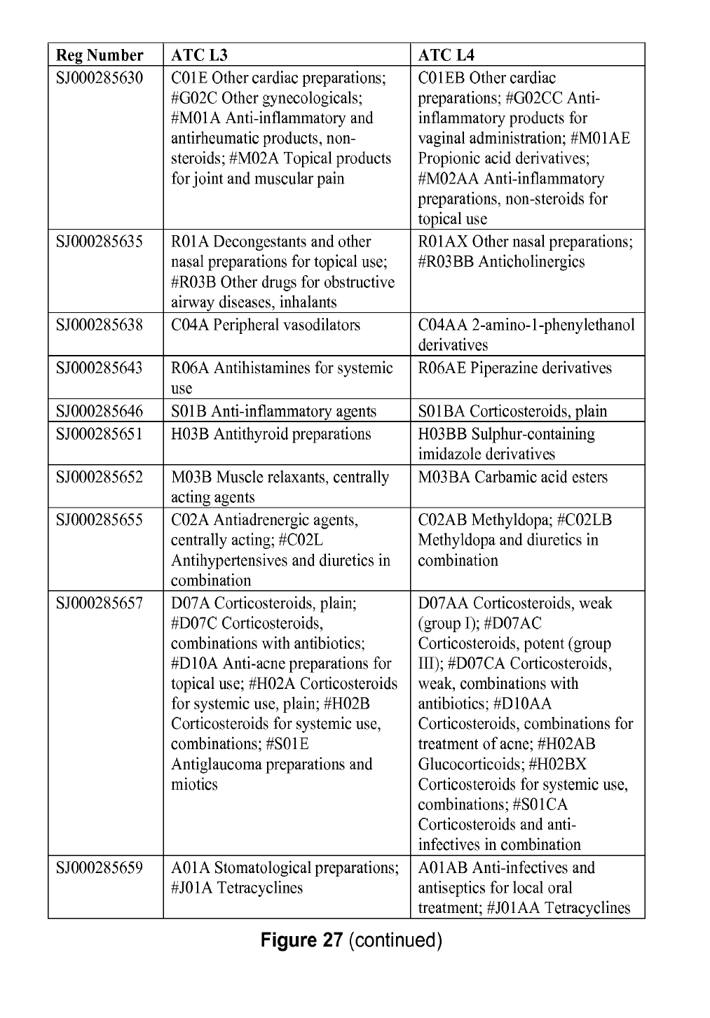

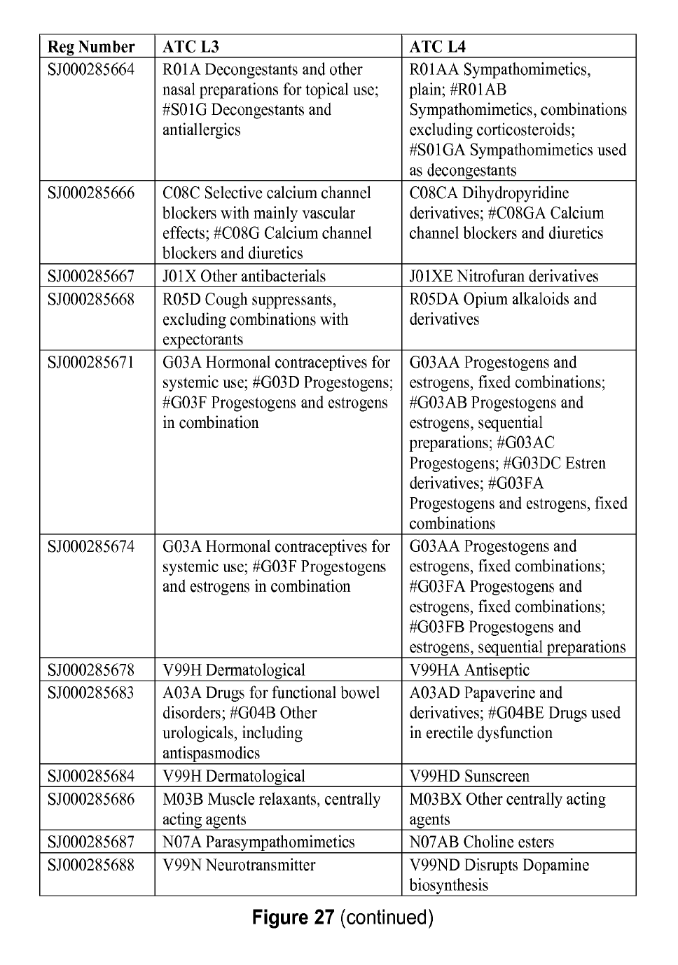

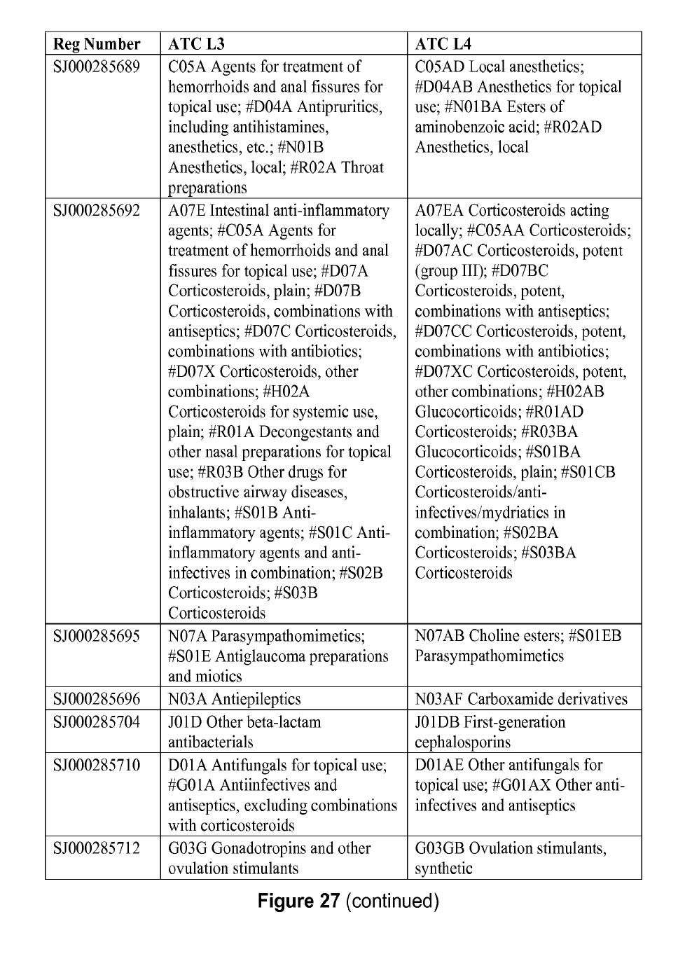

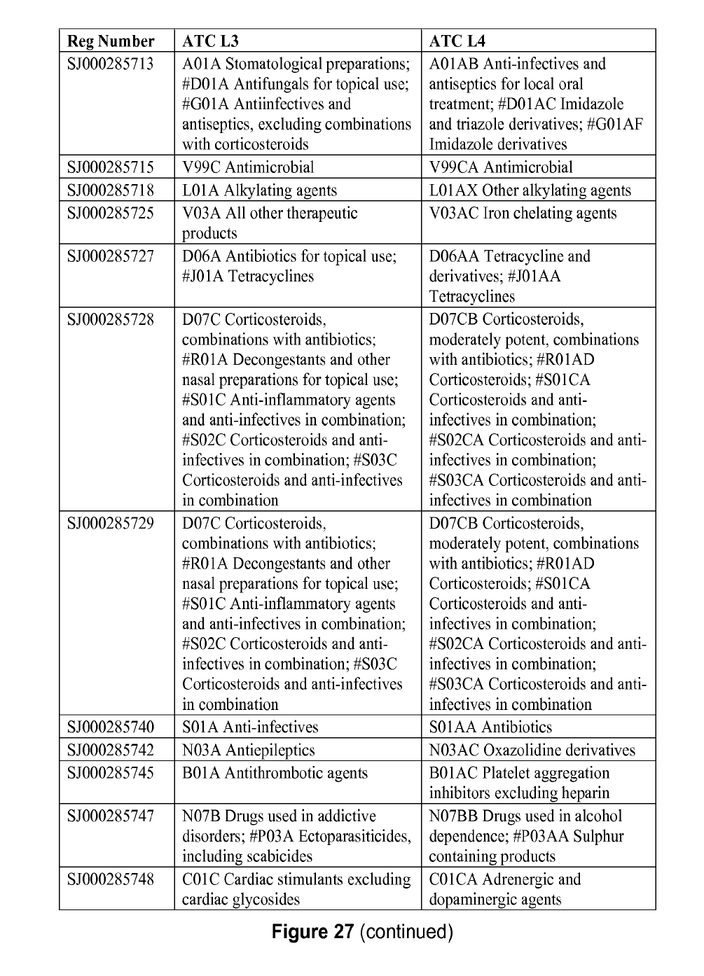

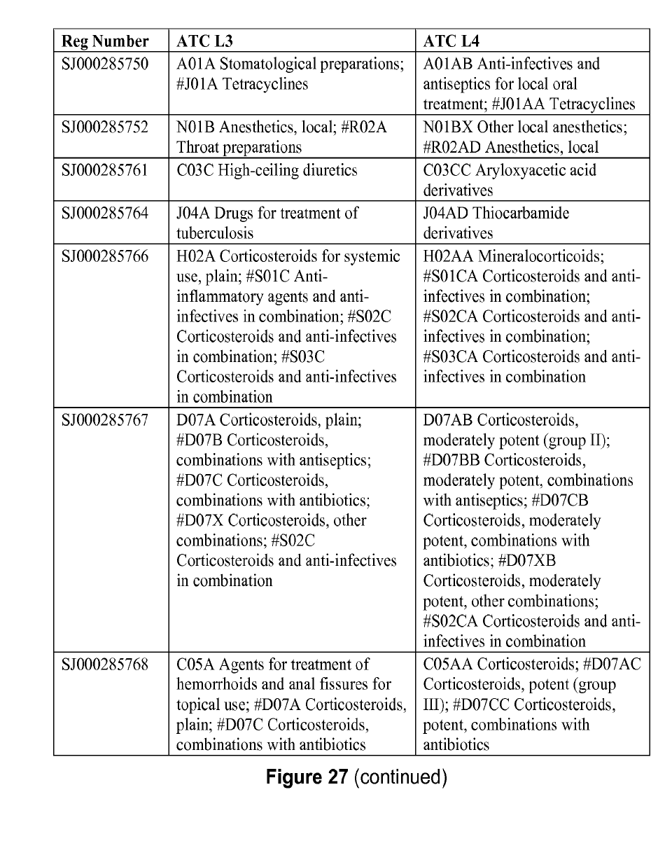

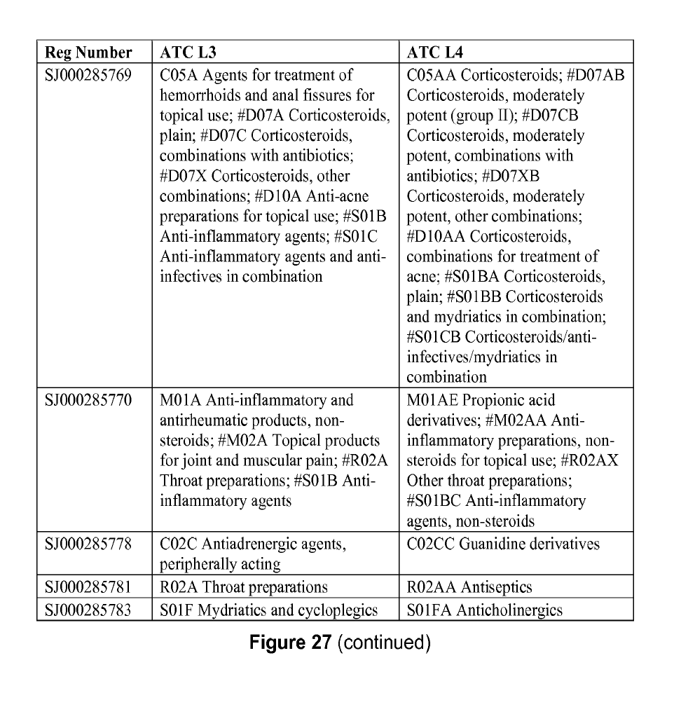

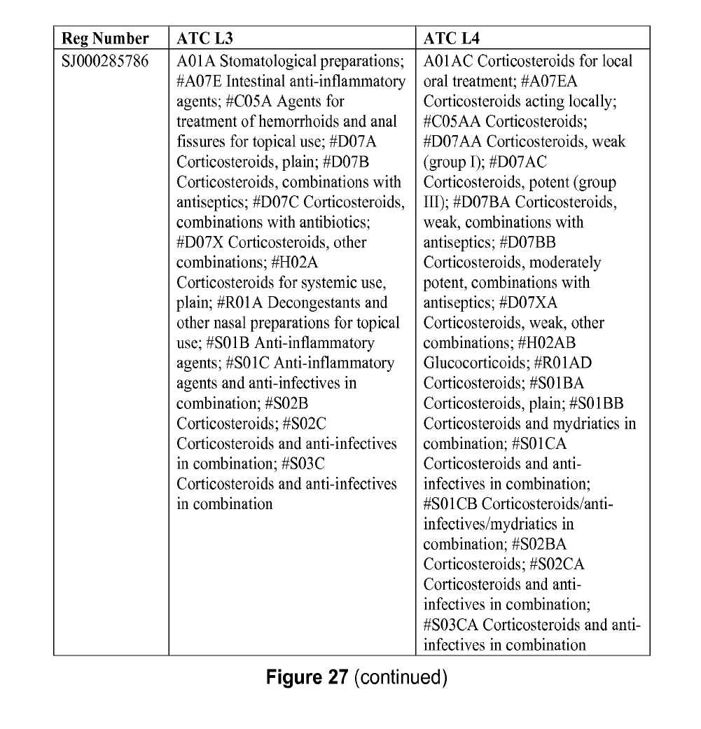

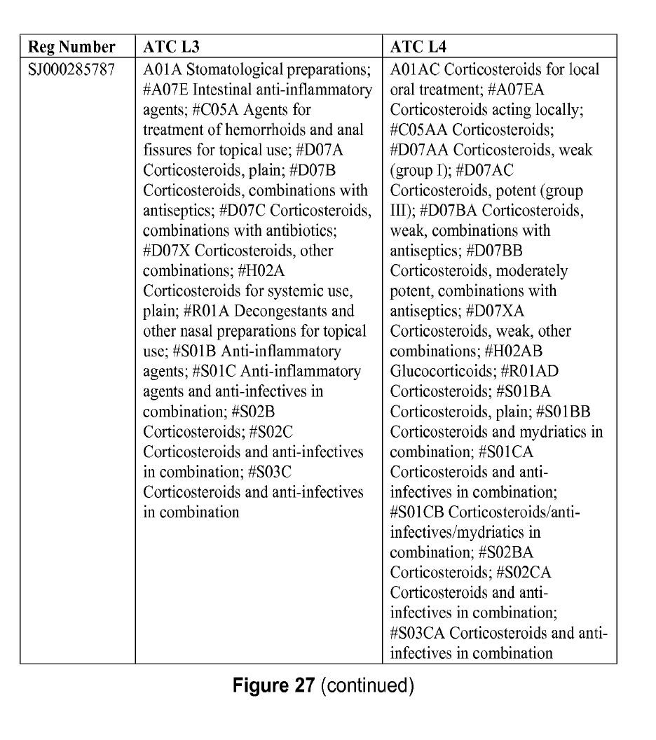

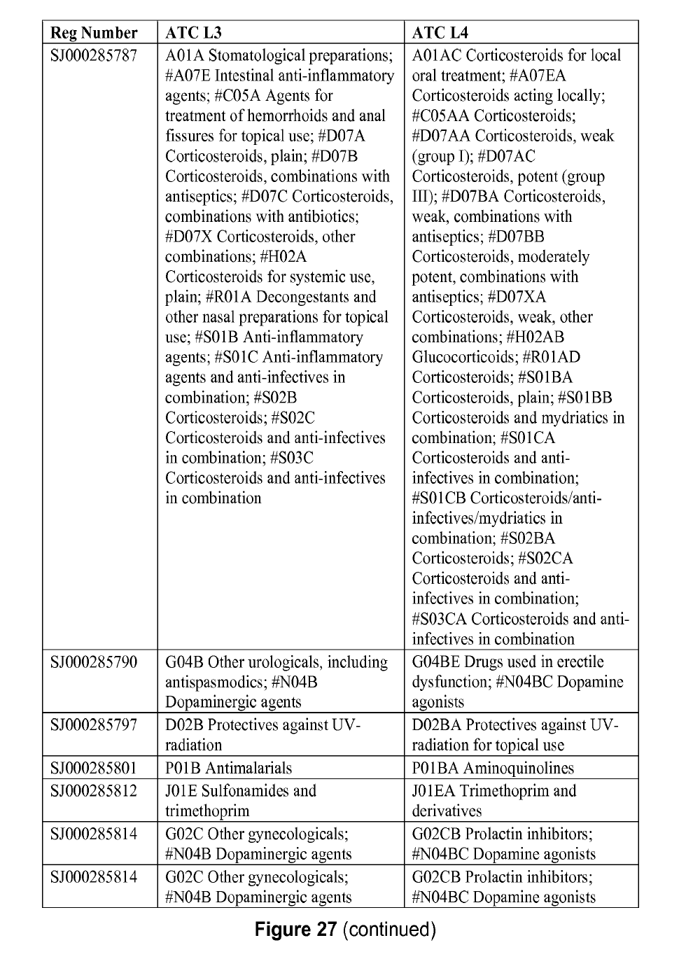

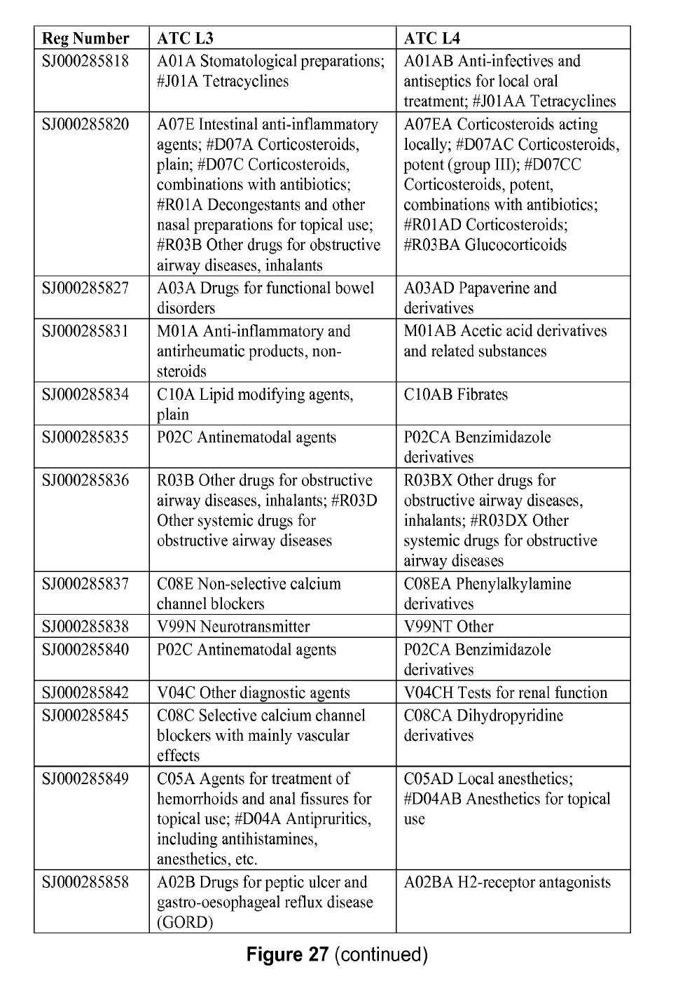

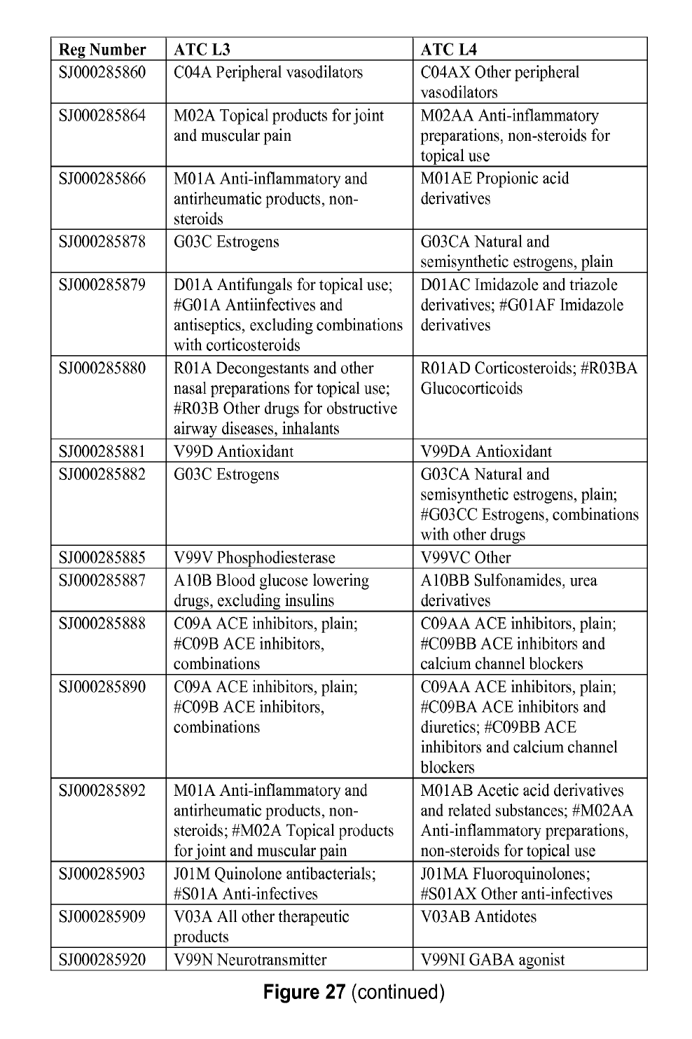

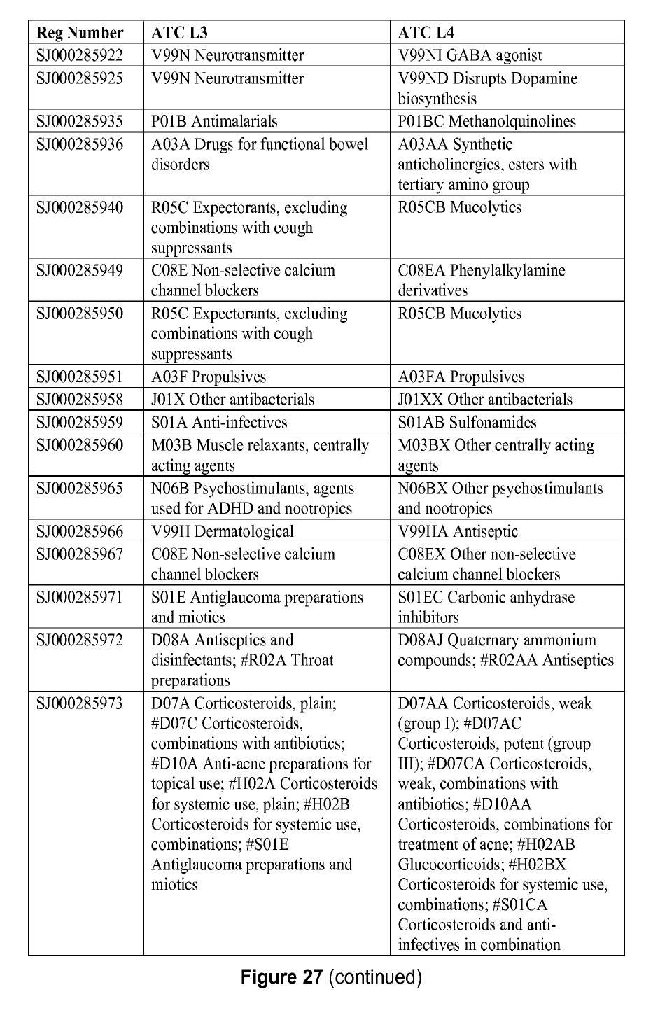

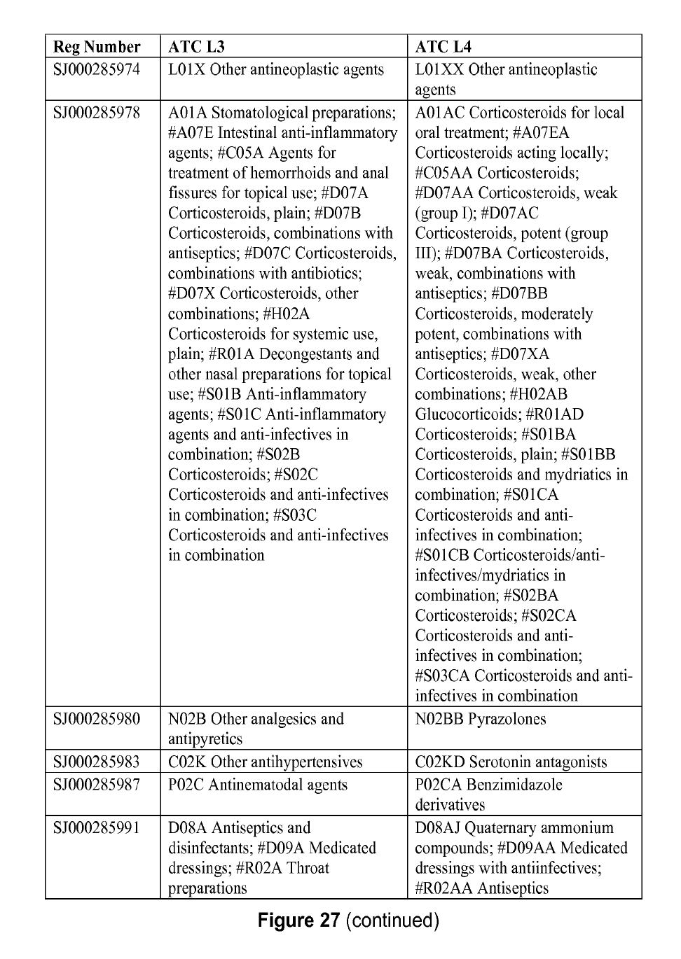

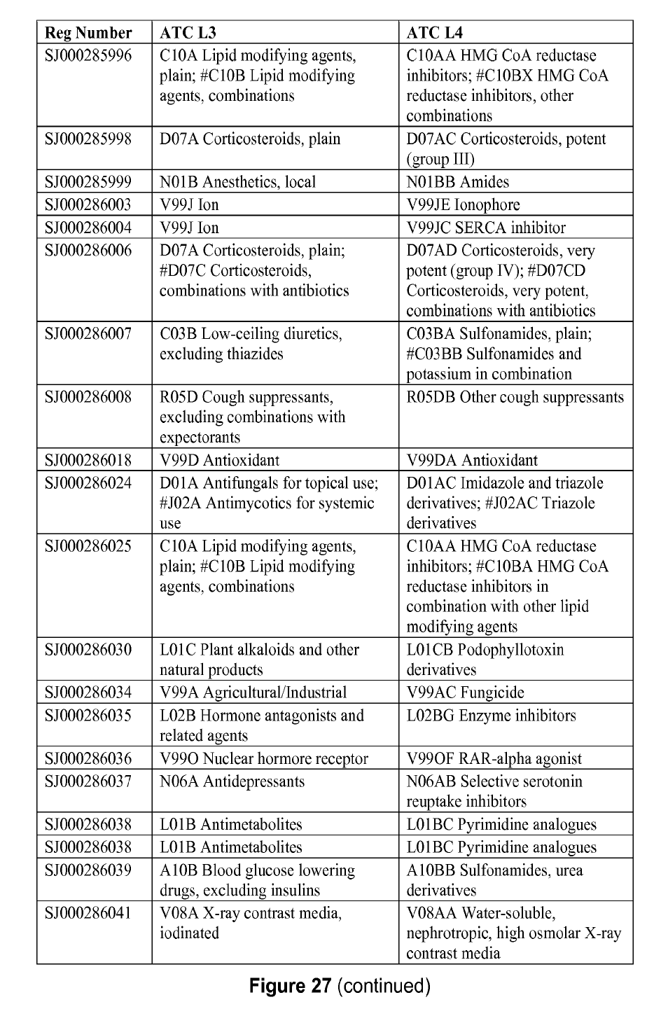

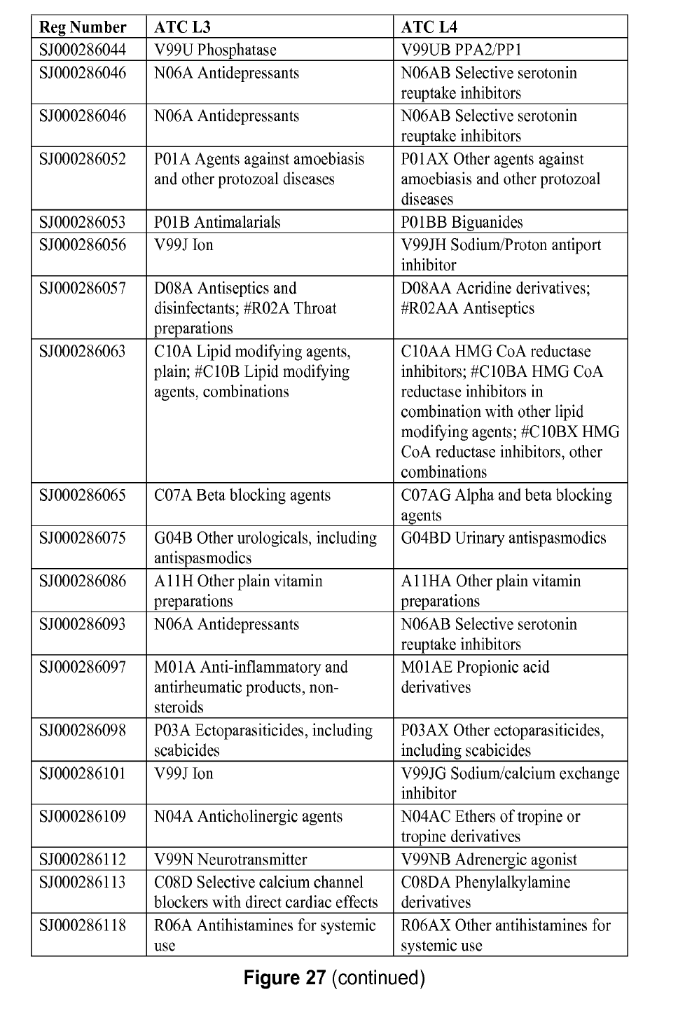

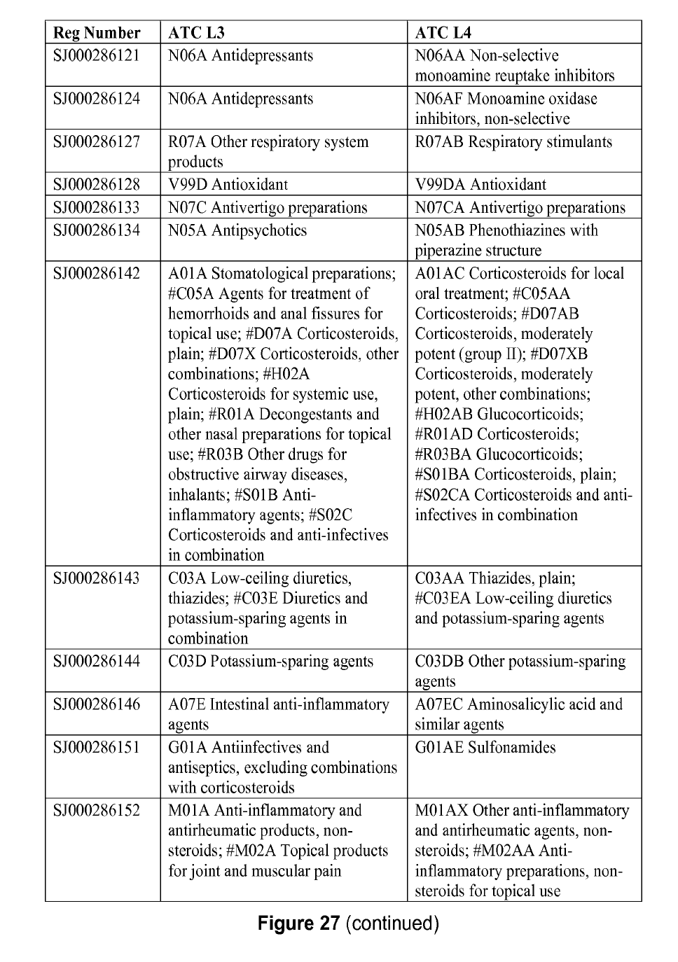

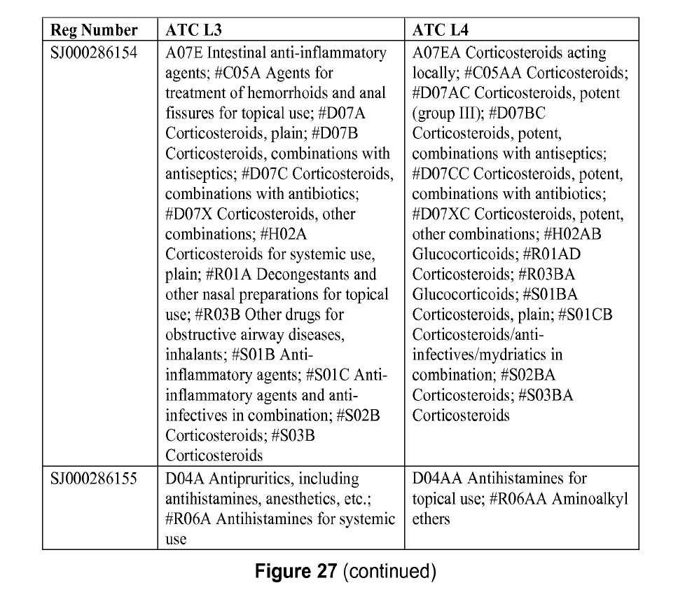

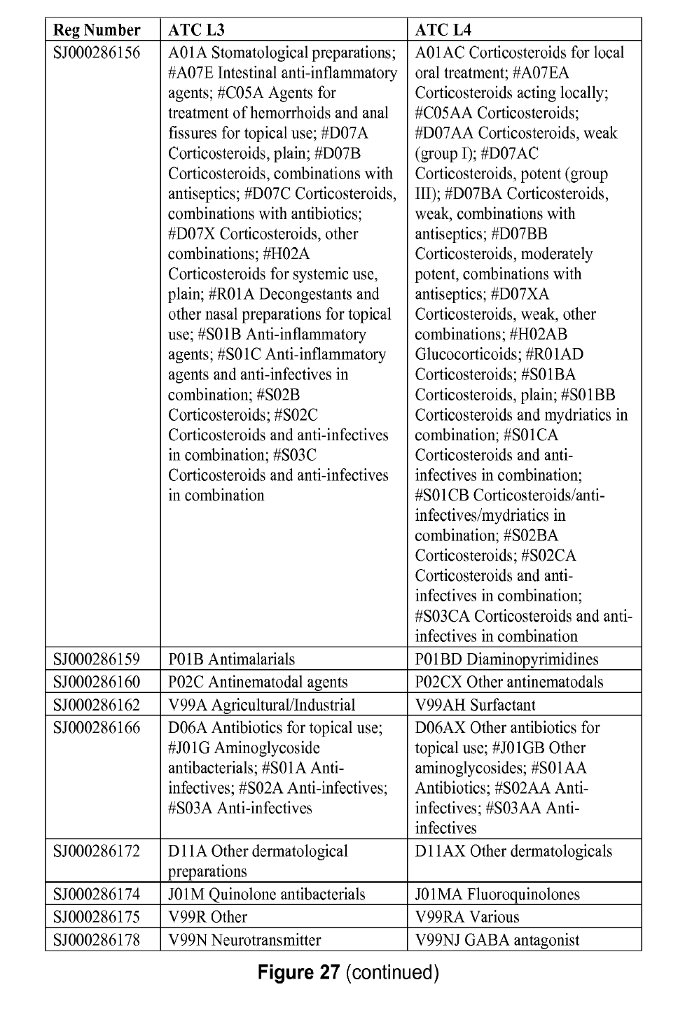

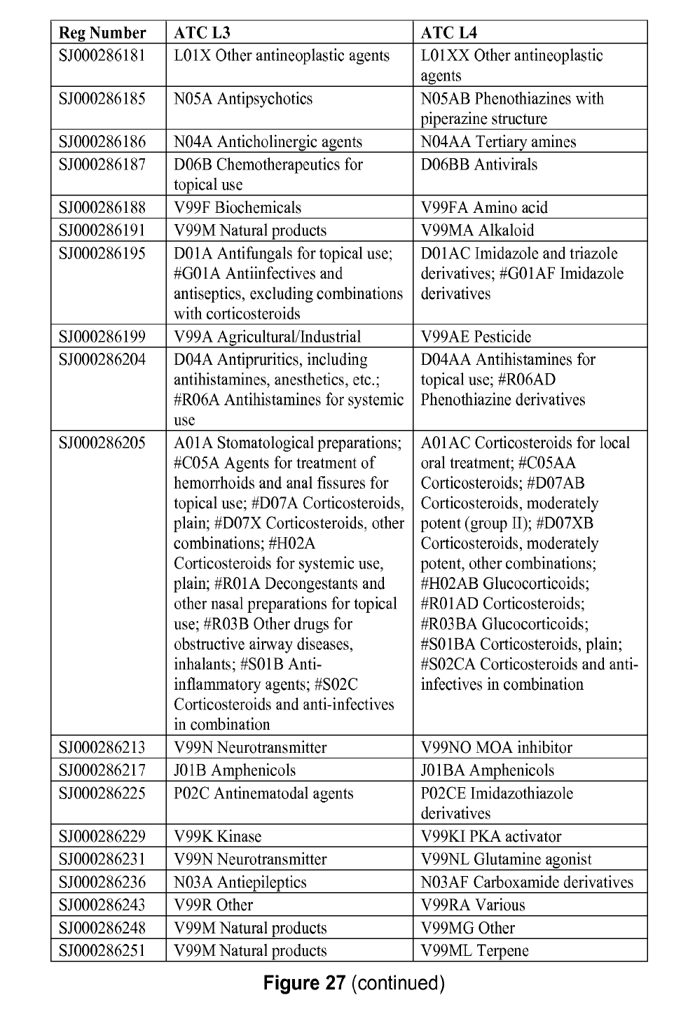

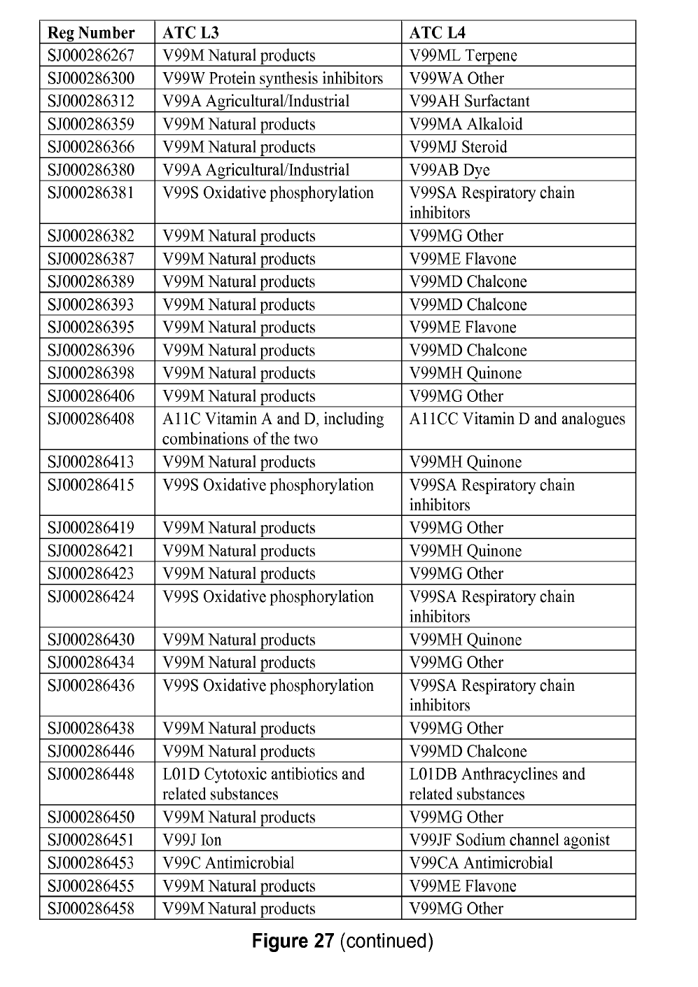

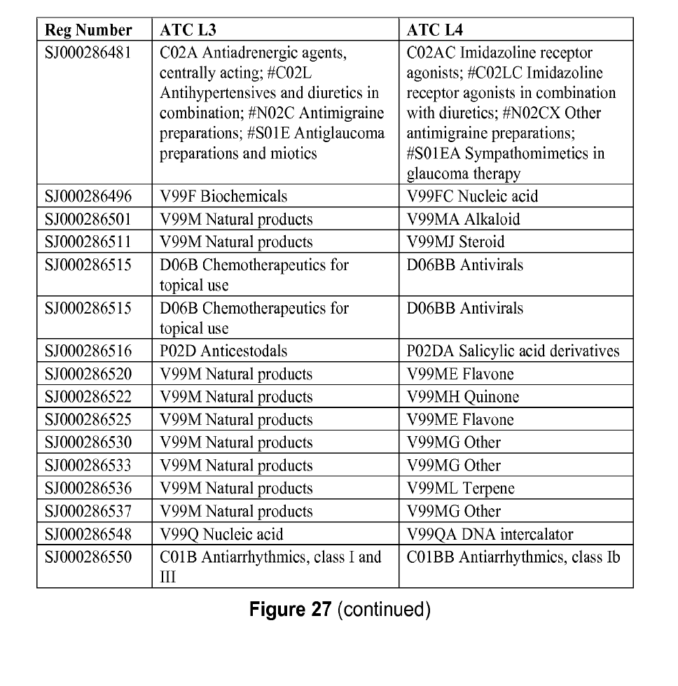

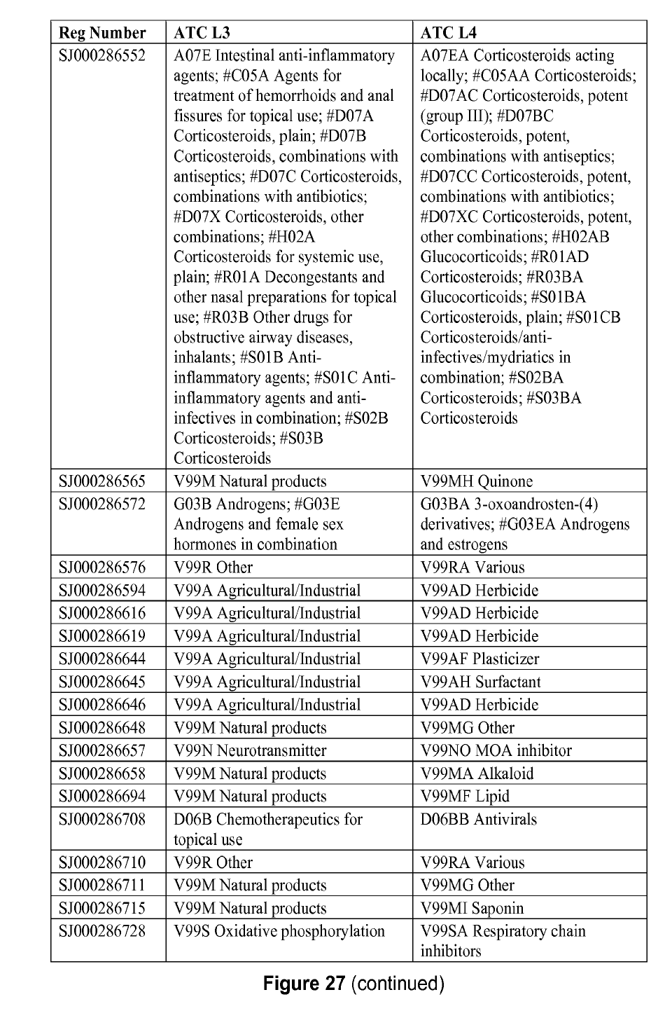

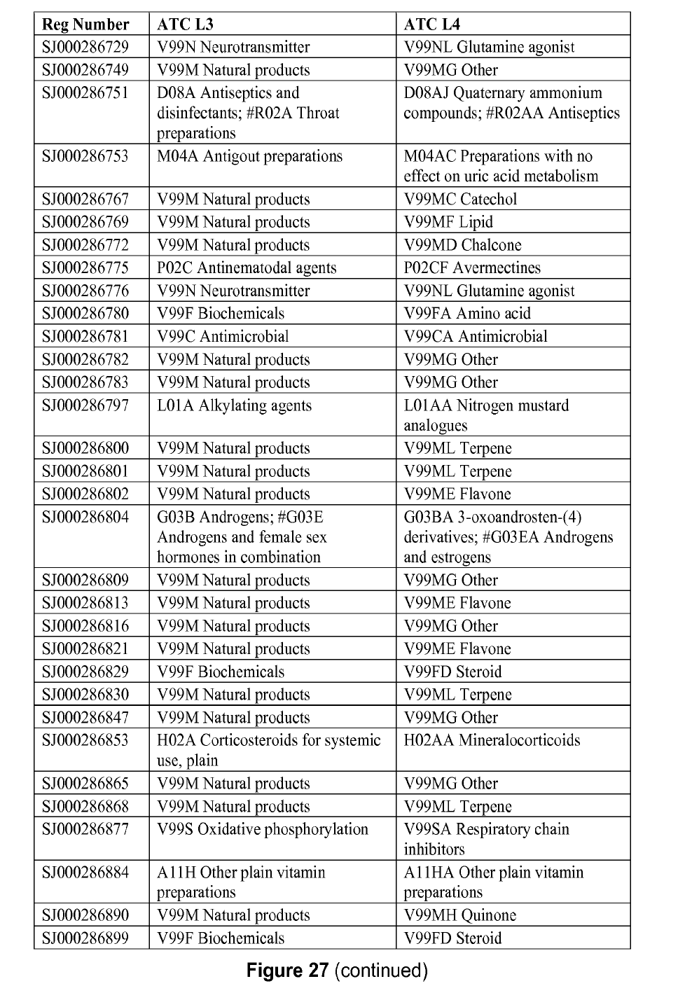

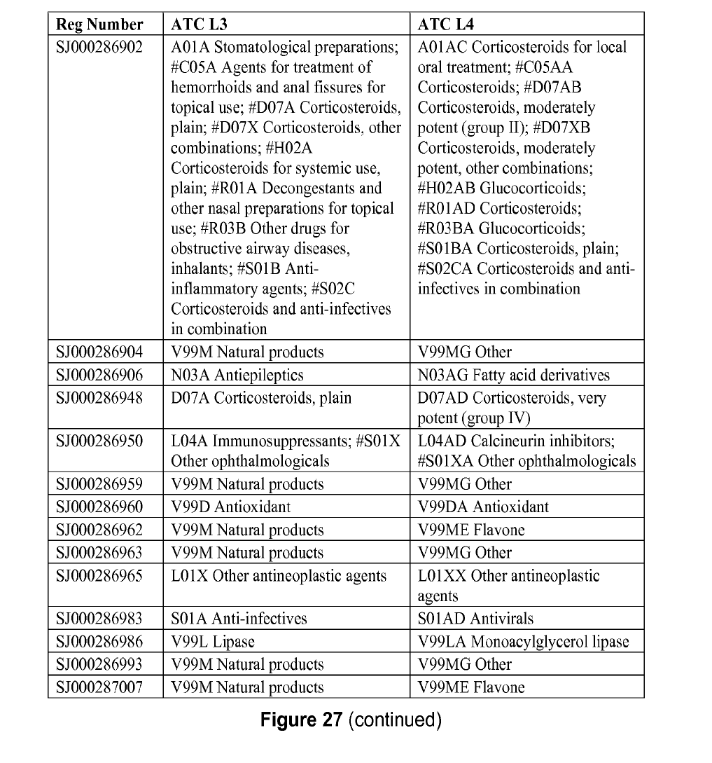

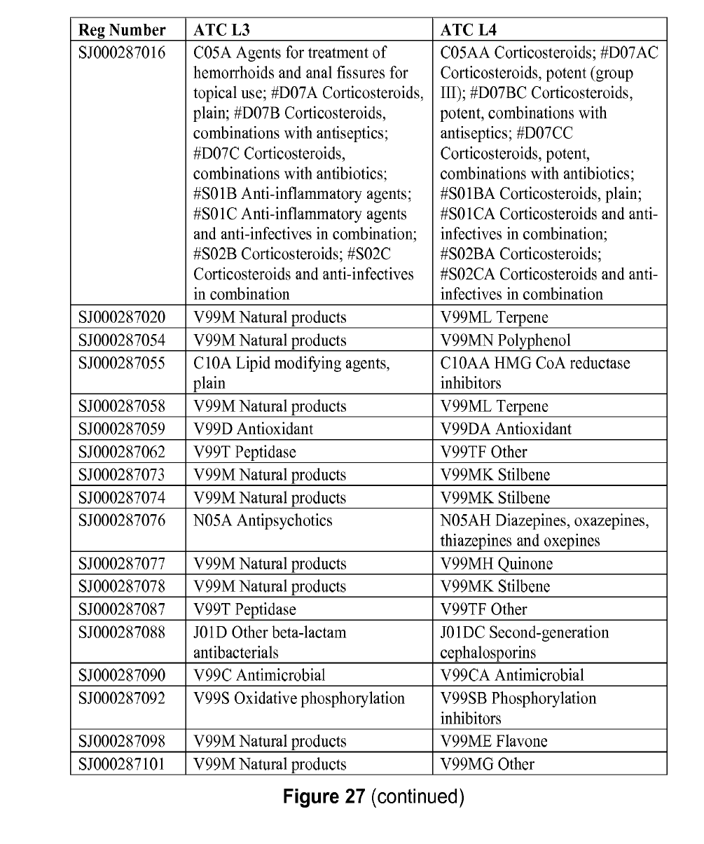

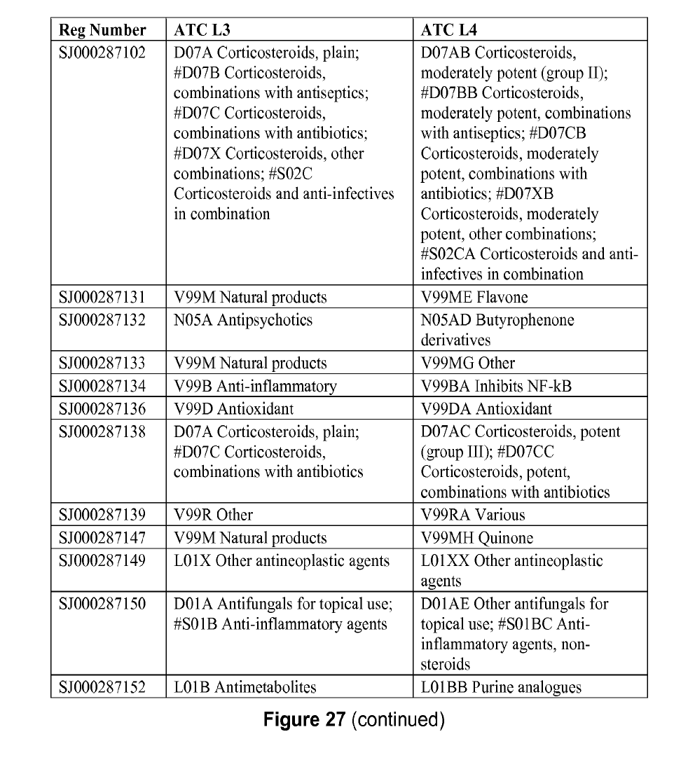

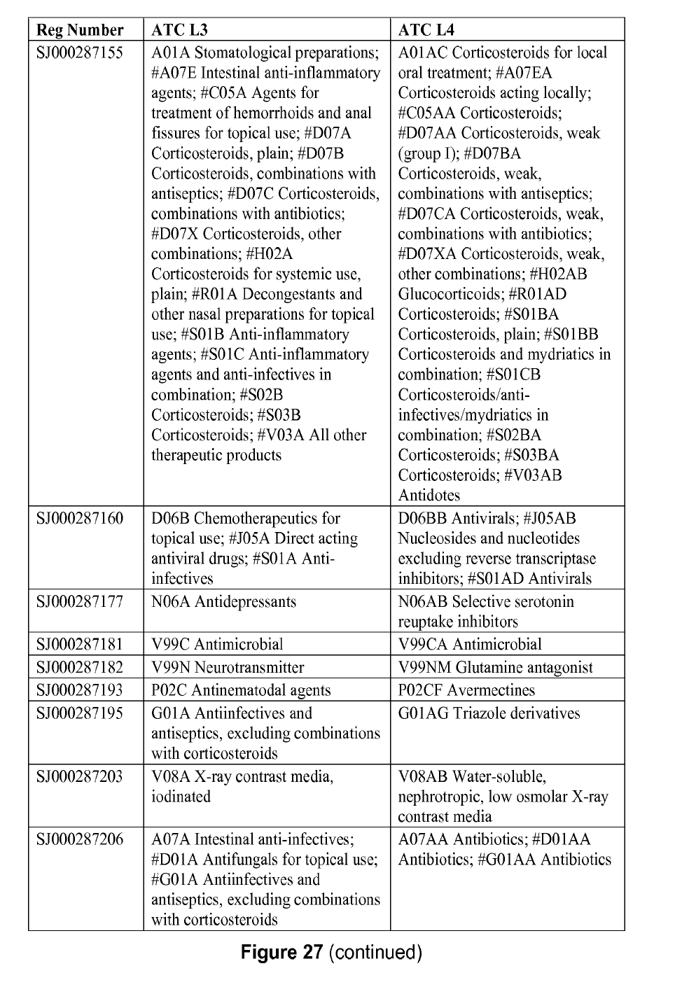

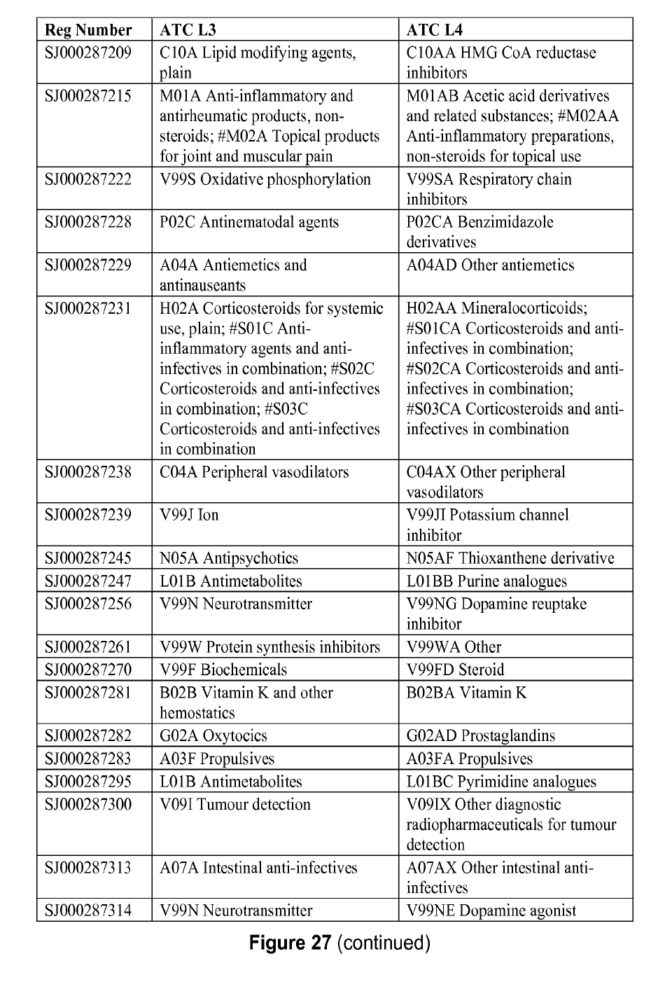

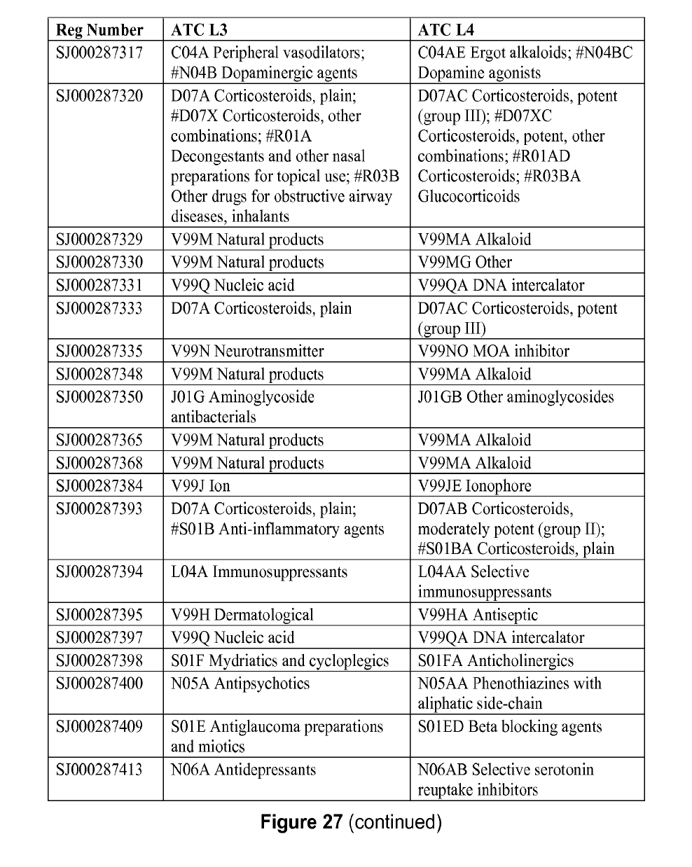

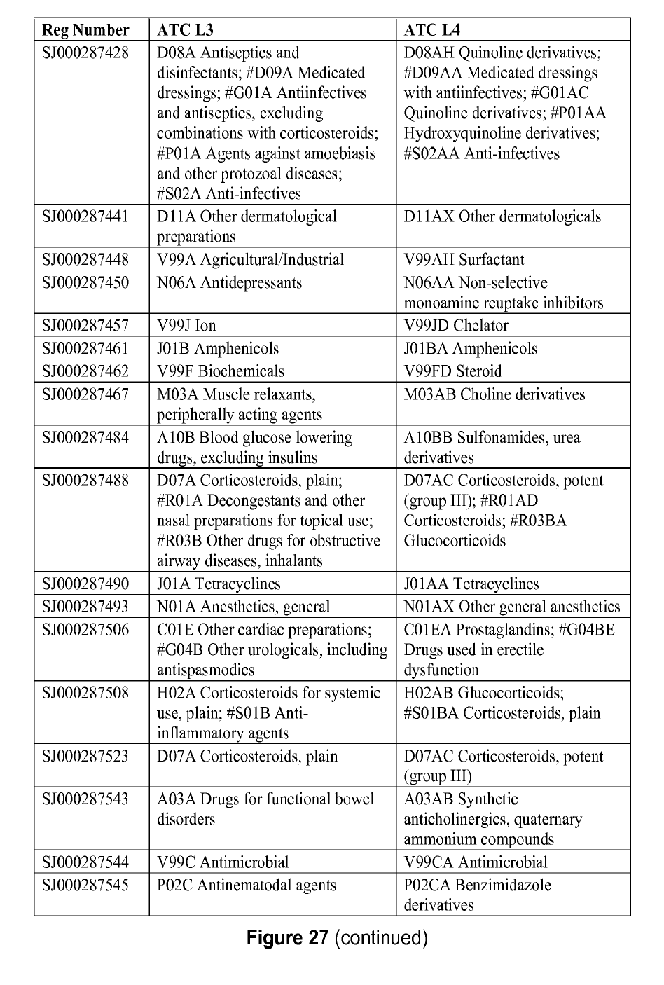

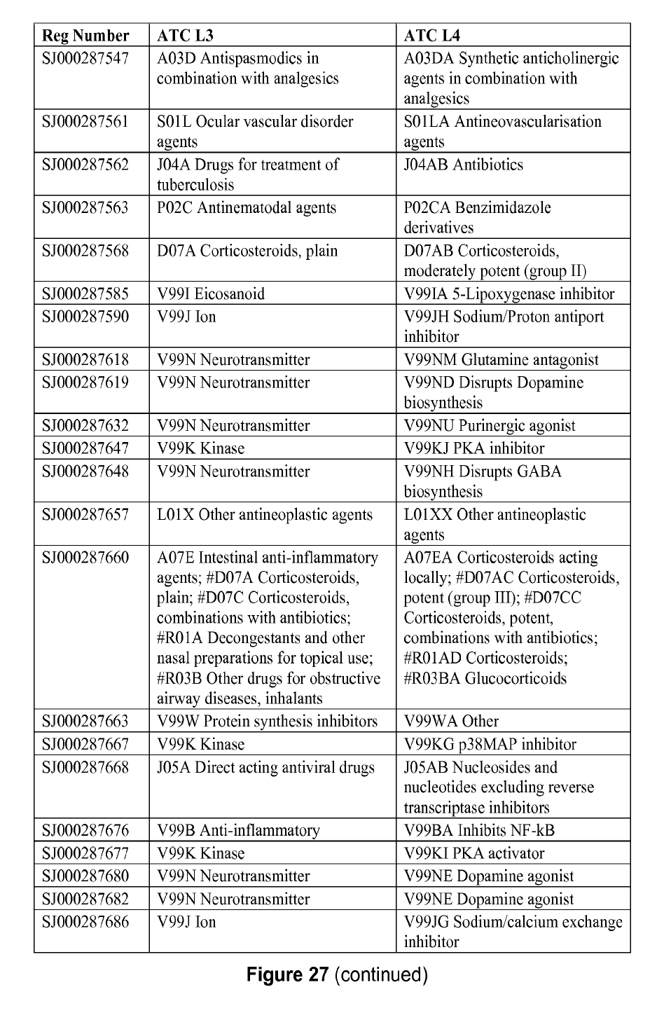

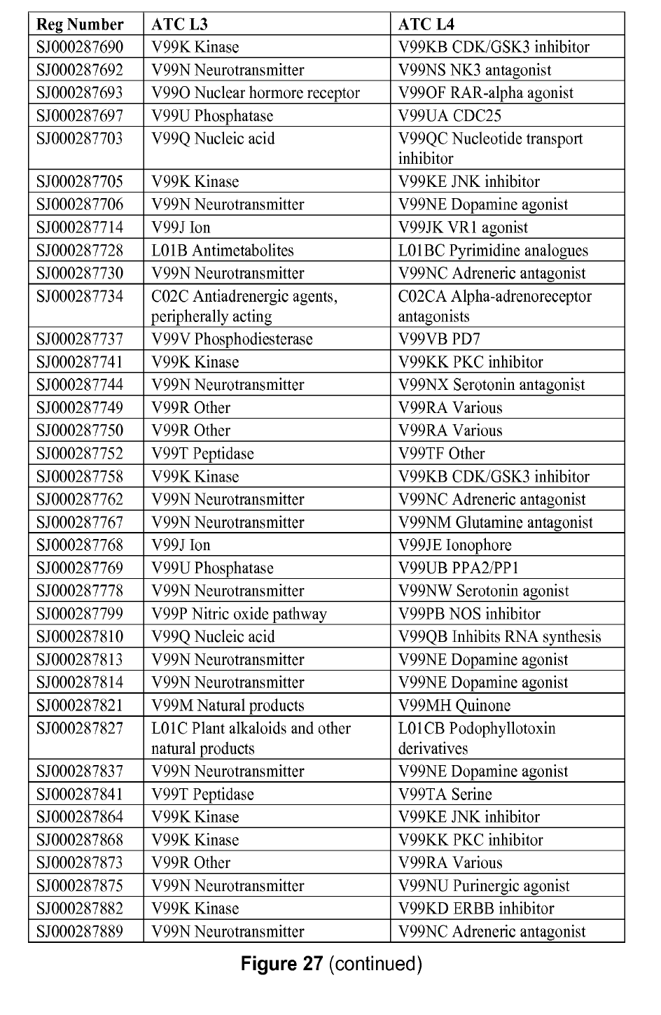

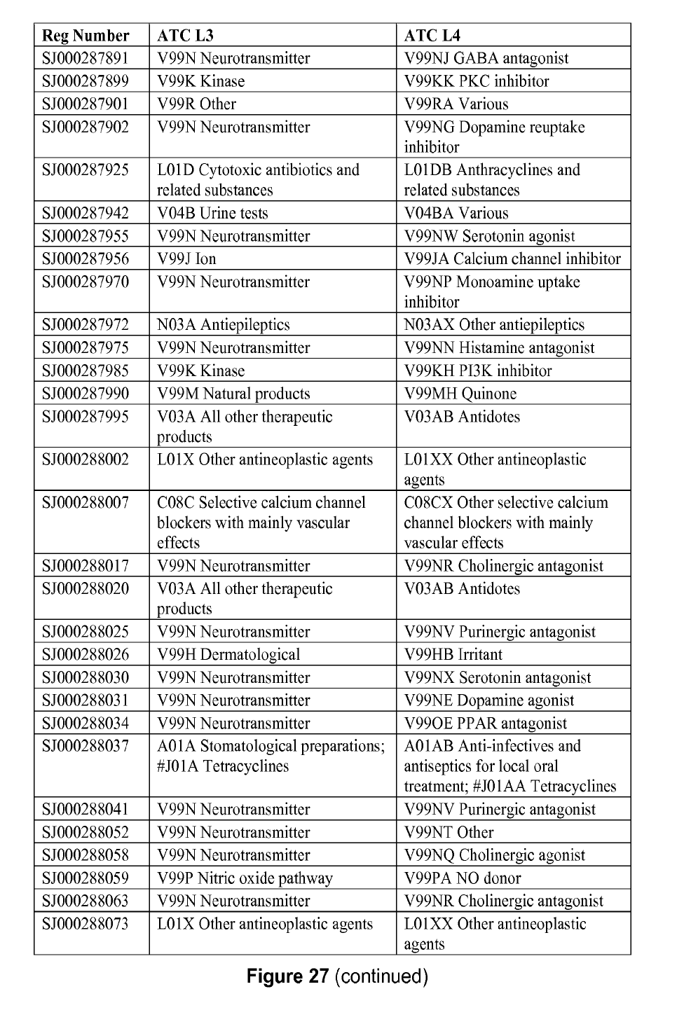

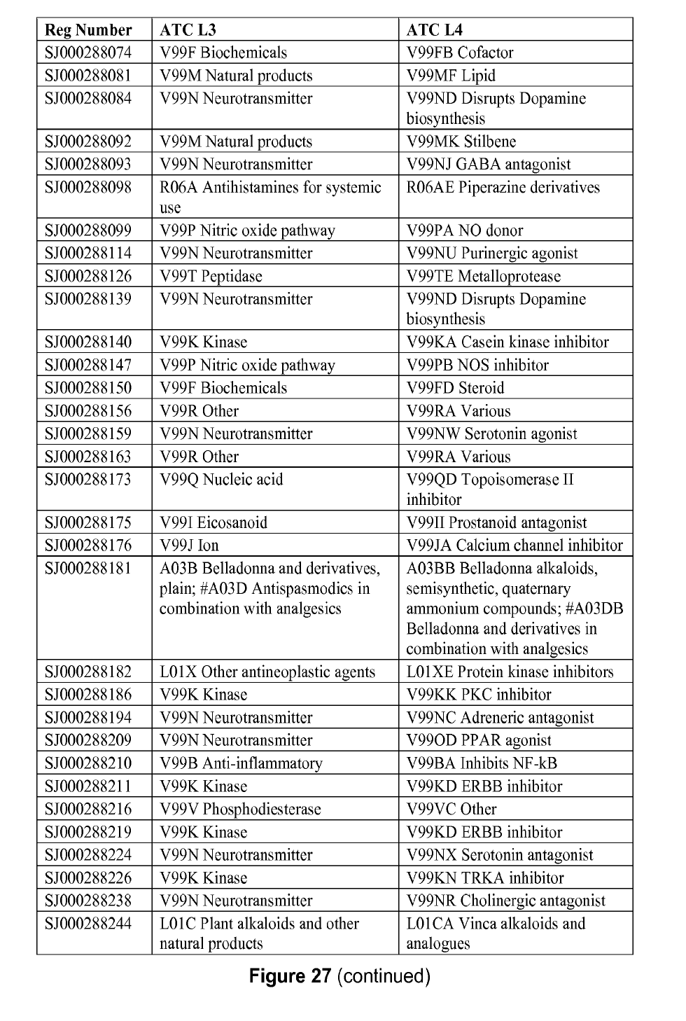

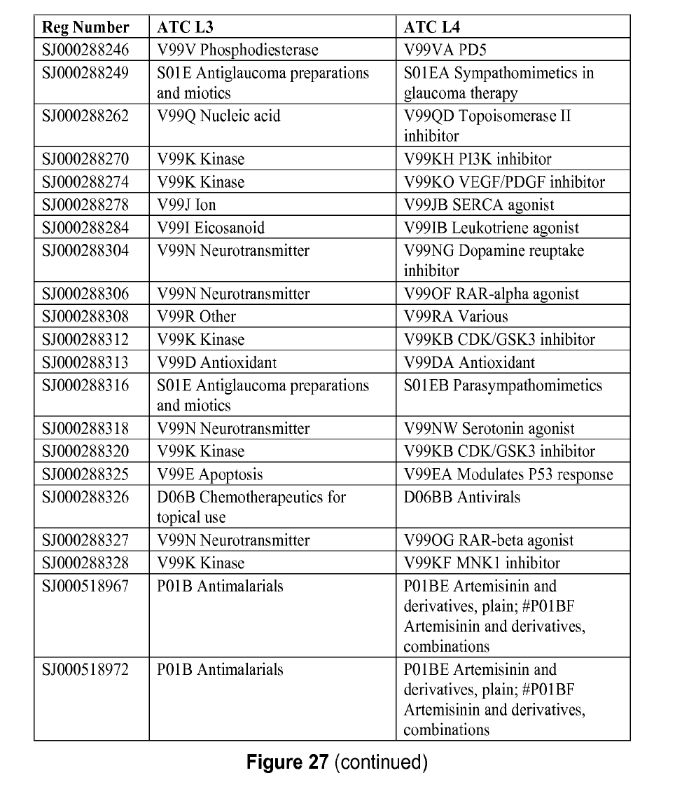

In one aspect, pharmacologic agents could be identified that would overcome IL7-induced BCR-ABL-TKI-resistance. A collection of 3200 mechanistically diverse bioactive agents, including approved oncology and anti-infective drugs, was screened against BCR-ABL^{WT} and BCR-ABL^{T315I} LICs in the presence of IL7 (0.85 ng/mL) at a fixed concentration (10 μM). The quality of the screen and minimum significant activity (>10% growth inhibition) was confirmed by receiver operating characteristic (ROC) analysis and other statistical metrics. 627 compounds showed statistically significant activity (≧10% growth inhibition). These hits were augmented with available structural analogs and drugs of the same pharmacologic classes to yield 706 active compounds, which are shown in FIG. 24. FIG. 11 shows the frequency of ATC Level 1 and Level 2 codes among the 706 compounds submitted to dose-response analysis. FIG. 27 provides the ATC classifications of the 706 active compounds. As shown in FIG. 26, concentration-response relationships were established for these compounds against BCR-ABL^{WT} and drug-resistant BCR-ABL^{T315I} LICs in the presence of IL7, giving 229 validated hits with reasonable potency. All validated hits were equipotent in inhibiting the growth of BCR-ABL^{WT} and BCR-ABL^{T315I} LICs. As shown in FIG. 25, this set included 15 conventional anti-leukemic drugs, some of which were observed to enhance BCR-ABL-TKI-therapy in vivo, thus appearing to validate the detection of appropriate phenotypic responses. Furthermore, cluster analysis (FIG. 3), demonstrates that this set effectively sampled the space of all existing therapeutic classes. A single compound in FIG. 3 can be represented more than once because of multiple therapeutic indications, as is the case for corticosteroids (highly active clusters in A, C, D, H, R, and S). This appears to further demonstrate that the active compounds come from drugs used to treat many indications.

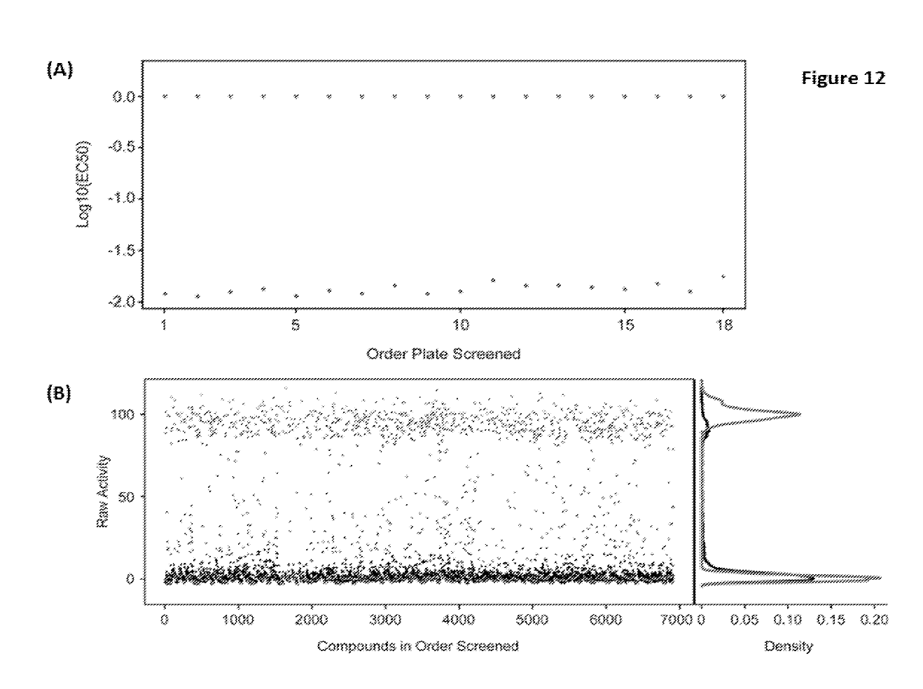

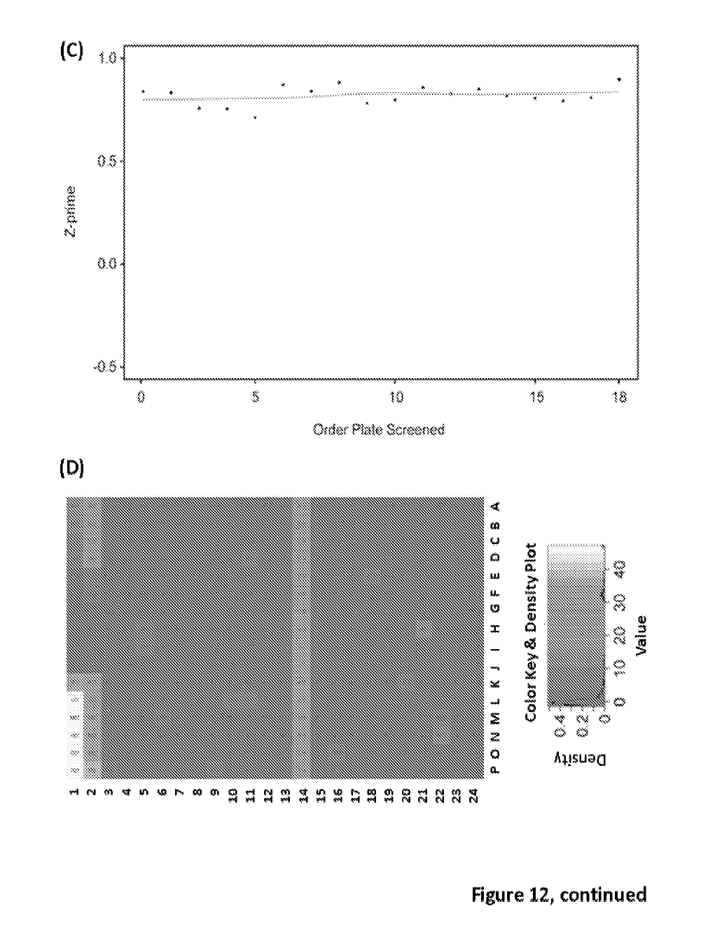

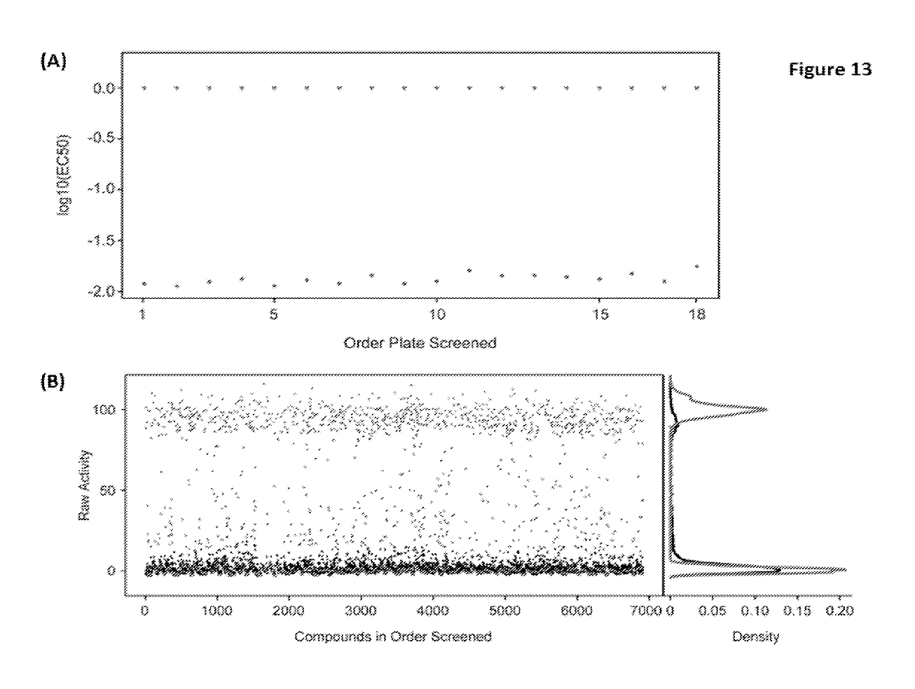

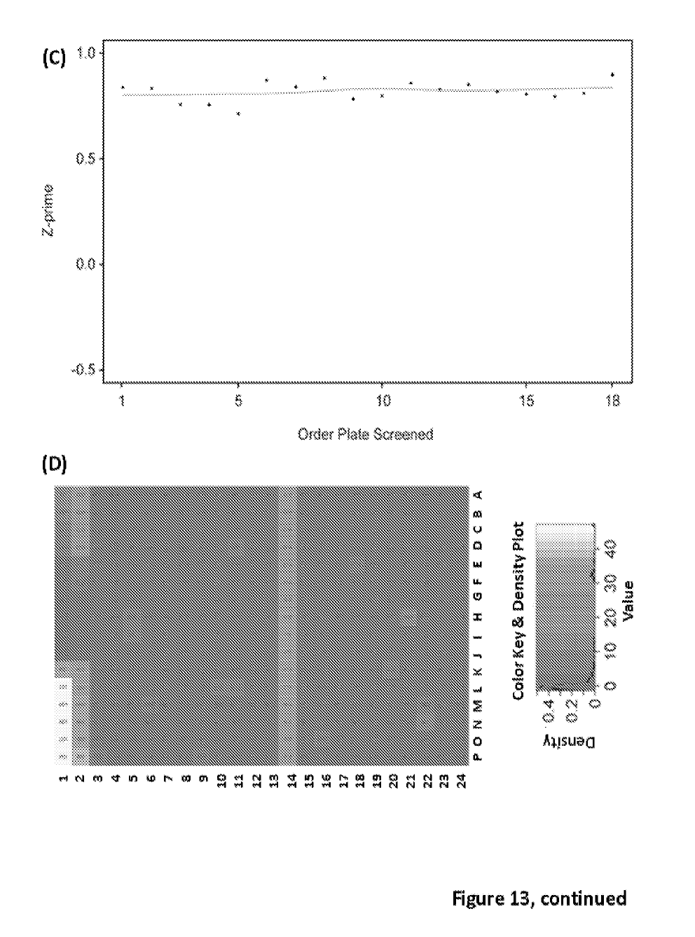

FIG. 12 shows the quality control used for the primary screen of 5600 agents against BCR-ABL^{WT} LICs performed in the presence of 0.85 ng/mL IL7. FIG. 12a shows the distribution of EC₅₀ of non-BCR-ABL-inhibiting reference compound staurosporine (calculated from wells 2A-2J in FIG. 12d). FIG. 12b shows the scatter plot of percent activity of the test compounds relative to controls. Good separation is seen between negative controls (DMSO-treated. max signal, column 13 in (FIG. 12d)) and positive controls (300 nM staurosporine, min signal, column 14 in (FIG. 12d)). FIG. 12b shows test compounds exhibited statistically significant activity relative to background noise (negative controls and background noise are depicted in grayscale; inactive test compounds are black). Densities for positive, negative, and test compounds are shown on the right. FIG. 12c shows the distribution of Z′ values for each plate in the primary screen (Z′ values throughout the run−average=0.82, lowest=0.71 and highest=0.9). FIG. 12d shows a heat map comparison of well activity averaged across all plates. Wells 1A-1J and 2A-2J show serial dilutions of dasatinib and staurosporine. In the presence of IL7, an average LIC growth inhibition of approximately 45% was achieved by 100 nM dasatinib (well 1A), the later approaching maximum concentration achieved in human plasma. FIG. 13 shows the quality control used for primary screen of 5600 agents against BCR-ABL^{T315I} LICs performed in the presence of 0.85 ng/mL IL7. FIG. 13a shows distribution of EC₅₀ of non-BCR-ABL-inhibiting reference compound staurosporine (calculated from wells 2A-2J in (FIG. 13d)). FIG. 13b shows a scatterplot of percent activity of the test compounds relative to controls. Good separation is seen between negative controls (DMSO-treated, max signal, column 13 in (FIG. 13d)) and positive controls (300 nM staurosporine, min signal, column 14 in (FIG. 13d)). FIG. 13b shows test compounds exhibited statistically significant activity relative to background noise (negative controls and background noise are depicted in grayscale; inactive test compounds are black). FIG. 13c shows distribution of Z′ values for each plate in the primary screen (Z′ values throughout the run−average=0.85, lowest=0.77, and highest=0.92). FIG. 13d shows a heat map comparison of well activity averaged across all plates. Wells 1A-1J and 2A-2J show serial dilutions of dasatinib and staurosporine tested against BCR-ABL^{T315I} LICs. Although 100 nM dasatinib (Well 1A) was totally ineffective, well 2A with the highest staurosporine concentration (300 nM) showed average LIC growth inhibition of approximately 100%.

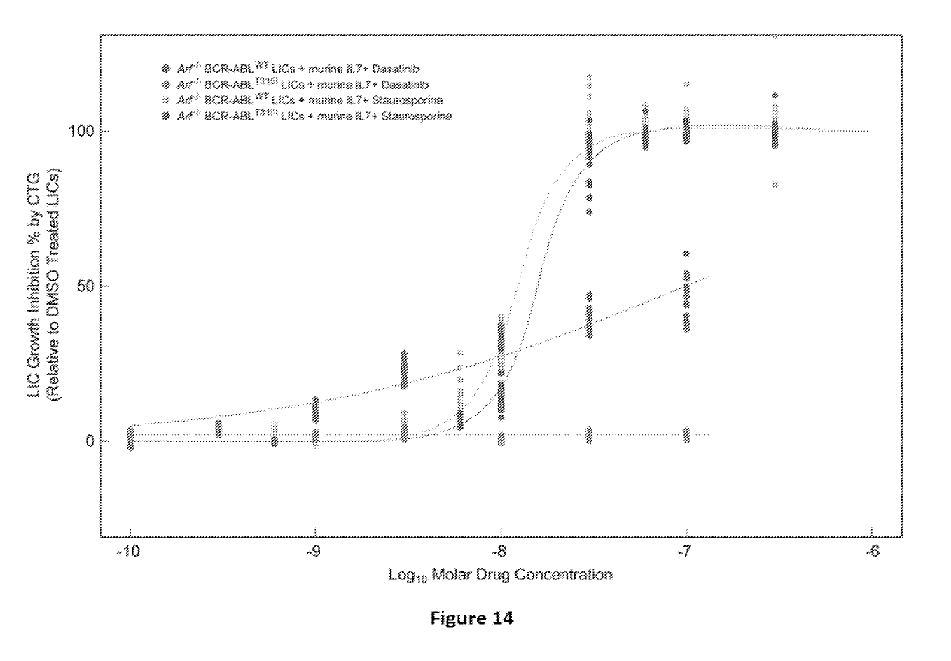

FIG. 14 shows distributions of independent dose response curves of dasatinib and staurosporine against BCR-ABL^{WT} and BCR-ABL^{T315I} LICs in the presence of 0.85 ng/mL IL7 calculated separately from all assay plates (wells 1A-1J and 2A-2J) of primary drug screening experiments. Both IL7-imparted dasatinib-resistant phenotype in BCR-ABL^{WT} LICs (EC₅₀ in nM: 95% CI=86-116, average>100) and BCR-ABL-mutation-imparted BCR-ABL-TKI-resistant phenotype in BCR-ABL^{T315I} LICs, similar to FIG. 5, were consistently captured across all plates. Low interplate variability in calculated EC₅₀ values was observed for the non-BCR-ABL-targeting reference compound staurosporine (95% CI for EC₅₀ in nM: WT=11-12, T315I=15-16) and the majority of the validated hits. This data suggests that the primary high-throughput drug screening experiments capture BCR-ABL-TKI drug-resistant phenotypes with high accuracy.

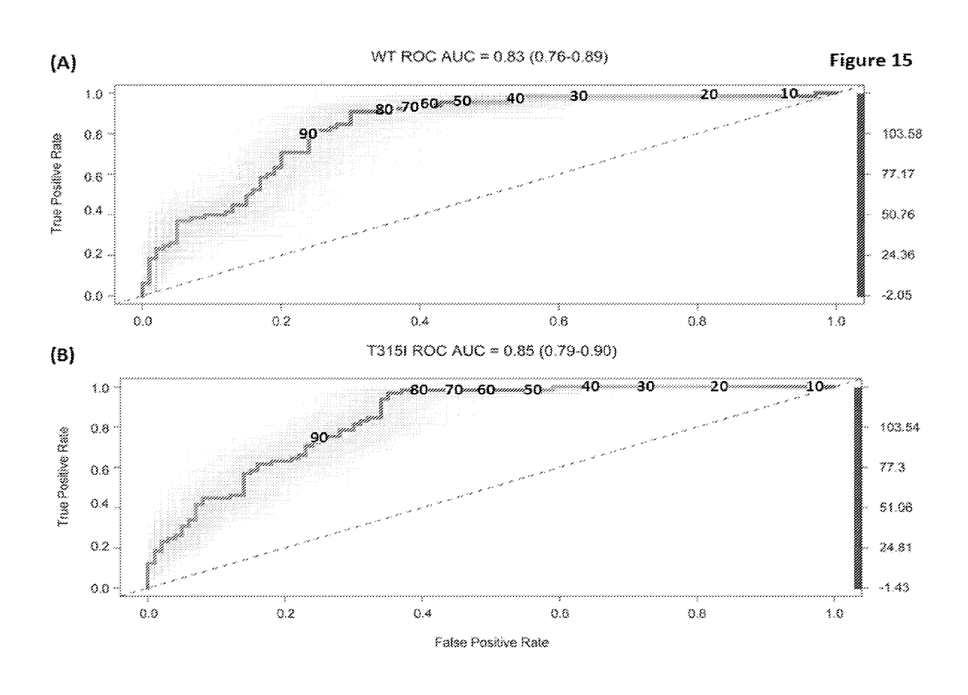

FIG. 15 shows Receiver Operating Characteristic (ROC) analysis of BCR-ABL^{WT} and BCR-ABL^{T315I} LIC-based assays. This data indicates that both (a) BCR-ABL^{WT} and (b) BCR-ABL^{T315I} LIC-based assays have similarly high discriminatory power (AUC 0.83 and 0.85, respectively). The ROC curve is plotted as a function of % activity, and is color coded according to the right Y-axis. The ROC curves in gray are calculated from 200 bootstrap simulations. The dashed line corresponds to a random assay (an ideal assay has an AUC of 1.0, whereas a random assay has an AUC of 0.5).

5. In Vitro and In Vivo Anti-PH+ All Activity of Dihydroartemisinin

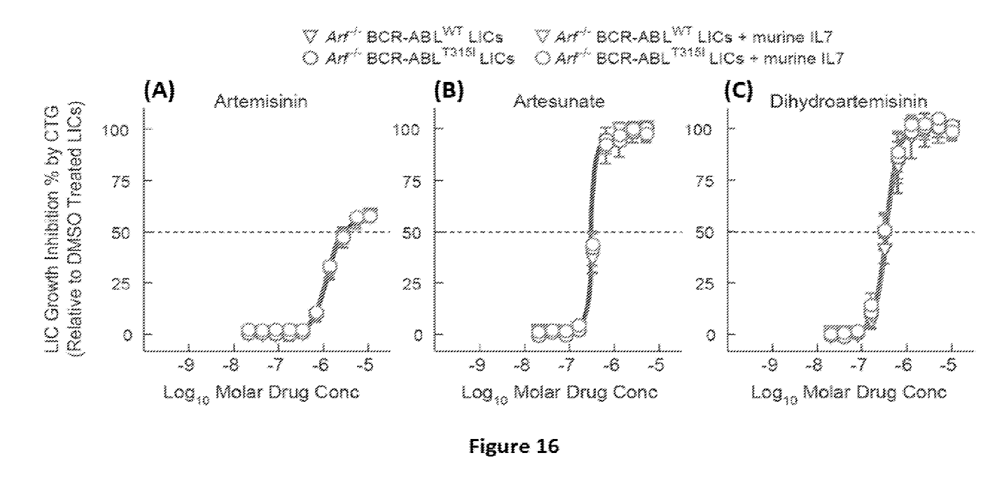

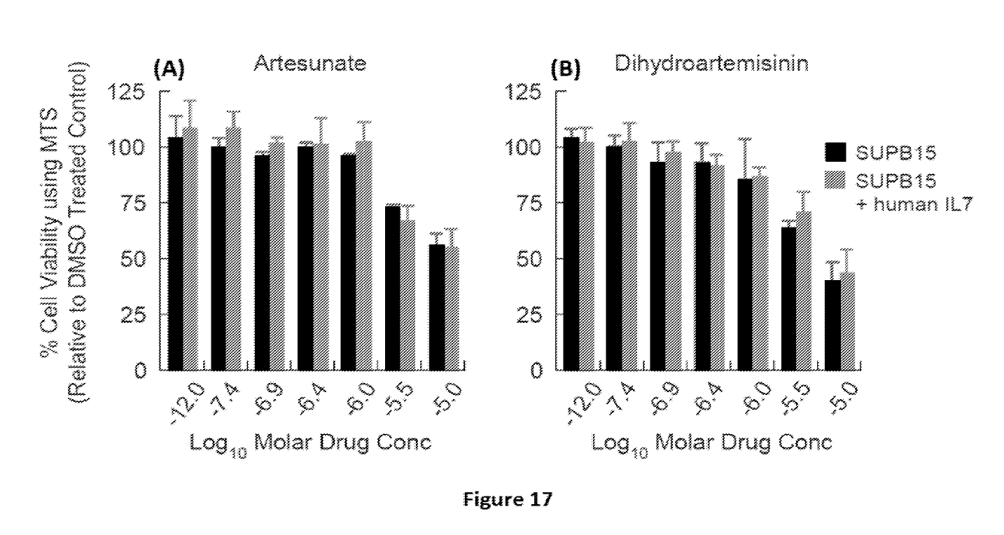

Validated hits included many compounds with varying mechanisms of action. Initial compound evaluation focused on approved drugs having favorable human pharmacokinetic and toxicity profiles that also had been previously used in pediatric populations. The drug class selected for further study was the artemisinin class of antimalarials (ARTs), which includes artemisinin, artesunate, and dihydroartemisinin (DHA). As shown in FIG. 16, Arf^{-/-} BCR-ABL^{WT} (triangles) and Arf^{-/-} BCR-ABL^{T315I} (circles) LICs were treated with artemisinin, artesunate, dihydroartemisinin, or DMSO for 72 h in the absence (dark gray) or presence (light gray) of 1 ng/mL murine IL7. For each drug concentration, LIC growth (measured by the CTG assay) was normalized to DMSO-treated LICs and fit using nonlinear regression (+/−sd). The ARTs as a class were observed to be equipotent against BCR-ABL^{WT} and BCR-ABL^{T315I} LICs, but the anti-LIC potency of artemisinin was lower than that of artesunate or DHA. As represented in FIG. 17, human Ph+ ALL SUPB15 cells were also treated with artesunate, dihydroartemisinin, or DMSO (no drug) for 72 h in the absence (dark gray) or presence (light gray) of 25 ng/mL human IL7. Viability was measured by the MTS assay. Values were normalized to DMSO-treated cells. DHA and artesunate were also observed to actively kill the human Ph+ ALL cell line SUP-B15 and showed similar anti-Ph+ ALL potency in the presence of IL7.

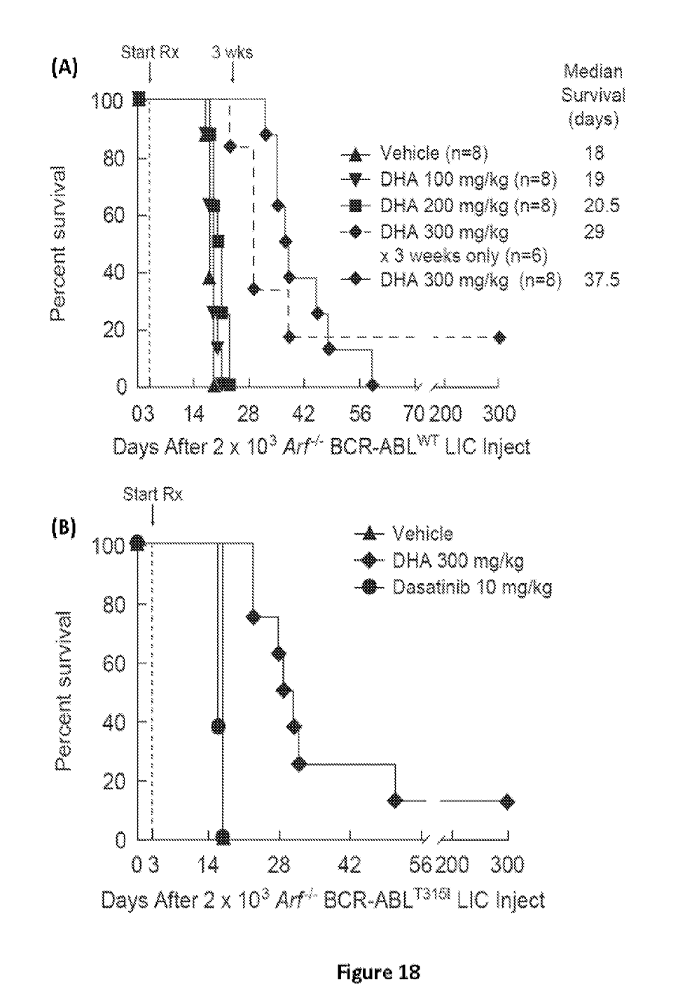

DHA was selected for further analysis because of its potency, cellular selectivity, oral formulation, favorable pharmacokinetics, known bioavailability in the hematopoietic system, and tolerability in all human age groups. FIG. 18 shows the Kaplan-Meier survival curves showing the overall survival in response to therapy (survival benefit) in IL7^{+/+} B16 host mice receiving day 0 tail vein injections of 2×10³ Arf^{-/-} BCR-ABL^{WT} Luc+ or BCR-ABL^{T315I} LICs. On day 3, the mice began once daily treatment for 5 days of each week. In mice bearing low burdens of BCR-ABL^{WT}-driven leukemia, treatment was once per day with either vehicle, 100 mg/kg DHA, 200 mg/kg DHA, 300 mg/kg DHA, or 300 mg/kg. In mice bearing low burdens of BCR-ABL^{T315I}-driven leukemia, treatment was either vehicle, 300 mg/kg DHA, or 10 mg/kg dasatinib. DHA monotherapy appears to confer a dose-dependent survival benefit to IL7^{+/+} host mice bearing low-burden BCR-ABL^{WT} leukemia (FIG. 18, panel A). BCR-ABL mutations, which are often selected with dasatinib treatment of mice bearing Arf ^{-/-} BCR-ABL^{WT} LICs in this model, were absent in leukemic samples collected from terminally moribund DHA-treated mice. DHA monotherapy was also observed to give a similar response in mice with low-burden BCR-ABL^{T315I} leukemia, which is completely refractory to dasatinib (FIG. 18, panel B). Thus, unlike BCR-ABL-TKIs, DHA appears to act against Ph+ ALL via a BCR-ABL kinase-independent mechanism. Without wishing to be bound by a particular theory, the data suggests the artemisinin family of antimalarial drugs circumvents IL7-induced resistance to dasatinib in Ph+ ALL cells in vitro and Ph+ ALL in IL7^{+/+} host mice.

6. DHA Synergistically Enhances Response to Dasatinib

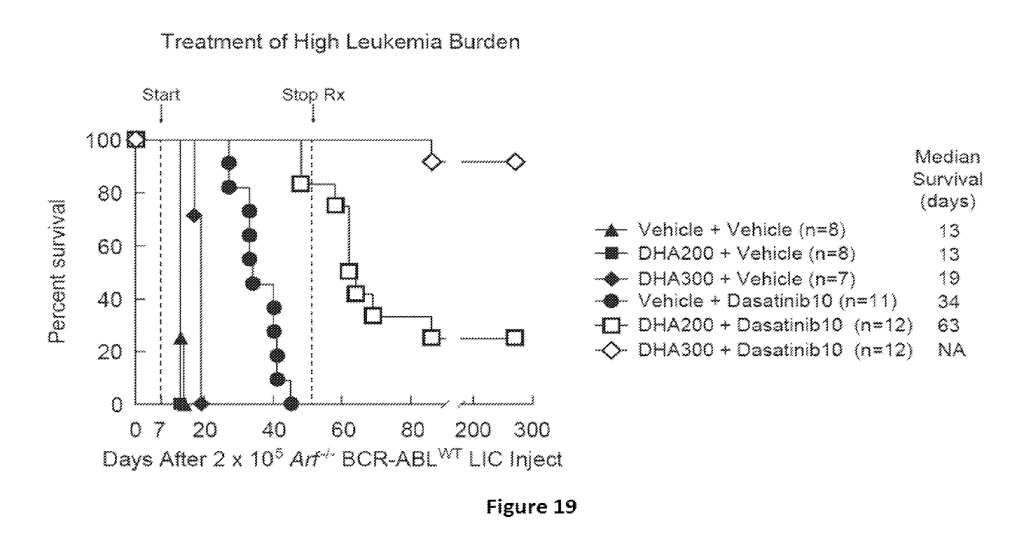

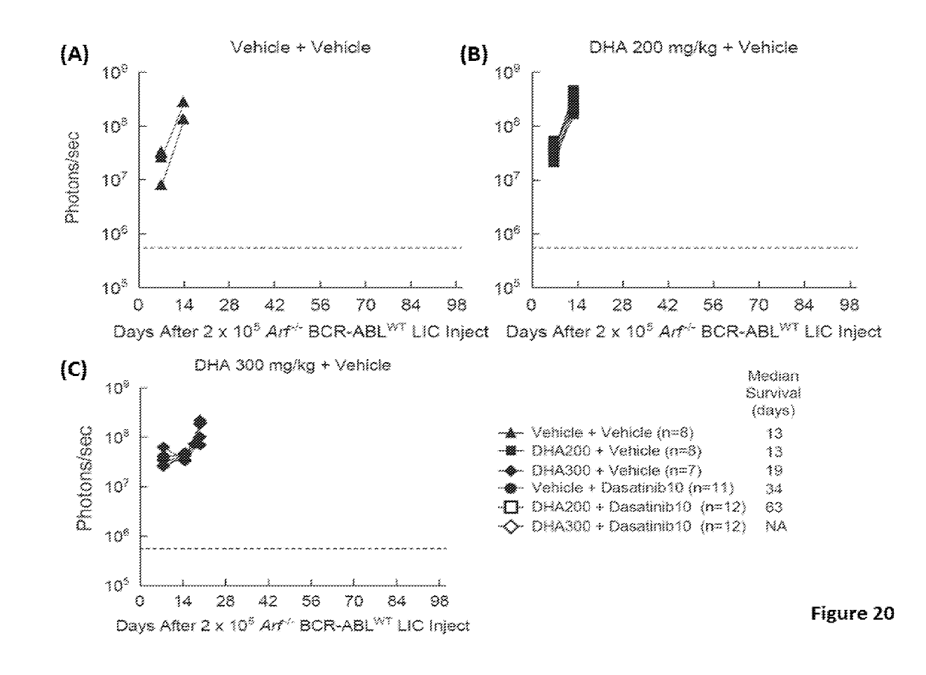

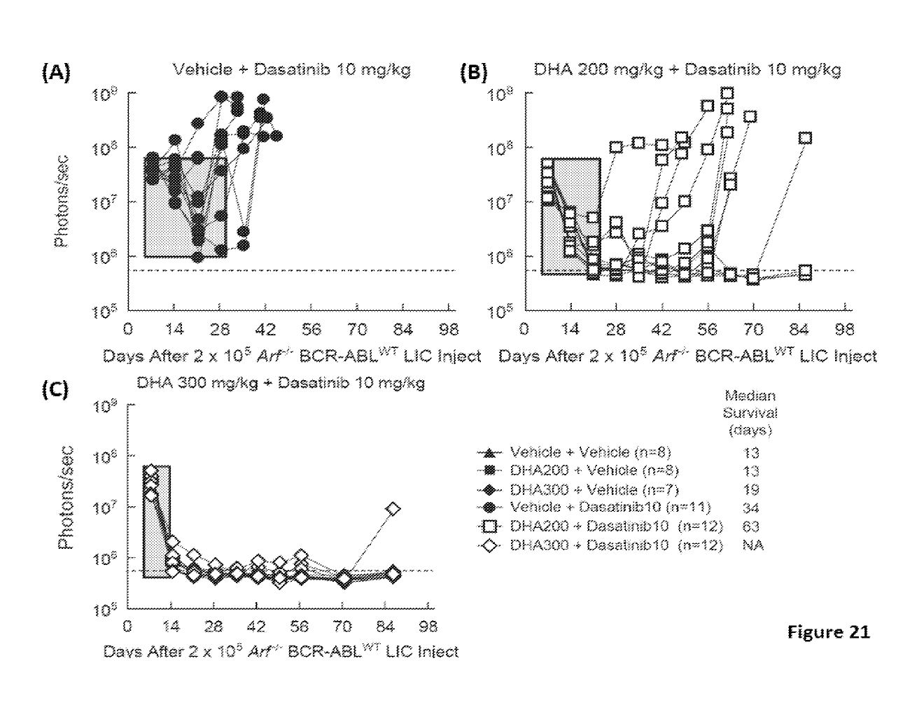

Because BCR-ABL^{WT} leukemias in IL7-deficient mice were found to be more sensitive to dasatinib and DHA displayed anti-Ph+ ALL activity in IL7^{+/+} host mice, the potential for synergy between the two drugs was evaluated. As represented in FIG. 19, cohorts of IL7^{+/+} immunocompetent mice bearing high burdens of Arf^{-/-} BCR-ABL^{WT}-driven leukemia were treated with dasatinib alone (10 mg/kg), DHA alone (200 or 300 mg/kg) or binary combinations. Continuous monotherapy treatment with dasatinib induced only weak initial responses, and all animals later relapsed with complete mortality within 6 weeks of initiating therapy. As represented in FIG. 20, DHA alone was observed to be completely ineffective (200 mg/kg) or poorly effective (300 mg/kg) in the high-burden leukemia model. However, the combination of DHA and dasatinib, represented in FIG. 21, provided a strong and rapid initial response, and led to significantly increased overall survival in comparison to the treatment with either of the two drugs alone. Of the 12 mice given 200 mg/kg DHA and 10 mg/kg dasatinib, 3 survived long-term. All mice given 300 mg/kg DHA and 10 mg/kg dasatinib (n=12) survived the treatment period. 4 weeks after completion of combination therapy, 1 mouse succumbed to an isolated CNS relapse, which may be related to the poor CNS availability of dasatinib. Remarkably, the 14 surviving mice taken off dasatinib-combined therapy ( 3/12 at 200 mg/kg and 11/12 at 300 mg/kg DHA) showed no leukemia during the one year follow-up period, the longest period monitored.

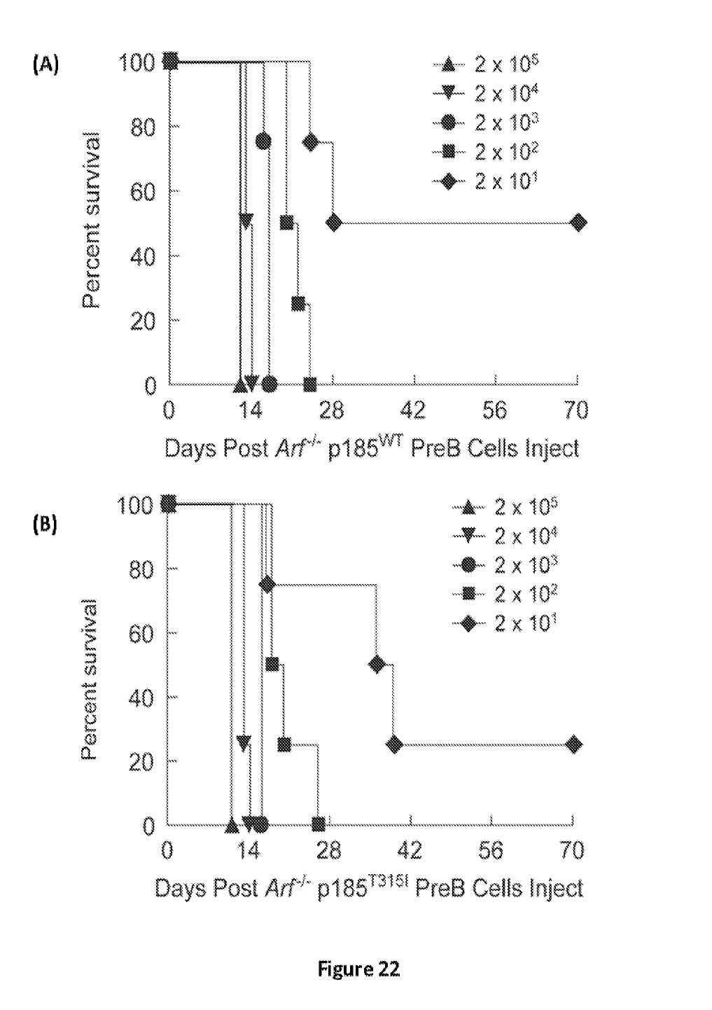

In comparison, FIG. 22 shows data obtained in the leukemia stem cell function (LSC) function assay comparing the in vivo leukemia initiating capacity of Arf^{-/-} p185^{WT} (left) and Arf^{-/-} p185^{T315I} preB (right) populations. The Kaplan-Meier curves shown represent the overall survival of nonconditioned immunocompetent C57B1/6J recipient mice that received serial log-fold dilutions (n=4 per arm) of LIC-number (as indicated in the inset legend) on day 0 in Arf^{-/-} p185^{WT} (FIG. 22, panel A) and Arf^{-/-} p185^{T3151} preB (FIG. 22. panel B) populations. The data show that all animals that remain untreated succumb to full blown leukemia within 4 weeks of injecting a very low numbers of LICs, suggesting that the drug combination cured the disease. All dasatinib-DHA regimens were well tolerated in leukemic and non-leukemic mice. Without wishing to be bound by a particular theory, the DHA-dasatinib combination appears more efficacious than the clinically-used triple combination between dasatinib, dexamethasone and asparaginase previously assessed in the same model.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A pharmaceutical composition comprising a therapeutically effective amount of a BCR-ABL tyrosine kinase inhibitor; and a therapeutically effective amount of at least one artemisinin analogue having a structure represented by a formula: 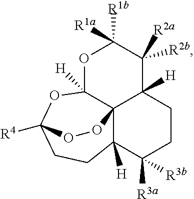
wherein R^{1a} and R^{1b} together are (C═O) or wherein each of R^{1a} and R^{1b} are independently selected from hydrogen, hydroxyl, —NH₂, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), C2-05 heterocycloalkyl, C3-C6 cycloalkyl, —O(C1-C8 alkyl)C3-C6 cycloalkyl, —O(C1-C8 alkyl)-(C═O)OH, —O(C═O)(C1-C6 alkyl)-(C═O)OH, —O(C1-C8 alkyl)-(C═O)O(C1-C6 alkyl), —O(C═O)(C1-C6 alkyl)-(C═O)O(C1-C6 alkyl), —O(C1-C8 alkyl)-aryl, and —O-aryl;
wherein each of aryl, C2-C5 heterocycloalkyl, C3-C6 cycloalkyl, and —O(C1-C8 alkyl)C3-C6 cycloalkyl, when present, is substituted with 0-3 groups selected from halogen, hydroxyl, —NH₂, —(C═O)OH, C1-C8 alkyl, C1-C8 alkoxy, C1-C8 monohaloalkyl, C1-C8 polyhaloalkyl, —NH(C1-C6 alkyl), —N(C1-C6 alkyl)(C1-C6 alkyl), —(C1-C6 alkyl)-NH(C1-C6 alkyl), and —(C1-C6 alkyl)-N(C1-C6 alkyl)(C1-C6 alkyl);
wherein R^{2a} and R^{2b} together are (C═CR^{5a}R^{5b}) or wherein each of R^{2a} and R^{2b} are independently selected from hydrogen, C1-C8 alkyl, C1-C8 monohaloalkyl, C1-C8 polyhaloalkyl, —(C1-C8 alkyl)-(C═O)OH, and —O(C═O)(C1-C6 alkyl)-(C═O)O(C1-C6 alkyl);
wherein each of R^{5a} and R^{5b} are independently selected from hydrogen, C1-C6 alkyl, C1-C6 monohaloalkyl, and C1-C6 polyhaloalkyl;
wherein each of R^{3a} and R^{3b} are independently selected from hydrogen, C1-C8 alkyl, C1-C8 monohaloalkyl, and C1-C8 polyhaloalkyl; and
wherein R⁴ is selected from hydrogen, C1-C8 alkyl, C1-C8 monohaloalkyl, C1-C8 polyhaloalkyl, —(C1-C8 alkyl)-(C═O)OH, and —O(C═O)(C1-C6 alkyl)-(C═O)O(C1-C6 alkyl);
or a pharmaceutically acceptable salt, hydrate, solvate, or polymorph thereof; and optionally a pharmaceutically acceptable carrier.

2. The composition of claim 1, wherein R^{1a} and R^{1b} together are (C═O).

3. The composition of claim 1, wherein R^{1a} is hydrogen and R^{1b} is hydroxyl.

4. The composition of claim 1, wherein R^{3b} is hydrogen and R^{3a} is methyl.

5. The composition of claim 1, wherein R⁴ is methyl.

6. The composition of claim 1, wherein the artemisinin analogue is selected from:

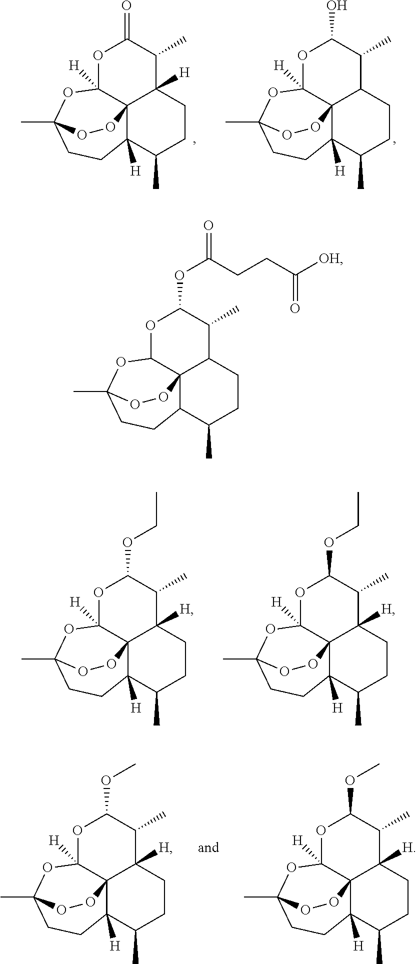

7. The composition of claim 1, wherein the artemisinin analogue is:

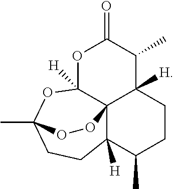

8. The composition of claim 1, wherein the artemisinin analogue is:

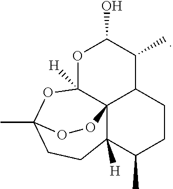

9. The composition of claim 1, wherein the artemisinin analogue is:

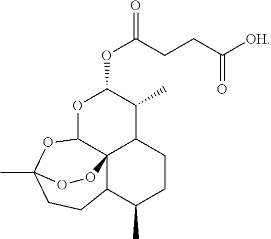 [SMILES: C[C@@H]1CCC2[C@@H](C)[C@@H](OC(=O)CCC(=O)O)OC3OC4(C)CCC1[C@]32OO4]

10. The composition of claim 1, wherein the BCR-ABL tyrosine kinase inhibitor is selected from imatinib, dasatinib, nilotinib, bosutinib, ponatinib, bafetinib, saracatinib, tozasertib and rebastinib.

11. The composition of claim 1, wherein the BCR-ABL tyrosine kinase inhibitor is imatinib.

12. The composition of claim 1, wherein the BCR-ABL tyrosine kinase inhibitor is dasatinib.

13. The composition of claim 10, wherein the artemisinin analogue is:

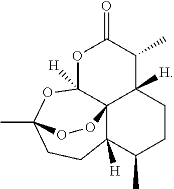

14. The composition of claim 10, wherein the artemisinin analogue is:

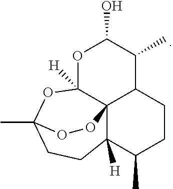

15. The composition of claim 10, wherein the artemisinin analogue is:

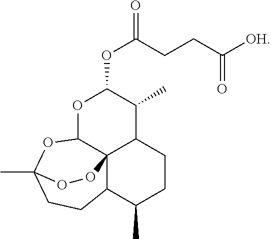 [SMILES: C[C@@H]1CCC2[C@@H](C)[C@@H](OC(=O)CCC(=O)O)OC3OC4(C)CCC1[C@]32OO4]

16. A method for the treatment of a Philadelphia chromosome positive leukemia in a patient comprising the step of administering to the patient a therapeutically effective amount of a BCR-ABL kinase inhibitor and a therapeutically effective amount of at least one disclosed artemisinin analogue, or a pharmaceutically acceptable salt, solvate, or polymorph thereof

17. The method of claim 16, wherein the BCR-ABL tyrosine kinase inhibitor and the artemisinin analogue are co-administered.

18. The method of claim 16, wherein the BCR-ABL tyrosine kinase inhibitor is administered prior to administration of the artemisinin analogue.

19. The method of claim 16, wherein the artemisinin analogue is administered prior to administration of the BCR-ABL tyrosine kinase inhibitor.

20. The method of claim 16, wherein the BCR-ABL tyrosine kinase inhibitor is administered in a first period; and the BCR-ABL tyrosine kinase inhibitor and the artemisinin analogue are co-administered in a second period.

21. The method of claim 20, wherein the Philadelphia chromosome positive leukemia during the first treatment period becomes refractory to treatment by the BCR-ABL tyrosine kinase inhibitor.

22. The method of claim 20, wherein the Philadelphia chromosome leukemia contains a mutation in the kinase domain in the BCR-ABL gene.

23. The method of claim 20, wherein the Philadelphia chromosome positive leukemia after the first treatment period comprises a mutation selected from H201L, Y232S, M237V, I242T, M244V, L248V, de1248-274, G250E, G250V, Q252H, Y253F, Y253H, E255K, E255V, E258D, L273M, D276G, E279K, E281X, V289I, E292V, L298V, V299L, F311I, F311L, T315I, F317L, Y342H, M351T, E355G, F359C, F359I, F359V, D363Y, L364I, A365V, A366G, V379I, L384M, L387M, M388L, Y393C, H396P, H396R, A397P, S417Y, 1418S, I418V, S438C, P441L, E450A, E450G, E450K, E450V, E453K, E453V, E459G, E459K, M472I, P480L, F486S, D504D, G514S, T240T, K247R, F311V, and E499E.

24. The method of claim 23, wherein the mutation is T315I.

25. The method of claim 16, wherein the BCR-ABL tyrosine kinase inhibitor is selected from imatinib, dasatinib, nilotinib, bosutinib, ponatinib, bafetinib, saracatinib, tozasertib and rebastinib.

26. The method of claim 25, wherein the BCR-ABL tyrosine kinase inhibitor is imatinib.

27. The method of claim 25, wherein the BCR-ABL tyrosine kinase inhibitor is dasatinib.

28. The method of claim 16, wherein the patient has been diagnosed with a need for treatment of a Philadelphia chromosome positive leukemia prior to the administering step.

29. The method of claim 16, further comprising the step of identifying a patient in need of treatment of a Philadelphia chromosome positive leukemia.

30. The method of claim 16, wherein the patient has been diagnosed with a Philadelphia chromosome positive leukemia refractory to treatment with a BCR-ABL kinase inhibitor.

31. A kit comprising a BCR-ABL kinase inhibitor, at least one disclosed artemisinin analogue, or a pharmaceutically acceptable salt, solvate, or polymorph thereof; and instructions for treatment of a disease of uncontrolled cellular proliferation; and wherein the instructions comprise administration of a BCR-ABL kinase inhibitor and the at least one artemisinin analogue to a patient that has become refractory to treatment with a BCR-ABL kinase inhibitor.

32. The kit of claim 31, wherein the BCR-ABL kinase inhibitor and the at least one disclosed artemisinin analogue are co-formulated.

33. The kit of claim 31, wherein the BCR-ABL kinase inhibitor and the at least one disclosed artemisinin analogue are co-packaged.
